(19) United States
(12) Patent Application Publication
(10) Pub. No.: US 2011/0269741 A1
(43) Pub. Date: Nov. 3, 2011

(54) NOVEL COMPOSITIONS AND METHODS OF USE

(75) Inventors: Huanming CHEN, Irvine, CA (US); Jianlin SONG, Cerritos, CA (US); Jean-Michel VERNIER, San Diego, CA (US); Anthony B. PINKERTON, San Diego, CA (US); Johnny Y. NAGASAWA, San Diego, CA (US)

(73) Assignee: Ardea Biosciences, Inc., San Diego, CA (US)

(21) Appl. No.: 13/073,964

(22) Filed: Mar. 28, 2011

Related U.S. Application Data
(63) Continuation of application No. 12/812,022, filed on Mar. 28, 2011.
(60) Provisional application No. 61/019,584, filed on Jan. 7, 2008.

(51) Int. Cl. A61K 31/4375; A61K 31/5377; C07D 215/56; A61P 31/18; A61K 31/4709; A61K 31/4725; C07D 401/10; C07D 413/10; C07D 471/04; A61K 31/47

(52) U.S. Cl. 514/210.21; 546/123; 514/300; 544/127; 514/234.5; 544/80; 514/232.5; 546/156; 514/312; 544/128; 514/235.2

(57) ABSTRACT

Described herein are novel enzyme inhibitors. In some embodiments the enzyme inhibitors are integrase inhibitors, particularly HIV integrase inhibitors. Also described herein are compositions containing them and methods of using them. Thus, the compounds and compositions described herein are useful for the in vitro and in vivo inhibition of HIV integrase as a method of treating or preventing HIV, AIDS or related disorders.

### CROSS-REFERENCE

This application claims the benefit of U.S. Provisional Application No. 61/019,584, filed Jan. 7, 2008, which application is incorporated herein by reference in its entirety.

### BACKGROUND OF THE INVENTION

Human immunodeficiency virus (HIV), particularly the HIV type-1 (HIV-1) and type-2 (HIV-2) strains of the virus, is the causative agent of acquired immunodeficiency syndrome (AIDS). HIV infected individuals are initially asymptomatic but then develop AIDS related complex (ARC, characterized by symptoms such as persistent generalized lymphadenopathy, fever and weight loss) and eventually progress to AIDS.

### SUMMARY OF THE INVENTION

Disclosed herein are compounds and their metabolites, pharmaceutically acceptable salts, prodrugs, solvates, polymorphs, tautomers and isomers. A compound of formula (I) or (II); formula (III) or (IV); or formula (V)(a), (V)(b) or (V)(c) are used to inhibit integrases. A compound of formula (I) or (II); formula (III) or (IV); or formula (V)(a), (V)(b) or (V)(c) are used to inhibit HIV integrases. Disclosed herein are also compositions comprising the compounds and their pharmaceutically acceptable salts, prodrugs, solvates, polymorphs, tautomers and isomers. Further disclosed herein are methods for inhibiting integrases. In some embodiments, the methods described herein are used for inhibiting HIV integrases. Additionally disclosed herein are methods useful in the treatment of diseases. The compounds and, compositions described herein are useful in the treatment of diseases. A compound of formula (I) or (II); formula (III) or (IV); or formula (V)(a), (V)(b) or (V)(c) are useful in the treatment of diseases such as viral infection, particularly infection with HIV.

Compounds of formula (I) and (II) and the metabolites, pharmaceutically acceptable salts, pharmaceutically active metabolites, pharmaceutically acceptable prodrugs, and pharmaceutically acceptable solvates thereof, modulate the activity of integrase enzymes; and, as such, are useful for treating diseases or conditions in which infection with a virus comprising an integrase enzyme contributes to the pathology and/or symptoms of a disease or condition.

Compounds of formula (III) and (IV) and the metabolites, pharmaceutically acceptable salts, pharmaceutically active metabolites, pharmaceutically acceptable prodrugs, and pharmaceutically acceptable solvates thereof, modulate the activity of integrase enzymes; and, as such, are useful for treating diseases or conditions in which infection with a virus comprising an integrase enzyme contributes to the pathology and/or symptoms of a disease or condition.

Compounds of formula (V)(a), (V)(b) and (V)(c) and the metabolites, pharmaceutically acceptable salts, pharmaceutically active metabolites, pharmaceutically acceptable prodrugs, and pharmaceutically acceptable solvates thereof, modulate the activity of integrase enzymes; and, as such, are useful for treating diseases or conditions in which infection with a virus comprising an integrase enzyme contributes to the pathology and/or symptoms of a disease or condition.

Disclosed herein, in certain embodiments, is a compound of formula (I) or formula (II) or a metabolite, pharmaceutically acceptable salt, solvate, polymorph, ester, tautomer or prodrug thereof:

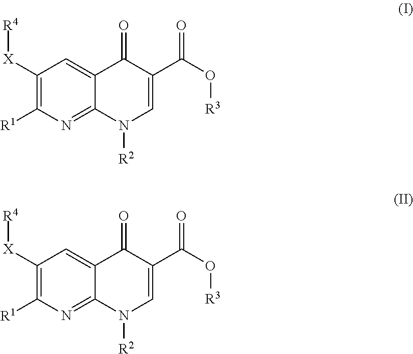

wherein: R¹ is H, F, Cl, Br, I, CFH₂, CF₂H, CF₃, CN, OH, NO₂, NH2, NH(alkyl) or N(alkyl)₂, SO₂CH₃, SO₂NH2, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl or optionally substituted heteroaryl;
R² is optionally substituted alkyl, optionally substituted cycloalkyl, optionally substituted heterocycloalkyl, optionally substituted aryl or optionally substituted heteroaryl; R³ is H, C1-6 alkyl or a pharmaceutically acceptable cation; and wherein X is O or N—R⁵; wherein R⁵ is H or optionally substituted C₁₋₄ alkyl;

R⁴ is

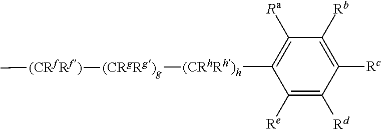

wherein each R^{f}, R^{f′}, R^{g}, R^{g′}, R^{h} and R^{h′} is H or optionally substituted C₁₋₁₀ alkyl; g is 0 or 1; h is 0 or 1; R^{a}, R^{b}, R^{c}, R^{d} and R^{e} are independently selected from H, F, Cl, Br, I, CF₃, CN, alkyl, cycloalkyl, cyclopropylmethyl, NH2, NHR′, NR′R″, OH, OR′, SH, SR′, C(O)R′, CO2H, COOR′, CONH₂, CONHR′, CONR′R″, SO₃H, S(O)₂R′, S(O)₂NH2, S(O)₂NHR′, S(O)₂NR′R″, aryl, heterocyclyl and heteroaryl; wherein R′ is methyl, ethyl, n-propyl, i-propyl, n-butyl, i-butyl, s-butyl, t-butyl, cyclopropyl, cyclobutyl, cyclopentyl, cyclohexyl or cyclopropylmethyl; R″ is methyl, ethyl, n-propyl, i-propyl, n-butyl, i-butyl, s-butyl, t-butyl, cyclopropyl, cyclobutyl, cyclopentyl, cyclohexyl or cyclopropylmethyl; or R′ and R″ together with the nitrogen atom to which they are attached form an optionally substituted 4-, 5- or 6-membered heterocyclic ring; or X is N and R⁵ and R^{f}, or R⁵ and R^{g}, or R⁵ and R^{h}, together with the N atom form an optionally substituted 4-, 5- or 6-membered heterocyclic ring, optionally containing 1 or 2 additional heteroatoms selected from O, N and S; and all alkyl, alkylene, cycloalkyl, heterocyclyl, aryl and heteroaryl moieties may be optionally further substituted. In some embodiments, R¹ is H, optionally substituted alkyl, optionally substituted alkoxy or optionally substituted heterocycle. In some embodiments, R¹ is alkoxy. In some embodiments, R¹ is methoxy. In some embodiments, R² is optionally substituted C₁₋₁₀ alkyl. In some embodiments, R² is substituted C₅ or C₆ alkyl. In some embodiments, C₅ or C₆ alkyl is substituted; with one OH group. In some embodiments, R² is 1-hydroxy-3,3-dimethylbutan-2-yl or 1-hydroxy-3-methylbutan-2-yl:

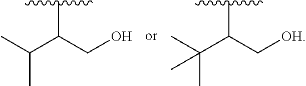

In some embodiments, R² comprises a chiral center. In some embodiments, the chiral center is in the (S) configuration. In some embodiments, R³ is H. In some embodiments, R¹ is alkoxy; R² is C₅ or C₆ alkyl substituted with one OH group; and R³ is H. In some embodiments, X is NH. In some embodiments, R⁴ is

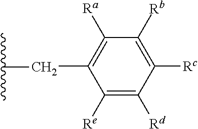

In some embodiments, X is NH and R⁴ is

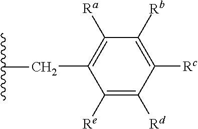

in some embodiments, R^{a}, R^{b}, R^{c}, R^{d} and R^{e} are independently selected from H, F and Cl.

Disclosed herein, in certain embodiments, is a compound selected from: (S)-1-(1-hydroxy-3-methylbutan-2-yl)-7-methoxy-4-oxo-6-(2,4,6-trifluorobenzylamino)-1,4-dihydro-1,8-naphthyridine-3-carboxylic acid; (S)-1-(1-hydroxy-3-methylbutan-2-yl)-7-methoxy-4-oxo-6-(2,4,6-trifluorobenzylamino)-1,4-dihydroquinoline-3-carboxylic acid; (S)-1-(1-hydroxy-3,3-dimethylbutan-2-yl)-7-methoxy-4-oxo-6-(2,4,6-trifluorobenzylamino)-1,4-dihydro-1,8-naphthyridine-3-carboxylic acid; (S)-6-(2,4-difluorobenzylamino)-1-(1-hydroxy-3-methylbutan-2-yl)-7-methoxy-4-oxo-1,4-dihydro-1,8-naphthyridine-3-carboxylic acid; (S)-1-(1-hydroxy-3-methylbutan-2-yl)-4-oxo-6-(2,4,6-trifluorobenzylamino)-1,4-dihydroquinoline-3-carboxylic acid; (R)-1-(1-hydroxy-3-methylbutan-2-yl)-7-morpholino-4-oxo-6-(2,4,6-trifluorobenzyloxy)-1,4-dihydro-1,8-naphthyridine-3-carboxylic acid; (S)-6-(2,6-difluorobenzylamino)-1-(1-hydroxy-3-methylbutan-2-yl)-7-methoxy-4-oxo-1,4-dihydroquinoline-3-carboxylic acid; (S)-6-(2,6-difluorobenzylamino)-1-(1-hydroxy-3-methylbutan-2-yl)-7-methoxy-4-oxo-1,4-dihydro-1,8-naphthyridine-3-carboxylic acid; (S)-6-(2,4-difluorobenzylamino)-1-(1-hydroxy-3-methylbutan-2-yl)-7-methoxy-4-oxo-1,4-dihydroquinoline-3-carboxylic acid; and (S)-1-(1-hydroxy-3,3-dimethylbutan-2-yl)-7-methyl-4-oxo-6-(2,4,6-trifluorobenzylamino)-1,4-dihydro-1,8-naphthyridine-3-carboxylic acid:

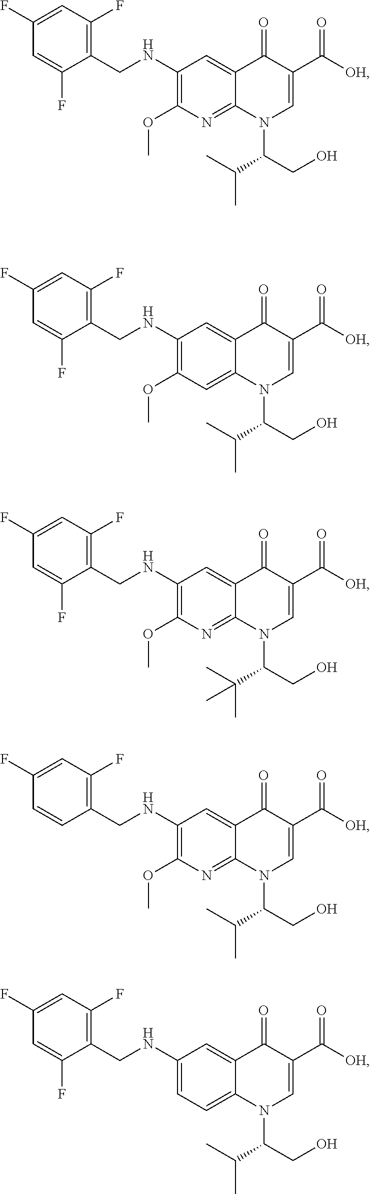

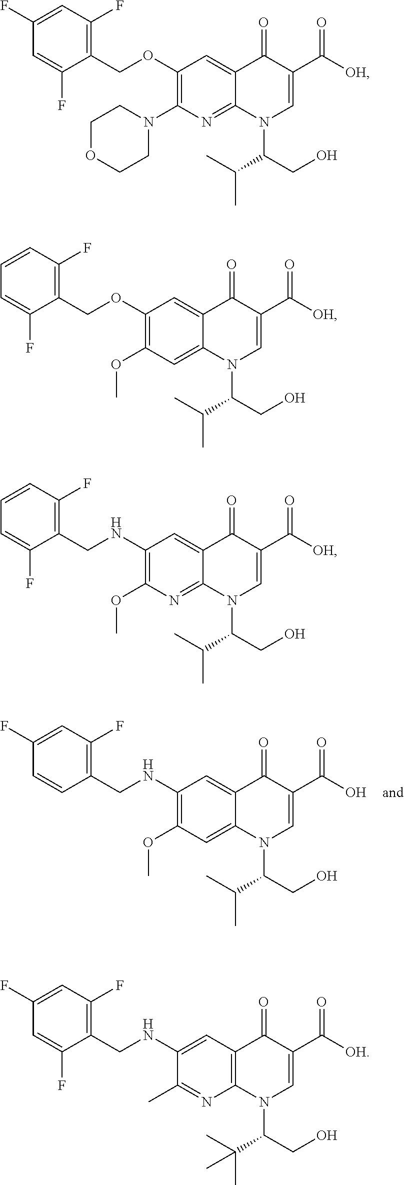

Disclosed herein, in certain embodiments, is a pharmaceutical composition comprising an effective amount a compound of formula (I) or formula (II), or a metabolite, pharmaceutically acceptable salt, solvate, polymorph, ester, tautomer or prodrug thereof. In some embodiments, the composition does not comprise a CYP3A4 inhibitor. In some embodiments, the composition further comprises a second therapeutic agent. In some embodiments, the composition further comprises a reverse transcriptase inhibitor, a viral protease inhibitor, a fusion inhibitor, a cytokine, a cytokine inhibitor, a glycosylation inhibitor, a viral mRNA processing inhibitor, an entry inhibitor, an integrase inhibitor or a maturation inhibitor or a combination thereof. In some embodiments, the composition further comprises adefovir, abacavir, amprenavir, atazanavir, apricitabine, bevirimat, darunavir, delavirdine, didanosine, efavirenz, emtricitabine, elvitegravir, enfuvirtide, etravirine, fosamprenavir, fuseon, indinavir, lamivudine, lopinavir, maraviroc, nelfinavir, nevirapine, racivir, raltegravir, reverset, ritonavir, saquinavir, stavudine, tenofovir, tipranavir, vicriviroc, zalcitabine, zidovudine, interferon-α, interferon-β or interferon-γ, or a combination of two or more thereof.

Disclosed herein, in certain embodiments, is a method of treating a viral infection in a patient in need thereof comprising administering to said patient an effective amount of a compound of formula (I) or formula (II), or a metabolite, pharmaceutically acceptable salt, solvate, polymorph, ester, tautomer or prodrug thereof. In some embodiments, the viral infection is caused by a virus selected from the group consisting of human immunodeficiency viruses 1 (HIV-1), human immunodeficiency viruses 2 (HIV-2), human T-cell leukemia viruses 1 (HTLV-1), human T-cell leukemia viruses 2 (HTLV-2), respiratory syncytial virus (RSV), human papilloma virus (HPV), adenovirus, hepatitis B virus (HBV), hepatitis C virus (HCV), Epstein-Barr virus (EBV), varicella zoster virus (VZV), cytomegalovirus (CMV), herpes simplex viruses 1 (HSV-1), herpes simplex viruses 2 (HSV-2), human herpes virus 8 (HHV-8) Yellow Fever virus, Dengue virus, Japanese Encephalitis and West Nile virus.

Disclosed herein, in certain embodiments, is a method of treating or preventing HIV infection, treating AIDS-related complex (ARC), prophylaxis of ARC, delaying the onset of ARC, treating AIDS, prophylaxis of AIDS or delaying the onset of AIDS in a subject in need thereof, comprising administering to the subject a therapeutically effective amount of a compound of formula (I) or formula (II), or a metabolite, pharmaceutically acceptable salt, solvate, polymorph, ester, tautomer or prodrug thereof. In some embodiments, the method does not comprise administration of a CYP3A4 inhibitor. In some embodiments, the subject is infected with HIV. In some embodiments, the subject is infected with HIV-1 or HIV-2. In some embodiments, the subject is infected with a drug resistant strain of HIV. In some embodiments, the subject is infected with a multidrug resistant strain of HIV. In some embodiments, the subject is infected with a strain of HIV that exhibits reduced susceptibility to reverse transcriptase inhibitors. In some embodiments, the subject is infected with a strain of HIV that exhibits at least one mutation compared to wild type HIV. In some embodiments, the mutation conveys resistance to an AIDS or HIV therapeutic. In some embodiments, the method further comprises administering an effective amount of a second therapeutic agent. In some embodiments, the method further comprises administering an effective amount of an anti HIV or AIDS drug. In some embodiments, the method further comprises administering an effective amount of a reverse transcriptase inhibitor, a viral protease inhibitor, a fusion inhibitor, a cytokine, a cytokine inhibitor, a glycosylation inhibitor, a viral mRNA processing inhibitor, an entry inhibitor, an integrase inhibitor or a maturation inhibitor or a combination thereof. In some embodiments, the method further comprises administering an effective amount of adefovir, abacavir, amprenavir, atazanavir, apricitabine, bevirimat, darunavir, delavirdine, didanosine, efavirenz, emtricitabine, elvitegravir, enfuvirtide, etravirine, fosamprenavir, fuseon, indinavir, lamivudine, lopinavir, maraviroc, nelfinavir, nevirapine, racivir, raltegravir, reverset, ritonavir, saquinavir, stavudine, tenofovir, tipranavir, vicriviroc, zalcitabine, zidovudine, interferon-α, interferon-β or interferon-γ, or a combination of two or more thereof. In some embodiments, the administration of a compound of formula (I) or formula (II), or a metabolite, pharmaceutically acceptable salt, solvate, polymorph, ester, tautomer or prodrug thereof and the second therapeutic agent is sequential. In some embodiments, the sequential administration is a cycling therapy. In some embodiments, the compound of formula (I) or formula (II), is administered before the second therapeutic agent. In some embodiments, the compound of formula (I) or formula (II), is administered after the second therapeutic agent. In some embodiments, the administration of a compound of formula (I) or formula (II), or a metabolite, pharmaceutically acceptable salt, solvate, polymorph, ester, tautomer or prodrug thereof and the second therapeutic agent is simultaneous.

Disclosed herein, in certain embodiments, is a method for treating HIV infection in a subject in need thereof with combination therapy, comprising administering to said patient an effective amount of a combination of at least one compound of formula (I) or formula (II) with a second therapeutic agent selected from the group consisting of reverse transcriptase inhibitors, viral protease inhibitors, cytokines, cytokine inhibitors, glycosylation inhibitors, viral mRNA processing inhibitors, entry inhibitors, integrase inhibitors, maturation inhibitors or a combination of two or more thereof.

Disclosed herein, in certain embodiments, is a method for treating HIV infection in a subject in need thereof with combination therapy, comprising administering to said patient an effective amount of a combination of at least one compound of formula (I) or formula (II), or a metabolite, pharmaceutically acceptable salt, solvate, polymorph, ester, tautomer or prodrug thereof; with a second therapeutic agent selected from the group consisting of adefovir, abacavir, amprenavir, atazanavir, apricitabine, bevirimat, darunavir, delavirdine, didanosine, efavirenz, emtricitabine, elvitegravir, enfuvirtide, etravirine, fosamprenavir, fuseon, indinavir, lamivudine, lopinavir, maraviroc, nelfinavir, nevirapine, racivir, raltegravir, reverset, ritonavir, saquinavir, stavudine, tenofovir, tipranavir, vicriviroc, zalcitabine, zidovudine, interferon-α, interferon-β or interferon-γ, or a combination of two or more thereof.

Disclosed herein, in certain embodiments, is a kit comprising a compound of formula (I) or formula (II), or a metabolite, pharmaceutically acceptable salt, solvate, polymorph, ester, tautomer or prodrug thereof. In some embodiments, the kit further comprises instructions for administration of the compound to a mammal to treat HIV infection, ARC or AIDS.

Disclosed herein, in certain embodiments, is a compound of formula (III) or formula (IV) or a metabolite, pharmaceutically acceptable salt, solvate, polymorph, ester, tautomer or prodrug thereof:

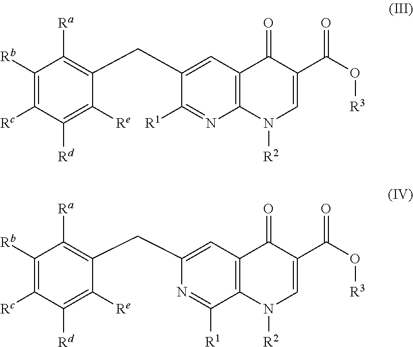

wherein: R¹ is H, F, Cl, Br, I, CFH₂, CF₂H, CF₃, CN, OH, NO₂, NH2, NH (optionally substituted alkyl) or N (optionally substituted alkyl)(optionally substituted alkyl), SO₂CH₃, SO2 NH2, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl, optionally substituted heteroaryl; R² is optionally substituted alkyl, optionally substituted cycloalkyl, optionally substituted heterocycloalkyl, optionally substituted aryl or optionally substituted heteroaryl; R³ is H, C1-6 alkyl or a pharmaceutically acceptable cation; and wherein R^{a}, R^{b}, R^{c}, R^{d} and R^{e} are independently selected from H, F, Cl, Br, I, CF₃, CN, alkyl; cycloalkyl, cyclopropylmethyl, NH2, NHR′, NR′R″, OH, OR′, SH, SR′, C(O)R′, CO₂H, COOR′, CONH2, CONHR′, CONR′R″, SO₃H, S(O)₂R′, S(O)₂NH2, S(O)₂NHR′, S(O)₂NR′R″, aryl, heterocyclyl and heteroaryl; wherein R′ is methyl, ethyl, n-propyl, i-propyl, n-butyl, i-butyl, s-butyl, t-butyl, cyclopropyl, cyclobutyl, cyclopentyl, cyclohexyl or cyclopropylmethyl; R″ is methyl, ethyl, n-propyl, propyl, n-butyl, i-butyl, s-butyl, t-butyl, cyclopropyl, cyclobutyl, cyclopentyl, cyclohexyl or cyclopropylmethyl; or R′ and R″ together with the nitrogen atom to which they are attached form an optionally substituted 4-, 5- or 6-membered heterocyclic ring; and all alkyl, alkylene, cycloalkyl, heterocyclyl, aryl and heteroaryl moieties may be optionally further substituted; and provided that the compound is not:

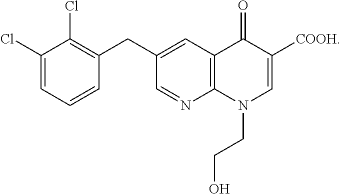

In some embodiments, R¹ is alkyl, substituted alkyl, alkoxy, substituted alkoxy, NH2, NH (optionally substituted alkyl), N (optionally substituted alkyl)(optionally substituted alkyl), heterocycle or substituted heterocycle. In some embodiments, R¹ is heterocyclyl, substituted alkyl, substituted alkoxy or NH (substituted alkyl), wherein the substituents are selected from hydroxy, hydroxyalkyl, alkoxyalkyl, aryl, aralkyl, heterocyclyl and alkylene-heterocyclyl. In some embodiments, R¹ is —CH₂—R^{1a}, —O—R^{1a} or —NH—R^{1a} wherein R^{1a} is methyl, ethyl, hydroxyethylene, hydroxypropylene, methoxyethylene, methoxypropylene, arylmethyl, heteroarylmethylene, heterocyclomethylene, heterocycloethylene or heterocyclopropylene. In some embodiments, R¹ is methoxy. In some embodiments, R² is optionally substituted C1-10 alkyl. In some embodiments, R² is optionally substituted C₅₋₈ alkyl. In some embodiments, the C₅₋₈ alkyl is substituted with one OH group. In some embodiments, R² is 1-hydroxy-3,3-dimethylbutan-2-yl or 1-hydroxy-3-methylbutan-2-yl:

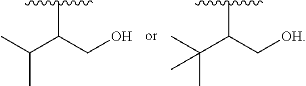

In some embodiments, R² comprises a chiral center. In some embodiments, the chiral center is in the (S) configuration. In some embodiments, R³ is H. In some embodiments, R¹ is heterocyclyl, substituted alkyl, substituted alkoxy or NH (substituted alkyl); R² is C₅₋₈ alkyl substituted with one OH group; and R³ is H. In some embodiments, R^{a}, R^{b}, R^{c}, R^{d} and R^{e} are independently selected from H, F and Cl. In some embodiments, one of R^{a}, R^{b}, R^{c}, R^{d} and R^{e} is F; one of R^{a}, R^{b}, R^{c}, R^{d} and R^{e} is Cl; and the rest of R^{a}, R^{b}, R^{c}, R^{d} and R^{e} are H. In some embodiments, R^{a} is F; R^{b} is Cl; and R^{c}, R^{d} and R^{e} are H.

Disclosed herein, in certain embodiments, is a compound selected from (S)-6-(3-chloro-2-fluorobenzyl)-1-(1-hydroxy-3-methylbutan-2-yl)-7-methoxy-4-oxo-1,4-dihydro-1,8-naphthyridine-3-carboxylic acid; 6-(3-chloro-2-fluorobenzyl)-1-((2S,3S)-1-hydroxy-3-methylpentan-2-yl)-7-methoxy-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-methoxy-4-oxo-1,4-dihydro-1,8-naphthyridine-3-carboxylic acid; (S)-6-(3-chloro-2-fluorobenzyl)-1-(1-hydroxy-4-methylpentan-2-yl)-7-methoxy-4-oxo-1,4-dihydro-1,8-naphthyridine-3-carboxylic acid; (S)-6-(3-chloro-2-fluorobenzyl)-1-(1-cyclohexyl-2-hydroxyethyl)-7-methoxy-4-oxo-1,4-dihydro-1,8-naphthyridine-3-carboxylic acid; (S)-6-(3-chloro-2-fluorobenzyl)-7-ethoxy-1-(1-hydroxy-3,3-dimethylbutan-2-yl)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(2-hydroxyethoxy)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(3-hydroxypropoxy)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(2-methoxyethoxy)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-4-oxo-7-(pyridin-3-ylmethoxy)-1,4-dihydro-1,8-naphthyridine-3-carboxylic acid; (S)-6-(3-chloro-2-fluorobenzyl)-1-(1-hydroxy-3,3-dimethylbutan-2-yl)-7-(2-hydroxyethylamino)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(2-methoxyethylamino)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(3-methoxypropylamino)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-morpholino-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(2-morpholinoethylamino)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(3-morpholinopropylamino)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-4-oxo-7-(3-(2-oxopyrrolidin-1-yl)propylamino)-1,4-dihydro-1,8-naphthyridine-3-carboxylic acid; (S)-6-(3-chloro-2-fluorobenzyl)-1-(1-hydroxy-3,3-dimethylbutan-2-yl)-4-oxo-7-(pyridin-2-ylmethylamino)-1,4-dihydro-1,8-naphthyridine-3-carboxylic acid; (S)-6-(3-chloro-2-fluorobenzyl)-1-(1-hydroxy-3,3-dimethylbutan-2-yl)-4-oxo-7-(pyridin-2-ylmethylamino)-1,4-dihydro-1,8-naphthyridine-3-carboxylic acid; and (S)-6-(3-chloro-2-fluorobenzyl)-1-(1-hydroxy-3,3-dimethylbutan-2-yl)-7-(3-hydroxypropyl)-4-oxo-1,4-dihydro-1,8-naphthyridine-3-carboxylic acid:

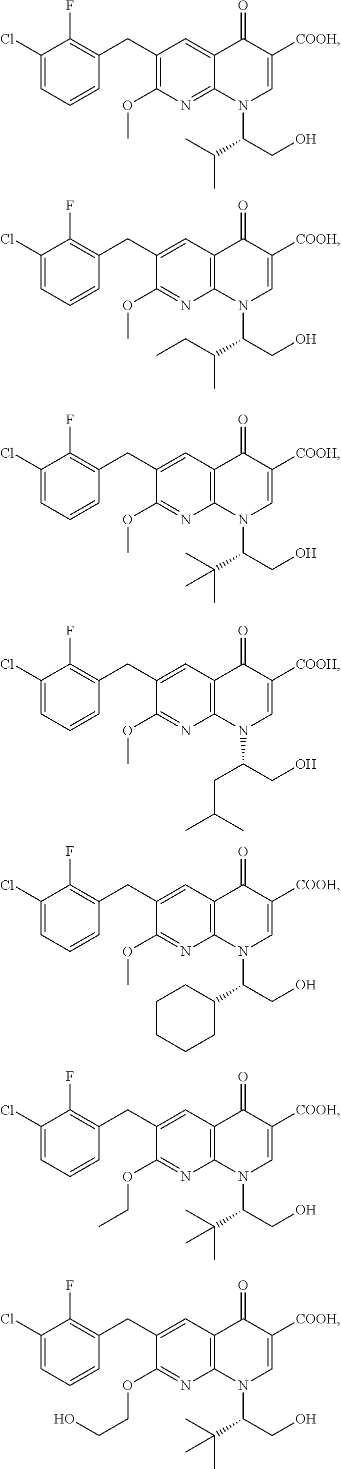

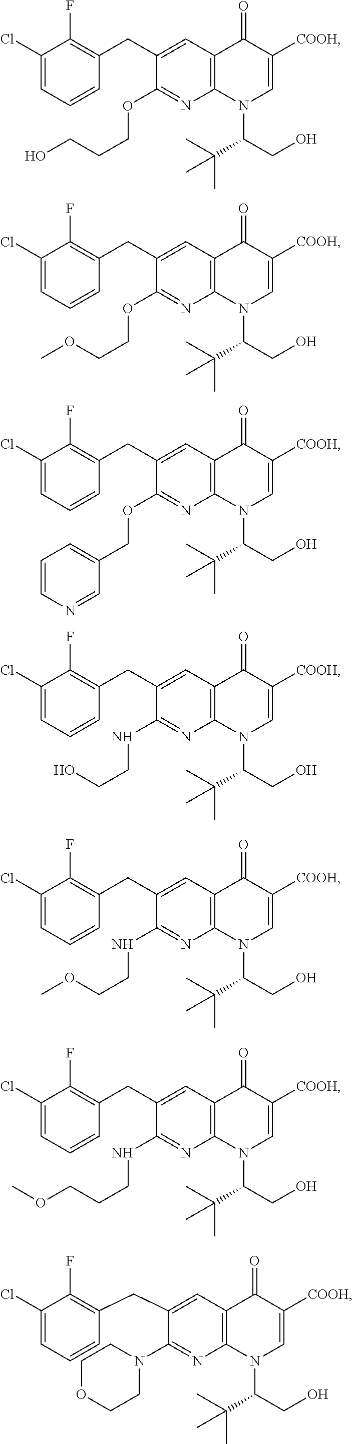

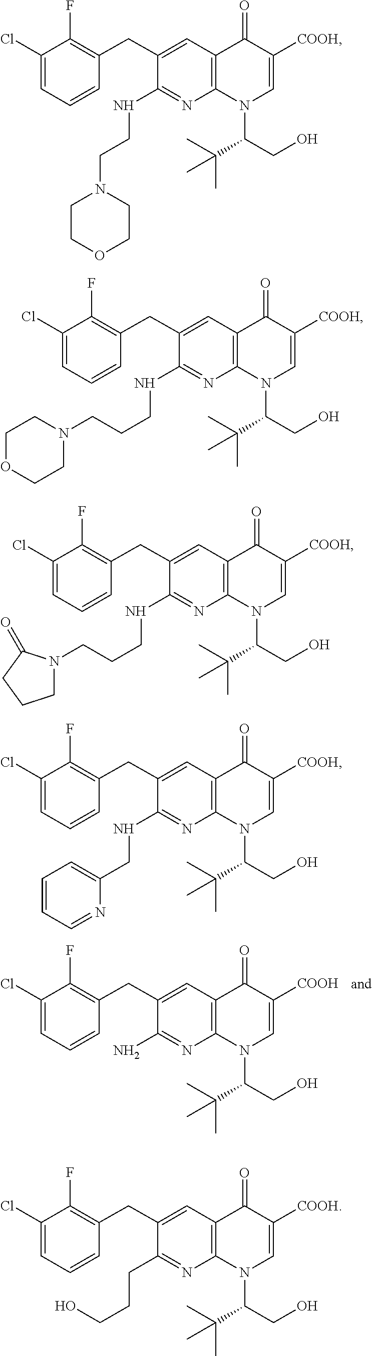

Disclosed herein, in certain embodiments, is a pharmaceutical composition, comprising an effective amount a compound of formula (III) or formula (IV), or a metabolite, pharmaceutically acceptable salt, solvate, polymorph, ester, tautomer or prodrug thereof. In some embodiments, the composition does not comprise a CYP3A4 inhibitor. In some embodiments, the composition further comprises a second therapeutic agent. In some embodiments, the composition further comprises a reverse transcriptase inhibitor, a viral protease inhibitor, a fusion inhibitor, a cytokine, a cytokine inhibitor, a glycosylation inhibitor, a viral mRNA processing inhibitor, an entry inhibitor, an integrase inhibitor or a maturation inhibitor or a combination thereof. In some embodiments, the composition further comprises adefovir, abacavir, amprenavir, atazanavir, apricitabine, bevirimat, darunavir, delavirdine, didanosine, efavirenz, emtricitabine, elvitegravir, enfuvirtide, etravirine, fosamprenavir, fuseon, indinavir, lamivudine, lopinavir, maraviroc, nelfinavir, nevirapine, racivir, raltegravir, reverset, ritonavir, saquinavir, stavudine, tenofovir, tipranavir, vicriviroc, zalcitabine, zidovudine, interferon-α, interferon-β or interferon-γ, or a combination of two or more thereof.

Disclosed herein, in certain embodiments, is a method of treating a viral infection in a patient in need thereof comprising administering to said patient an effective amount of a compound of formula (III) or formula (IV), or a metabolite, pharmaceutically acceptable salt, solvate, polymorph, ester, tautomer or prodrug thereof. In some embodiments, the viral infection is caused by a virus selected from the group consisting of human immunodeficiency viruses 1 (HIV-1), human immunodeficiency viruses 2 (HIV-2), human T-cell leukemia viruses 1 (HTLV-1), human T-cell leukemia viruses 2 (HTLV-2), respiratory syncytial virus (RSV), human papilloma virus (HPV), adenovirus, hepatitis B virus (HBV), hepatitis C virus (HCV), Epstein-Barr virus (EBV), varicella zoster virus (VZV), cytomegalovirus (CMV), herpes simplex viruses 1 (HSV-1), herpes simplex viruses 2 (HSV-2), human herpes virus 8 (HHV-8) Yellow Fever virus, Dengue virus, Japanese Encephalitis and West Nile virus.

Disclosed herein, in certain embodiments, is a method of treating or preventing HIV infection, treating AIDS-related complex (ARC), prophylaxis of ARC, delaying the onset of ARC, treating AIDS, prophylaxis of AIDS or delaying the onset of AIDS in a subject in need thereof, comprising administering to the subject a therapeutically effective amount of a compound of formula (III) or formula (IV), or a metabolite, pharmaceutically acceptable salt, solvate, polymorph, ester, tautomer or prodrug thereof. In some embodiments, the method does not comprise administration of a CYP3A4 inhibitor. In some embodiments, the subject is infected with HIV. In some embodiments, the subject is infected with HIV-1 or HIV-2. In some embodiments, the subject is infected with a drug resistant strain of HIV. In some embodiments, the subject is infected with a multidrug resistant strain of HIV. In some embodiments, the subject is infected with strain of HIV that exhibits reduced susceptibility to reverse transcriptase inhibitors. In some embodiments, the subject is infected with a strain of HIV that exhibits at least one mutation compared to wild type HIV. In some embodiments, the mutation conveys resistance to an AIDS or HIV therapeutic. In some embodiments, the method further comprises administering an effective amount of a second therapeutic agent. In some embodiments, the second therapeutic agent is an anti HIV or AIDS drug. In some embodiments, the second therapeutic agent is a reverse transcriptase inhibitor, a viral protease inhibitor, a fusion inhibitor, a cytokine, a cytokine inhibitor, a glycosylation inhibitor, a viral mRNA processing inhibitor, an entry inhibitor, an integrase inhibitor or a maturation inhibitor or a combination thereof. In some embodiments, the second therapeutic agent is adefovir, abacavir, amprenavir, atazanavir, apricitabine, bevirimat, darunavir, delavirdine, didanosine, efavirenz, emtricitabine, elvitegravir, enfuvirtide, etravirine, fosamprenavir, fuseon, indinavir, lamivudine, lopinavir, maraviroc, nelfinavir, nevirapine, racivir, raltegravir, reverset, ritonavir, saquinavir, stavudine, tenofovir, tipranavir, vicriviroc, zalcitabine, zidovudine, interferon-α, interferon-β or interferon-γ, or a combination of two or more thereof. In some embodiments, the administration of a compound of formula (III) or formula (IV), or a metabolite, pharmaceutically acceptable salt, solvate, polymorph, ester, tautomer or prodrug thereof and the second therapeutic agent is sequential. In some embodiments, the sequential administration is a cycling therapy. In some embodiments, the compound of formula (III) or formula (IV), is administered before the second therapeutic agent. In some embodiments, the compound of formula (III) or formula (IV), is administered after the second therapeutic agent. In some embodiments, the administration of a compound of formula (III) or formula (IV), or a metabolite, pharmaceutically acceptable salt, solvate, polymorph, ester, tautomer or prodrug thereof and the second therapeutic agent is simultaneous.

Disclosed herein, in certain embodiments, is a method for treating HIV infection in a subject in need thereof with combination therapy, comprising administering to said patient an effective amount of a combination of at least one compound of formula (III) or formula (IV) with a second therapeutic agent selected from the group consisting of reverse transcriptase inhibitors, viral protease inhibitors, cytokines, cytokine inhibitors, glycosylation inhibitors, viral mRNA processing inhibitors, entry inhibitors, integrase inhibitors, maturation inhibitors or a combination of two or more thereof.

Disclosed herein, in certain embodiments, is a method for treating HIV infection in a subject in need thereof with combination therapy, comprising administering to said patient an effective amount of a combination of at least one compound of formula (III) or formula (IV), or a metabolite, pharmaceutically acceptable salt, solvate, polymorph, ester, tautomer or prodrug thereof, and a second therapeutic agent selected from the group consisting of adefovir, abacavir, amprenavir, atazanavir, apricitabine, bevirimat, darunavir, delavirdine, didanosine, efavirenz, emtricitabine, elvitegravir, enfuvirtide, etravirine, fosamprenavir, fuseon, indinavir, lamivudine, lopinavir, maraviroc, nelfinavir, nevirapine, racivir, raltegravir, reverset, ritonavir, saquinavir, stavudine, tenofovir, tipranavir, vicriviroc, zalcitabine, zidovudine, interferon-α, interferon-β or interferon-γ, or a combination of two or more thereof.

Disclosed herein, in certain embodiments, is a kit comprising a compound of formula (III) or formula (IV), or a metabolite, pharmaceutically acceptable salt, solvate, polymorph, ester, tautomer or prodrug thereof. In some embodiments, the kit further comprises instructions for administration of the compound to a mammal to treat HIV infection, ARC or AIDS.

### INCORPORATION BY REFERENCE

All patents and patent applications cited in the application are hereby incorporated by reference for the subject matter to which they pertain unless otherwise indicated. All publications, portions of publications, documents, or portions of documents cited in the application including, without limitation, articles, books, manuals and treatises are hereby incorporated by reference for the subject matter to which they pertain unless otherwise indicated.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein are employed in practicing the invention. The novel features of the invention are set forth with particularity in the appended claims. It is intended that the claims define the scope of the invention and that methods and structures within the scope of these claims and their equivalents be, covered thereby.

A better understanding of the features and advantages of the present invention will be obtained by reference to the following detailed description that sets forth illustrative embodiments, in which the principles of the invention are utilized. The section headings used herein are for organizational purposes only and are not to be construed as limiting the subject matter described.

### Certain Chemical Terminology

Unless defined otherwise, all technical and scientific terms used herein have the same meaning as is commonly understood by one of skill in the art to which the claimed subject matter belongs. All patents, patent applications, published materials referred to throughout the entire disclosure herein are incorporated by reference for the subject matter to which they pertain unless otherwise indicated. In the event that there is a plurality of definitions for terms herein, those in this section prevail. Where reference is made to a URL or other such identifier or address, it is understood that such identifiers can change and particular information on the internet can come and go, but equivalent information is found by searching the Internet or other appropriate reference source. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting.

Definition of standard chemistry terms is found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques are used for chemical syntheses, chemical analyses, pharmaceutical preparation, formulation, and delivery, and treatment of patients. Reactions and purification techniques are performed e.g., using kits of manufacturer's specifications or as commonly accomplished in the art or as described herein. The foregoing techniques and procedures are generally performed of conventional methods well known in the art and as described in various general and more specific references that are cited and discussed throughout the present specification. Throughout the specification, groups and substituents thereof are chosen by one skilled in the field to provide stable moieties and compounds.

Where substituent groups are specified by their conventional chemical formulas, written from left to right, they equally encompass the chemically identical substituents that would result from writing the structure from right to left. As a non-limiting example, —CH₂O— is equivalent to —OCH₂—.

Unless otherwise noted, the use of general chemical terms, such as though not limited to “alkyl,” “amine,” “aryl,” are equivalent to their optionally substituted forms. For example, “alkyl,” as used herein, includes optionally substituted alkyl.

In some embodiments, he compounds presented herein possess one or more stereocenters. Ins some embodiments, each center exists in the R or S configuration, or combinations thereof. Likewise, in some embodiments, the compounds presented herein possess one or more double bonds. In some embodiments, each exists in the E (trans) or Z (cis) configuration, or combinations thereof. Presentation of one particular Stereoisomer, regioisomer, diastereomer, enantiomer or epimer should be understood to include all possible stereoisomers, regioisomers, diastereomers, enantiomers or epimers and mixtures thereof. Thus, the compounds presented herein include all separate configurational stereoisomeric, regioisomeric, diastereomeric, enantiomeric, and epimeric forms as well as the corresponding mixtures thereof. For techniques regarding inverting or leaving unchanged a particular stereocenter, and those for resolving mixtures of stereoisomers see, for example, Furniss et al. (eds.), VOGEL'S TEXTBOOK OF PRACTICAL ORGANIC CHEMISTRY 5^{th} Edition, Longman Scientific and Technical Ltd., Essex, 1991, 809-816.

The terms “moiety”, “chemical moiety”, “group” and “chemical group”, as used herein refer to a specific segment or functional group of a molecule. Chemical moieties are often recognized chemical entities embedded in or appended to a molecule.

The term “bond” or “single bond” refers to a chemical bond between two atoms, or two moieties when the atoms joined by the bond are considered to be part of larger substructure.

The term “catalytic group” refers to a chemical functional group that assists catalysis by acting to lower the activation barrier to reaction.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined below. Further, in some embodiments, an optionally substituted group is un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically; up to about 500 daltons (except in those instances where macromolecular substituents are clearly intended, e.g., polypeptides, polysaccharides, polyethylene glycols, DNA, RNA and the like).

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms, as well as the ranges C₁-C₂ and C₁-C₃. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Whenever it appears herein, a numerical range such as “1 to 10” refers to each integer in the given range; e.g., “1 to 10 carbon atoms” means that the group has 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The term “hydrocarbon” as used herein, alone or in combination, refers to a compound or chemical group containing only carbon and hydrogen atoms.

The terms “heteroatom” or “hetero” as used herein, alone or in combination, refer to an atom other than carbon or hydrogen. In some embodiments, heteroatoms are independently selected from among oxygen, nitrogen, sulfur, phosphorous, silicon, selenium and tin but are not limited to these atoms. In embodiments in which two or more heteroatoms are present, the two or more heteroatoms are the same as each another, or some or all of the two or more heteroatoms are each different from the others.

The term “alkyl” as used herein, alone or in combination, refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆ alkyl”, means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The term “alkylene” as used herein, alone or in combination, refers to a diradical derived from the above-defined monoradical, alkyl. Examples include, but are not limited to methylene (—CH₂—), ethylene (—CH₂CH₂—), propylene (—CH₂CH₂CH₂—), isopropylene (—CH(CH₃)CH₂—) and the like. It should be noted that although designated, for example —CH₂—, —CH₂CH₂—, or —CH₂CH₂CH₂—, it should be understood that these alkylene moieties also encompass their substituted equivalents, such as, by way of example only —CHCl—, —CH₂CHF—, —CHPhCH(OH)— and the like.

The term “alkenyl” as used herein, alone or in combination, refers to an optionally substituted straight-chain, or optionally substituted branched-chain hydrocarbon monoradical having one or more carbon-carbon double-bonds and having from two to about ten carbon atoms, more preferably two to about six carbon atoms. The group is in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆ alkenyl”, means that the alkenyl group consists of 2 carbon atoms, 3 carbon atoms, 4 carbon atoms, 5 carbon atoms or 6 carbon atoms, although the present definition also covers the occurrence of the term “alkenyl” where no numerical range is designated.

The term “alkenylene” as used herein, alone or in combination, refers to a diradical derived from the above-defined monoradical alkenyl. Examples include, but are not limited to ethenylene (—CH═CH—), the propenylene isomers (e.g., —CH₂CH═CH— and —C(CH₃)═CH—) and the like.

The term “alkynyl” as used herein, alone or in combination, refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₁₋₆ alkynyl”, means that the alkynyl group consists of 2 carbon atoms, 3 carbon atoms, 4 carbon atoms, 5 carbon atoms or 6 carbon atoms, although the present definition also covers the occurrence of the term “alkynyl” where no numerical range is designated.

The term “alkynylene” as used herein, alone or in combination, refers to a diradical derived from the above-defined monoradical, alkynyl. Examples include, but are not limited to ethynylene propargylene (—CH₂—C≡C—) and the like.

The term “aliphatic” as used herein, alone or in combination, refers to an optionally substituted, straight-chain or branched-chain, non-cyclic, saturated, partially unsaturated, or fully unsaturated nonaromatic hydrocarbon. Thus, the term collectively includes alkyl, alkenyl and alkynyl groups.

The terms “heteroalkyl”, “heteroalkenyl” and “heteroalkynyl” as used herein, alone or in combination, refer to optionally substituted alkyl, alkenyl and alkynyl structures respectively, as described above, in which one or more of the skeletal chain carbon atoms (and any associated hydrogen atoms, as appropriate) are each independently replaced with a heteroatom (i.e. an atom other than carbon, such as though not limited to oxygen, nitrogen, sulfur, silicon, phosphorous, tin or combinations thereof), or heteroatomic group such as though not limited to —O—O—, —S—S—, —O—S—, —S—O—, ═N—N═, —N═N—, —N═N—NH—, —P(O)₂—, —O—P(O)₂—, —P(O)₂—O—, —S(O)—, —SnH₂— and the like.

The terms “haloalkyl”, “haloalkenyl” and “haloalkynyl” as used herein, alone or in combination, refer to optionally substituted alkyl, alkenyl and alkynyl groups respectively, as defined above, in which one or more hydrogen atoms is replaced by fluorine, chlorine, bromine or iodine atoms, or combinations thereof. In some embodiments two or more hydrogen atoms are replaced with halogen atoms that are the same as each another (e.g. difluoromethyl); in other embodiments two or more hydrogen atoms are replaced with halogen atoms that are not all the same as each other (e.g. 1-chloro-1-fluoro-1-iodoethyl). Non-limiting examples of haloalkyl groups are fluoromethyl and bromoethyl. A non-limiting example of a haloalkenyl group is bromoethenyl. A non-limiting example of a haloalkynyl group is chloroethynyl.

The term “perhalo” as used herein, alone or in combination, refers to groups in which all of the hydrogen atoms are replaced by fluorines, chlorines, bromines, iodines, or combinations thereof. Thus, as a non-limiting example, the term “perhaloalkyl” refers to an alkyl group, as defined herein, in which all of the H atoms have been replaced by fluorines, chlorines, bromines or iodines, or combinations thereof. A non-limiting example of a perhaloalkyl group is bromo, chloro, fluoromethyl. A non-limiting example of a perhaloalkenyl group is trichloroethenyl. A non-limiting example of a perhaloalkynyl group is tribromopropynyl.

The term “carbon chain” as used herein, alone or in combination, refers to any alkyl, alkenyl, alkynyl, heteroalkyl, heteroalkenyl or heteroalkynyl group, which is linear, cyclic, or any combination thereof. If the chain is part of a linker and that linker comprises one or more rings as part of the core backbone, for purposes of calculating chain length, the “chain” only includes those carbon atoms that compose the bottom or top of a given, ring and not both, and where the top and bottom of the ring(s) are not equivalent in length, the shorter distance shall be used in determining the chain length. If the chain contains heteroatoms as part of the backbone, those atoms are not calculated as part of the carbon chain length.

The terms “cycle”, “cyclic”, “ring” and “membered ring” as used herein, alone or in combination, refer to any covalently closed structure, including alicyclic, heterocyclic, aromatic, heteroaromatic and polycyclic fused or non-fused ring systems as described herein. In some embodiments, rings are optionally substituted. In some embodiments, rings form part of a fused ring system. The term “membered” is meant to denote the number of skeletal atoms that constitute the ring. Thus, by way of example only, cyclohexane, pyridine, pyran and pyrimidine are six-membered rings and cyclopentane, pyrrole, tetrahydrofuran and thiophene are five-membered rings.

The term “fused” as used herein, alone or in combination, refers to cyclic structures in which two or more rings share one or more bonds.

The term “cycloalkyl” as used herein, alone or in combination, refers to an optionally substituted, saturated, hydrocarbon monoradical ring, containing from three to about fifteen ring carbon atoms or from three to about ten ring carbon atoms. In some embodiments, the term includes additional, non-ring carbon atoms as substituents (e.g. methylcyclopropyl). Whenever it appears herein, a numerical range such as “C₃-C₆ cycloalkyl” or “C₃₋₆ cycloalkyl”, means that the cycloalkyl group consists of 3 carbon atoms, 4 carbon atoms, 5 carbon atoms or 6 carbon atoms, i.e., is cyclopropyl, cyclobutyl, cyclopentyl or cyclohepty, although the present definition also covers the occurrence of the term “cycloalkyl” where no numerical range is designated. The term includes fused, non-fused, bridged and spiro radicals. A fused cycloalkyl contains from two to four fused rings where the ring of attachment is a cycloalkyl ring, and the other individual rings are alicyclic, heterocyclic, aromatic, heteroaromatic or any combination thereof. Examples include, but are not limited to cyclopropyl, cyclopentyl, cyclohexyl, decalinyl, and bicyclo [2.2.1]heptyl and adamantyl ring systems. Illustrative examples include, but are not limited to the following moieties:

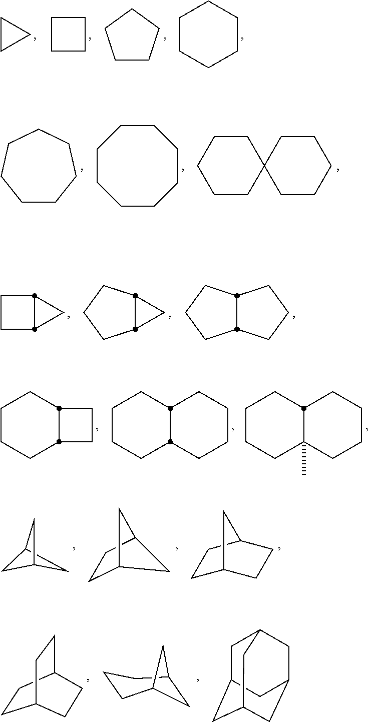

and the like.

The term “cycloalkenyl” as used herein, alone or in combination, refers to an optionally substituted hydrocarbon non-aromatic, monoradical ring, having one or more carbon-carbon double-bonds and from three to about twenty ring carbon atoms, three to about twelve ring carbon atoms, or from three to about ten ring carbon atoms. The term includes fused, non-fused, bridged and spiro radicals. In some embodiments, a fused cycloalkenyl contains from two to four fused rings where the ring of attachment is a cycloalkenyl ring, and the other individual rings are alicyclic, heterocyclic, aromatic, heteroaromatic or any combination thereof. In some embodiments, fused ring systems are fused across a bond that is a carbon-carbon single bond or a carbon-carbon double bond. Examples of cycloalkenyls include, but are not limited to cyclohexenyl, cyclopentadienyl and bicyclo[2.2.1]hept-2-ene ring systems. Illustrative examples include, but are not limited to the following moieties:

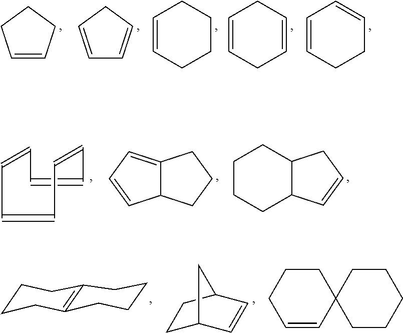

and the like.

The terms “alicyclyl” or “alicyclic” as used herein, alone or in combination, refer to an optionally substituted, saturated, partially unsaturated, or fully unsaturated nonaromatic hydrocarbon ring systems containing from three to about twenty ring carbon atoms, three to about twelve ring carbon atoms, or from three to about ten ring carbon atoms. Thus, the terms collectively include cycloalkyl and cycloalkenyl groups.

The terms “non-aromatic heterocyclyl” and “heteroalicyclyl” as used herein, alone or in combination, refer to optionally substituted, saturated, partially unsaturated, or fully unsaturated nonaromatic ring monoradicals containing from three to about twenty ring atoms, where one or more of the ring atoms are an atom other than carbon, independently selected from among oxygen, nitrogen, sulfur, phosphorous, silicon, selenium and tin but are not limited to these atoms. In embodiments in which two or more heteroatoms are present in the ring, the two or more heteroatoms are the same as each another, or some or all of the two or more heteroatoms are each different from the others. The terms include fused, non-fused, bridged and spiro radicals. In some embodiments, a fused non-aromatic heterocyclic radical contains from two to four fused rings where the attaching ring is a non-aromatic heterocycle, and the other individual rings are alicyclic, heterocyclic, aromatic, heteroaromatic or any combination thereof. In some embodiments, fused ring systems are fused across a single bond or a double bond, as well as across bonds that are carbon-carbon, carbon-hetero atom or hetero atom-hetero atom. The terms also include radicals having from three to about twelve skeletal ring atoms, as well as those having from three to about ten skeletal ring atoms. In some embodiments, attachment of a non-aromatic heterocyclic subunit to its parent molecule is via a heteroatom or a carbon atom. Likewise, in some embodiments, additional substitution is via a heteroatom or a carbon atom. As a non-limiting example, an imidazolidine non-aromatic heterocycle is attached to a parent molecule via either of its N atoms (imidazolidin-1-yl or imidazolidin-3-yl) or any of its carbon atoms (imidazolidin-2-yl, imidazolidin-4-yl or imidazolidin-5-yl). In certain embodiments, non-aromatic heterocycles contain one or more carbonyl or thiocarbonyl groups such as, for example, oxo- and thio-containing groups. Examples include, but are not limited to pyrrolidinyl, tetrahydrofuranyl, dihydrofuranyl, tetrahydrothienyl, tetrahydropyranyl, dihydropyranyl, tetrahydrothiopyranyl, piperidine, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Illustrative examples of heterocycloalkyl groups, also referred to as non-aromatic heterocycles, include:

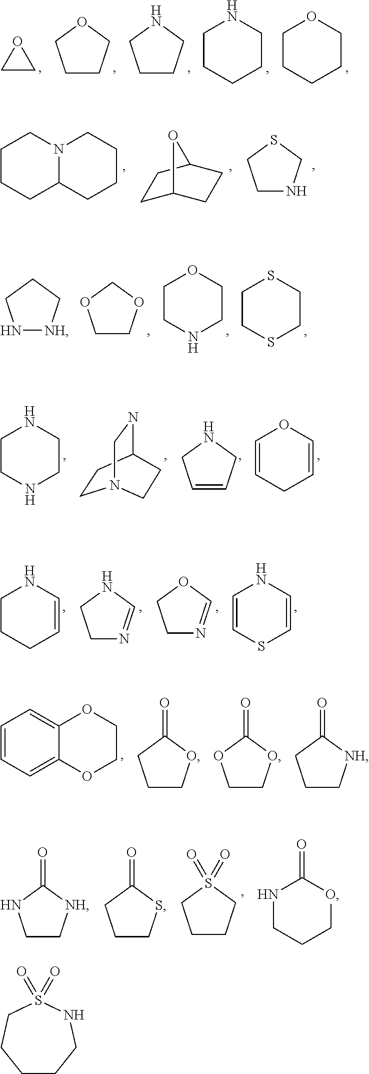

and the like.

The terms also include all ring forms of the carbohydrates, including but not limited to the monosaccharides, the disaccharides and the oligosaccharides.

The term “aromatic” as used herein, refers to a planar, cyclic or polycyclic, ring moiety having a delocalized π-electron system containing 4n+2 π electrons, where n is an integer. Aromatic rings are formed by five, six, seven, eight, nine, or more than nine atoms. In some embodiments, aromatics are optionally substituted and are monocyclic or fused-ring polycyclic. The term aromatic encompasses both all carbon containing rings (e.g., phenyl) and those rings containing one or more heteroatoms (e.g., pyridine).

The term “aryl” as used herein, alone or in combination, refers to an optionally substituted aromatic hydrocarbon radical of six to about twenty ring carbon atoms, and includes fused and non-fused aryl rings. In some embodiments, a fused aryl ring radical contains from two to four fused rings where the ring of attachment is an aryl ring, and the other individual rings are alicyclic, heterocyclic, aromatic, heteroaromatic or any combination thereof. Further, the term aryl includes fused and non-fused rings containing from six to about twelve ring carbon atoms, as well as those containing from six to about ten ring carbon atoms. A non-limiting example of a single ring aryl group includes phenyl; a fused ring aryl group includes naphthyl, phenanthrenyl, anthracenyl, azulenyl; and a non-fused bi-aryl group includes biphenyl.

The term “arylene” as used herein, alone or in combination, refers to a diradical derived from the above-defined monoradical, aryl. Examples include, but are not limited to 1,2-phenylene, 1,3-phenylene, 1,4-phenylene, 1,2-naphthylene and the like.

The term “heteroaryl” as used herein, alone or in combination, refers to optionally substituted aromatic monoradicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent O or S atoms. In embodiments in which two or more heteroatoms are present in the ring, the two or more heteroatoms are the same as each another, or some or all of the two or more heteroatoms are each different from the others. The term heteroaryl includes optionally substituted fused and non-fused heteroaryl radicals having at least one heteroatom. The term heteroaryl also includes fused and, non-fused heteroaryls having from five to about twelve skeletal ring atoms, as well as those having from five to about ten skeletal ring atoms. In some embodiments, bonding to a heteroaryl group is via a carbon atom or a heteroatom. Thus, as a non-limiting example, an imidiazole group is attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group is further substituted via any or all of its carbon atoms, and/or any or all of its heteroatoms. In some embodiments, a fused heteroaryl radical contains from two to four fused rings where the ring of attachment is a heteroaromatic ring and the other individual rings are alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, phenazinyl, benzimidazolyl, benzofuranyl, benzoxazolyl, benzothiazolyl, benzothiadiazolyl, benzothiophenyl, benzoxadiazolyl, benzotriazolyl, imidazolyl, indolyl, isoxazolyl, isoquinolinyl, indolizinyl, isothiazolyl, isoindolyloxadiazolyl, indazolyl, pyridyl, pyridazyl, pyrimidyl, pyrazinyl, pyrrolyl, pyrazinyl, pyrazolyl, purinyl, phthalazinyl, pteridinyl, quinolinyl, quinazolinyl, quinoxalinyl, triazolyl, tetrazolyl, thiazolyl, triazinyl, thiadiazolyl and the like, and their oxides, such as for example pyridyl-N-oxide. Illustrative examples of heteroaryl groups include the following moieties:

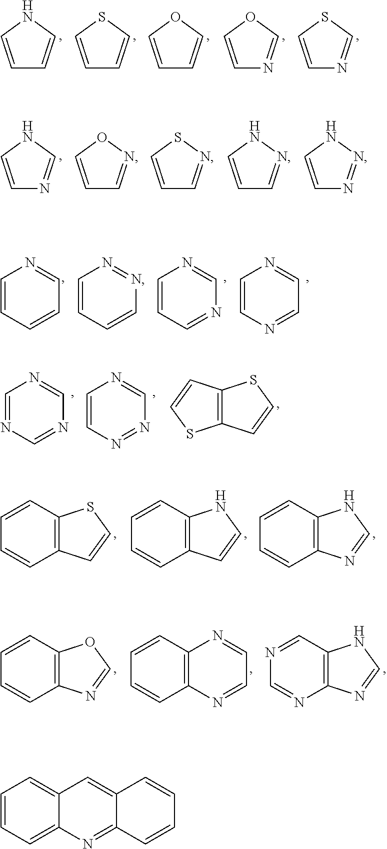

and the like.

The term “heteroarylene” as used herein, alone or in combination, refers to a diradical derived from the above-defined monoradical heteroaryl. Examples include, but are not limited to pyridinyl and pyrimidinyl.

The term “heterocyclyl” as used herein, alone or in combination, refers collectively to heteroalicyclyl and heteroaryl groups. Herein, whenever the number of carbon atoms in a heterocycle is indicated (e.g., C₁-C₆ heterocycle), at least one non-carbon atom (the heteroatom) must be present in the ring. Designations such as “C₁-C₆ heterocycle” refer only to the number of carbon atoms in the ring and do not refer to the total number of atoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). For heterocycles having two or more heteroatoms, those two or more heteroatoms are the same or different from one another. In some embodiments, heterocycles are optionally substituted. Non-aromatic heterocyclic groups include groups having only three atoms in the ring, while aromatic heterocyclic groups must have at least five atoms in the ring. In some embodiments, bonding (i.e. attachment to a parent molecule or further substitution) to a heterocycle is via a heteroatom or a carbon atom.

The term “carbocyclyl” as used herein, alone or in combination, refers collectively to alicyclyl and aryl groups; i.e. all carbon, covalently closed ring structures, which are saturated, partially unsaturated, fully unsaturated or aromatic. Carbocyclic rings are formed by three, four, five, six, seven, eight, nine, or more than nine carbon atoms. Carbocycles are optionally substituted. The term distinguishes carbocyclic from heterocyclic rings in which the ring backbone contains at least one atom which is different from carbon.

The terms “halogen”, “halo” or “halide” as used herein, alone or in combination refer to fluoro, chloro, bromo and iodo.

The term “hydroxy” as used herein, alone or in combination, refers to the monoradical —OH.

The term “cyano” as used herein, alone or in combination, refers to the monoradical —CN.

The term “cyanomethyl” as used herein, alone or in combination, refers to the monoradical —CH₂CN.

The term “nitro” as used herein, alone or in combination, refers to the monoradical —NO₂.

The term “oxy” as used herein, alone or in combination, refers to the diradical —O—.

The term “oxo” as used herein, alone or in combination, refers to the diradical ═O.

The term “carbonyl” as used herein, alone or in combination, refers to the diradical —C(═O)—, which are written as —C(O)—.

The terms “carboxy” or “carboxyl” as used herein, alone or in combination, refer to the moiety —C(O)OH, which are written as —COOH.

The term “alkoxy” as used herein, alone or in combination, refers to an alkyl ether radical, —O-alkyl, including the groups —O-aliphatic and —O-carbocyclyl, wherein the alkyl, aliphatic and carbocyclyl groups is optionally substituted, and wherein the terms alkyl, aliphatic and carbocyclyl areas defined herein. Non-limiting examples of alkoxy radicals include methoxy, ethoxy, n-propoxy, isopropoxy, n-butoxy, iso-butoxy, sec-butoxy, tert-butoxy and the like.

The term “sulfonyl” as used herein, alone or in combination, refers to the diradical —S(═O)—.

The term “sulfonyl” as used herein, alone or in combination, refers to the diradical —S(═O)₂—.

The terms “sulfonamide”, “sulfonamido” and “sulfonamidyl” as used herein, alone or in combination, refer to the diradical groups —S(═O)₂—NH— and —NH—S(═O)₂—.

The terms “sulfamide”, “sulfamido” and “sulfamidyl” as used herein, alone or in combination, refer to the diradical group —NH—S(═O)₂—NH—.

The term “reactant,” as used herein, refers to a nucleophile or electrophile used to create covalent linkages.

It is to be understood that in instances where two or more radicals are used in succession to define a substituent attached to a structure, the first named radical is considered to be terminal and the last named radical is considered to be attached to the structure in question. Thus, for example, the radical arylalkyl is attached to the structure in question by the alkyl group.

### Certain Pharmaceutical Terminology

The term “integrase inhibitor” as used herein refers to a compound that exhibits an IC₅₀ with respect to integrase activity, of no more than about 100 μM or not more than, about 50 μM. “IC₅₀” is that concentration of inhibitor which reduces the activity of an enzyme to half-maximal level. Compounds described herein have been discovered to exhibit inhibition against integrase. Compounds of formula (I) or (II); formula (III) or (IV); or formula (V)(a), (V)(b) or (V)(c) preferably exhibit an IC₅₀ with respect to integrase of no more than about 10 μM, more preferably, no more than about 5 μM, even more preferably not more than about 1 μM, and most preferably, not more than about 200 nM.

The term “subject”, “patient” or “individual” as used herein in reference to individuals suffering from a disorder, and the like, encompasses mammals and non-mammals. None of the terms requires the supervision of a medical professional (e.g., a doctor, nurse, orderly, physician's assistant, hospice worker) Mammals are any member of the Mammalian class, including but not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the subject is a mammal. In preferred embodiments, the subject is a human.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still be afflicted with the underlying disorder. For prophylactic benefit, the compositions are administered to a patient at risk of developing a particular disease, or to a patient reporting one or more of the physiological symptoms of a disease, even though a diagnosis of this disease has not been made.

The terms “administer,” “administering”, “administration,” and the like, as used herein, refer to the methods that are used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. In preferred embodiments, the compounds and compositions described herein are administered orally.

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. In some embodiments, the result is reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. In some embodiments, an appropriate “effective” amount differs from one individual to another. An appropriate “effective” amount in any individual case is determined using any suitable technique, such as a dose escalation study.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of a compound of formula (I) or (II); formula (III) or (IV); or formula (V)(a), (V)(b) or (V)(c), and is relatively nontoxic, i.e., the material is administered to an individual without causing undesirable biological effects or interacting in a deleterious manner with any of the components of the composition in which it is contained.

The term “prodrug” as used herein, refers to a drug precursor that, following administration to a subject and subsequent absorption, is converted to an active, or a more active species via some process, such as conversion by a metabolic pathway. Thus, the term encompasses any derivative of a compound, which, upon administration to a recipient, is capable of providing, either directly or indirectly, a compound of formula (I) or (II); formula (III) or (IV); or formula (V)(a), (V)(b) or (V)(c) or a pharmaceutically active metabolite or residue thereof. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they are easier to administer than the parent drug. In some embodiments, they are, for instance, bioavailable by oral administration whereas the parent is not. Particularly favored-derivatives or prodrugs are those that increase the bioavailability of the compounds of formula (I) or (II); formula (III) or (TV); or formula (V)(a), (V)(b) or (V)(c) when such compounds are administered to a patient (e.g. by allowing an orally administered compound to be more readily absorbed into the blood) or which enhance delivery of the parent compound to a biological compartment (e.g. the brain or lymphatic system).

The term “pharmaceutically acceptable salt” as used herein, refers to salts that retain the biological effectiveness of the free acids and bases of the specified compound and that are not biologically or otherwise undesirable. In some embodiments, compounds described herein possess acidic or basic groups and therefore react with any of a number of inorganic or organic bases, and inorganic and organic acids, to form a pharmaceutically acceptable salt. These salts are prepared in situ during the final isolation and purification of the compounds of formula (I) or (II); formula (III) or (IV); or formula (V)(a), (V)(b) or (V)(c), or by separately reacting a purified compound in its free base form with a suitable organic or inorganic acid, and isolating the salt thus formed.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, excipients and the like.

The term “carrier” as used herein, refers to relatively nontoxic chemical compounds or agents that facilitate the incorporation of a compound into cells or tissues.

The terms “pharmaceutical combination”, “administering an additional therapy”, “administering an additional therapeutic agent” and the like, as used herein, refer to a pharmaceutical therapy resulting from the mixing or combining of more than one active ingredient and includes both fixed and non-fixed combinations of the active ingredients. The term “fixed combination” means that at least one of a compound of formula (I) or (II); formula (III) or (IV); or formula (V)(a), (V)(b) or (V)(c), and at least one co-agent, are both administered to a patient simultaneously in the form of a single entity or dosage. The term “non-fixed combination” means that at least one of a compound of formula (I) or (II); formula (III) or (IV); or formula (V)(a), (V)(b) or (V)(c), and at least one co-agent, are administered to a patient as separate entities either simultaneously, concurrently or sequentially with variable intervening time limits, wherein such administration provides effective levels of the two or more compounds in the body of the patient. These also apply to cocktail therapies, e.g. the administration, of three or more active ingredients.

The terms “co-administration”, “administered in combination with” and their grammatical equivalents or the like, as used herein, are meant to encompass administration of the selected therapeutic agents to a single patient, and are intended to include treatment regimens in which the agents are administered by the same or different route of administration or at the same or different times. In some embodiments a compound of formula (I) or (II); formula (III) or (IV); or formula (V)(a), (V)(b) or (V)(c) will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the compounds of formula (I) or (II); formula (III) or (IV); or formula (V)(a), (V)(b) or (V)(c) and the other agent(s) are administered in a single composition. In some embodiments, compounds of formula (I) or (II); formula (III) or (IV); or formula (V)(a), (V)(b) or (V)(c) and the other agent(s) are admixed in the composition.

The term “metabolite,” as used herein, refers to a derivative of a compound which is formed when the compound is metabolized.

The term “active metabolite,” as used herein, refers to a biologically active derivative of a compound that is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, in certain instances, enzymes produce specific structural alterations to a compound. For example, cytochrome P450 catalyzes a variety of oxidative and reductive reactions while uridine diphosphate glucuronyltransferases catalyze the transfer, of an activated glucuronic-acid molecule to aromatic alcohols, aliphatic alcohols, carboxylic acids, amines and free sulphydryl groups. For further information on metabolism see Brunton (editor-in-chief), Goodman & Gilman's The Pharmacological of Therapeutics, 11th Edition, New York, N.Y., McGraw-Hill, 2006.

### Compounds

Disclosed herein, in certain embodiments, is a compound of formula (I) or formula (II) or a metabolite, pharmaceutically acceptable salt, solvate, polymorph, ester, tautomer or prodrug thereof:

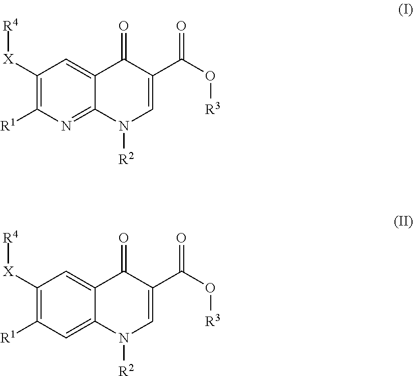

wherein: R¹ is H, F, Cl, Br, I, CFH₂, CF₂H, CF₃, CN, OH, NO₂, NH2, NH(alkyl) or N(alkyl)₂, SO₂CH₃, SO₂NH2, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl or optionally substituted heteroaryl; R² is optionally substituted alkyl, optionally substituted cycloalkyl, optionally substituted heterocycloalkyl, optionally substituted aryl or optionally substituted heteroaryl; R³ is H, C1-6 alkyl or a pharmaceutically acceptable cation; and wherein X is O or N—R⁵; wherein R⁵ is H or optionally substituted C₁₋₄ alkyl;

R⁴ is

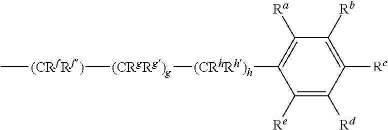

wherein each R^{f}, R^{f′}, R^{g}, R^{g′}, R^{h} and R^{h′} is H or optionally substituted C₁₋₁₀ alkyl; g is 0 or 1; h is 0 or 1; R^{a}, R^{b}, R^{c}, R^{d} and R^{e} are independently selected from H, F, Cl, Br, I, CF₃, CN, alkyl, cycloalkyl, cyclopropylmethyl, NH₂, NHR′, NR′R″, OH, OR′, SH, SR′, C(O)R′, CO2H, COOR′, CONH2, CONHR′, CONR′R″, SO₃H, S(O)₂R′, S(O)₂NH2, S(O)₂NHR′, S(O)₂NR′R″, aryl, heterocyclyl and heteroaryl; wherein R′ is methyl, ethyl, n-propyl, i-propyl, n-butyl, i-butyl, s-butyl, t-butyl, cyclopropyl, cyclobutyl, cyclopentyl, cyclohexyl or cyclopropylmethyl; R″ is methyl, ethyl, n-propyl, i-propyl, n-butyl, i-butyl, s-butyl, t-butyl, cyclopropyl, cyclobutyl, cyclopentyl, cyclohexyl or cyclopropylmethyl; or R′ and R″ together with the nitrogen atom to which they are attached form an optionally substituted 4-, 5- or 6-membered heterocyclic ring; or X is N and R⁵ and R^{f}, or R⁵ and R^{g}, or R⁵ and R^{h}, together with the N atom form an optionally substituted 4-, 5- or 6-membered heterocyclic ring, optionally containing 1 or 2 additional heteroatoms selected from O, N and S; and all alkyl, alkylene, cycloalkyl, heterocyclyl, aryl and heteroaryl moieties may be optionally further substituted.

In some embodiments, R¹ is H, optionally substituted alkyl, optionally substituted alkoxy or optionally substituted heterocycle. In some embodiments, R¹ is alkoxy. In some embodiments, R¹ is methoxy.

In some embodiments, R² is optionally substituted C₁₋₁₀ alkyl. In some embodiments, R² is substituted C₅ or C₆ alkyl. In some embodiments, C₅ or C₆ alkyl is substituted with one OH group. In some embodiments, R² is 1-hydroxy-3,3-dimethylbutan-2-yl or 1-hydroxy-3-methylbutan-2-yl:

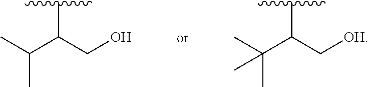

In some embodiments, R² comprises a chiral center. In some embodiments, the chiral center is in the (S) configuration.

In some embodiments, R³ is H.

In some embodiments, R¹ is alkoxy; R² is C₅ or C₆ alkyl substituted with one OH group; and R³ is H.

In some embodiments, X is NH.

In some embodiments, R⁴ is

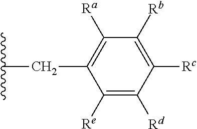

In some embodiments, X is NH and R⁴ is

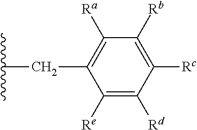

In some embodiments, R^{a}, R^{b}, R^{c}, R^{d} and R^{e} are independently selected from H, F and Cl.

Disclosed herein, in certain embodiments, is a compound selected from: (S)-1-(1-hydroxy-3-methylbutan-2-yl)-7-methoxy-4-oxo-6-(2,4,6-trifluorobenzylamino)-1,4-dihydro-1,8-naphthyridine-3-carboxylic acid; (S)-1-(1-hydroxy-3-methylbutan-2-yl)-7-methoxy-4-oxo-6-(2,4,6-trifluorobenzylamino)-1,4-dihydroquinoline-3-carboxylic acid; (S)-1-(1-hydroxy-3,3-dimethylbutan-2-yl)-7-methoxy-4-oxo-6-(2,4,6-trifluorobenzylamino)-1,4-dihydro-1,8-naphthyridine-3-carboxylic acid; (S)-6-(2,4-difluorobenzylamino)-1-(1-hydroxy-3-methylbutan-2-yl)-7-methoxy-4-oxo-1,4-dihydro-1,8-naphthyridine-3-carboxylic acid; (S)-1-(1-hydroxy-3-methylbutan-2-yl)-4-oxo-6-(2,4,6-trifluorobenzylamino)-1,4-dihydroquinoline-3-carboxylic acid; (R)-1-(1-hydroxy-3-methylbutan-2-yl)-7-morpholino-4-oxo-6-(2,4,6-trifluorobenzyloxy)-1,4-dihydro-1,8-naphthyridine-3-carboxylic acid; (S)-6-(2,6-difluorobenzylamino)-1-(1-hydroxy-3-methylbutan-2-yl)-7-methoxy-4-oxo-1,4-dihydroquinoline-3-carboxylic acid; (S)-6-(2,6-difluorobenzylamino)-1-(1-hydroxy-3-methylbutan-2-yl)-7-methoxy-4-oxo-1,4-dihydro-1,8-naphthyridine-3-carboxylic acid; (S)-6-(2,4-difluorobenzylamino)-1-(1-hydroxy-3-methylbutan-2-yl)-7-methoxy-4-oxo-1,4-dihydroquinoline-3-carboxylic acid; and (S)-1-(1-hydroxy-3,3-dimethylbutan-2-yl)-7-methyl-4-oxo-6-(2,4,6-trifluorobenzylamino)-1,4-dihydro-1,8-naphthyridine-3-carboxylic acid:

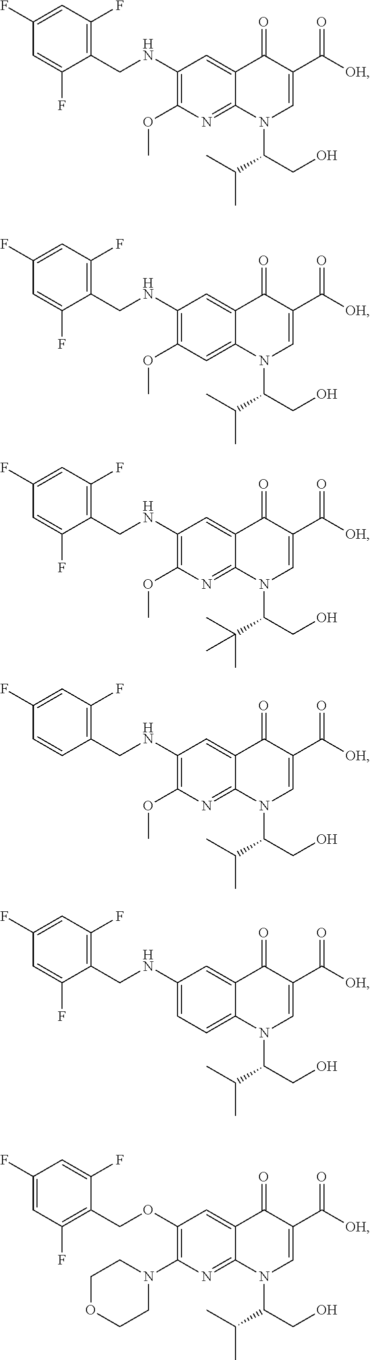

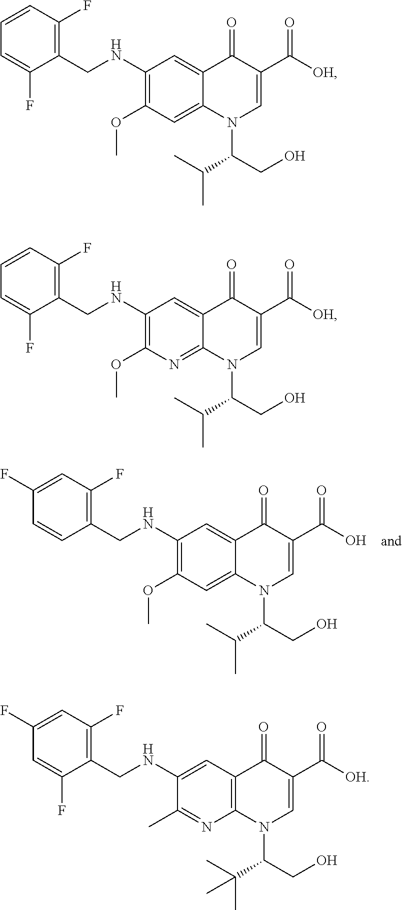

Disclosed herein, in certain embodiments, is a compound of formula (III) or formula (IV) or a metabolite, pharmaceutically acceptable salt, solvate, polymorph, ester, tautomer or prodrug thereof:

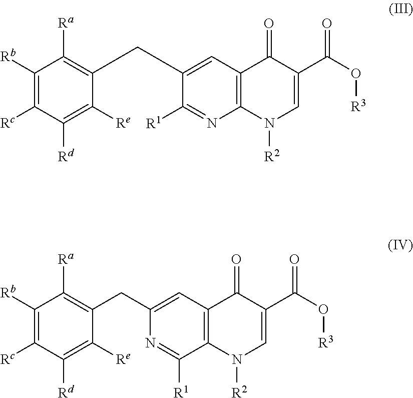

wherein: R¹ is H, F, Cl, Br, I, CFH₂, CF₂H, CF₃, CN, OH, NO₂, NH2, NH (optionally substituted alkyl) or N (optionally substituted alkyl)(optionally substituted alkyl), SO₂CH₃, SO₂NH2, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl, optionally substituted heteroaryl; R² is optionally substituted alkyl, optionally substituted cycloalkyl, optionally substituted heterocycloalkyl, optionally substituted aryl or optionally substituted heteroaryl; R³ is H, C1-6 alkyl or a pharmaceutically acceptable cation; and wherein R^{a}, R^{b}, R^{c}, R^{d} and R^{e} are independently selected from H, F, Cl, Br, I, CF₃, CN, alkyl, cycloalkyl, cyclopropylmethyl, NH2, NHR′, NR′R″, OH, OR′, SH, SR′, C(O)R′, CO₂H, COOR′, CONH2, CONHR′, CONR′R″, SO₃H, S(O)₂R′, S(O)₂NH2, S(O)₂NHR′, S(O)₂NR′R″, aryl, heterocyclyl and heteroaryl; wherein R′ is methyl, ethyl, n-propyl, i-propyl, n-butyl, i-butyl, s-butyl, t-butyl, cyclopropyl, cyclobutyl, cyclopentyl, cyclohexyl or cyclopropylmethyl; R″ is methyl, ethyl, n-propyl, propyl, n-butyl, i-butyl, s-butyl, t-butyl, cyclopropyl, cyclobutyl, cyclopentyl, cyclohexyl or cyclopropylmethyl; or R′ and R″ together with the nitrogen atom to which they are attached form an optionally substituted 4-, 5- or 6-membered heterocyclic ring; and all alkyl, alkylene, cycloalkyl, heterocyclyl, aryl and heteroaryl moieties may be optionally further substituted; and provided that the compound is not:

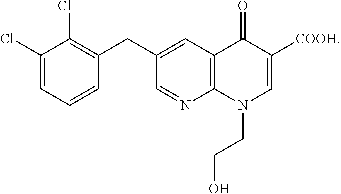

In some embodiments, R¹ is alkyl, substituted alkyl, alkoxy, substituted alkoxy, NH2, NH (optionally substituted alkyl), N (optionally substituted alkyl)(optionally substituted alkyl), heterocycle or substituted heterocycle. In some embodiments, R¹ is heterocyclyl, substituted alkyl, substituted alkoxy or NH (substituted alkyl), wherein the substituents are selected from hydroxy, hydroxyalkyl, alkoxyalkyl, aryl, aralkyl, heterocyclyl and alkylene-heterocyclyl. In some embodiments, R¹ is —CH₂—R^{1a}, —O—R^{1a} or —NH—R^{1a} wherein R^{1a} is methyl, ethyl, hydroxyethylene, hydroxypropylene, methoxyethylene, methoxypropylene, arylmethyl, heteroarylmethylene, heterocyclomethylene, heterocycloethylene or heterocyclopropylene. In some embodiments, R¹ is methoxy.

In some embodiments, R² is optionally substituted C1-10 alkyl. In some embodiments, R² is optionally substituted C₅₋₈ alkyl. In some embodiments, the C₅₋₈ alkyl is substituted with one OH group. In some embodiments, R² is 1-hydroxy-3,3-dimethylbutan-2-yl or 1-hydroxy-3-methylbutan-2-yl:

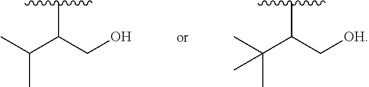

In some embodiments, R² comprises a chiral center. In some embodiments, the chiral center is in the (S) configuration.

In some embodiments, R³ is H.

in some embodiments, R¹ is heterocyclyl, substituted alkyl, substituted alkoxy or NH (substituted alkyl); R² is C₅₋₈ alkyl substituted with one OH group; and R³ is H.

In some embodiments, R^{a}, R^{b}, R^{c}, R^{d} and R^{e} are independently selected from H, F and Cl.

In some embodiments, one of R^{a}, R^{b}, R^{c}, R^{d} and R^{e} is F; one of R^{a}, R^{b}, R^{c}, R^{d} and R^{e} is Cl; and the rest of R^{a}, R^{b}, R^{c}, R^{d} and R^{e} are H.

In some embodiments, R^{a} is F; R^{b} is Cl; and R^{c}, R^{d} and R^{e} are H.

Disclosed herein, in certain embodiments, is a compound selected from (S)-6-(3-chloro-2-fluorobenzyl)-1-(1-hydroxy-3-methylbutan-2-yl)-7-methoxy-4-oxo-1,4-dihydro-1,8-naphthyridine-3-carboxylic acid; 6-(3-chloro-2-fluorobenzyl)-1-((2S,3S)-1-hydroxy-3-methylpentan-2-yl)-7-methoxy-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-methoxy-4-oxo-1,4-dihydro-1,8-naphthyridine-3-carboxylic acid; (S)-6-(3-chloro-2-fluorobenzyl)-1-(1-hydroxy-4-methylpentan-2-yl)-7-methoxy-4-oxo-1,4-dihydro-1,8-naphthyridine-3-carboxylic acid; (S)-6-(3-chloro-2-fluorobenzyl)-1-(1-cyclohexyl-2-hydroxyethyl)-7-methoxy-4-oxo-1,4-dihydro-1,8-naphthyridine-3-carboxylic acid; (S)-6-(3-chloro-2-fluorobenzyl)-7-ethoxy-1-(1-hydroxy-3,3-dimethylbutan-2-yl)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(2-hydroxyethoxy)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(3-hydroxypropoxy)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(2-methoxyethoxy)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-4-oxo-7-(pyridin-3-ylmethoxy)-1,4-dihydro-1,8-naphthyridine-3-carboxylic acid; (S)-6-(3-chloro-2-fluorobenzyl)-1-(1-hydroxy-3,3-dimethylbutan-2-yl)-7-(2-hydroxyethylamino)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(2-methoxyethylamino)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(3-methoxypropylamino)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-morpholino-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(2-morpholinoethylamino)-4-oxo-1,4-dihydro-1,8-naphthyridine-3-carboxylic acid; (S)-6-(3-chloro-2-fluorobenzyl)-1-(1-hydroxy-3,3-dimethylbutan-2-yl)-7-(3-morpholinopropylamino)-4-oxo-1,4-dihydro-1,8-naphthyridine-3-carboxyl is acid; (S)-6-(3-chloro-2-fluorobenzyl)-1-(1-hydroxy-3,3-dimethylbutan-2-yl)-4-oxo-7-(3-(2-oxopyrrolidin-1-yl)propylamino)-1,4-dihydro-1,8-naphthyridine-3-carboxylic acid; (S)-6-(3-chloro-2-fluorobenzyl)-1-(1-hydroxy-3,3-dimethylbutan-2-yl)-4-oxo-7-(pyridin-2-ylmethylamino)-1,4-dihydro-1,8-naphthyridine-3-carboxylic acid; (S)-6-(3-chloro-2-fluorobenzyl)-1-(1-hydroxy-3,3-dimethylbutan-2-yl)-4-oxo-7-(pyridin-2-ylmethylamino)-1,4-dihydro-1,8-naphthyridine-3-carboxylic acid; and (S)-6-(3-chloro-2-fluorobenzyl)-1-(1-hydroxy-3,3-dimethylbutan-2-yl)-7-(3-hydroxypropyl)-4-oxo-1,4-dihydro-1,8-naphthyridine-3-carboxylic acid:

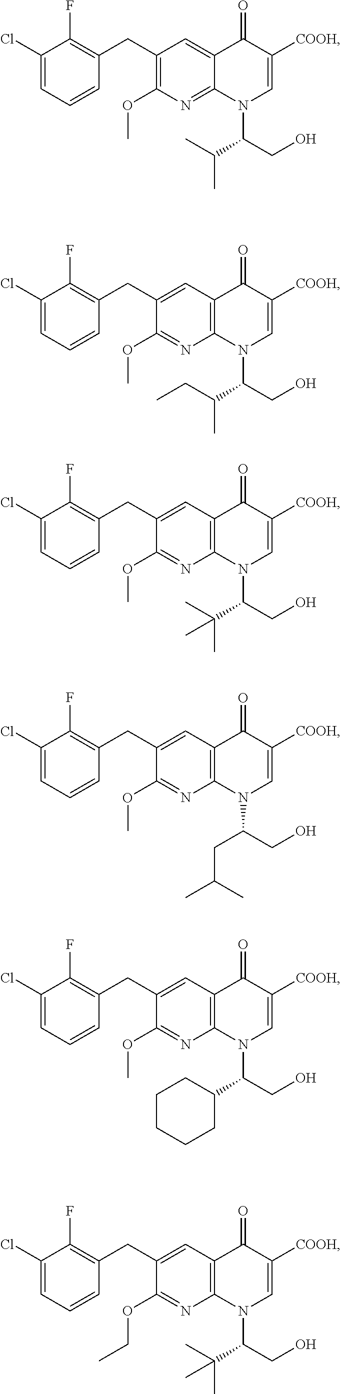

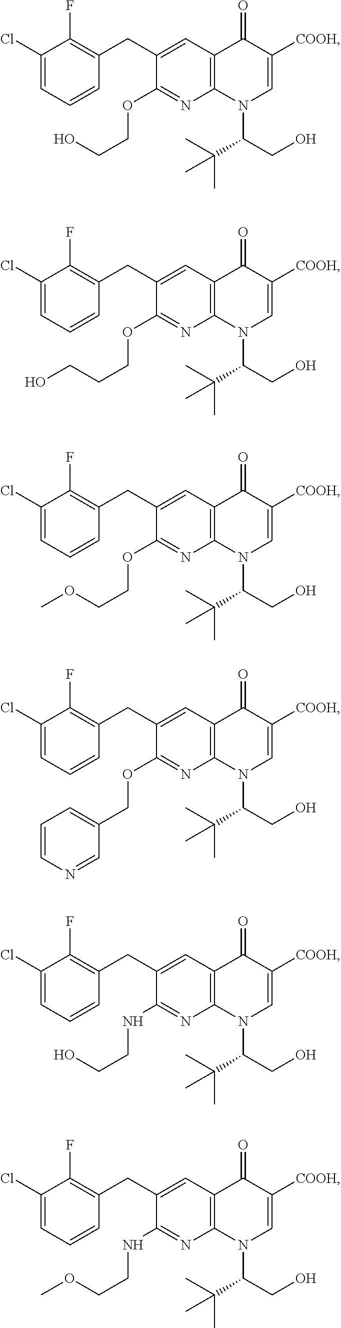

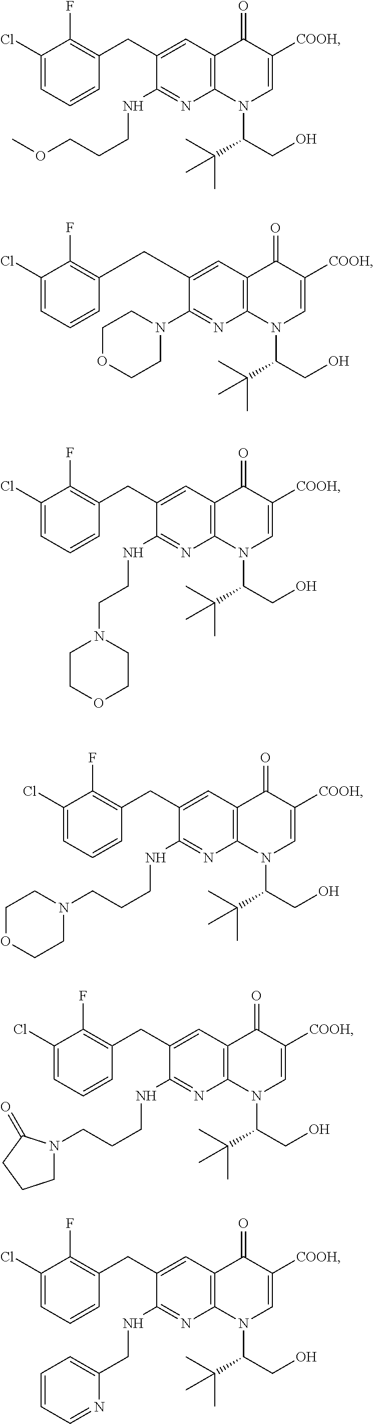

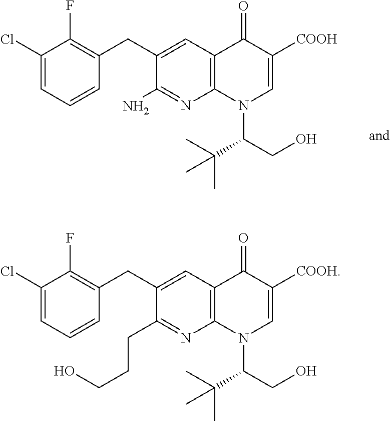

Described herein are compounds of formula (V)(a), metabolites, pharmaceutically acceptable salts, solvates, polymorphs, esters, tautomers or prodrugs thereof,

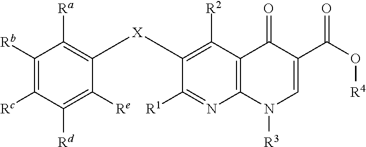

wherein
R¹ is H, F, Cl, Br, I, CFH₂, CF₂H, CF₃, CN, OH, NO₂, NH2, NH (alkyl) or N(alkyl)₂, SO₂CH₃, SO₂NH2, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl, optionally substituted heteroaryl;
R² is H, F, Cl, Br, I, CFH₂, CF₂H, CF₃, CN, OH, NO₂, NH(alkyl) or N(alkyl)₂, SO₂CH₃, SO₂NH2, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl, optionally substituted heteroaryl;
R³ is optionally substituted alkyl, optionally substituted cycloalkyl, optionally substituted heterocycloalkyl, optionally substituted aryl, optionally substituted heteroaryl;
R⁴ is H, alkyl or a pharmaceutically acceptable cation;
X is C(R^{x})(R^{x′}), O, S, S(O), S(O)₂, NH, NR, C(O), C(S), C(N-alkyl), CH₂CH₂, CH₂CH₂CH₂, OCH₂, CH₂O, CH₂OCH₂, OCH₂CH₂, CH₂CH₂O, SCH₂, CH₂S, CH₂SCH₂, SCH₂CH₂, CH₂CH₂S, NHCH₂, CH₂NH, CH₂NHCH₂, NHCH₂CH₂, CH₂CH₂NH, OC(O) or C(O)O;
   wherein
   R^{x} and R^{x′} are independently selected from H, optionally substituted C₁₋₁₀ alkyl, optionally substituted C₃₋₇ cycloalkyl, cyclopropylmethyl, optionally substituted aryl, optionally substituted heterocyclyl and optionally substituted heteroaryl;
   so long as at least one R^{x} is not H; or
   R^{x} and R^{x′} taken together with the C atom to which they are attached form a saturated or unsaturated, substituted or unsubstituted 3-7 member ring optionally comprising 1 or 2 heteroatoms selected from O, S and N; and
   each CH₂ or CH₂CH₂ group is further substituted;
R^{a}, R^{b}, R^{c}, R^{d} and R^{e} are independently selected from H, F, Cl, Br, I, CF₃, CN, alkyl, cycloalkyl, cyclopropylmethyl, NH₂, NHR′, NR′R″, OH, OR′, SH, SR′, C(O)R′, CO₂, COOR′, CONH₂, CONHR′, CONR′R″, SO₃H, S(O)₂R′, S(O)₂NH₂, S(O)₂NHR′, S(O)₂NR′R″, aryl, heterocyclyl and heteroaryl; wherein
   R′ is methyl, ethyl, n-propyl, i-propyl, n-butyl, i-butyl, s-butyl, t-butyl, cyclopropyl, cyclobutyl, cyclopentyl, cyclohexyl or cyclopropylmethyl;
   R″ is methyl, ethyl, n-propyl, i-propyl, n-butyl, i-butyl, s-butyl, t-butyl, cyclopropyl, cyclobutyl, cyclopentyl, cyclohexyl or cyclopropylmethyl; or
   R′ and R″ together with the nitrogen atom to which they are attached form an optionally substituted 4-, 5- or 6-membered heterocyclic ring; and
all CH₂, CH₂CH₂, alkyl, cycloalkyl, heterocyclyl, aryl and heteroaryl moieties are optionally further substituted.

Disclosed herein are compounds of formula (V)(a) and their pharmaceutically acceptable salts. In further or additional embodiments, the disclosed herein are are compounds of formula (V)(a) and their metabolites. In further or additional embodiments, disclosed herein are compounds of formula (V)(a) and their pharmaceutically acceptable solvates. In further or additional embodiments, described herein are Compounds of formula (V)(a) and their pharmaceutically acceptable polymorphs. In further or additional embodiments, disclosed herein are compounds of formula (V)(a) and their pharmaceutically acceptable esters. In further or additional embodiments, disclosed herein are compounds of formula (V)(a) and their pharmaceutically acceptable tautomers. In further or additional embodiments, disclosed herein are compounds of formula (V)(a) and their pharmaceutically acceptable prodrugs.

In some embodiments, X is C(R^{x})(R^{x′}). In further or additional embodiments, X is C(R^{x})(R^{x′}) and R^{x} and R^{x′} taken together with the C atom to which they are attached form a saturated or unsaturated, substituted or unsubstituted 3-7 member ring optionally comprising 1 or 2 heteroatoms selected from O, S and N. In further or additional embodiments, X is C(R^{x})(R^{x′}) and R^{x′} is H. In further or additional embodiments, X is C(R^{x})(R^{x′}), R^{x′} is H and R^{x} is alkyl. In further or additional embodiments, X is O, S, S(O), S(O)₂. In further or additional embodiments, X is CH₂CH₂ or CH₂CH₂CH₂. In further or additional embodiments, X comprises an O atom. In further or additional embodiments, X comprises a S atom. In some embodiments, at least one of R^{a}, R^{b}, R^{c}, R^{d} and R^{e} is F, Cl, Br or I. In further or additional embodiments, at least two of R^{a}, R^{b}, R^{c}, R^{d} and R^{e} are F, Cl, Br or I. In further or additional embodiments, at least three of R^{a}, R^{b}, R^{c}, R^{d} and R^{e} are F, Cl, Br or I. In further or additional embodiments, at least two of R^{a}, R^{b}, R^{c}, R^{d} and R^{e} are F, Cl, Br or I and the other three are H. In further or additional embodiments, one of R^{a}, R^{b}, R^{c}, R^{d} and R^{e} is F, one of R^{a}, R^{b}, R^{c}, R^{d} and R^{e} is Cl and the other three are H. In further or additional embodiments, R^{a} and R^{b} are F, Cl, Br or I and R^{c}, R^{d} and R^{e} are H. In further or additional embodiments, R^{a} and R^{b} are F or Cl and R^{c}, R^{d} and R^{e} are H. In further or additional embodiments, R^{a} is F, R^{b} is Cl and R^{c}, R^{d} and R^{e} are H. In further or additional embodiments, R^{a} is F, R^{b} is Cl, R^{c}, R^{d} and R^{e} are H and X is C(R^{x})(R^{x′}). In some embodiments, R¹ is alkoxy In further or additional embodiments, R¹ is methoxy. In further or additional embodiments, R¹ is ethoxy. In further or additional embodiments, R^{a} is F, R^{b} is Cl, R^{c}, R^{d} and R^{e} are H, X is C(R^{x})(R^{x′}) and R¹ is methoxy. In some embodiments, R² is H, CN, OH, or C₁₋₄ alkoxy. In further or additional embodiments, R² is H. In further or additional embodiments, R^{a} is F, R^{b} is Cl, R^{c}, R^{d} and R^{e} are H, X is C(R^{x})(R^{x′}), R¹ is methoxy and R² is H. In some embodiments, R³ is alkyl. In some embodiments, R³ is substituted alkyl. In further or additional embodiments, R³ is C₁₋₁₀ alkyl. In further or additional embodiments, R³ is substituted C₁₋₁₀ alkyl. In further or additional embodiments, R³ is C₃₋₇ alkyl. In further or additional embodiments, R³ is substituted C₃₋₇ alkyl. In further or additional embodiments, R³ is n-pentyl, iso-pentyl, neo-pentyl or tert-pentyl. In further or additional embodiments, R³ is substituted n-pentyl, iso-pentyl, neo-pentyl or tert-pentyl. In further or additional embodiments, R³ is alkyl substituted with one or more hydroxy or alkoxy groups. In further or additional embodiments, R³ is alkyl substituted with one or two hydroxy groups. In further or additional embodiments, R³ is pentyl substituted with one or more hydroxy or alkoxy groups. In further or additional embodiments, R³ is pentyl substituted with one or two hydroxy groups. In further or additional embodiments, R³ is pentyl substituted with one hydroxy group. In further or additional embodiments, R³ is 2-(3-methyl-1-hydroxybutyl). In further or additional embodiments, R³ is (R)-2-(3-methyl-1-hydroxybutyl). In further or additional embodiments, R³ is (S)-2-(3-methyl-1-hydroxybutyl). In further or additional embodiments, R³ is

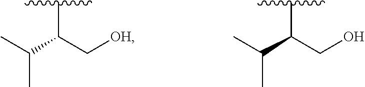

or a mixture of both. In further or additional embodiments, R^{a} is F, R^{b} is Cl, R^{c}, R^{d} and R^{e} are H, X is C(R^{x})(R^{x′}), R¹ is methoxy, R² is H and R³ is pentyl substituted with one hydroxy group. In further or additional embodiments, R⁴ is H or alkyl. In further or additional embodiments, R⁴ is H. In further or additional embodiments, R^{a} is F, R^{b} is Cl, R^{c}, R^{d} and R^{e} are H, X is C(R^{x})(R^{x′}), R¹ is methoxy, R² is H, R³ is pentyl substituted with one hydroxy group and R⁴ is H. In any of the embodiments described above, the compound of formula (V)(a) is less than about 50%, less than about 40%, less than about 30%, less than about 25%, less than about 20%, less than about 15%, less than about 10%, less than about 7.5%, less than about 5%, degraded after exposure to pooled human liver microsomes (protein: 1 mg/mL with CYP3A4 activity at about 7800 pmol/min/mg) at 37° C. for 60 minutes at pH 7.4 at a compound concentration of 1 μM in potassium phosphate buffer (100 mM) containing magnesium chloride (5 mM), EDTA (100 μM) and NADPH (1 mM). In any of the embodiments described above, the compound of formula (V)(a) is less than about 50%, less than about 40%, less than about 30%, less than about 25%, less than about 20%, less than about 15%, less than about 10%, less than about 7.5%, less than about 5%, degraded after exposure to 10 pmol CYP3A4 enzyme at 37° C. for 60 minutes at pH 7.4 in potassium phosphate buffer (100 mM) containing magnesium chloride (5 mM), EDTA (100 μM) and NADPH (1 mM).

Also described herein are compounds of formula (V)(b), metabolites, pharmaceutically acceptable salts, solvates, polymorphs, esters, tautomers or prodrugs thereof,

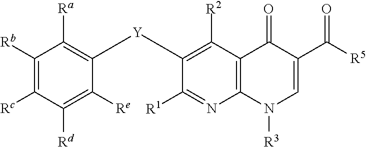

wherein
R¹ is H, F, Cl, Br, I, CFH₂, CF₂H, CF₃, CN, OH, NO₂, NH2, NH(alkyl) or N(alkyl)₂, SO₂CH₃, SO₂NH2, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl, optionally substituted heteroaryl;
R² is H, F, Cl, Br, I, CFH₂, CF₂H, CF₃, CN, OH, NO₂, NH₂, NH(alkyl) or N(alkyl)₂, SO₂CH₃, SO₂NH₂, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl, optionally substituted heteroaryl;
R³ is optionally substituted alkyl, optionally substituted cycloalkyl, optionally substituted heterocycloalkyl, optionally substituted aryl, optionally substituted heteroaryl;
R⁵ is CF₃, NH₂, NH(alkyl) or N(alkyl)₂, optionally substituted alkyl, optionally substituted alkenyl, optionally substituted S-alkyl, optionally substituted cycloalkyl, optionally substituted heterocycle, optionally substituted aryl, optionally substituted heteroaryl;
Y is C(R^{x})(R^{x′}), O, S, S(O), S(O)₂, NH, NR, C(O), C(S), C(N-alkyl), CH₂CH₂, CH₂CH₂CH₂, OCH₂, CH₂O, CH₂OCH₂, OCH₂CH₂, CH₂CH₂O, SCH₂, CH₂S, CH₂SCH₂, SCH₂CH₂, CH₂CH₂S, NHCH₂, CH₂NH, CH₂NHCH₂, NHCH₂CH₂, CH₂CH₂NH, OC(O) or C(O)O;
   wherein
   R^{x} and R^{x′} are independently selected from H, optionally substituted C₁₋₁₀ alkyl, optionally substituted C₃₋₇ cycloalkyl, cyclopropylmethyl, optionally substituted aryl, optionally substituted heterocyclyl and optionally substituted heteroaryl; or
   R^{x} and R^{x′} taken together with the C atom to which they are attached form a saturated or unsaturated, substituted or unsubstituted 3-7 member ring optionally comprising 1 or 2 heteroatoms selected from O, S and N;
R^{a}, R^{b}, R^{c}, R^{d} and R^{e} are independently selected from H, F, Cl, Br, I, CF₃, CN, alkyl, cycloalkyl, cyclopropylmethyl, NH₂, NHR′, NR′R″, OH, OR′, SH, SR′, C(O)R′, CO₂H, COOR′, CONH₂, CONHR′, CONR′R″, SO₃H, S(O)₂R′, S(O)₂NH₂, S(O)₂NHR′, S(O)₂NR′R″, aryl, heterocyclyl and heteroaryl; wherein
   R¹ is methyl, ethyl, n-propyl, i-propyl, n-butyl, i-butyl, s-butyl, t-butyl, cyclopropyl, cyclobutyl, cyclopentyl, cyclohexyl or cyclopropylmethyl;
   R″ is methyl, ethyl, n-propyl, i-propyl, n-butyl, i-butyl, s-butyl, t-butyl, cyclopropyl, cyclobutyl, cyclopentyl, cyclohexyl or cyclopropylmethyl; or
   R′ and R″ together with the nitrogen atom to which they are attached form an optionally substituted 4-, 5- or 6-membered heterocyclic ring; and
all CH₂, CH₂CH₂, alkyl, cycloalkyl, heterocyclyl, aryl and heteroaryl moieties are optionally further substituted.

Disclosed herein are compounds of formula (V)(b) and their pharmaceutically acceptable salts. In further or additional embodiments, disclosed herein are compounds of formula (V)(b) and their metabolites. In further or additional embodiments, disclosed herein are compounds of formula (V)(b) and their pharmaceutically acceptable solvates. In further or additional embodiments, disclosed herein are compounds of formula (V)(b) and their pharmaceutically acceptable polymorphs. In further or additional embodiments, disclosed herein are compounds of formula (V)(b) and their pharmaceutically acceptable esters. In further or additional embodiments, disclosed herein are compounds of formula (V)(b) and their pharmaceutically acceptable tautomers. In further or additional embodiments, disclosed herein are compounds of formula (V)(b) and their pharmaceutically acceptable prodrugs.

In some embodiments, Y is CH₂, CH₂CH₂ or CH₂CH₂CH₂. In further or additional embodiments, Y is CH₂. In further or additional embodiments, Y is C(R^{x})(R^{x′}). In further or additional embodiments, Y is O, S, S(O), S(O)₂. In further or additional embodiments, Y is C(R^{x})(R^{x′}) and R^{x} and R^{x′} taken together with the C atom to which they are attached form a saturated or unsaturated, substituted or unsubstituted 3-7 member ring optionally comprising 1 or 2 heteroatoms selected from O, S and N. In further or additional embodiments, Y is C(R^{x})(R^{x′}) and R^{x′} is H. In further or additional embodiments, Y is C(R^{x})(R^{x′}), R^{x′} is H and R^{x} is alkyl. In further or additional embodiments, Y is O, S, S(O), S(O)₂. In further or additional embodiments, Y comprises an O atom. In further or additional embodiments, Y comprises a S atom. In some embodiments, at least one of R^{a}, R^{b}, R^{c}, R^{d} and R^{e} is F, Cl, Br or I. In further or additional embodiments, at least two of R^{a}, R^{b}, R^{c}, R^{d} and R^{e} are F, Cl, Br or I. In further or additional embodiments, at least three of R^{a}, R^{b}, R^{c}, R^{d} and R^{e} are F, Cl, Br or I. In further or additional embodiments, at least two of R^{a}, R^{b}, R^{c}, R^{d} and R^{e} are F, Cl, Br or I and the other three are H. In further or additional embodiments, one of R^{a}, R^{b}, R^{c}, R^{d} and R^{e} is F, one of R^{a}, R^{b}, R^{c}, R^{d} and R^{e} is Cl and the other three are H. In further or additional embodiments, R^{a} and R^{b} are F, Cl, Br or I and R^{c}, R^{d} and R^{e} are H. In further or additional embodiments, R^{a} and R^{b} are F or Cl and R^{c}, R^{d} and R^{e} are H. In further or additional embodiments, R^{a} is F, R^{b} is Cl and R^{c}, R^{d} and R^{e} are H. In further or additional embodiments, R^{a} is F, R^{b} is Cl, R^{c}, R^{d} and R^{e} are H and Y is CH₂. In some embodiments, R¹ is alkoxy. In further or additional embodiments, R¹ is methoxy. In further or additional embodiments, R¹ is ethoxy. In further or additional embodiments, R^{a} is F, R^{b} is Cl, R^{c}, R^{d} and R^{e} are H, Y is CH₂ and R¹ is methoxy. In some embodiments, R² is H, CN, OH, or C₁₋₄ alkoxy. In further or additional embodiments, R² is H. In further or additional embodiments, R^{a} is F, R^{b} is Cl, R^{c}, R^{d} and R^{e} are H, Y is CH₂, R¹ is methoxy and R² is H. In some embodiments, R³ is alkyl. In some embodiments, R³ is substituted alkyl. In further or additional embodiments, R³ is C₁₋₁₀ alkyl. In further or additional embodiments, R³ is substituted C₁₋₁₀ alkyl. In further or additional embodiments, R³ is C₃₋₇ alkyl. In further or additional embodiments, R³ is substituted C₃₋₇ alkyl. In further or additional embodiments, R³ is n-pentyl, iso-pentyl, neo-pentyl or tert-pentyl. In further or additional embodiments, R³ is substituted n-pentyl, iso-pentyl, neo-pentyl or tert-pentyl. In further or additional embodiments, R³ is alkyl substituted with one or more hydroxy or alkoxy groups. In further or additional embodiments, R³ is alkyl substituted with one or two hydroxy groups. In further or additional embodiments, R³ is pentyl substituted with one or more hydroxy or alkoxy groups. In further or additional embodiments, R³ is pentyl substituted with one or two hydroxy groups. In further or additional embodiments, R³ is pentyl substituted with one hydroxy group. In further or additional embodiments, R³ is 2-(3-methyl-1-hydroxybutyl). In further or additional embodiments, R³ is (R)-2-(3-methyl-1-hydroxybutyl). In further or additional embodiments, R³ is (S)-2-(3-methyl-1-hydroxybutyl).

In further or additional embodiments, R³ is

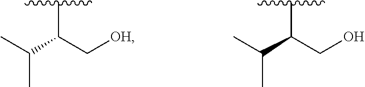

or a mixture of both. In further or additional embodiments, R^{a} is F, R^{b} is Cl, R^{c}, R^{d} and R^{e} are H, Y is CH₂, R¹ is methoxy, R² is H and R³ is pentyl substituted with one hydroxy group. In further or additional embodiments, R⁴ is 11 or alkyl. In further or additional embodiments, R⁴ is H. In further or additional embodiments, R^{a} is F, R^{b} is Cl, R^{c}, R^{d} and R^{e} are H, Y is CH₂, R¹ is methoxy, R² is H, R³ is pentyl substituted with one hydroxy group and R⁴ is H. In any of the embodiments described above, the compound of formula (V)(b) is less than about 50%, less than about 40%, less than about 30%, less than about 25%, less than about 20%, less than about 15%, less than about 10%, less than about 7.5%, less than about 5%, degraded after exposure to pooled human liver microsomes (protein: 1 mg/mL with CYP3A4 activity at about 7800 pmol/min/mg) at 37° C. for 60 minutes at pH 7.4 at a compound concentration of 1 μM in potassium phosphate buffer (100 mM) containing magnesium chloride (5 mM), EDTA (100 μM) and NADPH (1 mM). In any of the embodiments described above, the compound of formula (V)(b) is less than about 50%, less than about 40%, less than about 30%, less than about 25%, less than about 20%, less than about 15%, less than about 10%, less than about 7.5%, less than about 5%, degraded after exposure to 10 pmol CYP3A4 enzyme at 37° C. for 60 minutes at pH 7.4 in potassium phosphate buffer (100 mM) containing magnesium chloride (5 mM), EDTA (100 μM) and NADPH (1 mM).

Also described herein are compounds of formula (V)(c), metabolites, pharmaceutically acceptable salts, solvates, polymorphs, esters, tautomers or prodrugs thereof,

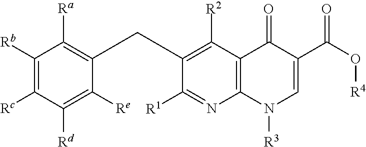

wherein
R¹ is H, F, Cl, Br, I, CFH₂, CF₂H, CF₃, CN, OH, NO₂, NH2, NH(alkyl) or N(alkyl)₂, SO₂CH₃, SO₂NH2, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl, optionally substituted heteroaryl;
R² is H, F, Cl, Br, I, CFH₂, CF₂H, CF₃, CN, OH, NO₂, NH2, NH(alkyl) or N(alkyl)₂, SO₂CH₃, SO₂NH2, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl, optionally substituted heteroaryl;
R³ is optionally substituted alkyl, optionally substituted cycloalkyl, optionally substituted heterocycloalkyl, optionally substituted aryl, optionally substituted heteroaryl;
R⁴ is H, alkyl or a pharmaceutically acceptable cation;
R^{a}, R^{b}, R^{c}, R^{d} and R^{e} are independently selected from H, F, Cl, Br, I, CF₃, CN, alkyl, cycloalkyl, cyclopropylmethyl, NH2, NHR′, NR′R″, OH, OR′, SH, SR′, C(O)R′, CO₂H, COOR′, CONH2, CONHR′, CONR′R″, SO₃H, S(O)₂R′, S(O)₂NH₂, S(O)₂NHR′, S(O)₂NR′R″, aryl, heterocyclyl and heteroaryl; wherein
   R′ is methyl, ethyl, n-propyl, i-propyl, n-butyl, i-butyl, s-butyl, t-butyl, cyclopropyl, cyclobutyl, cyclopentyl, cyclohexyl or cyclopropylmethyl;
   R″ is methyl, ethyl, n-propyl, n-butyl, i-butyl, s-butyl, t-butyl, cyclopropyl, cyclobutyl, cyclopentyl, cyclohexyl or cyclopropylmethyl; or
   R′ and R″ together with the nitrogen atom to which they are attached form an optionally substituted 4-, 5- or 6-membered heterocyclic ring; and
provided that the compound is not:

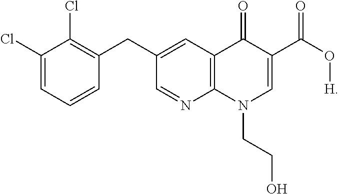

Disclosed herein are compounds of formula (V)(c) and their pharmaceutically acceptable salts. In further or additional embodiments, disclosed herein are compounds of formula (V)(c) and their metabolites. In further or additional embodiments, disclosed herein are compounds of formula (V)(c) and their pharmaceutically acceptable solvates. In further or additional embodiments, disclosed herein are compounds of formula (V)(c) and their pharmaceutically acceptable polymorphs. In further or additional embodiments, disclosed herein are compounds of formula (V)(c) and their pharmaceutically acceptable esters. In further or additional embodiments, disclosed herein are compounds of formula (V)(c) and their pharmaceutically acceptable tautomers. In further or additional embodiments, disclosed herein are compounds of formula (V)(c) and their pharmaceutically acceptable prodrugs.

In some embodiments, at least one of R^{a}, R^{b}, R^{c}, R^{d} and R^{e} is F, Cl, Br or I. In further or additional embodiments, at least two of R^{a}, R^{b}, R^{c}, R^{d} and R^{e} are F, Cl, Br or I. In further or additional embodiments, at least three of R^{a}, R^{b}, R^{c}, R^{d} and R^{e} are F, Cl, Br or I. In further or additional embodiments, at least two of R^{a}, R^{b}, R^{c}, R^{d} and R^{e} are F, Cl, Br or I and the other three are H. In further or additional embodiments, one of R^{a}, R^{b}, R^{c}, R^{d} and R^{e} is F, one of R^{a}, R^{b}, R^{c}, R^{d} and R^{e} is Cl and the other three are H. In further or additional embodiments, R^{a} and R^{b} are F, Cl, Br or I and R^{c}, R^{d} and R^{e} are H. In further or additional embodiments, R^{a} and R^{b} are F or Cl and R^{c}, R^{d} and R^{e} are H. In further or additional embodiments, R^{a} is F, R^{b} is Cl and R^{c}, R^{d} and R^{e} are H. In further or additional embodiments, R^{a} is F, R^{b} is Cl, R^{c}, R^{d} and R^{e} are H and Y is CH₂. In some embodiments, R¹ is alkoxy. In some embodiments, R¹ is alkoxy. In further or additional embodiments, R¹ is methoxy. In further or additional embodiments, R¹ is ethoxy. In further or additional embodiments, R^{a} is F, R^{b} is Cl, R^{c}, R^{d} and R^{e} are H and R¹ is methoxy. In some embodiments, R² is H, CN, OH, or C₁₋₄ alkoxy. In further or additional embodiments, R² is H. In further or additional embodiments, R^{a} is F, R^{b} is Cl, R^{c}, R^{d} and R^{e} are H, R¹ is methoxy and R² is H. In some embodiments, R³ is alkyl. In some embodiments, R³ is substituted alkyl. In further or additional embodiments, R³ is C₁₋₁₀ alkyl. In further or additional embodiments, R³ is substituted C₁₋₁₀ alkyl. In further or additional embodiments, R³ is C₃₋₇ alkyl. In further or additional embodiments, R³ is substituted C₃₋₇ alkyl. In further or additional embodiments, R³ is n-pentyl, iso-pentyl, neo-pentyl or tort-pentyl. In further or additional embodiments, R³ is substituted n-pentyl, iso-pentyl, neo-pentyl or tert-pentyl. In further or additional embodiments, R³ is alkyl substituted with one or more hydroxy or alkoxy groups. In further or additional embodiments, R³ is alkyl substituted with one or two hydroxy groups. In further or additional embodiments, R³ is pentyl substituted with one or more hydroxy or alkoxy groups. In further or additional embodiments, R³ is pentyl substituted with one or two hydroxy groups. In further or additional embodiments, R³ is pentyl substituted with one hydroxy group. In further or additional embodiments, R³ is 2-(3-methyl-1-hydroxybutyl). In further or additional embodiments, R³ is (R)-2-(3-methyl-1-hydroxybutyl). In further or additional embodiments, R³ is (S)-2-(3-methyl-1-hydroxybutyl). In further or additional embodiments, R³ is

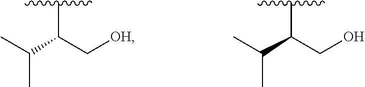

or a mixture of both. In further or additional embodiments, R^{a} is F, R^{b} is Cl, R^{c}, R^{d} and R^{e} are H, R¹ is methoxy, R² is H and R³ is pentyl substituted with one hydroxy group. In further or additional embodiments, R⁴ is H or alkyl. In further or additional embodiments, R⁴ is H. In further or additional embodiments, R^{a} is F, R^{b} is Cl, R^{c}, R^{d} and R^{e} are H, R¹ is methoxy, R² is H, R³ is pentyl substituted with one hydroxy group and R⁴ is H. In any of the embodiments described above, the compound of formula (V)(c) is less than about 50%, less than about 40%, less than about 30%, less than about 25%, less than about 20%, less than about 15%, less than about 10%, less than about 7.5%, less than about 5%, degraded after exposure to pooled human liver microsomes (protein: 1 mg/mL with CYP3A4 activity at about 7800 pmol/min/mg) at 37° C. for 60 minutes at pH 7.4 at a compound concentration of 1 μM in potassium phosphate buffer (100 mM) containing magnesium chloride (5 mM), EDTA (100 μM) and NADPH (1 mM). In any of the embodiments described above, the compound of formula (V)(c) is less than about 50%, less than about 40%, less than about 30%, less than about 25%, less than about 20%, less than about 15%, less than about 10%, less than about 7.5%, less than about 5%, degraded after exposure to 10 pmol CYP3A4 enzyme at 37° C. for 60 minutes at pH 7.4 in potassium phosphate buffer (100 mM) containing magnesium chloride (5 mM), EDTA (100 μM) and NADPH (1 mM).

In further or additional embodiments, the compound of formula (V)(c) is selected from

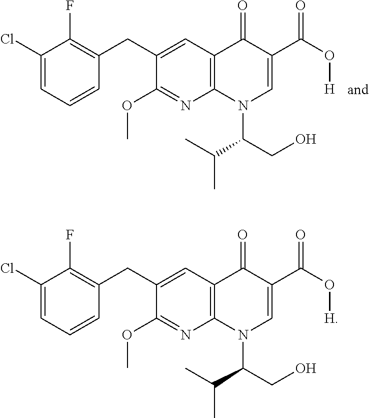

### Synthetic Procedures

In another aspect, methods for synthesizing a compound of formula (I) or (II); formula (III) or (IV); or formula (V)(a), (V)(b) or (V)(c) are provided. In some embodiments, a compound of formula (I) or (II); formula (III) or (IV); or formula (V)(a), (V)(b) or (V)(c) are prepared by the methods described below. The procedures and examples below are intended to illustrate those methods. Neither the procedures nor the examples should be construed as limiting the disclosures herein in any way. In some embodiments, compounds described herein are synthesized using any suitable method.

The starting materials used for the synthesis of the compounds as described herein are obtained from commercial sources, such as Aldrich Chemical Co. (Milwaukee, Wis.), Sigma Chemical Co. (St. Louis, Mo.), or the starting materials are synthesized. A compound of formula (I) or (II); formula (III) or (IV); or formula (V)(a), (V)(b) or (V)(c), and other related compounds having different substituents are synthesized using any suitable techniques and materials, such as described, for example, in March, ADVANCED ORGANIC CHEMISTRY 4^{th} Edition (John Wiley and Sons, 1992); Carey and Sundberg, ADVANCED ORGANIC CHEMISTRY 4^{th} Edition, Vols. A and B (Plenum, 2000, 2001), and Green and Wuts, PROTECTIVE GROUPS IN ORGANIC SYNTHESIS, 3^{rd} (Edition (John Wiley and Sons, 1999) (all of which are incorporated by reference for such subject matter). General methods for the preparation of compound as disclosed herein are derived from known reactions in the field, and the reactions are modified by the use of appropriate reagents and conditions for the introduction of the various moieties found in the formulae as provided herein. In some embodiments, the following synthetic methods are utilized.

Formation of Covalent Linkages by Reaction of an Electrophile with a Nucleophile

A compound of formula (I) or (II); formula (III) or (IV); or formula (V)(a), (V)(b) or (V)(c) are modified using various electrophiles or nucleophiles to form new functional groups or substituents. The table below entitled “Examples of Covalent Linkages and Precursors Thereof” lists selected examples of covalent linkages and precursor functional groups which yield and are used as guidance toward the variety of electrophiles and nucleophiles combinations available. Precursor functional groups are shown as electrophilic groups and nucleophilic groups.

| Covalent Linkage | | |
|---|---|---|
| Product | Electrophile | Nucleophile |
| Carboxamides | Activated esters | Amines/anilines |
| Carboxamides | Acyl azides | Amines/anilines |
| Carboxamides | Acyl halides | Amines/anilines |
| Esters | Acyl halides | Alcohols/phenols |
| Esters | Acyl nitriles | Alcohols/phenols |
| Carboxamides | Acyl nitriles | Amines/anilines |
| Imines | Aldehydes | Amines/anilines |
| Hydrazones | Aldehydes or | Hydrazines |
| | ketones | |
| Oximes | Aldehydes or | Hydroxylamines |
| | ketones | |
| Alkyl amines | Alkyl halides | Amines/anilines |
| Esters | Alkyl halides | Carboxylic acids |
| Thioethers | Alkyl halides | Thiols |
| Ethers | Alkyl halides | Alcohols/phenols |
| Thioethers | Alkyl sulfonates | Thiols |
| Esters | Alkyl sulfonates | Carboxylic acids |
| Ethers | Alkyl sulfonates | Alcohols/phenols |
| Esters | Anhydrides | Alcohols/phenols |
| Carboxamides | Anhydrides | Amines/anilines |
| Thiophenols | Aryl halides | Thiols |
| Aryl amines | Aryl halides | Amines |
| Thioethers | Aziridines | Thiols |
| Boronate esters | Boronates | Glycols |
| Carboxamides | Carboxylic acids | Amines/anilines |
| Esters | Carboxylic acids | Alcohols |
| Hydrazines | Hydrazides | Carboxylic acids |
| N-acylureas or | Carbodiimides | Carboxylic acids |
| Anhydrides | | |
| Esters | Diazoalkanes | Carboxylic acids |
| Thioethers | Epoxides | Thiols |
| Thioethers | Haloacetamides | Thiols |
| Ammotriazines | Halotriazines | Amines/anilines |
| Triazinyl ethers | Halotriazines | Alcohols/phenols |
| Amidines | Imido esters | Amines/anilines |
| Ureas | Isocyanates | Amines/anilines |
| Urethanes | Isocyanates | Alcohols/phenols |
| Thioureas | Isothiocyanates | Amines/anilines |
| Thioethers | Maleimides | Thiols |
| Phosphite esters | Phosphoramidites | Alcohols |
| Silyl ethers | Silyl halides | Alcohols |
| Alkyl amines | Sulfonate esters | Amines/anilines |
| Thioethers | Sulfonate esters | Thiols |
| Esters | Sulfonate esters | Carboxylic acids |
| Ethers | Sulfonate esters | Alcohols |
| Sulfonamides | Sulfonyl halides | Amines/anilines |
| Sulfonate esters | Sulfonyl halides | Phenols/alcohols |

### Examples of Covalent Linkages and Precursors Thereof

### Use of Protecting Groups

In some embodiments, it is necessary to protect reactive functional groups, for example hydroxy, amino, imino, thio or carboxy groups, where these are desired in the final product, to avoid their unwanted participation in the reactions. Protecting groups are used to block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups are removed by acid, base, and hydrogenolysis. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, or they are blocked with oxidatively-removable protective groups such as 2,4-dimethoxybenzyl, while co-existing amino groups are blocked with fluoride labile silyl carbamates.

Allyl blocking groups are useful in then presence of acid- and base-protecting groups since the former are stable and are subsequently removed by metal or pi-acid catalysts. For example, an allyl-blocked carboxylic acid are deprotected with a Pd-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and cannot react. Once released from the resin, the functional group is available to react.

Protecting or blocking groups are selected from:

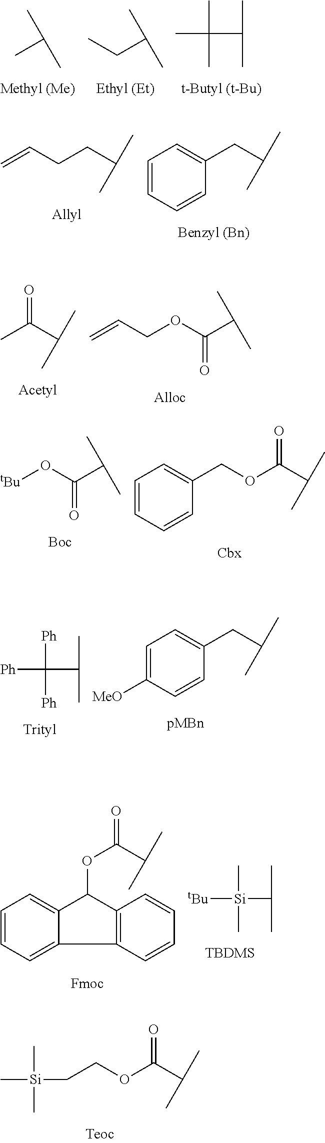

Other protecting groups, plus a detailed description of techniques applicable to the creation of protecting groups and their removal are described in Greene and Wuts, PROTECTIVE GROUPS IN ORGANIC SYNTHESIS, 3^{rd} Edition (John Wiley and Sons, 1999), and Kocienski, PROTECTIVE GROUPS (Thieme Verlag, 1994), which are incorporated herein by reference for such subject matter.

### Preparing Compounds of Formula (I) or (II); Formula (III) or (IV); or Formula (V)(a), (V)(b) or (V)(c)

Described herein are processes for the preparation of compounds of formula (I) or (II); formula (III) or (IV); or formula (V)(a), (V)(b) or (V)(c), which are synthesized according to the reaction schemes below.

### I. Preparation of Compounds of Formula (I)

### Example 1

### Compounds of Formula (I)

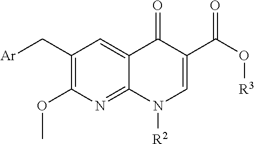

Compounds of formula (I) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

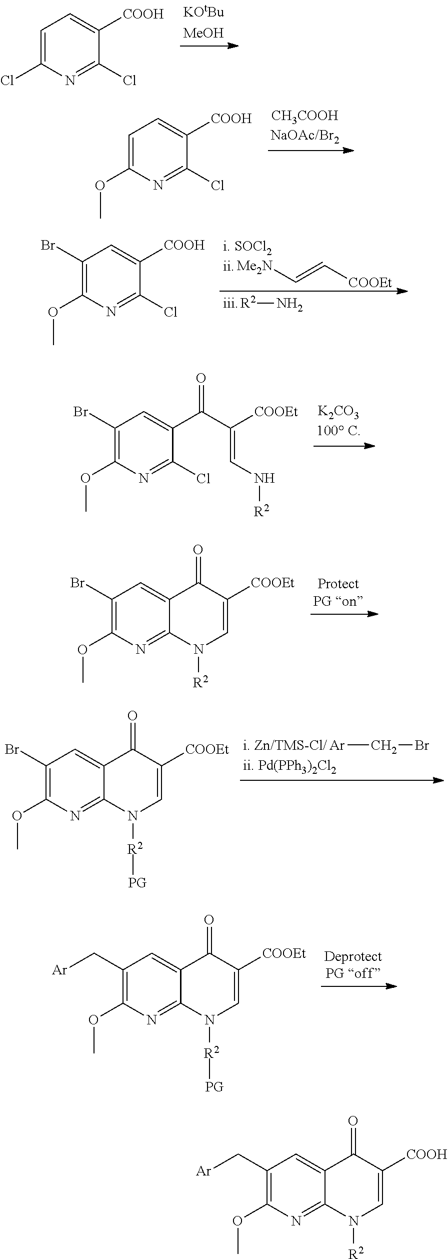

### Example 1A

### 6-(3-Chloro-2-fluoro-benzyl)-1-((S)-1-hydroxymethyl-2-methyl-propyl)-7-methoxy-4-oxo-1,4-dihydro-[1,8]naphthyridine-3-carboxylic acid

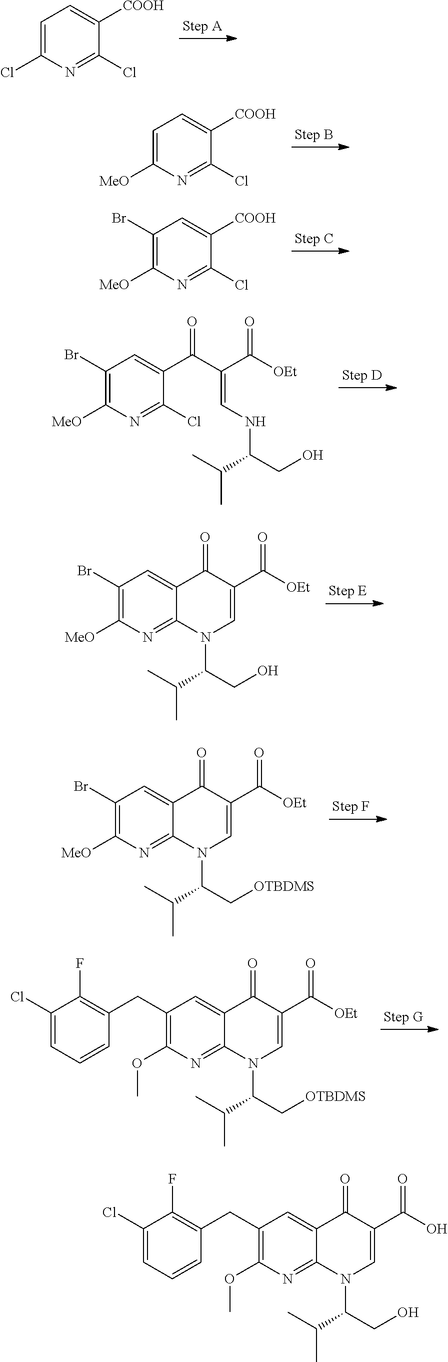

### Step A: 2-Chloro-6-methoxypyridine-3-carboxylic acid

A mixture of 2,6-dichloropyridine-3-carboxylic acid (6.5 g, 33 mmol), potassium tert-butoxide (11.4 g, 0.10 mol), and anhydrous methanol (300 mL) was heated to reflux for 4 days and cooled to room temperature. After evaporation of the solvent, the residue was diluted with water, and acidified with 35% aqueous hydrochloric acid. The resulting solid was collected by filtration, washed with water, and dried to give 4.8 g (84%) of 2-chloro-6-methoxy pyridine-3-carboxylic acid as a white solid.

### Step B: 2-Chloro-5-bromo-6-methoxypyridine-3-carboxylic acid

To a suspension of 2-chloro-6-methoxypyridine-3-carboxylic acid (4.69 g, 25 mmol) and sodium acetate (4.10 g, 50 mmol) in 200 ml of glacial acetic acid was added bromine (16.0, 100 mmol) at room temperature. The mixture was warmed to 80° C. overnight, cooled to room temperature and poured into 500 ml of ice-water with strong stirring. The solid was filtered and washed with water to give 5.2 g (78%) of pure product as a white solid.

### Step C: 2-(5-Bromo-2-chloro-6-methoxy-pyridine-3-carbonyl)-3-((S)-1-hydroxymethyl-2-methyl-propylamino)-acrylic acid ethyl ester

A mixture of 2-chloro-5-bromo-6-methoxypyridine-3-carboxylic acid (8.0 g, 30 mmol) and thionyl chloride (4.4 mL, 60 mmol) in 50 ml of anhydrous toluene and 0.5 ml of anhydrous DMF was refluxed for 2 h. The solvent was removed under reduced pressure to give a mobile oil residue which was azeoptoped with toluene (20 mL). The residue was dissolved in 20 ml of anhydrous THF. This solution was added dropwise to a solution of ethyl 3-(dimethylamino)acrylate (4.7 g, 33 mmol) and triethylamine (3.64 g, 36 mmol) in 20 ml of anhydrous THF under nitrogen and heated under reflux for 7 hours The mixture was allowed to cool to room temperature and concentrated under reduced pressure. Water (100 mL) and ethyl acetate (100 mL) was added to allow partitioning. The organic layer was washed with saturated aqueous sodium bicarbonate (×2), water, brine, dried over sodium sulfate and concentrated under reduced pressure. The crude product was purified by flash chromatography (ISCO, chloroform/methanol, 0-40%, 40 min) to give the pure product as yellow oil (7.3 g, 62%).

A solution of the above product (7.3 g, 18.6 mmol) and L-valinol (1.92 g, 18.6 mmol) in anhydrous THF (100 mL) was stirred for 30 min at room temperature and evaporated to dryness to give a crude product in a quantitative yield, which was used for next step without further purification. An analytically pure sample was prepared by silica gel chromatography (ISCO, Chloroform/methanol, 0-40%, 40 min) to give the pure compound as yellow oil.

### Step D: 6-Bromo-1-((S)-1-hydroxymethyl-2-methyl-propyl)-7-methoxy-4-oxo-1,4-dihydro-[1,8]naphthyridine-3-carboxylic acid ethyl ester

A mixture of 2-(5-bromo-2-chloro-6-methoxy-pyridine-3-carbonyl)-3-((S)-1-hydroxymethyl-2-methyl-propylamino)-acrylic acid ethyl ester (1.1 g, 2.5 mmol) and potassium carbonate (0.7 g, 5.0 mmol) in anhydrous DMF (15 mL) was stirred at 100° C. for 2 hours and evaporated to dryness under reduced pressure. The crude Material was purified by ISCO (Chloroform/methanol, 0-40%, 40 min) to give the title compound as a yellow solid (0.7 g, 68%).

### Step E: 6-Bromo-1-[(S)-1-(tert-butyl-dimethyl-silanyloxymethyl)-2-methyl-propyl]-7-methoxy-4-oxo-1,4-dihydro-[1,8]naphthyridine-3-carboxylic acid ethyl ester

To a mixture of 6-bromo-1-((S)-1-hydroxymethyl-2-methyl-propyl)-7-methoxy-4-oxo-1,4-dihydro-[1,8]naphthyridine-3-carboxylic acid ethyl ester (0.63 g, 1.5 mmol) and imidazole (1.04 g, 15.0 mmol) in 12 ml of anhydrous DMF was added tert-butyldimethylsilyl chloride (1.28 g, 7.5 mmol) under argon at room temperature. The resulting mixture was stirred at room temperature overnight and evaporated to dryness under reduced pressure. The resulting crude material was purified by ISCO (hexane/EtOAc, 0-90%, 40 min) to give the title compound as yellow oil (0.7 g, 89%).

### Step F: 1-[(S)-1-(tert-Butyl-dimethyl-silanyloxymethyl)-2-methyl-propyl]-6-(3-chloro-2-fluoro-benzyl)-7-methoxy-4-oxo-1,4-dihydro-[1,8]naphthyridine-3-carboxylic acid ethyl ester

Under an argon stream, zinc powder (240 mg, 3.67 mmol) was suspended in 0.5 ml of dry tetrahydrofuran and the suspension was heated at 60° C. 1,2-Dibromoethane (0.7 μl, 0.008 mmol) and trimethylsilyl chloride (2.0 μl, 0.016 mmol) were added at this temperature and the mixture was stirred for an additional 30 min followed by addition dropwise of a solution of 2-fluoro-3-chloro-benzyl bromide (176 mg, 0.79 mmol) in 1 ml of dry tetrahydrofuran. The mixture was stirred for an additional hour and allowed to cool to room temperature to give a solution of 2-fluoro-3-chloro-benzylzinc bromide in tetrahydrofuran. This solution was used in the next step.

6-Bromo-1-[(S)-1-(tert-butyl-dimethyl-silanyloxymethyl)-2-methyl-propyl]-7-methoxy-4-oxo-1,4-dihydro-[1,8]naphthyridine-3-carboxylic acid ethyl ester (320 mg, 0.61 mmol) and dichlorobis(triphenylphosphine)palladium(II) (17 mg, 0.024 mmol) were added to 9 ml of dry tetrahydrofuran under an argon stream. The solution prepared above was added at 60° C. and the mixture was stirred with heating at the same temperature for 1.5 hour. The reaction mixture was allowed to cool to room temperature, and 1 N hydrochloric acid was added. The resulting mixture was extracted three times with ethyl acetate. The organic layers were combined, washed with water, brine, dried over anhydrous sodium sulfate and concentrated under reduced pressure. The crude material was purified by silica gel chromatography (ISCO, 12 g of column, hexane/ethyl acetate, 0-30%, 25 min; 30-80%, 10 min; 80%, 5 min) to give 100 mg of the title product as a white solid.

### Step G: 6-(3-Chloro-2-fluoro-benzyl)-1-((S)-1-hydroxymethyl-2-methyl-propyl)-7-methoxy-4-oxo-1,4-dihydro-[1,8]naphthyridine-3-carboxylic acid

1-[(S)-1-(tert-Butyl-dimethyl-silanyloxymethyl)-2-methyl-propyl]-6-(3-chloro-2-fluoro-benzyl)-7-methoxy-4-oxo-1,4-dihydro-[1,8]naphthyridine-3-carboxylic acid ethyl ester (100 mg, 0.17 mmol) was dissolved in methanol (10 mL). A solution of 1 ml of 25% sodium methoxide in methanol and 2 ml of water was added. The mixture was refluxed for 4 hours, allowed to cool to room temperature and evaporated to a small volume under reduced pressure. Water (10 mL) was added and the resulting mixture was filtered. The filtrate was neutralized with 1 N hydrochloric acid. The solid was filtered and washed with water to give a pure product as an off-white solid (60 mg, 79%).

### II. Preparation of Compounds of Formula (II)

### Example 2

### Compounds of Formula (II)

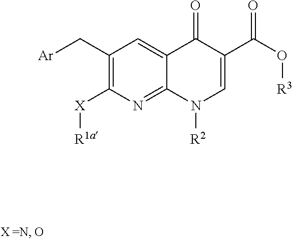

Compounds of formula (II) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

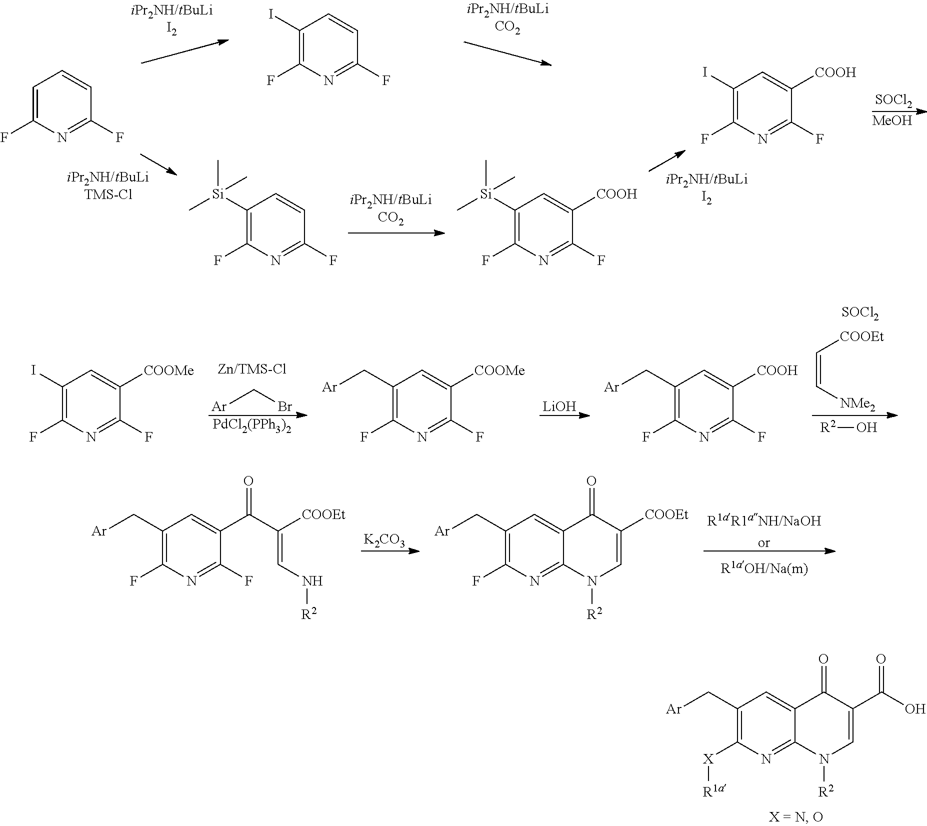

### Example 2A

### (S)-6-(3-chloro-2-fluorobenzyl)-7-morpholin-1-(1-hydroxy-3,3-dimethylbutan-2-yl)-4-oxo-1,4-dihydro-1,8-naphthyridine-3-carboxylic acid

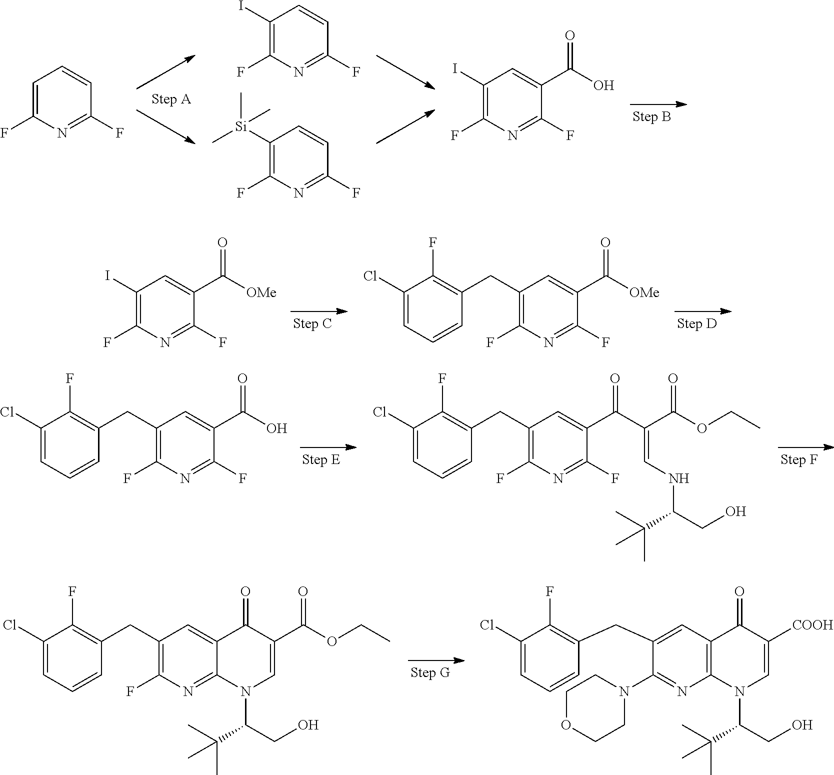

### Step A: 2,6-Difluoro-5-iodopyridine-3-carboxylic acid

### Method A:

(1) 2,6-Difluoro-3-iodopyridine

Diisopropylamine (141.3 mL, 101.19 g, 1 mol) and 2,6-difluoropyridine (115.08 g, 1 mol) were added consecutively to a solution of butyllithium (1.6M in hexane, 625 mL, 1 mol) in tetrahydrofuran (2000 mL) kept in a dry ice/methanol bath. After 1 h at −75° C., the mixture was treated with a solution of iodine (253.8 g, 1 mol) in tetrahydrofuran (1000 mL). The mixture was washed with a 10% aqueous solution (500 mL) of sodium sulfite, the organic phase was dried (MgSO4) and the volatiles were evaporated under reduced pressure. The residue was purified by vacuum distillation to give 169 g of colorless oily product, which was crystallized to afford colorless platelets. Yield: 70%.

(2) 2,6-Difluoro-5-iodopyridine-3-carboxylic acid

At −75° C., diisopropylamine (7.0 mL, 5.1 g, 50 mmol) and a solution of 2,6-difluoro-3-iodopyridine (12.1 g, 50 mmol) in tetrahydrofuran (50 mL) were consecutively added to butyllithium (1.6M in hexane, 31.3 mL, 50 mmol) in tetrahydrofuran (60 mL). After 15 min at −75° C., the mixture was poured on an excess of freshly crushed dry ice. The volatiles were evaporated and the residue was dissolved in a 2.0 N aqueous solution (50 mL) of sodium hydroxide. The aqueous phase was washed with diethyl ether (2×25 mL), acidified with hydrochloric acid to pH 2 and extracted with diethyl ether (3×50 mL). The combined organic layers were dried and concentrated under reduced pressure. The residue was recrystallized from water to give pure 2,6-difluoro-5-iodopyridine-3-carboxylic acid as colorless platelets. Water liquor contains 2,6-difluoro-5-iodopyridine-4-carboxylic acid.

### Method B:

(1) (2,6-Difluoropyridin-3-yl)trimethylsilane

Diisopropylamine (61.4 mL, 44.0 g, 434 mmol) and 2,6-difluoropyridine (50 g, 434 mmol) were added consecutively to a solution of butyllithium (1.6M in hexane, 271 mL, 434 mmol) in tetrahydrofuran (1000 mL), cooled in an acetone/dry ice bath. After 90 min at −75° C., chlorotrimethylsilane was added. The reaction mixture was warmed to room temperature and 200 mL of water was added. The water phase was extracted with diethyl ether twice and the combined organic layers were dried over anhydrous sodium sulfate and evaporated to dryness. The residue was purified by vacuum distillation (b.p. 75-77° C./20 Torr) to give pure products as a colorless liquid in an high yield.

(2) 2,6-Difluoro-5-iodo-pyridine-3-carboxylic acid

Diisopropylamine (14 mL, 10 g, 0.10 mol) and (2,6-difluoropyridin-3-yl)trimethylsilane (18.7 g, 0.10 mol) were added consecutively to a solution of butyllithium (0.10 mol) in tetrahydrofuran (200 mL) and cooled in an acetone/dry ice bath. After 90 min at −75° C., the mixture was poured on an excess of freshly crushed dry ice. At 25° C., 2.0 N ethereal hydrogen chloride (75 mL, 0.15 mol) was added and filtered and Washed with chloroform. The filtrate was evaporated to dryness under reduced pressure and the solid residue was extracted with hot chloroform, filtered, and concentrated to afford the crude 2,6-difluoro-5-(trimethylsilyl)pyridine-3-carboxylic acid as a white solid, which was used in the next step without further purification.

A solution of the dried above crude product and iodine monochloride (32 g, 0.20 mol) in tetrachloromethane (0.10 L) were heated under reflux for 20 h. The reaction mixture was cooled to room temperature, diluted with ether and washed with a saturated aqueous solution (100 mL) of sodium sulfite. The organic layer was separated and the water layer was neutralized with concentrated hydrochloric acid and extracted with ether. The combined organic layers were dried and concentrated under reduced pressure. The residue was recrystallized from water to give the desired product.

### Step B: 2,6-Difluoro-5-iodopyridine-3-carboxylic acid methyl ester

A mixture of 2,6-dichloro-5-iodo-pyridine-3-carboxylic acid (3.4 g, 11.9 mmol) and thionyl chloride (1.74 mL, 23.9 mmol) in 40 mL of anhydrous toluene and 0.1 mL of anhydrous DMF was refluxed for 2 h. The solvent was removed under reduced pressure and the residue was azeoptoped with toluene (2×20 mL). The residue was dissolved in 50 mL of anhydrous methanol refluxed for 30 min and cooled to room temperature. The solvent was removed under reduced pressure to give the crude product as a white solid.

### Step C: Methyl 5-(3-chloro-2-fluorobenzyl)-2,6-difluoronicotinate

Under an argon stream, zinc powder (3.6 g, 55 mmol) was suspended in 5 mL of dry THF. 1,2-Dibromoethane (0.01 mL, 0.12 mmol) and TMS-Cl (0.03 mL, 0.24 mmol) were added at 60° C. to the suspension, and the mixture was stirred at this temperature for 30 min. A solution of 2-fluoro-3-chlorobenzyl bromide (2.7 g, 12 mmol) in 10 mL of dry THF was added dropwise at 60° C. The mixture was stirred with heating for 1 hour and allowed to cool to room temperature. The resulting solution of 2-fluoro-3-chlorobenzylzinc bromide in THF is used for next step.

To a solution of 2,6-Difluoro-5-iodopyridine-3-carboxylic acid methyl ester (2.7 g, 9 mmol) in 40 mL of dry THF was added dichlorobis(triphenylphosphine)palladium (II) (253 mg, 0.36 mmol). The mixture was heated at 60° C. and a solution of the above. 2-fluoro-3-chlorobenzylzinc bromide in THF was added dropwise. The mixture was stirred with heating at the same temperature for 1 hour and was allowed to cool to room temperature. HCl (1 N, 75 mL) was added and the mixture was extracted with ethyl acetate (3×100 mL). The organic layers were combined, washed successively with water, brine, dried over anhydrous sodium sulfate and concentrated under reduced pressure. The crude product was purified by silica gel chromatography (ISCO, 40 g of column, hexane/ethyl acetate, 0-30%, 25 min, 30-80%, 10 mm, 80%, 5 min) to give 2.3 g of pure product as colorless oil.

### Step D: 5-(3-Chloro-2-fluorobenzyl)-2,6-difluoronicotinic acid

To a solution of methyl 5-(3-chloro-2-fluorobenzyl)-2,6-difluoronicotinate (3.7 g, 11.7 mmol) in 36 mL of THF was added dropwise an aqueous solution of LiOH (1 N, 35 mL, 35 mmol). The mixture was stirred at room temperature for 1 hour and evaporated under reduced pressure. The residue was dissolved in 50 mL of water and neutralized with 6 N HCl. The precipitate was filtered and washed with water to give 3.5 g of pure product as a white solid in a quantitative yield.

### Step E: (S)-ethyl 2-(5-(3-chloro-2-fluorobenzyl)-2,6-difluoronicotinoyl)-3-(1-hydroxy-3,3-dimethylbutan-2-ylamino)acrylate

A mixture of 5-(3-chloro-2-fluorobenzyl)-2,6-difluoronicotinic acid (12.1 g, 40 mmol) and thionyl chloride (5.84 mL, 80 mmol) in 160 mL of anhydrous toluene and 0.4 mL of anhydrous DMF was refluxed for 2 h. The solvent was removed under reduced pressure and the resulting oil was azeoptoped with toluene (2×80 mL). The residue was dissolved in 40 mL of anhydrous THF and added dropwise to a solution of ethyl 3-(dimethylamino)acrylate (6.24 g, 44 mmol) and triethylamine (4.88 g, 57.6 mmol) in 160 mL of anhydrous THF under nitrogen. The mixture was heated under reflux for 7 hours, allowed to cool to room temperature and (S)-tort-leucinal (5.16 g, 44 mmol) (or L-valinol) was added. The reaction mixture was stirred for 30 min at room temperature and evaporated to dryness under reduced pressure. Water (200 mL) and ethyl acetate (200 mL) were added and the organic layer was separated, washed successively with saturated aqueous sodium bicarbonate (×2), water, brine, and dried over sodium sulfate. The mixture was filtered and the filtrate was concentrated under reduced pressure. The crude product was purified by silica gel chromatography (ISCO, hexane/EtOAc, 330 g, 0-40%, 30 min; 40-100%, 10 min; 100%, 30 min) to give the desired material as an yellow oil.

### Step F: 6-(3-Chloro-2-fluoro-benzyl)-7-fluoro-1-((S)-1-hydroxymethyl-2,2-dimethyl-propyl)-4-oxo-1,4-dihydro-1,8-naphthyridine-3-carboxylic acid ethyl ester

A mixture of (S)-ethyl 2-(5-(3-chloro-2-fluorobenzyl)-2,6-difluoronicotinoyl)-3-(1-hydroxy-3,3-dimethylbutan-2-ylamino)acrylate (2.5 g, 5 mmol) and potassium carbonate (1.4 g, 10 mmol) in 30 mL of anhydrous DMF was stirred at 90° C. in an oil bath preheated to 90° C. for 10 min. Ice-water (300 mL) was added with stirring. The resulting precipitate was isolated by filtration and washed with water to give the desired product as white solid in almost quantitative yield (purity: 96%).

This product was treated with a THF solution of tetrabutylammonium fluoride to remove TBDMS group and then hydrolyzed in a solution of THF/1 N LiOH to give the desired product after purification by preparative HPLC.

### Step G: (S)-6-(3-chloro-2-fluorobenzyl)-7-morpholin-1-(1-hydroxy-3,3-dimethylbutan-2-yl)-4-oxo-1,4-dihydro-1,8-naphthyridine-3-carboxylic acid

A mixture of 1-[(S)-1-(tert-butyl-dimethyl-silanyloxymethyl)-2,2-dimethyl-propyl]-6-(3-chloro-2-fluoro-benzyl)-7-fluoro-4-oxo-1,4-dihydro-1,8-naphthyridine-3-carboxylic acid ethyl ester (1 g, 2.1 mmol), morpholine (0.37 g, 4.2 mmol) in methanol (15 mL) was stirred at room temperature for 3 days, followed by the addition of 10 mL of 1 N sodium hydroxide. The resulting mixture, was stirred at 80° C. for 1 hour and concentrated under reduced pressure. The residue was dissolved in 20 mL of water and filtered. The filtrate was neutralized with 6 N HCl and the precipitate was isolated and washed with water. The crude material was recrystallized from ethyl acetate to give the desired compound as white crystals (1.04 g, 96%; Purity: 96%).

### Example 2B

### (S)-6-(3-chloro-2-fluorobenzyl)-7-hydroxy-1-(1-hydroxy-3,3-dimethylbutan-2-yl)-4-oxo-1,4-dihydro-1,8-naphthyridine-3-carboxylic acid

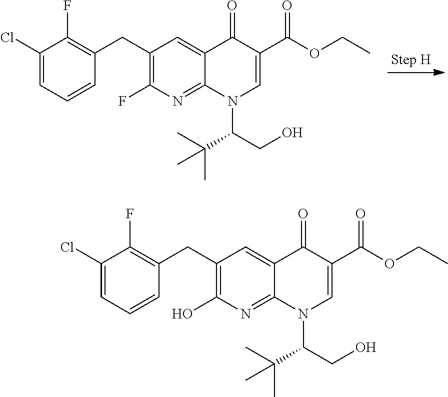

Step I: A suspension of (S)-ethyl 6-(3-chloro-2-fluorobenzyl)-7-fluoro-1-(1-hydroxy-3,3-dimethylbutan-2-yl)-4-oxo-1,4-dihydro-1,8-naphthyridine-3-carboxylate (300 mg) in 15 mL of 1 N sodium hydroxide was stirred at 80° C. for 1 hour. The reaction mixture was cooled to room temperature and filtered. The filtrate was neutralized with 6 N HCl and the precipitate was filtered and washed with water to give the desired product as a white solid.

### Examples 2C-2O

### (S)-6-(3-chloro-2-fluorobenzyl)-1-(1-hydroxy-3,3-dimethylbutan-2-yl)-4-oxo-7-alkyloxy-1,4-dihydro-1,8-naphthyridine-3-carboxylic acid

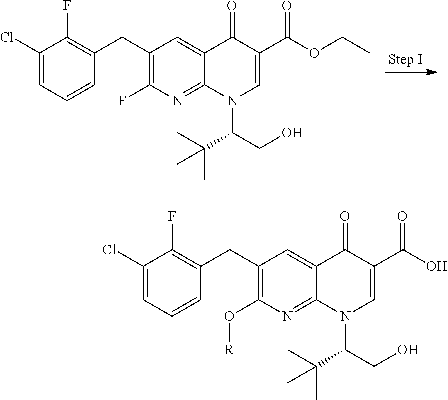

Step I: 50 mg of sodium metal was added to 2 g of the corresponding alcohol under argon at room temperature and the resulting mixture was stirred at 80° C. until sodium was dissolved (about 1-2 hours). 300 mg of (S)-ethyl 6-(3-chloro-2-fluorobenzyl)-7-fluoro-1-(1-hydroxy-3,3-dimethylbutan-2-yl)-4-oxo-1,4-dihydro-1,8-naphthyridine-3-carboxylate added to this alcoholic sodium solution and stirred at 80° C. overnight and then 15 mL of 1 N sodium hydroxide was added and stirred at 80° C. for 1 hour. The reaction mixture was cooled to room temperature and filtered if necessary. The filtrate was neutralized with 2 N HCl to pH<7. The precipitate was filtered and washed with water to give the desired product which was purified by preparative HPLC if needed.

### III. Preparation of Compounds of Formula (III)

### Example 3

### Compounds of Formula (III)

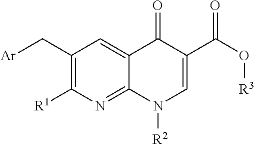

Compounds of formula (III) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

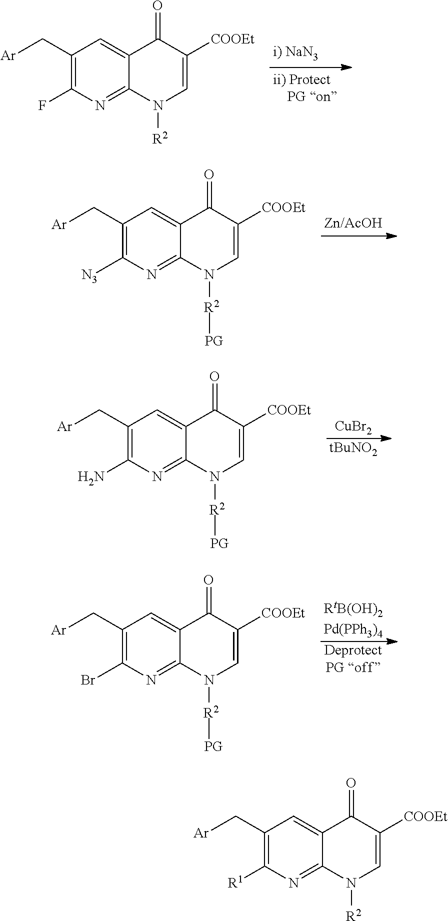

### Example 3A

### (S)-7-Amino-6-(3-chloro-2-fluorobenzyl)-1-(1-hydroxy-3,3-dimethylbutan-2-yl)-4-oxo-1,4-dihydro-1,8-naphthyridine-3-carboxylic acid

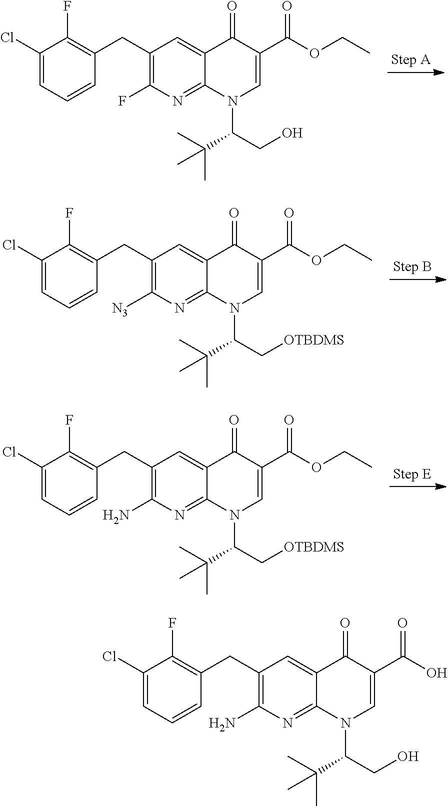

### Step A: 7-Azido-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester

A mixture of 6-(3-chloro-2-fluoro-benzyl)-7-fluoro-1-((S)-1-hydroxymethyl-2,2-dimethyl-propyl)-4-oxo-1,4-dihydro-1,8-naphthyridine-3-carboxylic acid ethyl ester (4.79 g, 10 mmol) and sodium azide (1.3 g, 20 mmol) in anhydrous DMF (10 mL) was stirred overnight at room temperature followed by the addition of imidazole (6.81 g, 100 mmol) and TBDMS-Cl (7.54 g, 50 mmol). The mixture was stirred an additional 18 hours at room temperature and the solvent was evaporated under reduced pressure. The residue was purified by ISCO (hexane/EtOAc, 0-30%, 20 min, 40-100%, 10 min, 100%, 10 min) to give the pure product as a yellow oil (6.2 g, 100%).

### Step B: 7-Amino-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester

Zinc powder (2.7 g, 41.5 mmol) was added to a solution of 7-azido-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester (5.1 g, 8.3 mmol) in 3:1 dichloroform/acetic acid (80 mL). After 15 min the reaction mixture was poured into 300 mL of ethyl acetate and the resulting solution was washed with water, saturated sodium bicarbonate and brine. The organic solution was dried over sodium sulfate, filtered, and concentrated in vacuo to provide the desired product as a yellow oil in quantitative yield (purity: 97%).

### Step E: (S)-7-Amino-6-(3-chloro-2-fluorobenzyl)-1-(1-hydroxy-3,3-dimethylbutan-2-yl)-4-oxo-1,4-dihydro-1,8-naphthyridine-3-carboxylic acid

7-Amino-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester was hydrolyzed using the same method described in step E below.

### Example 3B

### 6-(3-Chloro-2-fluorobenzyl)-1-((S)-1-hydroxymethyl-2,2-dimethyl-propyl)-7-methyl-4-oxo-1,4-dihydro-1,8-naphthyridine-3-carboxylic acid

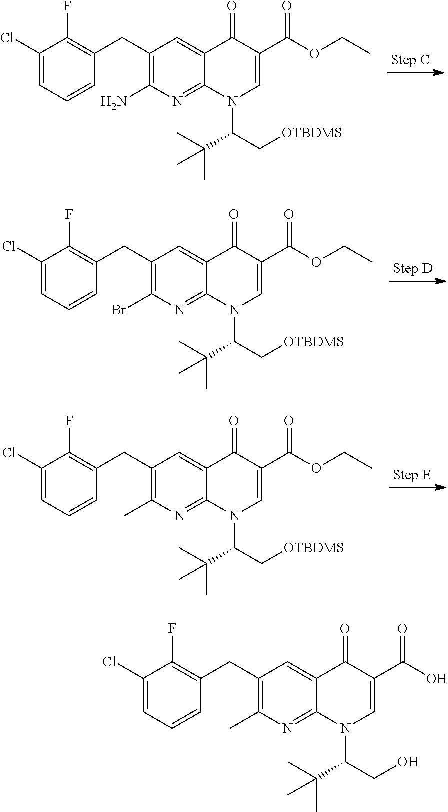

### Step C: 7-Bromo-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester

A mixture of copper bromide (1.7 g, 7.6 mmol), tert-butyl nitrite (1.0 g, 9.5 mmol) in bromoform (5 mL) and anhydrous acetonitrile (20 mL) was warmed to 60° C. under argon and then a solution of 7-amino-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester (3.7 g, 6.3 mmol) in 10 mL of anhydrous acetonitrile was added dropwise. The mixture was stirred at the same temperature for 20 min. The reaction mixture was cooled to room temperature and filtered through Celite and washed with ethyl acetate. The filtrate was evaporated to dryness under reduced pressure and the residue was purified by ISCO (hexane/ethyl acetate, 0%, 5 min; 0-30%, 25 min; 30-100%, 10 min) to give the pure product as an yellowish solid (2.6 g, 63%).

### Step D: 1-[(S)-1-(tert-Butyl-dimethyl-silanyloxymethyl)-2,2-dimethyl-propyl]-6-(3-chloro-2-fluoro-benzyl)-7-methyl-4-oxo-1,4-dihydro-1,8-naphthyridine-3-carboxylic acid ethyl ester

7-bromo-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester (300 mg, 0.46 mmol) was dissolved in 10 mL of 1,2-dimethoxyethane and methylboronic acid (55 mg, 0.92 mmol), tetrakis(triphenylphosphine)palladium(0) (35 mg, 0.03 mmol) and 2M sodium carbonate (0.5 mL) were added. The reaction mixture was stirred at 80° C. for 48 hours. After cooling to room temperature, saturated aqueous ammonium chloride and ethyl acetate were added to the reaction mixture. The organic layer was washed with water, brine, dried over sodium sulfate and concentrated under reduced pressure. The crude residue was purified by ISCO (hexane/ethyl acetate: 0%, 5 min; 0-30%, 30 min; 30-100%, 10 min) to give pure compound as an oil (170 mg, 63%).

### Step E: 6-(3-Chloro-2-fluoro-benzyl)-1-((S)-1-hydroxymethyl-2,2-dimethyl-propyl)-7-methyl-4-oxo-1,4-dihydro-1,8-naphthyridine-3-carboxylic acid

A mixture of 1-[(S)-1-(tert-Butyl-dimethyl-silanyloxymethyl)-2,2-dimethyl-propyl]-6-(3-chloro-2-fluoro-benzyl)-7-methyl-4-oxo-1,4-dihydro-1,8-naphthyridine-3-carboxylic acid ethyl ester (100 mg, 0.17 mmol), 28% sodium methoxide (2 mL) and water (1 mL) in methanol (15 mL) was stirred at 80° C. for 5 hour. The reaction mixture was cooled at room temperature and the solvent was evaporated under reduced pressure. The residue was dissolved in 10 mL of water and filtered. The filtrate was neutralized with 6 N HCl and the precipitate was filtered and washed with water to give pure product as a white solid.

### Example 3C

### 6-(3-Chloro-2-fluoro-benzyl)-1-((S)-1-hydroxymethyl-2,2-dimethyl-propyl)-7-(3-hydroxy-propyl)-4-oxo-1,4-dihydro-1,8-naphthyridine-3-carboxylic acid

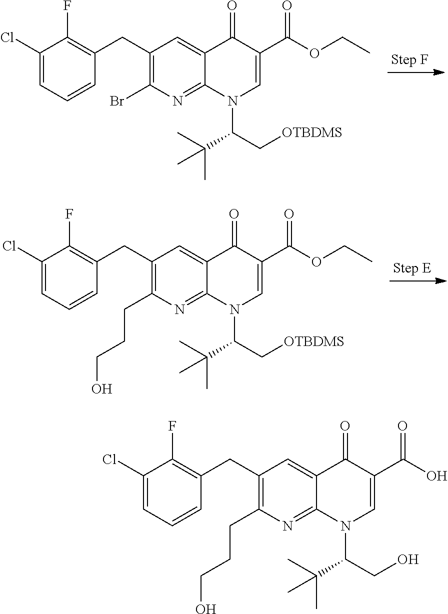

### Step F: (S)-ethyl 1-(1-(tert-butyldimethylsilyloxy)-3,3-dimethylbutan-2-yl)-6-(3-chloro-2-fluorobenzyl)-7-(3-hydroxypropyl)-4-oxo-1,4-dihydro-1,8-naphthyridine-3-carboxylate

7-Bromo-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester (300 mg, 0.46 mmol) was dissolved in 10 mL of 1,2-dimethoxyethane. 3-Bromopropylboronic acid pinacol ester (229 mg, 0.92 mmol), tetrakis(triphenylphosphine)palladium(0) (35 mg, 0.03 mmol), and 2M sodium carbonate (0.5 mL) were added. The reaction mixture was stirred at 80° C. for 48 hours. After cooling to room temperature, saturated aqueous ammonium chloride and ethyl acetate were added to the reaction mixture. The organic layer was washed with water, brine, and dried over sodium sulfate. The solution was concentrated under reduced pressure and the residue was purified by ISCO (hexane/ethyl acetate: 0%, 5 min; 0-30%, 30 min; 30-100%, 10 min) to give the desired compound as an oil.

### Step E: 6-(3-Chloro-2-fluoro-benzyl)-1-((S)-1-hydroxymethyl-2,2-dimethyl-propyl)-7-(3-hydroxy-propyl)-4-oxo-1,4-dihydro-1,8-naphthyridine-3-carboxylic acid

(S)-ethyl 1-(1-(tert-butyldimethylsilyloxy)-3,3-dimethylbutan-2-yl)-6-(3-chloro-2-fluorobenzyl)-7-(3-hydroxypropyl)-4-oxo-1,4-dihydro-1,8-naphthyridine-3-carboxylate was hydrolyzed according to the procedure described in step E above to give the desired product as a white solid. Yield: 71%, Purity: 96%.

### IV. Preparation of Compounds of Formula (IV)

### Example 4

### Compounds of Formula (IV)

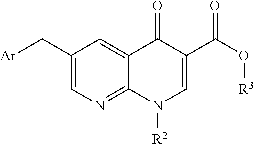

Compounds of formula (IV) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

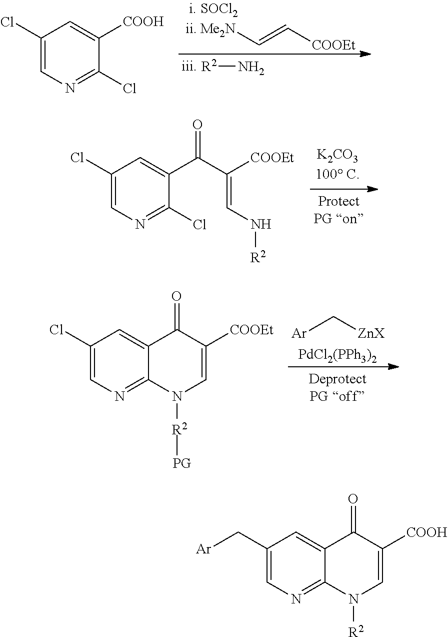

### Example 4A

### (S)-6-(3-chloro-2-fluorobenzyl)-1-(1-hydroxy-3-methylbutan-2-yl)-4-oxo-1,4-dihydro-1,8-naphthyridine-3-carboxylic acid

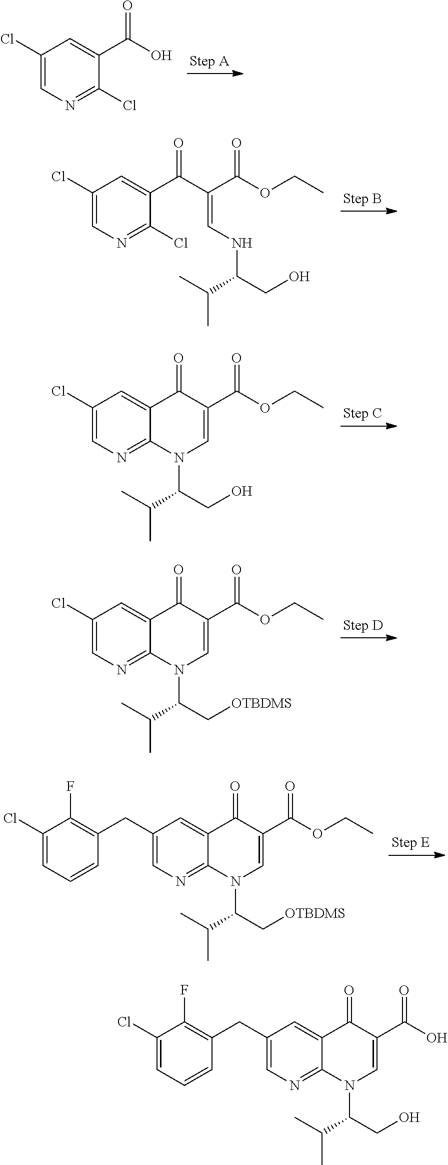

### Step A: (S)-ethyl 2-(2,5-dichloronicotinoyl)-3-(1-hydroxy-3-methylbutan-2-ylamino)acrylate

This compound was prepared according to the procedure described above for example 1A.

### Step B: (S)-ethyl 6-chloro-1-(1-hydroxy-3-methylbutan-2-yl)-4-oxo-1,4-dihydro-1,8-naphthyridine-3-carboxylate

This compound was prepared according to the procedure described above for example 1A.

### Step C: (S)-ethyl 1-(1-(tert-butyldimethylsilyloxy)-3-methylbutan-2-yl)-6-chloro-4-oxo-1,4-dihydro-1,8-naphthyridine-3-carboxylate

This compound was prepared according to the procedure described above for example 1A.

### Step D: (S)-ethyl 1-(1-(tert-butyldimethylsilyloxy)-3-methylbutan-2-yl)-6-(3-chloro-2-fluorobenzyl)-4-oxo-1,4-dihydro-1,8-naphthyridine-3-carboxylate

This compound was prepared according to the procedure described above for example 1A.

### Step E: (S)-6-(3-chloro-2-fluorobenzyl)-1-(1-hydroxy-3-methylbutan-2-yl)-4-oxo-1,4-dihydro-1,8-naphthyridine-3-carboxylic acid

This compound was prepared according to the procedure described above for example 1A.

### V. Preparation of Compounds of Formula (V)(a)

Compounds of formula I, wherein X is C(R^{x})(R^{x′}), R^{x} is H, R¹ is O—R^{1a} and R², R³, R⁴, R^{a}, R^{b}, R^{c}, R^{d} and R^{e} are as defined herein, are prepared according to the following synthetic scheme. When appropriate, protecting groups are used prior to performing the reaction outlined below, and may or may not be removed upon completion of the synthesis. The individual starting materials are synthesized according to methods known in the art or are commercially available.

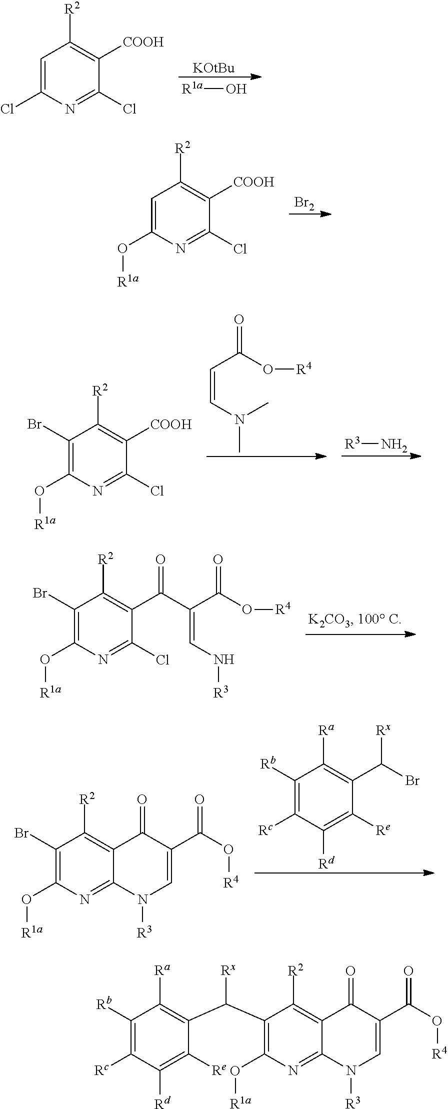

### VI. Preparation of Compounds of Formula (V)(b)

Compounds of formula (V)(b), wherein Y is CH₂, R¹ is O—R^{1a} and R², R³, R⁵, R^{a}, R^{b}, R^{c}, R^{d} and R^{e} are as defined herein, are prepared according to the following synthetic scheme. When appropriate, protecting groups are used prior to performing the reaction outlined below, and may or may not be removed upon completion of the synthesis. The individual starting materials are synthesized according to methods known in the art or are commercially available.

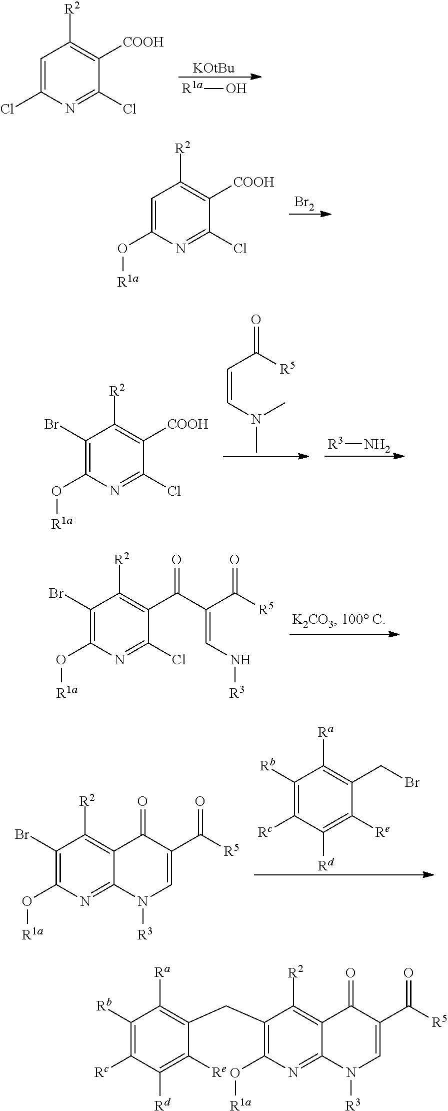

### Further Forms of Compounds of Formula (I) or (II); Formula (III) or (IV); or Formula (V)(a), (V)(b) or (V)(c)

### Isomers of Compounds of Formula (I) or (II); Formula (III) or (IV); or Formula (V)(a), (V)(b) or (V)(c)

In some embodiments, a compound of formula (I) or (II); formula (III) or (IV); or formula (V)(a), (V)(b) or (V)(c) exists as geometric isomers. In some embodiments, a compound of formula (I) or (II); formula (III) or (IV); or formula (V)(a), (V)(b) or (V)(c) possesses one or more double bonds. Compounds of formula (I) or (II); formula (III) or (IV); or formula (V)(a), (V)(b) or (V)(c) include all cis, trans, syn, anti, entgegen (E), and zusammen (Z) isomers as well as the corresponding mixtures thereof. In some situations, compounds exist as tautomers. Compounds of formula (I) or (II); formula (III) or (IV); or formula (V)(a), (V)(b) or (V)(c) include all possible tautomers within the formulas described herein. In some embodiments, a compound of formula (I) or (II); formula (III) or (IV); or formula (V)(a), (V)(b) or (V)(c) possesses one or more chiral centers and each center exists in the R or S configuration. Compounds of formula (I) or (II); formula (III) or (IV); or formula (V)(a), (V)(b) or (V)(c) include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. Compounds of formula (I) or (II); formula (III) or (IV); or formula (V)(a), (V)(b) or (V)(c) are prepared as their individual stereoisomers by reacting a racemic mixture of the compound with an optically active resolving agent to form a pair of diastereoisomeric compounds, separating the diastereomers and recovering the optically pure enantiomers. While resolution of enantiomers is carried out using covalent diastereomeric derivatives of a compound of formula (I) or (II); formula (III) or (IV); or formula (V)(a), (V)(b) or (V)(c), dissociable complexes, are preferred (e.g., crystalline diastereomeric salts). Diastereomers have distinct physical properties (e.g., melting points, boiling points, solubilities, reactivity, etc.) and are readily separated by taking advantage of these dissimilarities. In some embodiments, the diastereomers are separated by chiral chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture is found in Jacques et al, “ENANTIOMERS, RACEMATES AND RESOLUTIONS” (John Wiley And Sons, 1981), herein incorporated by reference for such subject matter.

### Labeled Compounds of Formula (I) or (II); Formula (III) or (IV); or Formula (V)(a), (V)(b) or (V)(c)

In some embodiments, compounds Of formula (I) or (II); formula (III) or (IV); or formula (V)(a), (V)(b) or (V)(c) exist in their isotopically-labeled forms. Disclosed herein are, in certain instances, are methods of treating diseases by administering such isotopically-labeled compounds. Further disclosed herein are methods of treating diseases by administering such isotopically-labeled compounds as pharmaceutical compositions. Thus, compounds of formula (I) or (II); formula (III) or (IV); or formula (V)(a), (V)(b) or (V)(c) also include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of formula (I) or (II); formula (III) or (IV); or formula (V)(a), (V)(b) or (V)(c) include isotopes of hydrogen, carbon, nitrogen, oxygen, phosphorous, sulfur, fluorine and chloride, such as ²H, ³H, ¹³C, ¹⁴C, ¹⁵N, ¹⁸0, ¹⁷O, ³¹P, ³²P, ³⁵S, ¹⁸F, and ³⁶Cl, respectively. Compounds of formula (I) or (II); formula (III) or (IV); or formula (V)(a), (V)(b) or (V)(c), and the pharmaceutically acceptable salts, esters, prodrugs, solvate, hydrates or derivatives thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated (i.e., ³H and carbon-14, i.e., ¹⁴C) isotopes are particularly preferred for their ease of preparation and detectability. Further, substitution with heavy isotopes such as deuterium, i.e., ²H, affords certain therapeutic advantages resulting from greater metabolic stability, for example increased in vivo half-life or reduced dosage requirements and, hence, are preferred in some circumstances. Isotopically labeled compounds, pharmaceutically acceptable salt, ester, prodrug, solvate, hydrate or derivative thereof are generally prepared by carrying out procedures described herein, by substituting a readily available isotopically labeled reagent for a non-isotopically labeled reagent.

In some embodiments, a compound of formula (I) or (II); formula (III) or (IV); or formula (V)(a), (V)(b) or (V)(c) is labeled by other means, including, but not limited to, the use of chromophores or fluorescent moieties, bioluminescent labels, or chemiluminescent labels.

### Pharmaceutically Acceptable Salts of Compounds of Formula (I) or (II); Formula (III) or (IV); or Formula (V)(a), (V)(b) or (V)(c)

In some embodiments, a compound of formula (I) or (II); formula (III) or (IV); or formula (V)(a), (V)(b) or (V)(c) exists as their pharmaceutically acceptable salts. Disclosed herein, in certain instances, are methods of treating diseases by administering such pharmaceutically acceptable salts. Further disclosed herein, in certain instances, are methods of treating diseases by administering such pharmaceutically acceptable salts as pharmaceutical compositions.

In some embodiments, a compound of formula (I) or (II); formula (III) or (IV); or formula (V)(a), (V)(b) or (V)(c) possesses acidic or basic groups and therefore react with any of a number of inorganic or organic bases, and inorganic and organic acids, to form a pharmaceutically acceptable salt. In some embodiments, these salts are prepared in situ during the final isolation and purification of the compounds of formula (I) or (II); formula (III) or (IV); or formula (V)(a), (V)(b) or (V)(c), or by separately reacting a purified compound in its free form with a suitable acid or base, and isolating the salt thus formed.

Examples of pharmaceutically acceptable salts include those Salts prepared by reaction of a compound of formula (I) or (II); formula (III) or (IV); or formula (V)(a), (V)(b) or (V)(c) with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate.

Further, in some embodiments, compounds of formula (I) or (II); formula (III) or (IV); or formula (V)(a), (V)(b) or (V)(c) are prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds of formula (I) or (II); formula (III) or (IV); or formula (V)(a), (V)(b) or (V)(c) and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that compounds of formula (I) or (II); formula (III) or (IV); or formula (V)(a), (V)(b) or (V)(c) include the quaternization of any basic nitrogen-containing groups they contain. In some embodiments, water or oil-soluble or dispersible products are obtained by such quaternization. In some embodiments, a compound of formula (I) or (II); formula (III) or (IV); or formula (V)(a), (V)(b) or (V)(c) is prepared as pharmaceutically acceptable salt formed when an acidic proton present in the parent compound either is replaced by a metal ion, for example an alkali metal ion, an alkaline earth ion, or an aluminum ion; or coordinates with an organic base. In some embodiments, base addition salts are prepared by reacting the free acid form of a compound of formula (I) or (II); formula (III) or (IV); or formula (V)(a), (V)(b) or (V)(c) with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide; and the like. In addition, in some embodiments, the salt forms of the disclosed compounds are prepared using salts of the starting materials or intermediates. For additional information on pharmaceutical salts see for example Berge et al., J. Pharm. Sci. 1977, 66, 1-19.

### Solvates of Compounds of Formula (I) or (II); Formula (III) or (IV); or Formula (V)(a), (V)(b) or (V)(c)

In some embodiments, compounds of formula (I) or (II); formula (III) or (IV); or formula (V)(a), (V)(b) or (V)(c) exist as solvates. Disclosed herein, in certain instances, are methods of treating diseases by administering such solvates. Further disclosed herein, in certain instances, are methods of treating diseases by administering such solvates as pharmaceutical compositions.

Solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. In some embodiments, solvates of a compound of formula (I) or (II); formula (III) or (IV); or formula (V)(a), (V)(b) or (V)(c) are prepared or formed during the processes described herein. By way of example only, hydrates of a compound of formula (I) or (II); formula (III) or (IV); or formula (V)(a), (V)(b) or (V)(c) are prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Polymorphs of Compounds of Formula (I) or (II); Formula (III) or (IV); or Formula (V)(a), (V)(b) or (V)(c)

In some embodiments, compound of formula (I) or (II); formula (III) or (IV); or formula (V)(a), (V)(b) or (V)(c) exist as polymorphs. Disclosed herein, in certain instances, are methods of treating diseases by administering such polymorphs. Further disclosed herein, in certain instances, are methods of treating diseases by administering such polymorphs as pharmaceutical compositions.

Compounds of formula (I) or (II); formula (III) or (IV); or formula (V)(a), (V)(h) or (V)(c) include all their crystalline forms, known as polymorphs. Polymorphs include the different crystal packing arrangements of the same elemental composition of a compound. In some embodiments, polymorphs have different X-ray diffraction patterns, infrared spectra, melting points, density, hardness, crystal shape, optical and electrical properties, stability, and solubility. Factors such as the recrystallization solvent, rate of crystallization, and storage temperature affect which crystal or crystals dominate.

### Prodrugs of Compounds of Formula (I) or (II); Formula (III) or (IV); or Formula (V)(a), (V)(b) or (V)(c)

In some embodiments, a compound of formula (I) or (II); formula (III) or (IV); or formula (V)(a), (V)(b) or (V)(c) exists in prodrug form. Disclosed herein, in certain instances, are methods of treating diseases by administering such prodrugs. Further disclosed herein, in certain instances, are methods of treating diseases by administering such prodrugs as pharmaceutical compositions.

Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they are easier to administer than the parent drug. For example, prodrugs are bioavailable by oral administration whereas the parent is not. In some embodiments, the prodrug has improved solubility in pharmaceutical compositions over the parent drug. An example, without limitation, of a prodrug would be a compound as described herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyamino acid) bonded to an acid group where the peptide is metabolized to reveal the active moiety. Various forms of prodrugs are well known in the art. (See for example Bundgaard, “Design and Application of Prodrugs” in A textbook of Drug Design and Development, Krosgaard-Larsen and Bundgaard, Ed., 1991, Chapter 5, 113-191, which is incorporated herein by reference).

In some embodiments, prodrugs are designed as reversible drug derivatives, for use as modifiers to enhance drug transport to site-specific tissues. The design of prodrugs to date has been to increase the effective water solubility of the therapeutic compound for targeting to regions where water is the principal solvent.

Additionally, in some embodiments, prodrug derivatives of compounds described herein are prepared by any suitable method (for further details see Saulnier et al., Bioorganic and Medicinal Chemistry Letters, 1994, 4, 1985). By way of example only, appropriate prodrugs are prepared by reacting a non-derivatized compound with a suitable carbamylating agent, such as, but not limited to, 1,1-acyloxyalkylcarbanochloridate, para-nitrophenyl carbonate, or the like. Prodrug forms of the herein described compounds, wherein the prodrug is metabolized in vivo to produce a derivative as set forth herein are included within the scope of the claims. Indeed, some of the herein-described compounds are a prodrug for another derivative or active compound.

In some embodiments, prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula (I) or (II); formula (III) or (IV); or formula (V)(a), (V)(b) or (V)(c). The amino acid residues include but are not limited to the 20 naturally occurring amino acids and also includes 4-hydroxyproline, hydroxylysine, demosine, isodemosine, 3-methylhistidine, norvaline, beta-alanine, gamma-aminobutyric acid, cirtulline, homocysteine, homoserine, ornithine and methionine sulfone. In other embodiments, prodrugs include compounds wherein a nucleic acid residue, or an oligonucleotide of two or more (e.g., two, three or four) nucleic acid residues is covalently joined to a compound of formula (I) or (II); formula (III) or (IV); or formula (V)(a), (V)(b) or (V)(c).

Pharmaceutically acceptable prodrugs of a compound of formula (I) or (II); formula (III) or (IV); or formula (V)(a), (V)(b) or (V)(c) also include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. In some embodiments, compounds having free amino, amino, hydroxy or carboxylic groups are converted into prodrugs. For instance, free carboxyl groups are derivatized as amides or alkyl esters. In some embodiments, a prodrug moiety incorporates groups including but not limited to ether, amine and carboxylic acid functionalities.

Hydroxy prodrugs include esters, such as though not limited to, acyloxyalkyl (e.g. acyloxymethyl, acyloxyethyl) esters, alkoxycarbonyloxyalkyl esters, alkyl esters, aryl esters, phosphate esters, sulfonate esters, sulfate esters and disulfide containing esters; ethers, amides, carbamates, hemisuccinates, dimethylaminoacetates and phosphoryloxymethyloxycarbonyls, as outlined in Advanced Drug Delivery Reviews 1996, 19, 115.

Amine derived prodrugs include, but are not limited to the following groups and combinations of groups:

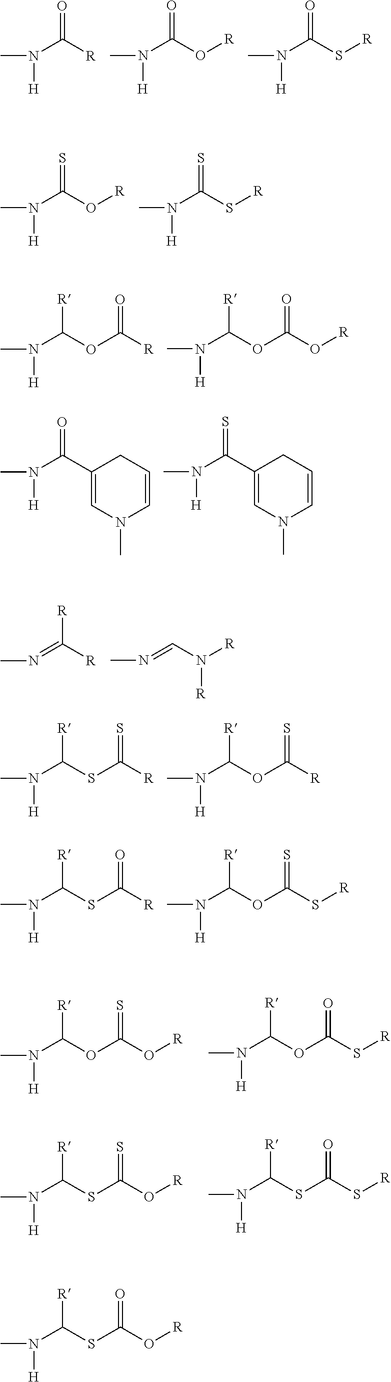

as well as sulfonamides and phosphonamides.

In some embodiments, sites on any aromatic ring portions are susceptible to various metabolic reactions. In some embodiments, incorporation of an appropriate substituent on the aromatic ring structures reduce, minimize or eliminate this metabolic pathway.

### Pharmaceutical Compositions

Described herein are pharmaceutical compositions. In some embodiments, the pharmaceutical compositions comprise an effective amount of a compound of formula (I) or (II); formula (III) or (IV); or formula (V)(a), (V)(b) or (V)(c), or a metabolite, pharmaceutically acceptable salt, ester, prodrug, solvate, hydrate or derivative thereof. In some embodiments, the pharmaceutical compositions comprise an effective amount of a compound formula (I) or (II); formula (III) or (IV); or formula (V)(a), (V)(b) or (V)(c), or a metabolite, pharmaceutically acceptable salt, ester, prodrug, solvate, hydrate or derivative thereof and at least one pharmaceutically acceptable carrier. In some embodiments the pharmaceutical compositions are for the treatment of disorders. In some embodiments the pharmaceutical compositions are for the treatment of disorders in a mammal. In some embodiments the pharmaceutical compositions are for the treatment of disorders in a human. In some embodiments the pharmaceutical compositions are for the treatment of infections. In some embodiments the pharmaceutical compositions are for the treatment of viral infections. In some embodiments the pharmaceutical compositions are for the treatment of HIV infection, including the prevention of HIV infection.

### Integrase Modulation

Also described herein are methods of modulating integrase activity by contacting the integrase with an amount of a compound of formula (I) or (II); formula (III) or (IV); or formula (V)(a), (V)(b) or (V)(c) sufficient to modulate the activity of the integrase. In some embodiments, modulate means inhibiting or activating the integrase activity. In some embodiments, modulating integrase activity comprises contacting integrase with an amount of a compound of formula (I) or (II); formula (III) or (IV); or formula (V)(a), (V)(b) or (V)(c) sufficient to inhibit the activity of integrase. In some embodiments, inhibiting integrase activity in a solution by comprises contacting said solution with an amount of a compound of formula (I) or (II); formula (III) or (IV); or formula (V)(a), (V)(b) or (V)(c) sufficient to inhibit the activity of integrase in said solution. In some embodiments inhibiting integrase activity in a cell comprises contacting said cell with an amount of a compound of formula (I) or (II); formula (III) or (IV); or formula (V)(a), (V)(b) or (V)(c) sufficient to inhibit the activity of integrase in said cell. In some embodiments, inhibiting integrase activity in a tissue comprises contacting said tissue with an amount of a compound of formula (I) or (II); formula (III) or (IV); or formula (V)(a), (V)(b) or (V)(c) sufficient to inhibit the activity of integrase in said tissue. In some embodiments, inhibiting integrase activity in an organism comprises contacting said organism with an amount of a compound of formula (I) or (II); formula (III) or (IV); or formula (V)(a), (V)(b) or (V)(c) sufficient to inhibit the activity of integrase in said organism. In some embodiments, inhibiting integrase activity in an animal comprises contacting said animal with an amount of a compound of formula (I) or (II); formula (III) or (IV); or formula (V)(a), (V)(b) or (V)(c) sufficient to inhibit the activity of integrase in said animal. In some embodiments, inhibiting integrase activity in a mammal comprises contacting said mammal with an amount of a compound of formula (I) or (II); formula (III) or (IV); or formula (V)(a), (V)(b) or (V)(c) sufficient to inhibit the activity of integrase in said mammal. In some embodiments, inhibiting integrase activity in a human comprises contacting said human with an amount of a compound of formula (I) or (II); formula (III) or (IV); or formula (V)(a), (V)(b) or (V)(c) sufficient to inhibit the activity of integrase in said human.

In some embodiments, the integrase is an HIV integrase. In some embodiments, the integrase is an HIV-1 integrase, while in further or additional embodiments the integrase is an HIV-2 integrase. In some embodiments, the integrase is a wild type integrase. In some embodiments, the integrase is a mutated integrase.

### Compound Metabolism, Degradation and Stability

In certain instances, the metabolic profile of a compound influences the ability of the compound to serve as a useful and convenient medication. In human metabolism, the cytochrome P450 (CYP) family of enzymes is the most important contributor to oxidative metabolism. Hepatic CYP enzymes are involved in the metabolism of many drug substances, and in particular, CYP3A4 is noteworthy for its wide range of substrates and high expression in the liver. Facile CYP3A4 metabolism often results is low serum levels of drug substance. In some embodiments, compounds of formula (I) or (II); formula (III) or (IV); or formula (V)(a), (V)(b) or (V)(c) are not significantly degraded or metabolized by CYP3A4, and are thus of particular interest as therapeutics. The term “significantly degraded” as used in this context, should be understood to refer to a compound that upon administration to a subject would not require the aid of a CYP inhibitor to boost serum concentrations. In some embodiments, the degree of CYP3A4 degradation is determined by any suitable method. One such assay is described herein and thus the degree of degradation is measured by exposing a compound to pooled human liver microsomes (protein: 1 mg/mL with CYP3A4 activity at about 4000 pmol/min/mg) at 37° C. for 60 minutes at pH 7.4 at a compound concentration of 1 μM in potassium phosphate buffer (100 mM) containing magnesium chloride (5 mM), EDTA (100 μM) and NADPH (1 mM). The activity of the CYP3A4 enzyme is usually determined separately, in a standard assay, prior to performing the degradation assay. (Indeed it is often provided as part of the spec sheet by the enzyme supplier. It should be noted that in this assay an average CYP3A4 activity is given). Alternatively, in some embodiments, an isolated enzyme assay is performed and thus the degree of degradation is measured by exposing a compound to 10 pmol CYP3A4 enzyme at 37° C. for 60 minutes at pH 7.4 in potassium phosphate buffer (100 mM) containing magnesium chloride (5 mM), EDTA (100 μM) and NADPH (1 mM).

Thus, in some embodiments, compounds of formula (I) or (II); formula (III) or (IV); or formula (V)(a), (V)(b) or (V)(c) are less than about 30% degraded after exposure to pooled human liver microsomes (protein: 1 mg/mL with CYP3A4 activity at about 7800 pmol/min/mg) at 37° C. for 60 minutes at pH 7.4 at a compound concentration of 1 μM in potassium phosphate buffer (100 mM) containing magnesium chloride (5 mM), EDTA (100 μM) and NADPH (1 mM). In further or additional embodiments, compounds of formula (I) or (II); formula (III) or (IV); or formula (V)(a), (V)(b) or (V)(c) are less than about 30% degraded after exposure to 10 pmol CYP3A4 enzyme at 37° C. for 60 minutes at pH 7.4 in potassium phosphate buffer (100 mM) containing magnesium chloride (5 mM), EDTA (100 μM) and NADPH (1 mM).

### Diseases

Described herein are methods of treating a disease in an individual suffering from said disease comprising administering to said individual an effective amount of a composition comprising a compound of formula (I) or (II); formula (III) or (IV); or formula (V)(a), (V)(b) or (V)(c), or a metabolite, pharmaceutically acceptable salt, solvate, polymorph, ester, tautomer or prodrug thereof.

Further disclosed herein, in certain embodiments, is a method of treating or preventing HIV infection, treating AIDS-related complex (ARC), prophylaxis of ARC, delaying the onset of ARC, treating AIDS, prophylaxis of AIDS or delaying the onset of AIDS.

Also described herein are methods of preventing or delaying onset of a disease in an individual at risk for developing said disease comprising administering to said individual an effective amount to prevent or delay onset of said disease, of a composition comprising a compound of formula (I) or (II); formula (III) or (IV); or formula (V)(a), (V)(b) or (V)(c) or a metabolite, pharmaceutically acceptable salt, solvate, polymorph, ester, tautomer or prodrug thereof.

The methods disclosed herein also encompass the prophylaxis or treatment of any disease or disorder in which HIV integrase plays a role including, without limitation, HIV integrase in a human or other mammal. In some embodiments, a compound of formula (I) or (II); formula (III) or (IV); or formula (V)(a), (V)(b) or (V)(c) or a metabolite, pharmaceutically acceptable salt, solvate, polymorph, ester, tautomer or prodrug thereof is used for the manufacture of a medicament for treating such diseases or disorders. Further, in some embodiments, a method disclosed herein comprises administering a human an effective amount of compound of formula (I) or (II); formula (III) or (IV); or formula (V)(a), (V)(b) or (V)(c) for treating any such disease or disorder.

Additionally, in certain instances, a method disclosed herein is used to treat or prevent infection with HIV-1 or HIV-2. In some embodiments, a method disclosed herein is used to treat or prevent infection with a drug resistant strain of HIV. In some embodiments, a method disclosed herein is used to treat or prevent infection with a multidrug resistant strain of HIV. In some embodiments, a method disclosed herein is used to treat or prevent infection with a strain of HIV that exhibits reduced susceptibility to reverse transcriptase inhibitors. In some embodiments, a method disclosed herein is used to treat or prevent infection with a strain of HIV that exhibits at least one mutation compared to wild type HIV. In some embodiments, the mutation conveys resistance to an AIDS or HIV therapeutic.

In some embodiments, patients that are treated with a compound of formula (I) or (II); formula (III) or (IV); or formula (V)(a), (V)(b) or (V)(c), or a metabolite, pharmaceutically acceptable salt, ester, prodrug, solvate, hydrate or derivative of said compounds, according to the methods disclosed herein include, for example, patients that have been diagnosed as having a viral infection.

Disclosed herein, in certain embodiments, is a method of treating a viral infection in a patient in need thereof comprising administering to said patient an effective amount of a compound of formula (I) or formula (II), or a metabolite, pharmaceutically acceptable salt, solvate, polymorph, ester, tautomer or prodrug thereof. In some embodiments, the viral infection is caused by a virus selected from the group consisting of human immunodeficiency viruses 1 (HIV-1), human immunodeficiency viruses 2 (HIV-2), human T-cell leukemia viruses 1 (HTLV-1), human T-cell leukemia viruses 2 (HTLV-2), respiratory syncytial virus (RSV), human papilloma virus (HPV), adenovirus, hepatitis B virus (HBV), hepatitis C virus (HCV), Epstein-Barr virus (EBV), varicella zoster virus (VZV), cytomegalovirus (CMV), herpes simplex viruses 1 (HSV-1), herpes simplex viruses 2 (HSV-2), human herpes virus 8 (HHV-8) Yellow Fever virus, Dengue virus, Japanese. Encephalitis and West Nile virus.

### Viral Infections

Disclosed herein, in certain instances, are methods for treating viral infections, and/or preventing or delaying the onset of conditions related to viral infections. In some embodiments, a compound of formula (I) or (II); formula (III) or (IV); or formula (V)(a), (V)(b) or (V)(c) is used to treat infections or conditions associated with viruses, including, for example, human immunodeficiency viruses 1 and 2 (HIV-1 and HIV-2) including drug resistant strains, human T-cell leukemia viruses 1 and 2 (HTLV-1 and HTLV-2), respiratory syncytial virus (RSV), human papilloma virus (HPV), adenovirus, hepatitis B virus (HBV), hepatitis C virus (HCV), Epstein-Barr virus (EBV), varicella zoster virus (VZV), cytomegalovirus (CMV), herpes simplex viruses 1 and 2 (HSV-1 and HSV-2), human herpes virus 8 (HHV-8, also known as Kaposi's sarcoma-associated virus) and flaviviruses, including Yellow Fever virus, Dengue virus, Japanese Encephalitis and West Nile viruses. Preferably, a compound of formula (I) or (II); formula (III) or (IV); or formula (V)(a), (V)(b) or (V)(c) or a metabolite, pharmaceutically acceptable salt, solvate, polymorph, ester, tautomer or prodrug thereof is used to treat HIV infections. In addition, the present compounds are used to prevent and/or reduce the likelihood of a viral infection such as an HIV infection or a condition which that occurs secondary to a viral infection, such as AIDS, EBV-related lymphoma or HHV-8 associated cancer (sarcoma) will actually occur.

### HIV and AIDS

Over 40 million individuals worldwide are currently infected with human immunodeficiency virus (HIV), with over 14,000 new infections daily and 3 million deaths annually from HIV-related causes. While advances in HIV and AIDS therapy have resulted in fewer AIDS related deaths, the number of HIV-infected individuals continues to rise.

The human immunodeficiency virus (HIV), particularly type-1 (HIV-1) and type-2 (HIV-2) strains, is the causative agent of acquired immunodeficiency syndrome (AIDS). Individuals infected with HIV are initially asymptomatic but eventually undergo the gradual destruction of the immune system, (particularly CD4⁺ T-cells), with a resultant debilitating and ultimately fatal susceptibility to opportunistic infections. Prior to the onset of AIDS, infected individuals MAY experience a precursor AIDS-related complex (ARC), a syndrome characterized by symptoms such as persistent generalized lymphadenopathy, fever and weight loss.

Replication of HIV in a host cell requires integration of the HIV genome into the host cell's DNA. Upon completion of this integration event, integrated proviral DNA is then translated using host cell machinery into viral proteins. Viral protein precursors are then processed by the viral protease to produce the protease, reverse transcriptase, endonuclease/integrase and mature structural proteins of the virus core.

Integration of the HIV genome into the host cell's DNA is performed by the HIV integrase enzyme. HIV integrase has two known enzymatic functions. The enzyme performs 3′-end processing in which two deoxynucleotides are removed from the 3′ ends of the viral DNA. In addition, HIV integrase performs the strand transfer reaction in which the processed 3′ ends of the viral DNA are covalently ligated to the host chromosomal DNA. Clearly, compounds that inhibit HIV integration will inhibit HIV replication in infected cells and would thus be useful in the treatment of HIV infection. In addition, compounds that inhibit HIV integration will prevent HIV infection in uninfected, normal cells and would thus be useful in the prophylaxis of HIV infection.

### Combination Therapy

The development of resistance to a single HIV therapy occurs. In certain instances, the administration of a combination of anti-HIV medications is utilized to suppress HIV replication. In certain instances, an individual receives a nucleoside-type reverse transcriptase inhibitor (NRTI), a non-nucleoside reverse transcriptase inhibitor (NNRTI) or a protease inhibitor, typically in combination. HIV treatment now includes combination therapies (drug cocktails) that involve the dual administration of NRTIs with protease inhibitors or NNRTIs with protease inhibitors and triple combinations of NRTIs, NNRTIs and protease inhibitors. In some embodiments, an effective amount of a compound of formula (I) or (II); formula (III) or (IV); or formula (V)(a), (V)(b) or (V)(c) is administered in combination with other HIV inhibitors selected from NRTIs, NNRTIs or protease inhibitors.

### Metabolism and Pharmacokinetic Profile

In certain instances, the metabolic profile of a compound influence the ability of the compound to serve as a useful and convenient medication. In human metabolism, the cytochrome P450 (CYP) family of enzymes is the most important contributor to oxidative metabolism. Hepatic CYP enzymes are involved in the metabolism of thousands of substrates, including toxic compounds and drug substances. In particular, CYP3A4 is noteworthy for its wide range of substrates and high expression in the liver. As a result, CYP3A4 metabolism is commonly encountered in the development of small molecule drugs. Facile CYP3A4 metabolism often results is low serum levels of drug substance. To achieve efficacy, a readily metabolized drug substance must then be given at higher doses and at shorter intervals. This results in a greater likelihood of drug toxicity and reduced patient compliance. One potential solution to this problem is the co-administration of a CYP3A4 inhibitor to boost the serum levels of the drug substance. However, this potential solution introduces the complexity of a second pharmacological agent and the attendant issues of toxicity, undesired pharmacology, drug-drug interactions and increased burden on the patient. For disease indications requiring combination therapy, the metabolic fate of the other components of the combination therapy must also be considered. In some embodiments, an effective amount of a compound of formula (I) or (II); formula (III) or (IV); or formula (V)(a), (V)(b) or (V)(c) is administered without the aid of a CYP inhibitor to boost serum concentrations. In some embodiments, an effective amount of a compound of formula (I) or (II); formula (III) or (IV); or formula (V)(a), (V)(b) or (V)(c) is administered in combination with other HIV inhibitors selected from NRTIs, NNRTIs or protease inhibitors, without the aid of a CYP inhibitor to boost serum concentrations.

### Modes of Administration and Dosage Forms

In some embodiments, the compounds and compositions described herein are administered either alone or in combination with pharmaceutically acceptable carriers, excipients or diluents, in a pharmaceutical composition, according to standard pharmaceutical practice. In some embodiments, administration of the compounds and compositions described herein is effected by any method that enables delivery of the compounds to the site of action. These methods include, though are not limited to, delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration. The route of administration depends upon for example the condition and disorder of the recipient. In preferred embodiments the compounds and compositions described herein are administered orally. See for example, Goodman et al., in “Goodman and Gilman's: The Pharmacological Basis of Therapeutics”, 9th edition, McGraw-1-Till, New York, N.Y., 1996 and Gennaro, (Ed.), in “Remington's Pharmaceutical Sciences”, 18th edition, Mack Publishing Co., Easton, Pa., 1990). In some embodiments, the pharmaceutical compounds and compositions described herein are in unit dosage forms suitable for single administration of precise dosages. Alternatively, in some embodiments, the pharmaceutical compounds and compositions are presented in multi-dose form in multi-dose containers with one or more added preservatives as required.

In some embodiments, a compound of formula (I) or (II); formula (III) or (IV); or formula (V)(a), (V)(b) or (V)(c) is administered locally to the area in need of treatment, by for example, local infusion during surgery, topical application such as creams or ointments, injection, catheter, or implant, said implant made for example, out of a porous, non-porous, or gelatinous material, including membranes, such as sialastic membranes, or fibers. In some embodiments, the administration is by direct injection at the site of a diseased tissue or organ.

In some embodiments, the compounds and pharmaceutical compositions described herein are in a form suitable for oral administration. In some embodiments, pharmaceutical preparations which are used orally include but are not limited to tablets, troches, lozenges, pills, powders, granules, cachets, capsules including push-fit capsules made of gelatin, as well as soft, sealed capsules made of gelatin and a plasticizer, such as glycerol or sorbitol. Such forms are typically presented as discrete units containing a predetermined amount of the active ingredient. Other pharmaceutical preparations which are used orally include, but are not limited to, syrups, elixirs, solutions or suspensions in aqueous or non-aqueous liquids, oil-in-water liquid emulsions or water-in-oil liquid emulsions. In some embodiments, such preparations are presented in discrete, single-unit dosage forms suitable for single administration of precise dosages containing a predetermined amount of the active ingredient, or in multi-unit form in multi-dose containers with one or more added preservatives as required. In some embodiments, tablets are prepared according to any suitable method (e.g., by compression, or molding, optionally with one or more accessory ingredients). In some embodiments, compressed tablets are prepared by compressing in a suitable machine the active ingredient in a free-flowing form such as a powder or granules, optionally mixed with binders, inert diluents, or lubricating, surface active or dispersing agents. In some embodiments, molded tablets are made by molding in a suitable machine a mixture of the powdered compound moistened with an inert liquid diluent. In some embodiments, the tablets are coated or scored. In some embodiments, the tablets are formulated so as to provide immediate, slow or controlled release of the active ingredient therein. In some embodiments, the push-fit capsules contain the active ingredients in admixture with filler such as lactose, binders such as starches, and/or lubricants such as talc or magnesium stearate and, optionally, stabilizers. In soft capsules, the active compounds is dissolved or suspended in suitable liquids, such as fatty oils, liquid paraffin, or liquid polyethylene glycols. In addition, in some embodiments, stabilizers are added. Dragee cores are provided with suitable coatings. In some embodiments, concentrated sugar solutions are used. In some embodiments, the concentrated sugar solution contains gum arabic, talc, polyvinyl pyrrolidone, carbopol gel, polyethylene glycol, and/or titanium dioxide, lacquer solutions, and suitable organic solvents or solvent mixtures. In some embodiments, dyestuffs or pigments are added to the tablets or Dragee coatings for identification or to characterize different combinations of active compound doses. In some embodiments, pharmaceutical compositions intended for oral administration contain one or more sweetening, flavoring or coloring agents in order to provide palatable and elegant preparations.

In some embodiments, a compound of formula (I) or (II); formula (III) or (IV); or formula (V)(a), (V)(b) or (V)(c) is administered parenterally. Pharmaceutical formulations which are used for parenteral administration include aqueous and non-aqueous sterile solutions, suspensions or emulsions of one or more active compounds in sterile aqueous or oily vehicles, such as, though not limited to water, aqueous propylene glycol, dextrose solutions and the like. Such dosage forms are suitably buffered, if desired. In some embodiments, the compositions contain formulatory agents such as though not limited to suspending, dispersing, thickening and stabilizing agents, antioxidants, buffers, bacteriostats and the like. In some embodiments, formulatory agents useful for rendering the formulation isotonic with the blood of the intended recipient are employed. Suitable lipophilic solvents or vehicles include fatty oils such as sesame oil, or synthetic fatty acid esters, such as ethyl oleate or triglycerides, or liposomes. In some embodiments, aqueous injection suspensions contain substances which increase the viscosity of the suspension, such as sodium carboxymethyl cellulose, sorbitol, or dextran. In some embodiments, the suspension also contains suitable stabilizers or agents which increase the solubility of the compounds to allow for the preparation of highly concentrated solutions. In some embodiments, pharmaceutical preparations are formulated for parenteral administration by injection, for example by bolus injection or continuous infusion. In some embodiments, formulations for parenteral administration are presented in unit dosage form, suitable for single administration of precise dosages, for example in sealed containers, ampoules or vials. Alternatively, the formulations for parenteral administration are presented in multi-dose form in multi-dose containers with one or more added preservatives as required. Additionally, in some embodiments, the formulations for parenteral administration are stored in powder form or in a freeze-dried (lyophilized) condition requiring the addition of the sterile liquid carrier, for example, saline or sterile pyrogen-free water, immediately prior to use. In some embodiments, extemporaneous injection solutions and suspensions are prepared from sterile powders, granules and tablets of the kind previously described.

In some embodiments, pharmaceutical preparations are formulated as a depot preparation. In some embodiments, depot preparations are administered by implantation (for example subcutaneously or intramuscularly) or by intramuscular injection. Thus, in some embodiments, the compounds are formulated with suitable polymeric or hydrophobic materials (for example as an emulsion in an acceptable oil) or ion exchange resins, or as sparingly soluble derivatives, for example, as a sparingly soluble salt.

For buccal or sublingual administration, in some embodiments, the compositions take the form of tablets, lozenges, pastilles, or gels formulated in conventional manner. In some embodiments, such compositions comprise the active ingredient in a flavored basis such as sucrose and acacia or tragacanth.

In some embodiments, pharmaceutical preparations are also formulated in rectal compositions such as suppositories or retention enemas, e.g., containing conventional suppository bases such as cocoa butter, polyethylene glycol, or other glycerides.

In some embodiments, pharmaceutical preparations are administered topically, that is by non-systemic administration. This includes the application of a compound of formula (I) or (II); formula (III) or (IV); or formula (V)(a), (V)(b) or (V)(c) is administered externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical preparations suitable for topical administration include liquid or semi-liquid preparations suitable for penetration through the skin to the site of inflammation such as gels, liniments, lotions, creams, ointments or pastes, and drops suitable for administration to the eye, ear or nose. In some embodiments, a formulation for topical administration comprises from about 0.001% to about 10% w/w, or from about 1% to about 2% by weight of the active ingredient. In some embodiments, a formulation for topical administration comprises about 10% w/w, but preferably less than about 5% w/w, more preferably from about 0.1% to about 1% w/w Of the active ingredient.

Pharmaceutical preparations for administration by inhalation are delivered from an insufflator, nebulizer pressurized packs or other convenient means of delivering an aerosol spray. In some embodiments, pressurized packs comprise a suitable propellant such as dichlorodifluoromethane, trichlorofluoromethane, dichlorotetrafluoroethane, carbon dioxide or other suitable gas. In the case of a pressurized aerosol, in some embodiments, the dosage unit is determined by providing a valve to deliver a metered amount. In some embodiments, for administration by inhalation or insufflation, pharmaceutical preparations take the form of a dry powder composition, for example a powder mix of the compound and a suitable powder base such as lactose or starch. In some embodiments, the powder composition is presented in unit dosage form, in for example, capsules, cartridges, gelatin or blister packs from which the powder is administered with the aid of an inhalator or insufflator.

### Formulations

The pharmaceutical compositions described herein contain at least one compound described herein in admixture with one or more non-toxic, pharmaceutically acceptable excipients (such as, though not limited to pharmaceutical carriers, excipients, adjuvants, and the like, as well as other medicinal or pharmaceutical agents) which are suitable for the manufacture and administration of the composition, formulated as appropriate for the desirable mode of administration. a formulation for topical administration comprises the pharmaceutical compositions described herein contain the active ingredient in a form suitable for oral administration, for example, as tablets, troches, lozenges, aqueous or oily suspensions, dispersible powders or granules, emulsions, hard or soft capsules, or syrups or elixirs. In some embodiments, compositions intended for oral use are prepared according to any suitable method, and such compositions contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, such as though not limited to inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. In some embodiments, the tablets are un-coated or coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate is employed as appropriate. In some embodiments, formulations for oral use are presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

In some embodiments, the compounds or Compositions described herein are delivered in a vesicle, such as a liposome. In some embodiments, the compounds and pharmaceutical compositions described herein are delivered in a controlled release system, or a controlled release system is placed in proximity of the therapeutic target. In one embodiment, a pump is used.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents are a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. In some embodiments, an aqueous suspensions contains one or more preservatives, for example ethyl, or n-propyl p-hydroxybenzoate, one or more coloring agents, one or more flavoring agents, and one or more sweetening agents, such as sucrose, saccharin or aspartame.

Suitable pharmaceutical carriers include inert diluents or fillers, water and various organic solvents. In some embodiments, the pharmaceutical compositions contain additional ingredients such as flavorings, binders, excipients and the like. In some embodiments, tablets containing various excipients, such as citric acid are employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. In some embodiments, solid compositions of a similar type are employed in soft and hard filled gelatin capsules. Preferred materials, therefore, include lactose or milk sugar and high molecular weight polyethylene glycols. In some embodiments, when aqueous suspensions or elixirs are desired for oral administration, the active compound therein is combined with various sweetening or flavoring agents, coloring matters or dyes and, if desired, emulsifying agents or suspending agents, together with diluents such as water, ethanol, propylene glycol, glycerin, or combinations thereof.

In some embodiments, oily suspensions are formulated by suspending the active ingredient in a vegetable oil, for example arachis oil, olive oil, sesame oil or coconut oil, or in mineral oil such as liquid paraffin. In some embodiments, the oily suspensions contain a thickening agent, for example beeswax, hard paraffin or cetyl alcohol. In some embodiments, sweetening agents such as those set forth above, and flavoring agents are added to provide a palatable oral preparation. In some embodiments, these compositions are preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. In some embodiments, additional excipients, for example sweetening, flavoring and coloring agents, are present. In some embodiments, these compositions are preserved by the addition of an anti-oxidant such as ascorbic acid.

In some embodiments, pharmaceutical compositions are in the form of oil-in-water emulsions. In some embodiments, the oily phase is a vegetable oil, for example olive oil or arachis oil, or a mineral oil, for example liquid paraffin or mixtures of these. Suitable emulsifying agents include naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. In some embodiments, the emulsions also contain sweetening agents, flavoring agents, preservatives and antioxidants.

In some embodiments, syrups and elixirs are formulated with sweetening agents, for example glycerol, propylene glycol, sorbitol or sucrose. In some embodiments, such formulations also contain a demulcent, a preservative, flavoring and coloring agents and antioxidant.

In some embodiments, pharmaceutical compositions are in the form of a sterile injectable aqueous solution. Among the acceptable vehicles and solvents that are employed are water, Ringer's solution and isotonic sodium chloride solution. In some embodiments, the sterile injectable preparation is also a sterile injectable oil-in-water microemulsion where the active ingredient is dissolved in the oily phase. For example, the active ingredient is first dissolved in a mixture of soybean oil and lecithin. The oil solution then introduced into a water and glycerol mixture and processed to form a microemulsion. In some embodiments, the injectable solutions or microemulsions are introduced into a patient's blood-stream by local bolus injection. Alternatively, it is advantageous to administer the solution or microemulsion in such a way as to maintain a constant circulating concentration of the instant compound. In some embodiments, a continuous intravenous delivery device is utilized. An example of such a device is the Deltec CADD-PLUS™ model 5400 intravenous pump. In some embodiments, the pharmaceutical compositions are in the form of a sterile injectable aqueous or oleagenous suspension for intramuscular and subcutaneous administration. Suspensions are formulated using any suitable dispersing or wetting agents and suspending agents which have been mentioned above. In some embodiments, a sterile injectable preparation is a sterile injectable solution or suspension in a non-toxic parenterally-acceptable diluent or solvent; for example as a solution in 1,3-butane diol. In some embodiments, sterile, fixed oils are employed as a solvent or suspending medium. Any bland fixed oil is employed including synthetic mono- or diglycerides. In addition, fatty acids such as oleic acid find use in the preparation of injectables.

In some embodiments, pharmaceutical compositions are also administered in the form of suppositories for rectal administration of the drug. In some embodiments, these compositions are prepared by mixing the active ingredient with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

In some embodiments, creams, ointments, jellies, solutions or suspensions, etc., containing a compound of formula (I) or (II); formula (III) or (IV); or formula (V)(a), (V)(b) or (V)(c) is administered are used. As used herein, topical application includes mouth washes and gargles.

In some embodiments, pharmaceutical compositions are administered in intranasal form via topical use of suitable intranasal vehicles and delivery devices, or via transdermal routes, using for example transdermal skin patches. To be administered in the form of a transdermal delivery system, the dosage administration will, of course, be continuous rather than intermittent throughout the dosage regimen.

In some embodiments, the formulations are presented in unit dosage form and are prepared by any of the methods well known in the art of pharmacy. All methods include the step of bringing into association a compound of formula (I) or (II); formula (III) or (IV); or formula (V)(a), (V)(b) or (V)(c) is administered or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation. Methods of preparing various pharmaceutical compositions with a specific amount of active compound are known, or will be apparent, to those skilled in this art.

It should be understood that in addition to the ingredients particularly mentioned above, the compounds and compositions described herein may include other agents conventional in the art having regard to the type of formulation in question.

### Doses

The amount of pharmaceutical composition administered depends on a variety of factors. The amount will firstly be dependent on the mammal being treated. In the instances where pharmaceutical compositions are administered to a human subject, the daily dosage will normally be determined by the prescribing physician with the dosage generally varying according to the age, sex, diet, weight, general health and response of the individual patient, the severity of the patient's symptoms, the precise indication or condition being treated, the severity of the indication or condition being treated, time of administration, route of administration, the disposition of the composition, rate of excretion, drug combination, and the discretion of the prescribing physician. In some embodiments, the route of administration varies depending on the condition and its severity. Preferably, the pharmaceutical composition is in unit dosage form. In such form, the preparation is subdivided into unit doses containing appropriate quantities of the active component, e.g., an effective amount to achieve the desired purpose. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. In some embodiments, the total daily dosage is divided and administered in portions during the day if desired. The amount and frequency of administration of a compound of formula (I) or (II); formula (III) or (IV); or formula (V)(a), (V)(b) or (V)(c), and if applicable other therapeutic agents and/or therapies, is regulated according to the judgment of the attending clinician (physician) considering such factors as described above. In some embodiments, administration occurs in an amount of between about 0.001 mg/kg of body weight to about 100 mg/kg of body weight per day (administered in single or divided doses), more preferably at least about 0.1 mg/kg of body weight per day. In some embodiments, a therapeutic dosage is, e.g., from about 0.01 mg to about 7000 mg of compound, and preferably includes, e.g., from about 0.05 mg to about 2500 mg. In some embodiments, the quantity of active compound in a unit dose of preparation is varied or adjusted from about 0.1 mg to 1000 mg, preferably from about 1 mg to 300 mg, more preferably 10 mg to 200 mg, according to the particular application. In some instances, dosage levels below the lower limit of the aforesaid range are used, while in other cases larger doses are employed without causing any harmful side effect, e.g. by dividing such larger doses into several small doses for administration throughout the day. The amount administered will vary depending on the particular IC₅₀ value of the compound used. In combinational applications in which the compound is not the sole therapy, in some embodiments, it is possible to administer lesser amounts of compound and still have therapeutic or prophylactic effect.

### Combination Therapies

In some embodiments, a compound of formula (I) or (II); formula (III) or (IV); or formula (V)(a), (V)(b) or (V)(c) or a metabolite, pharmaceutically acceptable salt, solvate, polymorph, ester, tautomer or prodrug thereof is administered as a sole therapy. In some embodiments, a compound of formula (I) or (II); formula (III) or (IV); or formula (V)(a), (V)(b) or (V)(c) or a metabolite, pharmaceutically acceptable salt, solvate, polymorph, ester, tautomer or prodrug thereof is administered in combination with another therapy or therapies.

In certain instances, the therapeutic effectiveness of one of a compound of formula (I) or (II); formula (III) or (IV); or formula (V)(a), (V)(b) or (V)(c) is enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit experienced by a patient is increased by administering one of a compound of formula (I) or (II); formula (III) or (IV); or formula (V)(a), (V)(b) or (V)(c) with a second therapeutic agent (which also includes a therapeutic regimen) that also has therapeutic benefit. By way of example only, in a treatment for viral infection involving administration of a compound of formula (I) or (II); formula (III) or (IV); or formula (V)(a), (V)(b) or (V)(c), increased therapeutic benefit results by providing the patient with another therapeutic agent for viral infection. Or, by way of example only, if one of the side effects experienced by a patient upon receiving one of a compound of formula (f) or (II); formula (III) or (IV); or formula (V)(a), (V)(b) or (V)(c) is nausea, then it is appropriate to administer an anti-nausea agent in combination with the compound. Additional therapy or therapies include, but are not limited to physiotherapy, psychotherapy, radiation therapy, application of compresses to a diseased area, rest, altered diet, and the like. Regardless of the disease, disorder or condition being treated, the overall benefit experienced by the patient is additive of the two therapies or therapeutic agents, or the patient experiences a synergistic benefit.

In the instances where a compound of formula (I) or (II); formula (III) or (IV); or formula (V)(a), (V)(b) or (V)(c) is administered with other therapeutic agents, a compound of formula (I) or (II); formula (III) or (IV); or formula (V)(a), (V)(b) or (V)(c) need not be administered in the same, pharmaceutical composition as other therapeutic agents. In some embodiments, a compound of formula (I) or (II); formula (III) or (IV); or formula (V)(a), (V)(b) or (V)(c) is administered by a different route. For example, the compounds/compositions are administered orally to generate and maintain good blood levels thereof, while the other therapeutic agent are administered intravenously.

In some embodiments, a compound of formula (I) or (II); formula (III) or (IV); or formula (V)(a), (V)(b) or (V)(c) is administered concurrently (e.g., simultaneously, essentially simultaneously or within the same treatment protocol), sequentially or dosed separately to other therapeutic agents. In some embodiments, the administration of a compound of formula (I) or formula (II), or a metabolite, pharmaceutically acceptable salt, solvate, polymorph, ester, tautomer or prodrug thereof and the second therapeutic agent is sequential. In some embodiments, the sequential administration is a cycling therapy. In some embodiments, the compound of formula (I) or formula (II), is administered before the second therapeutic agent. In some embodiments, the compound of formula (I) or formula (II), is administered after the second therapeutic agent. In some embodiments, the administration of a compound of formula (I) or formula (II), or a metabolite, pharmaceutically acceptable salt, solvate, polymorph, ester, tautomer or prodrug thereof and the second therapeutic agent is simultaneous.

The determination of the timing and mode of administration and the advisability of administration, where possible, in the same pharmaceutical composition, is within the knowledge of the skilled clinician. In some embodiments, the initial administration is made according to established protocols known in the art, and then, based upon the observed effects, the dosage, modes of administration and times of administration is modified by the skilled clinician. The particular choice of compound and other therapeutic agent will depend upon the diagnosis of the attending physicians and their judgment of the condition of the patient and the appropriate treatment protocol.

In some embodiments, he compounds described herein or a metabolite, pharmaceutically acceptable salt, solvate, polymorph, ester, tautomer or prodrug thereof are administered in combination with an anti HIV or AIDS drug. In some embodiments, he compounds described herein or a metabolite, pharmaceutically acceptable salt, solvate, polymorph, ester, tautomer or prodrug thereof are administered in combination with a reverse transcriptase inhibitor, a viral protease inhibitor, a fusion inhibitor, a cytokine, a cytokine inhibitor; a glycosylation inhibitor, a viral mRNA processing inhibitor, an entry inhibitor, an integrase inhibitor or a maturation inhibitor or a combination thereof. In some embodiments, he compounds described herein or a metabolite, pharmaceutically acceptable salt, solvate, polymorph, ester, tautomer or prodrug thereof are administered in combination with adefovir, abacavir, amprenavir, atazanavir, apricitabine, bevirimat, darunavir, delavirdine, didanosine, efavirenz, emtricitabine, elvitegravir, enfuvirtide, etravirine, fosamprenavir, fuseon, indinavir, lamivudine, lopinavir, maraviroc, nelfinavir, nevirapine, racivir, raltegravir, reverset, ritonavir, saquinavir, stavudine, tenofovir, tipranavir, vicriviroc, zalcitabine, zidovudine, interferon-α, interferon-β or interferon-γ, or a combination of two or more thereof.

### Kits

The compounds, compositions and methods described herein provide kits for the treatment of disorders, such as the ones described herein. These kits comprise a compound, compounds or compositions described herein in a container and, optionally, instructions teaching the use of the kit according to the various methods and approaches described herein. In some embodiments, a kit includes information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information is based on the results of various studies, for example, studies using experimental animals involving in vivo models and studies based on human clinical trials. Kits described herein are provided, marketed and/or promoted to health providers, including physicians, nurses, pharmacists, formulary officials, and the like. In some embodiments, a kit is marketed directly to the consumer.

In some embodiments, a compound of formula (I) or (II); formula (III) or (IV); or formula (V)(a), (V)(b) or (V)(c) is utilized for diagnostics and as research reagents. For example, a compound of formula (I) or (II); formula (III) or (IV); or formula (V)(a), (V)(b) or (V)(a), either alone or in combination with other compounds, is used as tools in differential and/or combinatorial analyses to elucidate expression patterns of genes expressed within cells and tissues. As one non-limiting example, expression patterns within cells or tissues treated with one or more compounds are compared to control cells or tissues not treated with compounds and the patterns produced are analyzed for differential levels of gene expression as they pertain, for example, to disease association, signaling pathway, cellular localization, expression level, size, structure or function of the genes examined. In some embodiments, these analyses are performed on stimulated or unstimulated cells and in the presence or absence of other compounds which affect expression patterns.

Besides being useful for human treatment, a compound of formula (I) or (II); formula (III) or (IV); or formula (V)(a); (V)(b) or (V)(c) and formulations thereof is also useful for veterinary treatment of companion animals, exotic animals and farm animals, including mammals, rodents, and the like. More preferred animals include horses, dogs, and cats.

The examples and preparations provided below further illustrate and exemplify the compounds of formula (I) or (II); formula (III) or (IV); or formula (V)(a), (V)(b) or (V)(c) and methods of preparing such compounds. It is to be understood that the scope of the present disclosures is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers.

### EXAMPLES

### I. Chemical Syntheses

### Example 1

### Compounds of Formula (I)

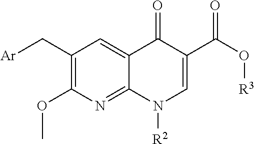

Compounds of formula (I) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

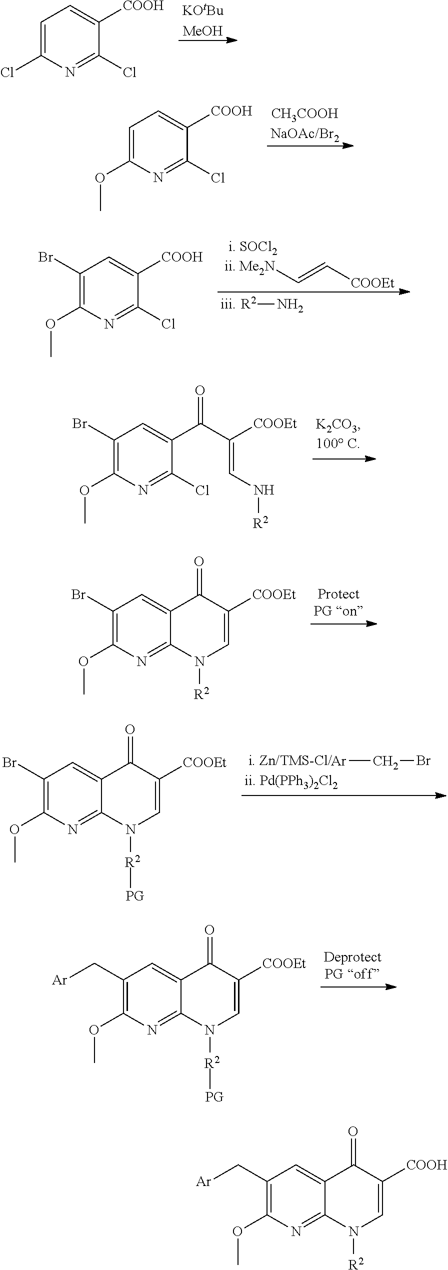

### Example 1A

### 6-(3-Chloro-2-fluoro-benzyl)-1-((S)-1-hydroxymethyl-2-methyl-propyl)-7-methoxy-4-oxo-1,4-dihydro-[1,8]naphthyridine-3-carboxylic acid

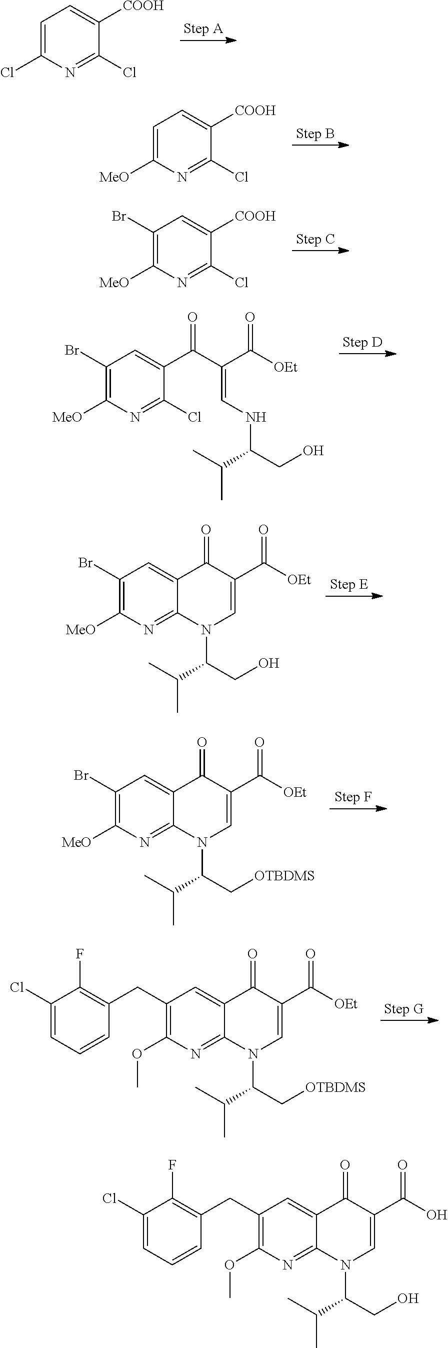

### Step A: 2-Chloro-6-methoxypyridine-3-carboxylic acid

A mixture of 2,6-dichloropyridine-3-carboxylic acid (6.5 g, 33 mmol), potassium tert-butoxide (11.4 g, 0.10 mol), and anhydrous methanol (300 mL) was heated to reflux for 4 days and cooled to room temperature. After evaporation of the solvent, the residue was diluted with water and acidified with 35% aqueous hydrochloric acid. The resulting solid was collected by filtration, washed with water, and dried to give 4.8 g (84%) of 2-chloro-6-methoxypyridine-3-carboxylic acid as a white solid.

¹H NMR (DMSO-d₆, 400 MHz): δ (13.33 (brs, 1H, OH, exchangeable with D₂O), 8.19 (d, J=8.5 Hz, 1H), 6.92 (d, J=8.5 Hz, 1H), 3.92 (s, 3H).

### Step B: 2-Chloro-5-bromo-6-methoxypyridine-3-carboxylic acid

To a suspension of 2-chloro-6-methoxypyridine-3-carboxylic acid (4.69 g, 25 mmol) and sodium acetate (4.10 g, 50 mmol) in 200 ml of glacial acetic acid was added bromine (16.0, 100 mmol) at room temperature. The mixture was warmed to 80° C. overnight, cooled to room temperature and poured into 500 ml of ice-water with strong stirring. The solid was filtered and washed with water to give 5.2 g (78%) of pure product as a white solid.

¹H NMR (DMSO-d₆, 400 MHz): δ 8.51 (s, 1H), 3.93 (s, 3H).

MS: 266 (M−1).

### Step C: 2-(5-Bromo-2-chloro-6-methoxy-pyridine-3-carbonyl)-3-((S)-1-hydroxymethyl-2-methyl-propylamino)-acrylic acid ethyl ester

A mixture of 2-chloro-5-bromo-6-methoxypyridine-3-carboxylic acid (8.0 g, 30 mmol) and thionyl chloride (4.4 mL, 60 mmol) in 50 ml of anhydrous toluene and 0.5 ml of anhydrous DMF was refluxed for 2 h. The solvent was removed under reduced pressure to give a mobile oil residue which was azeoptoped with toluene (20 mL). The residue was dissolved in 20 ml of anhydrous THF. This solution was added dropwise to a solution of ethyl 3-(dimethylamino)acrylate (4.7 g, 33 mmol) and triethylamine (3.64 g, 36 mmol) in 20 ml of anhydrous THF under nitrogen and heated under reflux for 7 hours The mixture was allowed to cool to room temperature and concentrated under reduced pressure. Water (100 mL) and ethyl acetate (100 mL) was added to allow partitioning. The organic layer was washed with saturated aqueous sodium bicarbonate (×2), water, brine, dried over sodium sulfate and concentrated under reduced pressure. The crude product was purified by flash chromatography (ISCO, chloroform/methanol, 0-40%, 40 min) to give the pure product as yellow oil (7.3 g, 62%).

A solution of the above product (7.3 g, 18.6 mmol) and L-valinol (1.92 g, 18.6 mmol) in anhydrous THF (100 mL) was stirred for 30 min at room temperature and evaporated to dryness to give a crude product in a quantitative yield, which was used for next step without further purification. An analytically pure sample was prepared by silica gel chromatography (ISCO, Chloroform/methanol, 0-40%, 40 min) to give the pure compound as yellow oil.

hu 1H NMR (DMSO-d₆, 400 MHz): δ 10.95 (dd, J=9.6 and 13.8 Hz, 1H, NH, exchangeable with D₂O), 8.24 (d, J=14.3 Hz, 1H, it becomes singlet after D₂O exchange), 7.98 (s, 1H), 5.05 (t, J=5.1 Hz, 1H, OH, exchangeable with D₂O), 3.95 (s, 3H), 3.91 (q, J=7.0 Hz, 2H), 3.59 (m, 2H), 3.36 (m, 1H), 1.93 (m, 1H), 0.95 (d, J=6.6 Hz, 3H), 0.91 (d, J=6.6 Hz, 3H), 0.90 (t, J=7.0 Hz, 3H).

MS: 449, 451 (M+1).

### Step D: 6-Bromo-1-((S)-1-hydroxymethyl-2-methyl-propyl)-7-methoxy-4-oxo-1,4-dihydro-[1,8]naphthyridine-3-carboxylic acid ethyl ester

A mixture of 2-(5-bromo-2-chloro-6-methoxy-pyridine-3-carbonyl)-3-((S)-1-hydroxymethyl-2-methyl-propylamino)-acrylic acid ethyl ester (1.1 g, 2.5 mmol) and potassium carbonate (0.7 g, 5.0 mmol) in anhydrous DMF (15 mL) was stirred at 100° C. for 2 hours and evaporated to dryness under reduced pressure. The crude material was purified by ISCO (Chloroform/methanol, 0-40%, 40 min) to give the title compound as a yellow solid (0.7 g, 68%). 1H NMR (DMSO-d6, 400 MHz): δ 8.73 (s, 1H), 8.58 (s, 1H), 5.25 (m, 1H), 5.11 (brs, 1H, OH, exchangeable with D2O), 4.24 (q, J=7.1 Hz, 2H), 4.08 (s, 3H), 3.94 (m, 1H), 3.91 (m, 1H), 2.27 (m, 1H), 1.28 (t, J=7.1 Hz, 3H), 1.10 (d, J=6.2 Hz, 3H), 0.74 (d, J=6.2 Hz, 3H).

MS: 413, 415 (M+1).

### Step E: 6-Bromo-1-[(S)-1-(tert-butyl-dimethyl-silanyloxymethyl)-2-methyl-propyl]-7-methoxy-4-oxo-1,4-dihydro-[1,8]naphthyridine-3-carboxylic acid ethyl ester

To a mixture of 6-bromo-1-((S)-1-hydroxymethyl-2-methyl-propyl)-7-methoxy-4-oxo-1,4-dihydro-[1,8]naphthyridine-3-carboxylic acid ethyl ester (0.63 g, 1.5 mmol) and imidazole (1.04 g, 15.0 mmol) in 12 ml of anhydrous DMF was added tert-butyldimethylsilyl chloride (1.28 g, 7.5 mmol) under argon at room temperature. The resulting mixture was stirred at room temperature overnight and evaporated to dryness under reduced pressure. The resulting crude material was purified by ISCO (hexane/EtOAc, 0-90%, 40 min) to give the title compound as yellow oil (0.7 g, 89%).

1H NMR (DMSO-d6, 400 MHz): δ 8.72 (s, 1H), 8.61 (s, 1H), 5.33 (m, 1H), 4.26 (q, J=7.1 Hz, 2H), 4.07 (s, 3H), 4.05 (m, 1H), 3.94 (m, 1H), 2.36 (m, 1H), 1.30 (t, J=7.1 Hz, 3H), 1.16 (d, J=6.2 Hz, 3H), 0.79 (d, J=6.2 Hz, 3H), 0.77 (s, 9H), 0.02 (s, 6H).

MS: 527, 529 (M+1).

### Step F: 1-[(S)-1-(tert-Butyl-dimethyl-silanyloxymethyl)-2-methyl-propyl]-6-(3-chloro-2-fluoro-benzyl)-7-methoxy-4-oxo-1,4-dihydro-[1,8]naphthyridine-3-carboxylic acid ethyl ester

Under an argon stream, zinc powder (240 mg, 3.67 mmol) was suspended in 0.5 ml of dry tetrahydrofuran and the suspension was heated at 60° C. 1,2-Dibromoethane (0.7 μl, 0.008 mmol) and trimethylsilyl chloride (2.0 μl, 0.016 mmol) were added at this temperature and the mixture was stirred for an additional 30 min followed by addition dropwise of a solution of 2-fluoro-3-chloro-benzyl bromide (176 mg, 0.79 mmol) in 1 ml of dry tetrahydrofuran. The mixture was stirred for an additional hour and allowed to cool to room temperature to give a solution of 2-fluoro-3-chloro-benzylzinc bromide in tetrahydrofuran. This solution was used in the next step.

6-Bromo-1-[(S)-1-(tert-butyl-dimethyl-silanyloxymethyl)-2-methyl-propyl]-7-methoxy-4-oxo-1,4-dihydro-[1,8]naphthyridine-3-carboxylic acid ethyl ester (320 mg, 0.61 mmol) and dichlorobis(triphenylphosphine)palladium(II) (17 mg, 0.024 mmol) were added to 9 ml of dry tetrahydrofuran under an argon stream. The solution prepared above was added at 60° C. and the mixture was stirred with heating at the same temperature for 1.5 hour. The reaction mixture was allowed to cool to room temperature, and 1 N hydrochloric acid was added. The resulting mixture was extracted three times with ethyl acetate. The organic layers were combined, washed with water; brine, dried over anhydrous sodium sulfate and concentrated under reduced pressure. The crude material was purified by silica gel chromatography (ISCO, 12 g of column, hexane/ethyl acetate, 0-30%, 25 min; 30-80%, 10 min; 80%, 5 min) to give 100 mg of the title product as a white solid.

1H NMR (DMSO-d6, 400 MHz): δ 8.71 (s, 1H), 8.19 (s, 1H), 7.55 (dt, J=2.0 and 7.8 Hz, 1H), 7.32 (dt, J=2.0 and 7.8 Hz, 1H), 7.25 (t, J=7.8 Hz, 1H), 5.41 (m, 1H), 4.26 (q, J=7.1 Hz, 2H), 4.11 (s, 2H), 4.08 (s, 3H), 4.02 (m, 1H), 3.94 (m, 1H), 2.35 (m, 1H), 1.31 (t, J=7.1 Hz, 3H), 1.17 (d, J=6.2 Hz, 3H), 0.81 (d, J=6.2 Hz, 3H), 0.76 (s, 9H), 0.03 (s, 6H).

MS: 592 (M+1).

### Step G: 6-(3-Chloro-2-fluoro-benzyl)-1-((S)-1-hydroxymethyl-2-methyl-propyl)-7-methoxy-4-oxo-1,4-dihydro-[1,8]naphthyridine-3-carboxylic acid

1-[(S)-1-(tert-Butyl-dimethyl-silanyloxymethyl)-2-methyl-propyl]-6-(3-chloro-2-fluoro-benzyl)-7-methoxy-4-oxo-1,4-dihydro-[1,8]naphthyridine-3-carboxylic acid ethyl ester (100 mg, 0.17 mmol) was dissolved in methanol (10 mL). A solution of 1 ml of 25% sodium methoxide in methanol and 2 ml of water was added. The mixture was refluxed for 4 hours, allowed to cool to room temperature and evaporated to a small volume under reduced pressure. Water (10 mL) was added and the resulting mixture was filtered. The filtrate was neutralized with 1 N hydrochloric acid. The solid was filtered and washed with water to give a pure product as an off-white solid (60 mg, 79%).

¹H NMR (DMSO-d₆, 400 MHz): δ 15.16 (brs, 1H, OH, exchangeable with D₂O), 9.01 (s, 1H), 8.32 (s, 1H), 7.55 (td, J=7.9 Hz, 1H), 7.35 (t, J=7.9 Hz, 1H), 7.25 (t, J=7.9 Hz, 1H), 5.50 (brs, 1H), 5.19 (brs, 1H, OH, exchangeable with D₂O), 4.16 (s, 2H), 4.12 (s, 3H), 4.05 (m, 1H), 3.85 (m, 1H), 2.38 (m, 1H), 1.15 (d, J=6.28 Hz, 3H), 0.82 (d, J=6.28 Hz, 3H).

MS: 449 (M+1).

### Examples 1B-1R

Examples 1B-1R were prepared according to the procedure described above for example 1A.

| | | Ana- |
|---|---|---|
| Com- | | lytical |
| pound | Structure | Data |
| 1A | 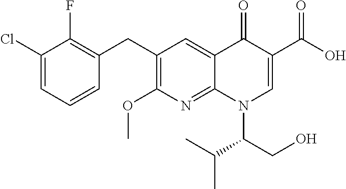 | MS: 449 (M + H)⁺ |
| 1B | 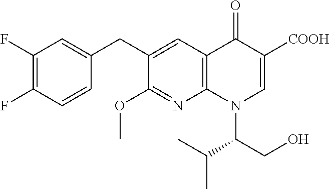 | MS: 433 (M + H)⁺ |
| 1C | 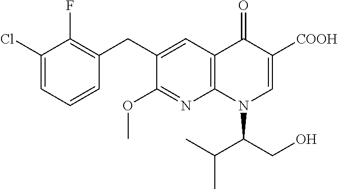 | MS: 449 (M + H)⁺ |
| 1D | 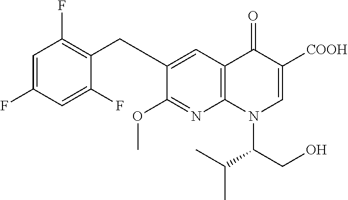 | MS: 451 (M + H)⁺ |
| 1E | 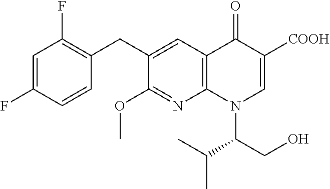 | MS: 433 (M + H)⁺ |
| 1F | 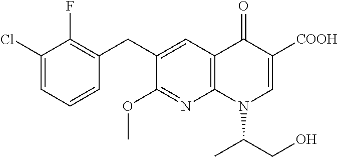 | MS: 421 (M + H)⁺ |
| 1G | 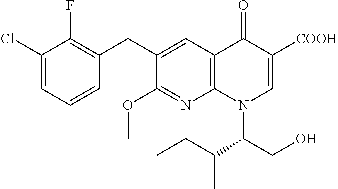 | MS: 463 (M + H)⁺ |
| 1H | 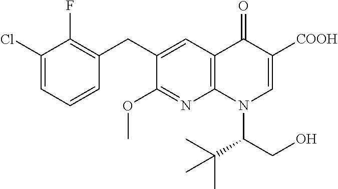 | MS: 463 (M + H)⁺ |
| 1I | 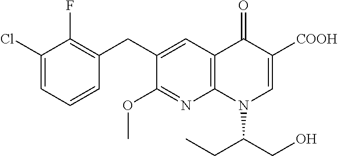 | MS: 435 (M + H)⁺ |
| 1J | 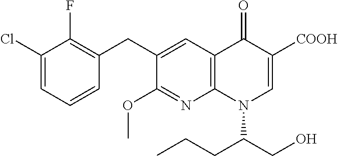 | MS: 449 (M + H)⁺ |
| 1K | 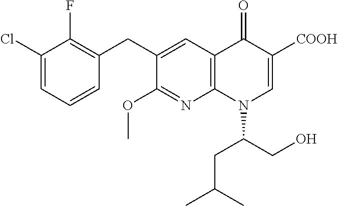 | MS: 463 (M + H)⁺ |
| 1L | 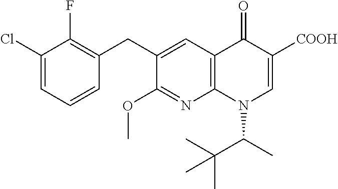 | MS: 447 (M + H)⁺ |
| 1M | 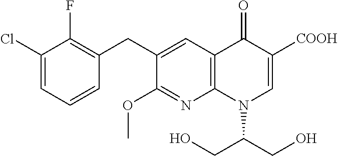 | MS: 437 (M + H)⁺ |
| 1N | 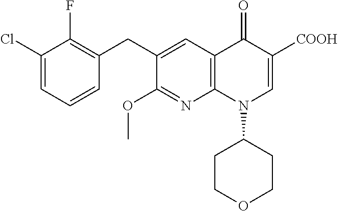 | MS: 447 (M + H)⁺ |
| 1O | 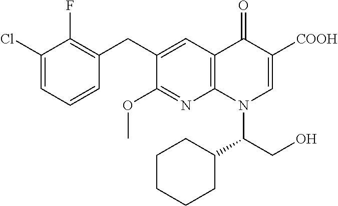 | MS: 489 (M + H)⁺ |
| 1P | 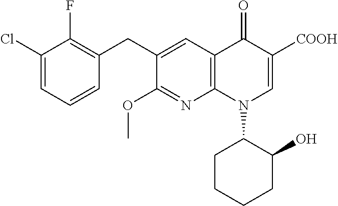 | MS: 461 (M + H)⁺ |
| 1Q | 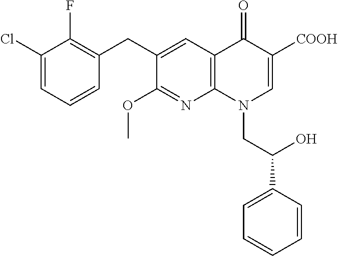 | MS: 483 (M + H)⁺ |
| 1R | 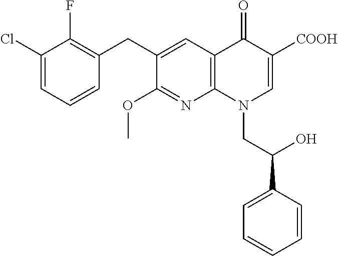 | MS: 483 (M + H)⁺ |

### Example 2

### Compounds of Formula (II)

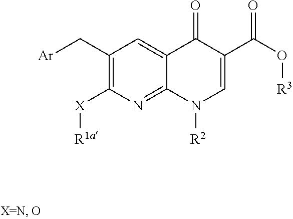

Compounds of formula (II) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

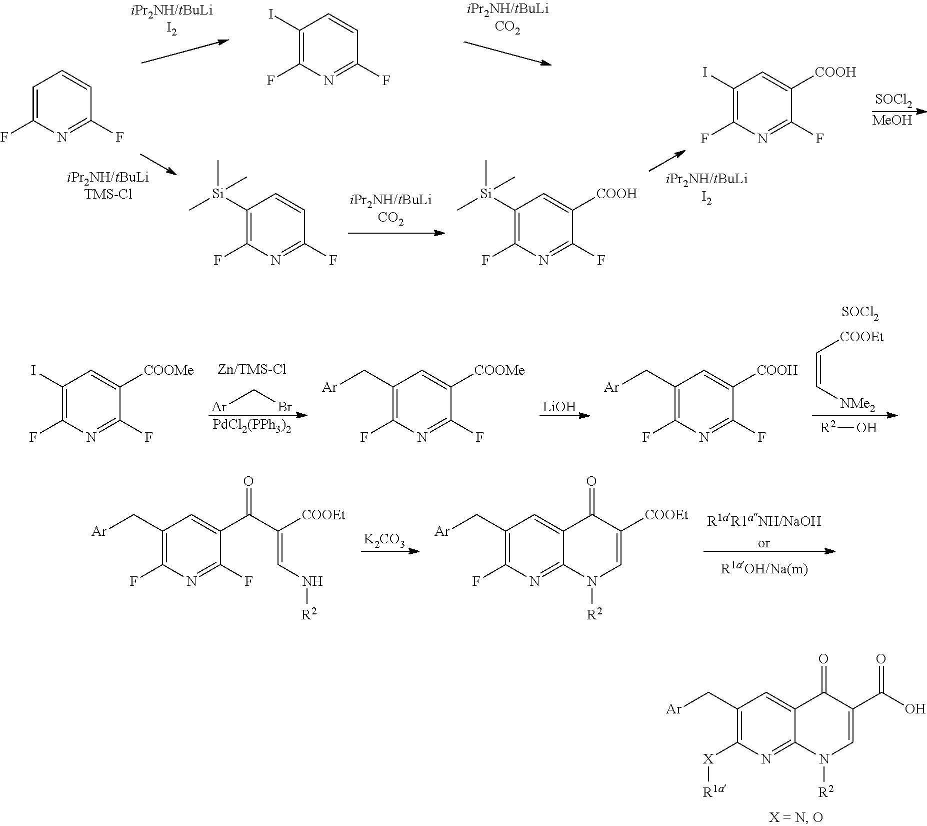

### Example 2A

### (S)-6-(3-chloro-2-fluorobenzyl)-7-morpholin-1-(1-hydroxy-3,3-dimethylbutan-2-yl)-4-oxo-1,4-dihydro-1,8-naphthyridine-3-carboxylic acid

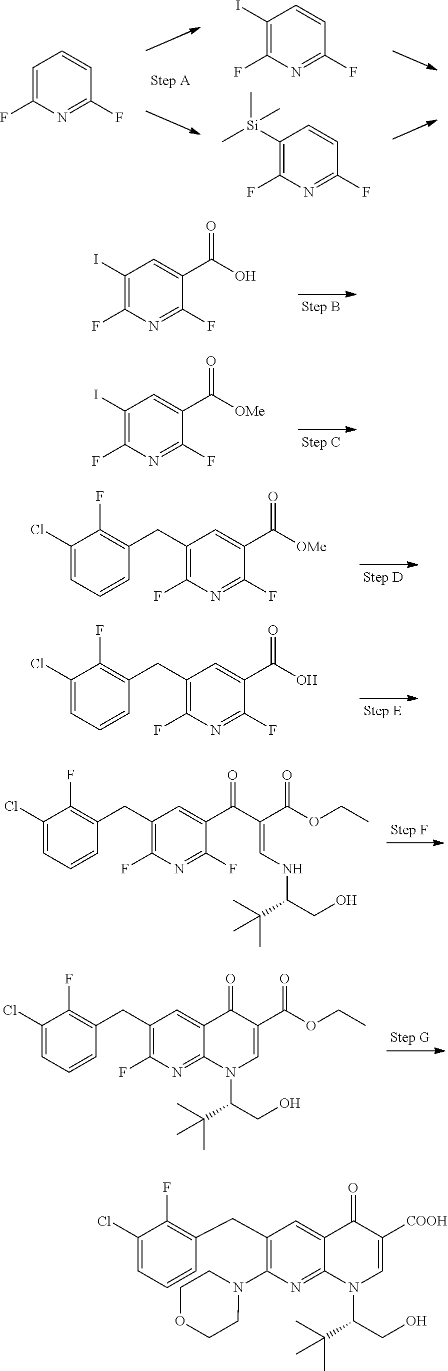

### Step A: 2,6-Difluoro-5-iodopyridine-3-carboxylic acid

### Method A:

(1) 2,6-Difluoro-3-iodopyridine

Diisopropylamine (141.3 mL, 101.19 g, 1 mol) and 2,6-difluoropyridine (115.08 g, 1 mol) were added consecutively to a solution of butyllithium (1.6M in hexane, 625 mL, 1 mol) in tetrahydrofuran (2000 mL) kept in a dry ice/methanol bath. After 1 h at −75° C., the mixture was treated with a solution of iodine (253.8 g, mol) in tetrahydrofuran (1000 mL). The mixture was washed with a 10% aqueous solution (500 mL) of sodium sulfite, the organic phase was dried (MgSO4) and the volatiles were evaporated under reduced pressure. The residue was purified by vacuum distillation to give 169 g of colorless oily product, which was crystallized to afford colorless platelets. Yield: 70%.

1H NMR (400 MHz, CDCl3): δ 8.20 (dd, J=8.1 and 15.6 Hz, 1H), 6.70 (dd, J=2.9 and 8.2 Hz, 1H).

(2) 2,6-Difluoro-5-iodopyridine-3-carboxylic acid

At −75° C., diisopropylamine (7.0 mL, 5.1 g, 50 mmol) and a solution of 2,6-difluoro-3-iodopyridine (12.1 g, 50 mmol) in tetrahydrofuran (50 mL) were consecutively added to butyllithium (1.6M in hexane, 31.3 mL, 50 mmol) in tetrahydrofuran (60 mL). After 15 min at −75° C., the mixture was poured on an excess of freshly crushed dry ice. The volatiles were evaporated and the residue was dissolved in a 2.0 N aqueous solution (50 mL) of sodium hydroxide. The aqueous phase was washed with diethyl ether (2×25 mL), acidified with hydrochloric acid to pH 2 and extracted with diethyl ether (3×50 mL). The combined organic layers were dried and concentrated under reduced pressure. The residue was recrystallized from water to give pure 2,6-difluoro-5-iodopyridine-3-carboxylic acid as colorless platelets. Water liquor contains 2,6-difluoro-5-iodopyridine-4-carboxylic acid.

1H NMR (400 MHz, DOSO-d6): δ 13.90 (brs, 1H, exchangeable with D2O, COOH), 8.82 (t, J=8.3 Hz, 1H).

### Method B:

(1) (2,6-Difluoropyridin-3-yl)trimethylsilane

Diisopropylamine (61.4 mL, 44.0 g, 434 mmol) and 2,6-difluoropyridine (50 g, 434 mmol) were added consecutively to a solution of butyllithium (1.6M in hexane, 271 mL, 434 mmol) in tetrahydrofuran (1000 mL), cooled in an acetone/dry ice bath. After 90 min at −75° C., chlorotrimethylsilane was added. The reaction mixture was warmed to room temperature and 200 mL of water was added. The water phase was extracted with diethyl ether twice and the Combined organic layers were dried over anhydrous sodium sulfate and evaporated to dryness. The residue was purified by vacuum distillation (b.p. 75-77° C./20 Torr) to give pure products as a colorless liquid in an high yield.

1H NMR (400 MHz, CDCl3): δ 7.92 (q, J=8.0 Hz, 1H), 6.83 (ddd, J=7.8, 2.2, 1.8 Hz, 1H), 0.34 (s, 9H); High boiling point fraction (b.p. 100-105° C./20 Torr) is (2,6-Difluoropyridin-3,5-diyl)bis(trimethylsilane):

1H NMR (400 MHz, CDCl3): δ 7.91 (t, J=8.8 Hz, 1H), 0.35 (s, 18H).

(2) 2,6-Difluoro-5-iodo-pyridine-3-carboxylic acid

Diisopropylamine (14 mL, 10 g, 0.10 mol) and (2,6-difluoropyridin-3-yl)trimethylsilane (18.7 g, 0.10 mol) were added consecutively to a solution of butyllithium (0.10 mol) in tetrahydrofuran (200 mL) and cooled in an acetone/dry ice bath. After 90 min at −75° C., the mixture was poured on an excess of freshly crushed dry ice. At 25° C., 2.0 N ethereal hydrogen chloride (75 mL, 0.15 mol) was added and filtered and washed with chloroform. The filtrate was evaporated to dryness under reduced pressure and the solid residue was extracted with hot chloroform, filtered, and concentrated to afford the crude 2,6-difluoro-5-(trimethylsilyl)pyridine-3-carboxylic acid as a white solid, which was used in the next step without further purification.

1H NMR (400 MHz, DMSO-d6): δ 8.46 (dd, J=9.9, 7.7 Hz, 1H), 0.34 (s, 9H).

A solution of the dried above crude product and iodine monochloride (32 g, 0.20 mol) in tetrachloromethane (0.10 L) were heated under reflux for 20 h. The reaction mixture was cooled to room temperature, diluted with ether and washed with a saturated aqueous solution (100 mL) of sodium sulfite. The organic layer was separated and the water layer was neutralized with concentrated hydrochloric acid and extracted with ether. The combined organic layers were dried and concentrated under reduced pressure. The residue was recrystallized from water to give the desired product.

### Step B: 2,6-Difluoro-5-iodopyridine-3-carboxylic acid methyl ester

A mixture of 2,6-dichloro-5-iodo-pyridine-3-carboxylic acid (3.4 g, 11.9 mmol) and thionyl chloride (1.74 mL, 23.9 mmol) in 40 mL of anhydrous toluene and 0.1 mL of anhydrous DMF was refluxed for 2 h. The solvent was removed under reduced pressure and the residue was azeoptoped with toluene (2×20 mL). The residue was dissolved in 50 mL of anhydrous methanol refluxed for 30 min and cooled to room temperature. The solvent was removed under reduced pressure to give the crude product as a white solid.

1H NMR (400 MHz, CDCl3): δ 8.83 (t, J=8.2 Hz, 1H), 3.99 (s, 3H).

### Step C: Methyl 5-(3-chloro-2-fluorobenzyl)-2,6-difluoronicotinate

Under an argon stream, zinc powder (3.6 g, 55 mmol) was suspended in 5 mL of dry THF. 1,2-Dibromoethane (0.01 mL, 0.12 mmol) and TMS-Cl (0.03 mL, 0.24 mmol) were added at 60° C. to the suspension, and the mixture was stirred at this temperature for 30 min. A solution of 2-fluoro-3-chlorobenzyl bromide (2.7 g, 12 mmol) in 10 mL of dry THF was added dropwise at 60° C. The mixture was stirred with heating for 1 hour and allowed to cool to room temperature. The resulting solution of 2-fluoro-3-chlorobenzylzinc bromide in THF is used for next step.

To a solution of 2,6-Difluoro-5-iodopyridine-3-carboxylic acid methyl ester (2.7 g, 9 mmol) in 40 mL of dry THF was added dichlorobis(triphenylphosphine)palladium (II) (253 mg, 0.36 mmol). The mixture was heated at 60° C. and a solution of the above 2-fluoro-3-chlorobenzylzinc bromide in THF was added dropwise. The mixture was stirred with heating at the same temperature for 1 hour and was allowed to cool to room temperature. HCl (1 N, 75 mL) was added and the mixture was extracted with ethyl acetate (3×100 mL). The organic layers were combined, washed successively with water, brine, dried over anhydrous sodium sulfate and concentrated under reduced pressure. The crude product was purified by silica gel chromatography (ISCO, 40 g of column, hexane/ethyl acetate, 0-30%, 25 min, 30-80%, 10 min, 80%, 5 min) to give 2.3 g of pure product as colorless oil.

### Step D: 5-(3-Chloro-2-fluorobenzyl)-2,6-difluoronicotinic acid

To a solution of methyl 5-(3-chloro-2-fluorobenzyl)-2,6-difluoronicotinate (3.7 g, 11.7 mmol) in 36 mL of THF was added dropwise an aqueous solution of LiOH (1 N, 35 mL, 35 mmol). The mixture was stirred at room temperature for 1 hour and evaporated under reduced pressure. The residue was dissolved in 50 mL of water and neutralized with 6 N HCl. The precipitate was filtered and washed with water to give 3.5 g of pure product as a white solid in a quantitative yield.

### Step E: (S)-ethyl 2-(5-(3-chloro-2-fluorobenzyl)-2,6-difluoronicotinoyl)-3-(1-hydroxy-3,3-dimethylbutan-2-ylamino)acrylate

A mixture of 5-(3-chloro-2-fluorobenzyl)-2,6-difluoronicotinic acid (12.1 g, 40 mmol) and thionyl chloride (5.84 mL, 80 mmol) in 160 mL of anhydrous toluene and 0.4 mL of anhydrous DMF was refluxed for 2 h. The solvent was removed under reduced pressure and the resulting oil was azeoptoped with toluene (2×80 mL). The residue was dissolved in 40 mL of anhydrous THF and added dropwise to a solution of ethyl 3-(dimethylamino)acrylate (6.24 g, 44 mmol) and triethylamine (4.88 g, 57.6 mmol) in 160 mL of anhydrous THF under nitrogen. The mixture was heated under reflux for 7 hours, allowed to cool to room temperature and (S)-tert-leucinal (5.16 g, 44 mmol) (or L-valinol) was added. The reaction mixture was stirred for 30 min at room temperature and evaporated to dryness under reduced pressure. Water (200 mL) and ethyl acetate (200 mL) were added and the organic layer was separated, washed successively with saturated aqueous sodium bicarbonate (×2), water, brine, and dried over sodium sulfate. The mixture was filtered and the filtrate was concentrated under reduced pressure. The crude product was purified by silica gel chromatography (ISCO, hexane/EtOAc, 330 g, 0-40%, 30 min; 40-100%, 10 min; 100%, 30 min) to give the desired material as an yellow oil.

### Step F: 6-(3-Chloro-2-fluoro-benzyl)-7-fluoro-1-((S)-1-hydroxymethyl-2,2-dimethyl-propyl)-4-oxo-1,4-dihydro-1,8-naphthridine-3-carboxylic acid ethyl ester

A mixture of (S)-ethyl 2-(5-(3-chloro-2-fluorobenzyl)-2,6-difluoronicotinoyl)-3-(1-hydroxy-3,3-dimethylbutan-2-ylamino)acrylate (2.5 g, 5 mmol) and potassium carbonate (1.4 g, 10 mmol) in 30 mL of anhydrous DMF was stirred at 90° C. in an oil bath preheated to 90° C. for 10 min. Ice-water (300 mL) was added with stirring. The resulting precipitate was isolated by filtration and washed with water to give the desired product as white solid in almost quantitative yield (purity: 96%).

This product was treated with a THF solution of tetrabutylammonium fluoride to remove TBDMS group and then hydrolyzed in a solution of THF/1 N LiOH to give the desired product after purification by preparative HPLC.

### Step G: (S)-6-(3-chloro-2-fluorobenzyl)-7-morpholin-1-(1-hydroxy-3,3-dimethylbutan-2-yl)-4-oxo-1,4-dihydro-1,8-naphthyridine-3-carboxylic acid

A mixture of 1-[(S)-1-(tert-butyl-dimethyl-silanyloxymethyl)-2,2-dimethyl-propyl]-6-(3-chloro-2-fluoro-benzyl)-7-fluoro-4-oxo-1,4-dihydro-1,8-naphthyridine-3-carboxylic acid ethyl ester (1 g, 2.1 mmol), morpholine (0.37 g, 4.2 mmol) in methanol (15 mL) was stirred at room temperature for 3 days, followed by the addition of 10 mL of 1 N sodium hydroxide. The resulting mixture was stirred at 80° C. for 1 hour and concentrated under reduced pressure. The residue was dissolved in 20 mL Of water and filtered. The filtrate was neutralized with 6 N HCl and the precipitate was isolated and washed with water. The crude material was recrystallized from ethyl acetate to give the desired compound as white crystals (1.04 g, 96%; Purity: 96%).

¹H NMR (DMSO-d₆, 400 MHz): δ 15.12 (brs, 1H, OH, exchangeable with D₂O), 8.81 (s, 1H), 8.02 (s, 1H), 7.58 (dt, J=1.7 and 7.9 Hz, 1H), 7.35 (dt, J=1.7 and 7.9 Hz, 1H), 7.28 (d, J=7.9 Hz, 1H), 5.80 (dd, J=5.0 and 8.9 Hz, 1H), 5.05 (t, J=5.0 Hz, 1H, exchangeable with D₂O), 4.24 (s, 2H), 4.06 (m, 2H), 3.78 (m, 4H), 3.51 (m, 4H), 0.99 (s, 9H). MS: 518 (M+1).

### Example 2B

### (S)-6-(3-chloro-2-fluorobenzyl)-7-hydroxy-1-(1-hydroxy-3,3-dimethylbutan-2-yl)-4-oxo-1,4-dihydro-1,8-naphthyridine-3-carboxylic acid

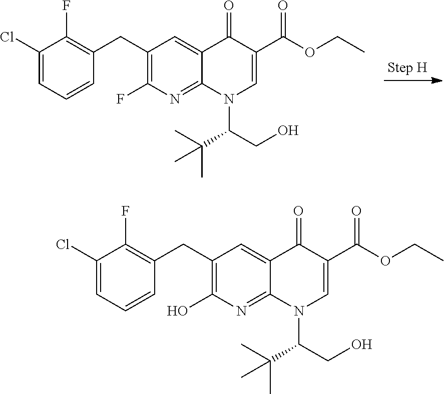

Step I: A suspension of (S)-ethyl 6-(3-chloro-2-fluorobenzyl)-7-fluoro-1-(1-hydroxy-3,3-dimethylbutan-2-yl)-4-oxo-1,4-dihydro-1,8-naphthyridine-3-carboxylate (300 mg) in 15 mL of 1 N sodium hydroxide was stirred at 80° C. for 1 hour. The reaction mixture was cooled to room temperature and filtered. The filtrate was neutralized with 6 N HCl and the precipitate was filtered and washed with water to give the desired product as a white solid.

### Examples 2C-2O

### (S)-6-(3-chloro-2-fluorobenzyl)-1-(1-hydroxy-3,3-dimethylbutan-2-yl)-4-oxo-7-alkyloxy-1,4-dihydro-1,8-naphthyridine-3-carboxylic acid

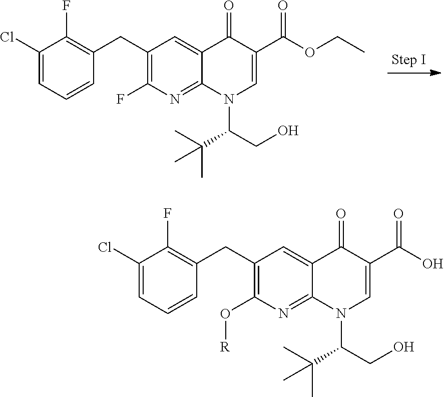

Step 1: 50 mg of sodium metal was added to 2 g of the corresponding alcohol under argon at room temperature and the resulting mixture was stirred at 80° C. until sodium was dissolved (about 1-2 hours). 300 mg of (S)-ethyl 6-(3-chloro-2-fluorobenzyl)-7-fluoro-1-(1-hydroxy-3,3-dimethylbutan-2-yl)-4-oxo-1,4-dihydro-1,8-naphthyridine-3-carboxylate added to this alcoholic sodium solution and stirred at 80° C. overnight and then 15 mL of 1 N sodium hydroxide was added and stirred at 80° C. for 1 hour. The reaction mixture was cooled to room temperature and filtered if necessary. The filtrate was neutralized with 2 N HCl to pH<7. The precipitate was filtered and washed with water to give the desired product which was purified by preparative HPLC if needed.

### Examples 2C-2II

Examples 2C-2II were prepared according to the procedures described above for examples 2A and 2B.

| Compound | Structure | Analytical Data |
|---|---|---|
| 2A | 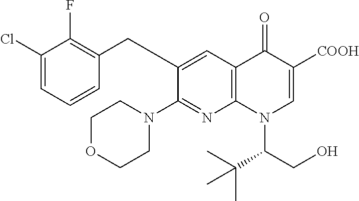 | MS: 518 (M + H)⁺ |
| 2B | 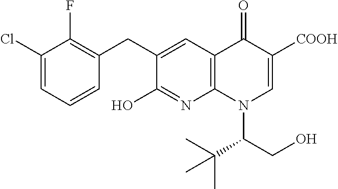 | MS: 449 (M + H)⁺ |
| 2C | 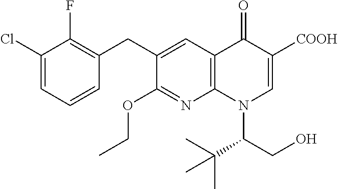 | MS: 477 (M + H)⁺ |
| 2D | 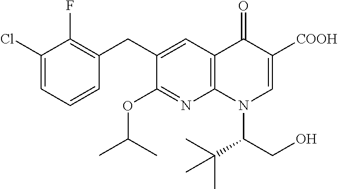 | MS: 491 (M + H)⁺ |
| 2E | 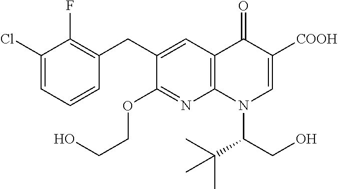 | MS: 493 (M + H)⁺ |
| 2F | 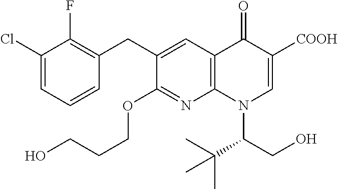 | MS: 507 (M + H)⁺ |
| 2G | 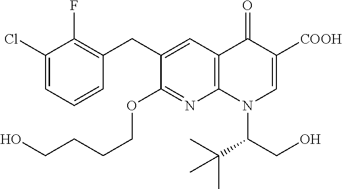 | MS: 521 (M + H)⁺ |
| 2H | 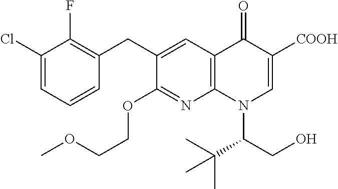 | MS: 507 (M + H)⁺ |
| 2I | 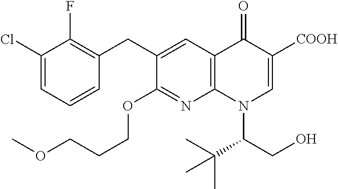 | MS: 521 (M + H)⁺ |
| 2J | 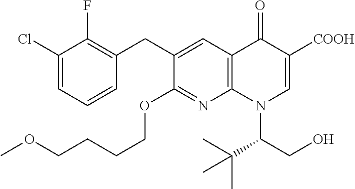 | MS: 535 (M + H)⁺ |
| 2K | 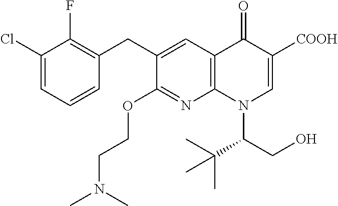 | MS: 520 (M + H)⁺ |
| 2L | 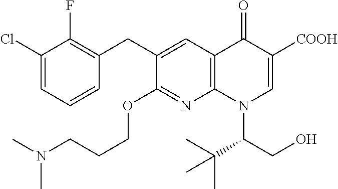 | MS: 534 (M + H)⁺ |
| 2M | 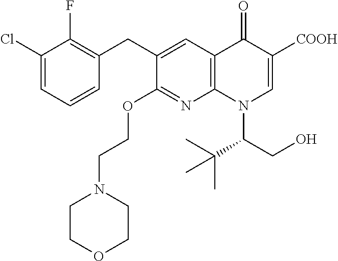 | MS: 562 (M + H)⁺ |
| 2N | 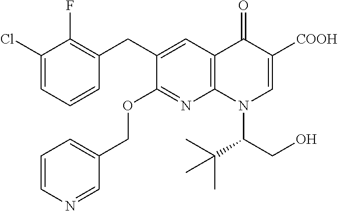 | MS: 540 (M + H)⁺ |
| 2O | 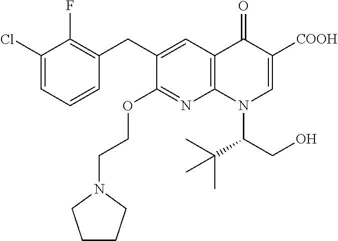 | MS: 546 (M + H)⁺ |
| 2P | 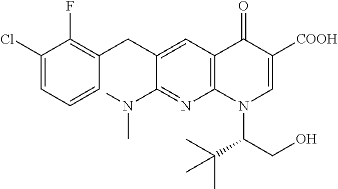 | MS: 476 (M + H)⁺ |
| 2Q | 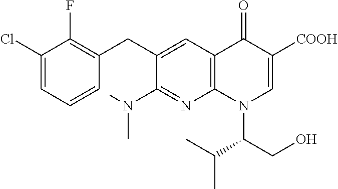 | MS: 466 (M + H)⁺ |
| 2R | 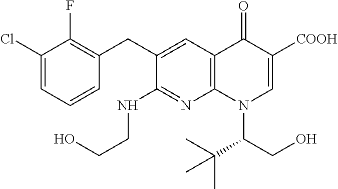 | MS: 492 (M + H)⁺ |
| 2S | 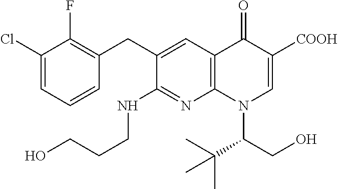 | MS: 506 (M + H)⁺ |
| 2T | 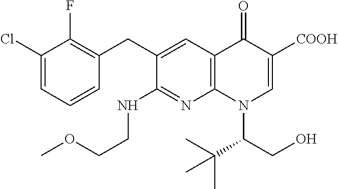 | MS: 506 (M + H)⁺ |
| 2U | 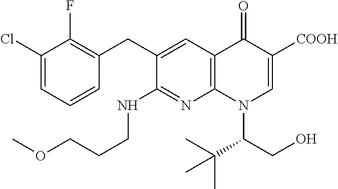 | MS: 520 (M + H)⁺ |
| 2V | 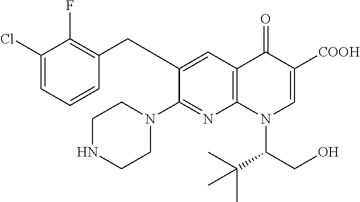 | MS: 517 (M + H)⁺ |
| 2W | 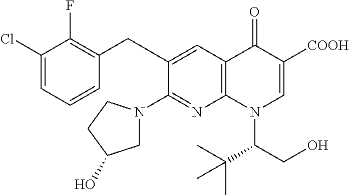 | MS: 518 (M + H)⁺ |
| 2X | 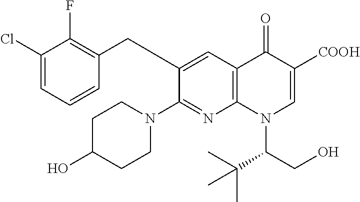 | MS: 532 (M + H)⁺ |
| 2Y | 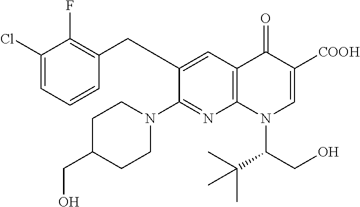 | MS: 546 (M + H)⁺ |
| 2Z | 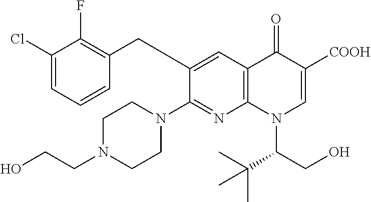 | MS: 561 (M + H)⁺ |
| 2AA | 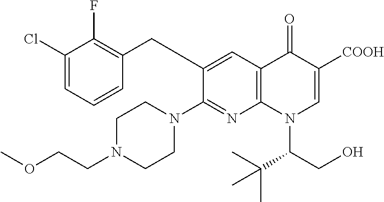 | MS: 575 (M + H)⁺ |
| 2BB | 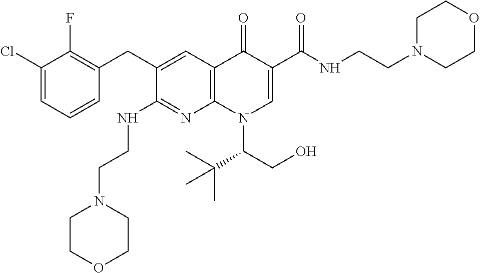 | MS: 513 (M + H)⁺ |
| 2CC | 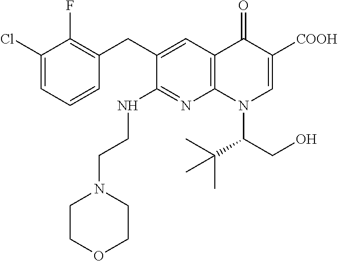 | MS: 561 (M + H)⁺ |
| 2DD | 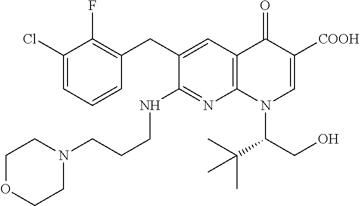 | MS: 575 (M + H)⁺ |
| 2EE | 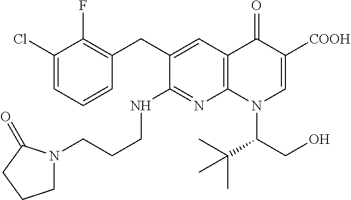 | MS: 573 (M + H)⁺ |
| 2FF | 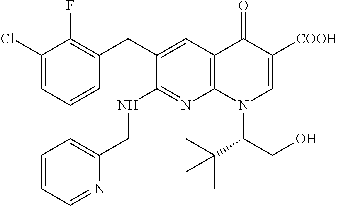 | MS: 539 (M + H)⁺ |
| 2GG | 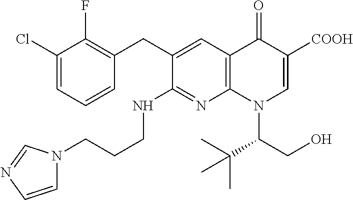 | MS: 556 (M + H)⁺ |
| 2HH | 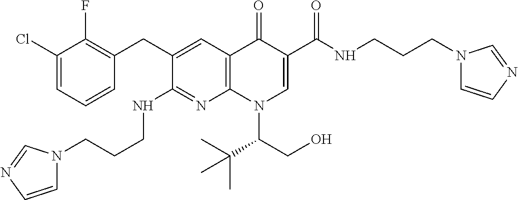 | MS: 663 (M + H)⁺ |
| 2II | 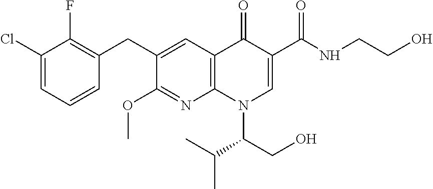 | MS: 492 (M + H)⁺ |
| 2JJ | 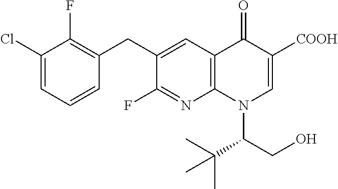 | MS: 451 (M + H)⁺ |
| 2KK | 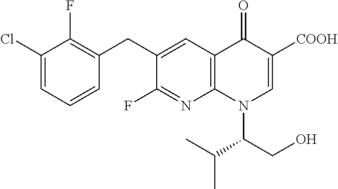 | MS: 437 (M + H)⁺ |

### Example 3

### Compounds of Formula (III)

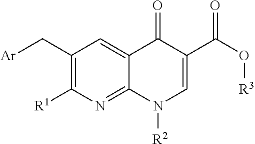

Compounds of formula (III) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

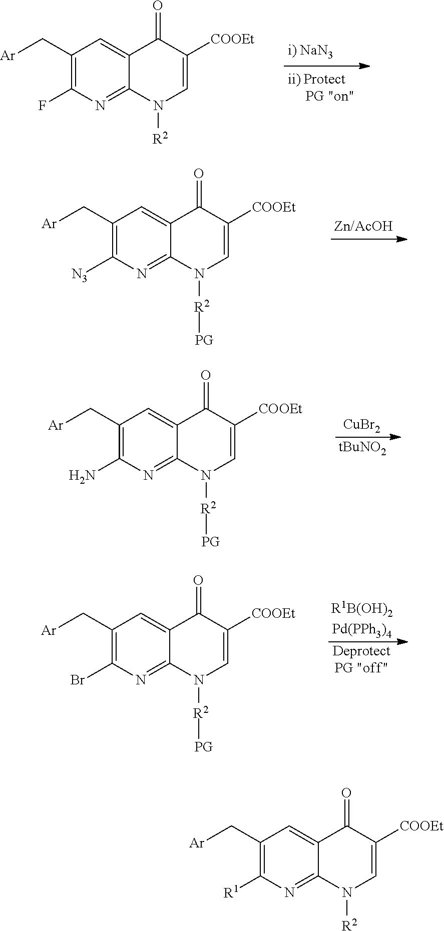

### Example 3A

### (S)-7-Amino-6-(3-chloro-2-fluorobenzyl)-1-(1-hydroxy-3,3-dimethylbutan-2-yl)-4-oxo-1,4-dihydro-1,8-naphthyridine-3-carboxylic acid

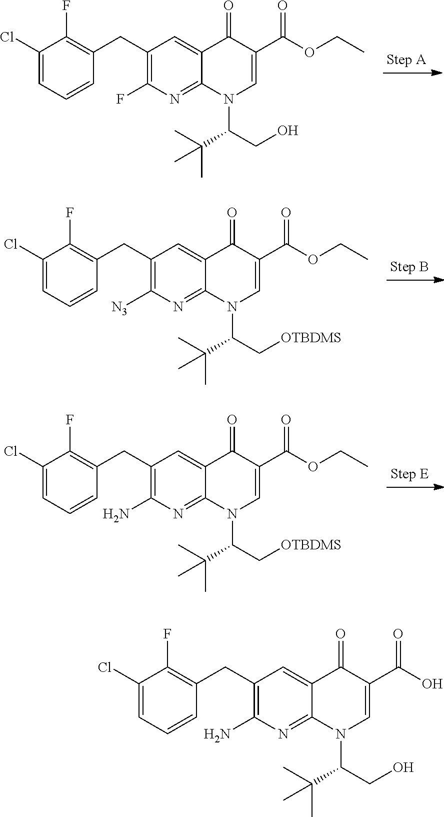

### Step A: 7-Azido-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester

A mixture of 6-(3-chloro-2-fluoro-benzyl)-7-fluoro-1-((S)-1-hydroxymethyl-2,2-dimethyl-propyl)-4-oxo-1,4-dihydro-1,8-naphthyridine-3-carboxylic acid ethyl ester (4.79 g, 10 mmol) and sodium azide (1.3 g, 20 mmol) in anhydrous DMF (10 mL) was stirred overnight at room temperature followed by the addition of imidazole (6.81 g, 100 mmol) and TBDMS-Cl (7.54 g, 50 mmol). The mixture was stiffed an additional 18 hours at room temperature and the solvent was evaporated under reduced pressure. The residue was purified by ISCO (hexane/EtOAc, 0-30%, 20 min, 40-100%, 10 min, 100%, 10 min) to give the pure product as a yellow oil (6.2 g, 100%).

1H NMR (CDCl3, 400 MHz): δ 8.81 (s, 1H), 8.51 (s, 1H), 7.34 (m, 1H), 7.04 (m, 2H), 5.73 (dd, J=4.4 and 8.6 Hz, 1H), 4.42 (q, J=7.0 Hz, 2H), 4.15 (m, 3H), 4.03 (m, 1H), 1.43 (t, J=7.1 Hz, 3H), 1.09 (s, 9H), 0.70 (s, 9H), 0.04 (s, 6H). MS: 616 (M+1).

### Step B: 7-Amino-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester

Zinc powder (2.7 g, 41.5 mmol) was added to a solution of 7-azido-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester (5.1 g, 8.3 mmol) in 3:1 dichloroform/acetic acid (80 mL). After 15 min the reaction mixture was poured into 300 mL of ethyl acetate and the resulting solution was washed with water, saturated sodium bicarbonate and brine. The organic solution was dried over sodium sulfate, filtered, and concentrated in vacuo to provide the desired product as a yellow oil in quantitative yield (purity: 97%).

1H NMR (CDCl3, 400 MHz): δ 8.18 (s, 1H), 7.84 (s, 1H), 6.78 (dt, J=2.2 and 7.5 Hz, 1H), 6.48 (t, J=7.5 Hz, 1H), 6.44 (dt, J=1.2 and 7.5 Hz, 1H), 5.17 (dd, J=4.4 and 8.6 Hz, 1H), 4.40 (brs, 2H, NH2), 3.85 (q, J=7.0 Hz, 2H), 3.54 (m, 2H), 3.41 (s, 2H), 0.87 (t, J=7.1 Hz, 3H), 0.50 (s, 0.16 (s, 9H), 0.04 (s, 6H). MS: 590 (M+1).

### Step E: (S)-7-Amino-6-(3-chloro-2-fluorobenzyl)-1-(1-hydroxy-3,3-dimethylbutan-2-yl)-4-oxo-1,4-dihydro-1,8-naphthyridine-3-carboxylic acid

7-Amino-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester was hydrolyzed using the same method described in step E below.

### Example 3B

### 6-(3-Chloro-2-fluorobenzyl)-1-((5)-1-hydroxymethyl-2,2-dimethyl-propyl)-7-methyl-4-oxo-1,4-dihydro-1,8-naphthyridine-3-carboxylic acid

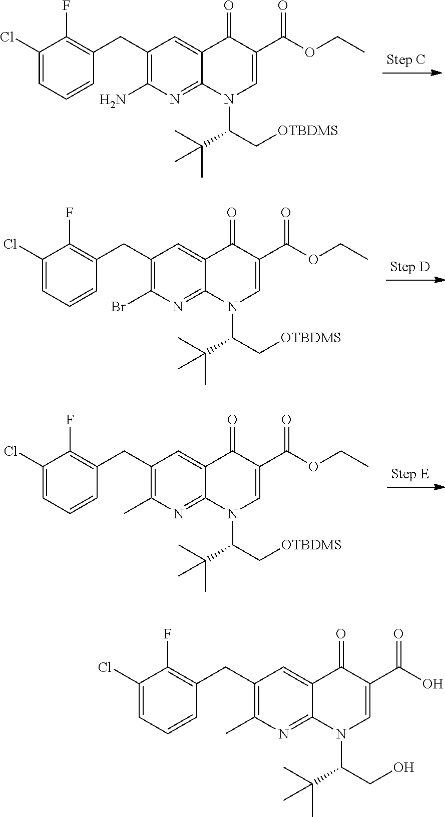

### Step C: 7-Bromo-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester

A mixture of copper bromide (1.7 g, 7.6 mmol), tert-butyl nitrite (1.0 g, 9.5 mmol) in bromoform (5 mL) and anhydrous acetonitrile (20 mL) was warmed to 60° C. under argon and then a solution of 7-amino-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester (3.7 g, 6.3 mmol) in 10 mL of anhydrous acetonitrile was added dropwise. The mixture was stirred at the same temperature for 20 min. The reaction mixture was cooled to room temperature and filtered through Celite and washed with ethyl acetate. The filtrate was evaporated to dryness under reduced pressure and the residue was purified by ISCO (hexane/ethyl acetate, 0%, 5 min; 0-30%, 25 min; 30-100%, 10 min) to give the pure product as an yellowish solid (2.6 g, 63%).

1H NMR (CDCl3, 400 MHz): δ 8.87 (s, 1H), 8.45 (s, 1H), 7.36 (dt, J=2.2 and 7.5 Hz, 1H), 7.06 (dt, J=0.6 and 7.5 Hz, 1H), 7.01 (dt, J=2.2 and 7.5 Hz, 1H), 5.72 (dd, J=4.2 and 8.6 Hz, 1H), 4.42 (q, J=7.0 Hz, 2H), 4.24 (m, 2H), 4.14 (m, 2H), 1.43 (t, J=7.1 Hz, 3H), 1.09 (s, 9H), 0.71 (s, 9H), 0.04 (s, 6H).

MS: 653, 655, 656 (M+1).

### Step D: 1-[(S)-1-(tert-Butyl-dimethyl-silanyloxymethyl)-2,2-dimethyl-propyl]-6-(3-chloro-2-fluoro-benzyl)-7-methyl-4-oxo-1,4-dihydro-1,8-naphthyridine-3-carboxylic acid ethyl ester

7-bromo-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester (300 mg, 0.46 mmol) was dissolved in 10 mL of 1,2-dimethoxyethane and methylboronic acid (55 mg, 0.92 mmol), tetrakis(triphenylphosphine)palladium(0) (35 mg, 0.03 mmol) and 2M sodium carbonate (0.5 mL) were added. The reaction mixture was stirred at 80° C. for 48 hours. After cooling to room temperature, saturated aqueous ammonium Chloride and ethyl acetate were added to the reaction mixture. The organic layer was washed with water, brine, dried over sodium sulfate and concentrated under reduced pressure. The crude residue was purified by ISCO (hexane/ethyl acetate: 0%, 5 min; 0-30%, 30 min; 30-100%, 10 min) to give pure compound as an oil (170 mg, 63%).

1H NMR (CDCl3, 400 MHz): δ 8.89 (s, 1H), 8.48 (s, 1H), 7.32 (dt, J=1.7 and 7.8 Hz, 1H), 7.01 (dt, J=1.0 and 7.8 Hz, 1H), 6.89 (dt, J=1.7 and 7.8 Hz, 1H), 6.00 (dd, J=4.2 and 8.6 Hz, 1H), 4.42 (q, J=7.0 Hz, 2H), 4.15 (m, 4H), 2.61 (s, 3H), 1.43 (t, J=7.1 Hz, 3H), 1.08 (s, 9H), 0.69 (s, 9H), 0.02 (s, 3H), 0.01 (s, 3H). MS: 589 (M+1).

### Step E: 6-(3-Chloro-2-fluoro-benzyl)-1-((S)-1-hydroxymethyl-2,2-dimethyl-propyl)-7-methyl-4-oxo-1,4-dihydro-1,8-naphthyridine-3-carboxylic acid

A mixture of 1-[(S)-1-(tert-Butyl-dimethyl-silanyloxymethyl)-2,2-dimethyl-propyl]-6-(3-chloro-2-fluoro-benzyl)-7-methyl-4-oxo-1,4-dihydro-1,8-naphthyridine-3-carboxylic acid ethyl ester (100 mg, 0.17 mmol), 28% sodium methoxide (2 mL) and water (1 mL) in methanol (15 mL) was stirred at 80° C. for 5 hour. The reaction mixture was cooled at room temperature and the solvent was evaporated under reduced pressure. The residue was dissolved in 10 mL of water and filtered. The filtrate was neutralized with 6 N HCl and the precipitate was filtered and washed with water to give pure product as a white solid.

1H NMR (DMSO-d6, 400 MHz): δ 14.81 (brs, 1H, OH, exchangeable with D2O), 8.94 (s, 1H), 8.31 (s, 1H), 7.58 (dt, J=2.1 and 7.3 Hz, 1H), 7.29 (m, 2H), 6.03 (dd, J=4.2 and 8.6 Hz, 1H), 5.08 (t, J=5.0 Hz, 1H, exchangeable with D₂O), 4.33 (s, 2H), 4.11 (m, 2H), 2.73 (s, 3H), 0.98 (s, 9H). MS: 447 (M+1).

### Example 3C

### 6-(3-Chloro-2-fluoro-benzyl)-1-((S)-1-hydroxymethyl-2,2-dimethyl-propyl)-7-(3-hydroxy-propyl)-4-oxo-1,4-dihydro-1,8-naphthyridine-3-carboxylic acid

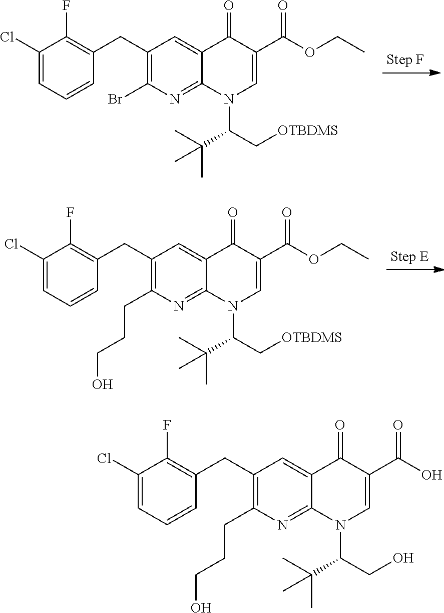

### Step F: (S)-ethyl 1-(1-(tert-butyldimethylsilyloxy)-3,3-dimethylbutan-2-yl)-6-(3-chloro-2-fluorobenzyl)-7-(3-hydroxypropyl)-4-oxo-1,4-dihydro-1,8-naphthyridine-3-carboxylate

7-Bromo-1-[(S)-1-(tert-butyl-dimethyl-silanyloxymethyl)-2,2-dimethyl-propyl]-6-(3-chloro-2-fluoro-benzyl)-4-oxo-1,4-dihydro-1,8-naphthyridine-3-carboxylic acid ethyl ester (300 mg, 0.46 mmol) was dissolved in 10 mL of 1,2-dimethoxyethane. 3-Bromopropylboronic acid pinacol ester (229 mg, 0.92 mmol), tetrakis(triphenylphosphine)palladium(0) (35 mg, 0.03 mmol), and 2M sodium carbonate (0.5 mL) were added. The reaction mixture was stirred at 80° C. for 48 hours. After cooling to room temperature, saturated aqueous ammonium chloride and ethyl acetate were added to the reaction mixture. The organic layer was washed with water, brine, and dried over sodium sulfate. The solution was concentrated under reduced pressure and the residue was purified by ISCO (hexane/ethyl acetate: 0%, 5 min; 0-30%, 30 min; 30-100%, 10 min) to give the desired compound as an oil.

1H NMR (CDCl3, 400 MHz): δ 8.88 (s, 1H), 8.46 (s, 1H), 7.31 (dt, J=1.7 and 7.8 Hz, 1H), 7.00 (dt, J=1.0 and 7.8 Hz, 1H), 6.88 (dt, J=1.7 and 7.8 Hz, 1H), 5.97 (dd, J=4.2 and 8.6 Hz, 1H), 4.41 (q, J=7.0 Hz, 2H), 4.16 (m, 4H), 3.75 (t, J=6.2 Hz, 2H), 3.00 (m, 2H), 2.09 (m, 2H), 1.76 (brs, 1H, exchangeable with D2O), 1.42 (t, J=7.1 Hz, 3H), 1.07 (s, 9H), 0.66 (s, 9H), 0.01 (s, 3H), −0.04 (s, 3H).

MS: 633 (M+1).

### Step E: 6-(3-Chloro-2-fluoro-benzyl)-1-((S)-1-hydroxymethyl-2,2-dimethyl-propyl)-7-(3-hydroxy-propyl)-4-oxo-1,4-dihydro-1,8-naphthyridine-3-carboxylic acid

(S)-ethyl 1-(1-(tert-butyldimethylsilyloxy)-3,3-dimethylbutan-2-yl)-6-(3-chloro-2-fluorobenzyl)-7-(3-hydroxypropyl)-4-oxo-1,4-dihydro-1,8-naphthyridine-3-carboxylate was hydrolyzed according to the procedure described in step E above to give the desired product as a white solid. Yield: 71%, Purity: 96%.

1H NMR (DMSO-d6, 400 MHz): δ 14.81 (brs, 1H, OH, exchangeable with D2O), 8.95 (s, 1H), 8.30 (s, 1H), 7.66 (m, 1H), 7.58 (m, 1H), 7.28 (m, 1H), 6.03 (dd, J=4.4 and 9.7 Hz, 1H), 5.08 (t, J=5.0 Hz, 1H, exchangeable with D2O), 4.62 (t, J=5.1 Hz, 1H, exchangeable with D2O), 4.35 (s, 2H), 4.10 (m, 2H), 3.55 (m, 2H), 3.08 (m, 2H), 1.99 (m, 2H), 0.99 (s, 9H). MS: 491 (M+1).

| Compound | Structure | Analytical Data |
|---|---|---|
| 3A | 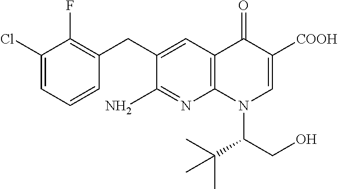 | MS: 448 (M + H)⁺ |
| 3B | 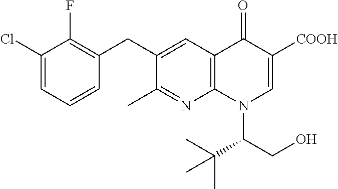 | ¹H NMR (DMSO-d₆, 400 MHz): δ 14.81 (brs, 1H, OH, exchangeable with D₂O), 8.94 (s, 1H), 8.31 (s, 1H), 7.58 (dt, J = 2.1 and 7.3 Hz, 1H), 7.29 (m, 2H), 6.03 (dd, J = 4.2 and 8.6 Hz, 1H), 5.08 (t, J = 5.0 Hz, 1H, exchangeable with D₂O), 4.33 (s, 2H), 4.11 (m, 2H), 2.73 (s, 3H), 0.98 (s, 9H). MS: 447 (M + 1). |
| 3C | 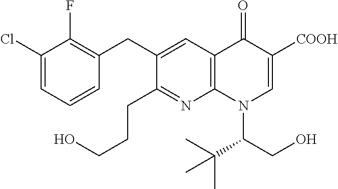 | ¹H NMR (DMSO-d₆, 400 MHz): δ 14.81 (brs, 1H, OH, exchangeable with D₂O), 8.95 (s, 1H), 8.30 (s, 1H), 7.66 (m, 1H), 7.58 (m, 1H), 7.28 (m, 1H), 6.03 (dd, J = 4.4 and 9.7 Hz, 1H), 5.08 (t, J = 5.0 Hz, 1H, exchange- able with D₂O), 4.62 (t, J = 5.1 Hz, 1H, exchangeable with D₂O), 4.35 (s, 2H), 4.10 (m, 2H), 3.55 (m, 2H), 3.08 (m, 2H), 1.99 (m, 2H), 0.99 (s, 9H). MS: 491 (M + 1). |

### Example 4

### Compounds of Formula (IV)

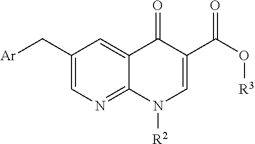

Compounds of formula (IV) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

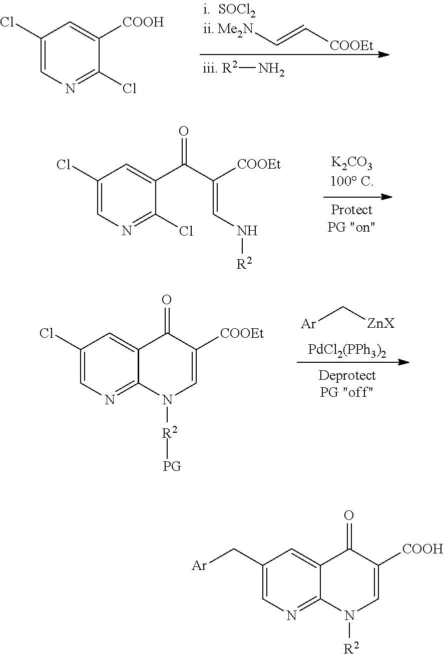

### Example 4A

### (S)-6-(3-chloro-2-fluorobenzyl)-1-(1-hydroxy-3-methylbutan-2-yl)-4-oxo-1,4-dihydro-1,8-naphthyridine-3-carboxylic acid

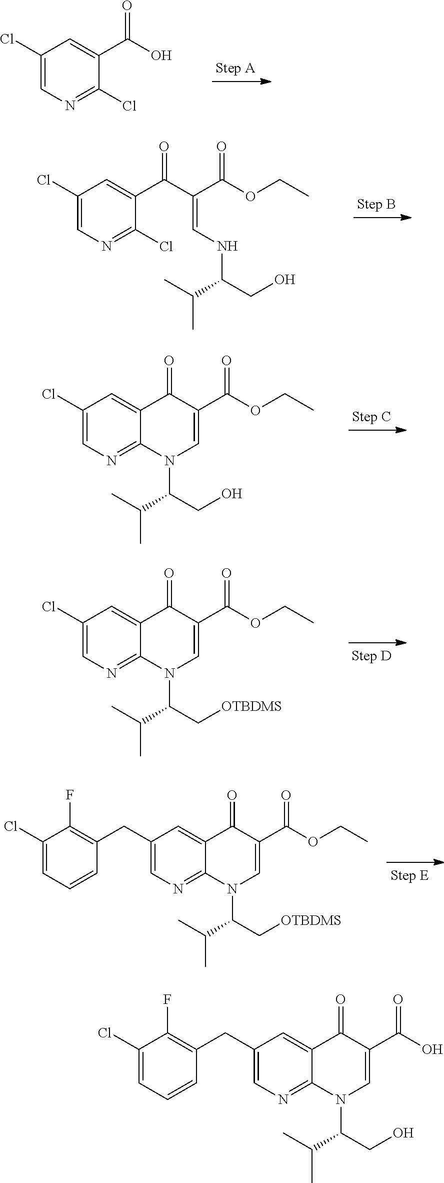

### Step A: (S)-ethyl 2-(2,5-dichloronicotinoyl)-3-(1-hydroxy-3-methylbutan-2-ylamino)acrylate

This compound was prepared according to the procedure described above for example 1A.

1H NMR (DMSO-d6, 400 MHz): δ 10.97 (dd, J=9.6 and 13.8 Hz, 1H, NH, exchangeable with D2O), 8.48 (d, J=2.6 Hz, 1H), 8.28 (d, J=14.3 Hz, 1H, it becomes singlet after D2O exchange), 7.94 (d, J=2.6 Hz, 1H), 5.07 (t, J=5.1 Hz, 1H, OH, exchangeable with D2O), 3.89 (q, J=7.0 Hz, 2H), 3.59 (m, 2H), 3.39 (m, 1H), 1.95 (m, 1H), 1.17 (t, J=7.0 Hz, 3H), 0.92 (d, J=6.6 Hz, 3H), 0.88 (d, J=6.61 Hz, 3H).

MS: 375, 377 (M+1).

### Step B: (S)-ethyl 6-chloro-1-(1-hydroxy-3-methylbutan-2-yl)-4-oxo-1,4-dihydro-1,8-naphthyridine-3-carboxylate

This, compound was prepared according to the procedure described above for example 1A.

1H NMR (CDCl3, 400 MHz): δ 8.83 (s, 1H), 8.63 (d, J=2.6 Hz, 1H), 8.02 (d, J=2.6 Hz, 1H), 5.30 (m, 1H), 4.36 (q, J=7.1 Hz, 2H), 4.19 (d, J=3.4 Hz, 2H), 2.56 (m, 1H), 1.41 (t, J=7.1 Hz, 3H), 1.25 (d, J=6.2 Hz, 3H), 0.75 (d, J=6.2 Hz, 3H).

MS: 339 (M+1).

### Step C: (S)-ethyl 1-(1-(tert-butyldimethylsilyloxy)-3-methylbutan-2-yl)-6-chloro-4-oxo-1,4-dihydro-1,8-naphthyridine-3-carboxylate

This compound was prepared according to the procedure described above for example 1A.

1H NMR (CDCl3, 400 MHz): δ 8.78 (s, 1H), 8.66 (d, J=2.6 Hz, 1H), 8.50 (d, J=2.6 Hz, 1H), 5.35 (d, J=10.2 Hz, 1H), 4.30 (q, J=7.1 Hz, 2H), 3.98 (m, 1H), 3.67 (d, J=10.2 Hz, 1H), 2.37 (m, 1H), 1.30 (t, J=7.1 Hz, 3H), 1.08 (d, J=6.2 Hz, 3H), 0.72 (s, 9H), 0.68 (d, J=6.2 Hz, 3H), −0.11 (s, 3H), −0.14 (s, 3H).

MS: 453 (M+1).

### Step D: (S)-ethyl 1-(1-(tert-butyldimethylsilyloxy)-3-methylbutan-2-yl)-6-(3-chloro-2-fluorobenzyl)-4-oxo-1,4-dihydro-1,8-naphthyridine-3-carboxylate

This compound was prepared according to the procedure described above for example 1A.

1H NMR (CD3OD, 400 MHz): δ 8.97 (s, 1H), 8.77 (d, J=2.6 Hz, 1H), 8.56 (d, J=2.6 Hz, 1H), 7.38 (dt, J=2.0 and 7.8 Hz, 1H), 7.31 (dt, J=2.0 and 7.8 Hz, 1H), 7.14 (t, J=7.8 Hz, 1H), 5.69 (d, J=10.2 Hz, 1H), 4.33 (q, J=7.1 Hz, 2H), 4.25 (s, 2H), 4.10 (m, 1H), 3.87 (d, J=10.2 Hz, 1H), 2.45 (m, 1H), 1.36 (t, J=7.1 Hz, 3H), 1.24 (d, J=6.2 Hz, 3H), 0.72 (s, 9H), 0.80 (d, J=6.2 Hz, 3H), −0.01 (s, 3H), −0.05 (s, 3H).

MS: 561 (M+1).

### Step E: (S)-6-(3-chloro-2-fluorobenzyl)-1-(1-hydroxy-3-methylbutan-2-yl)-4-oxo-1,4-dihydro-1,8-naphthyridine-3-carboxylic acid

This compound was prepared according to the procedure described above for example 1A.

¹H NMR (CD₃OD, 400 MHz): δ 9.16 (s, 1H), 8.89 (d, J=2.6 Hz, 1H), 8.63 (d, J=2.6 Hz, 1H), 7.40 (dt, J=2.0 and 7.8 Hz, 1H), 7.34 (dt, J=2.0 and 7.8 Hz, 1H), 7.16 (t, J=7.8 Hz, 1H), 5.76 (brs, 1H), 4.30 (s, 2H), 4.12 (m, 1H), 3.89 (m, 2H), 2.47 (m, 1H), 1.20 (d, J=6.2 Hz, 3H), 0.76 (d, J=6.2 Hz, 3H).

MS: 418 (M+1).

### Examples 4B-4E

Examples 4B-4E were prepared according to the procedure described above for example 4A.

| Compound | Structure | Analytical Data |
|---|---|---|
| 4A | 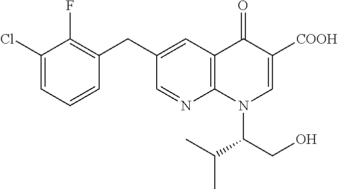 | ¹H NMR (CD₃OD, 400 MHz): δ 9.16 (s, 1H), 8.89 (d, J = 2.6 Hz, 1H), 8.63 (d, J = 2.6 Hz, 1H), 7.40 (dt, J = 2.0 and 7.8 Hz, 1H), 7.34 (dt, J = 2.0 and 7.8 Hz, 1H), 7.16 (t, J = 7.8 Hz, 1H), 5.76 (brs, 1H), 4.30 (s, 2H), 4.12 (m, 1H), 3.89 (m, 2H), 2.47 (m, 1H), 1.20 (d, J = 6.2 Hz, 3H), 0.76 (d, J = 6.2 Hz, 3H). MS: 418 (M + 1). |
| 4B | 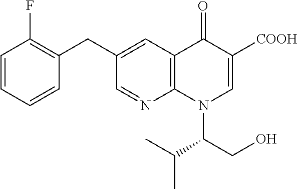 | MS: 385 (M + H)⁺ |
| 4C | 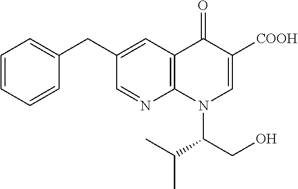 | MS: 367 (M + H)⁺ |
| 4D | 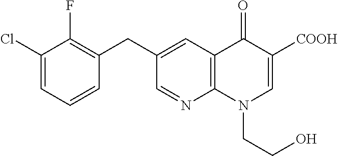 | MS: 377 (M + H)⁺ |
| 4E | 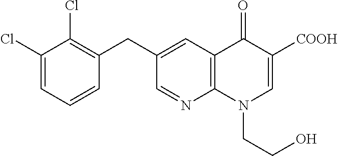 | MS: 393 (M + H)⁺ |

### Example 5

### Compounds of Formula (V)

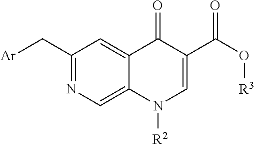

Compounds of formula (V) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

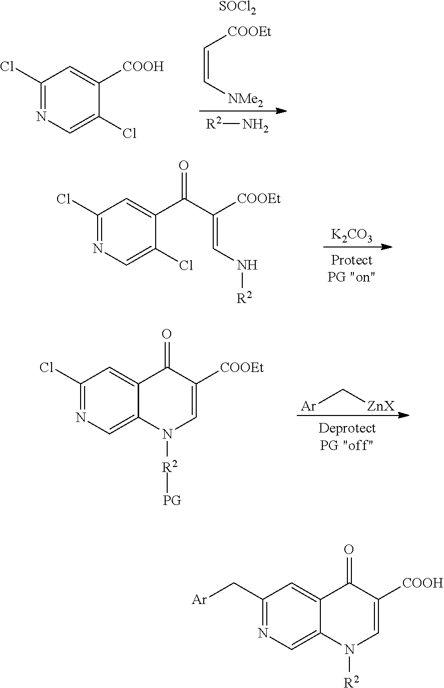

### Example 5A

### 6-(3-Chloro-2-fluoro-benzyl)-1-((S)-1-hydroxymethyl-2-methyl-propyl)-4-oxo-1,4-dihydro-[1,7]naphthyridine-3-carboxylic acid

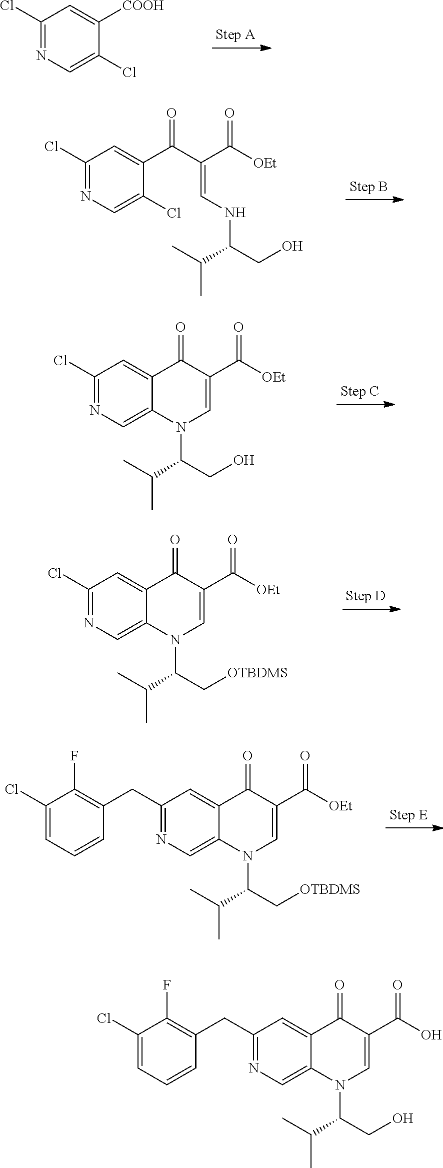

### Step A: 2-(2,5-Dichloro-pyridine-4-carbonyl)-3-((S)-1-hydroxymethyl-2-methyl-propylamino)-acrylic acid ethyl ester

2,5-Dichloro-4-pyridinecarboxylic acid was prepared according to known procedures (see Eur. J. Org. Chem. 2001, 1371-1376), as follows: At −75° C., 2,5-dichloropyridine (3.7 g, 25 mmol) was added to a solution of butyllithium (1.6M in hexane) (25 mmol) and N,N,N′,N″,N″-pentamethyldiethylenetriamine (5.3 mL, 4.3 g, 25 mmol) in tetrahydrofuran (50 mL). After 211 at −75° C., the mixture was poured onto an excess of freshly crushed dry ice. Water (50 mL) was added, the aqueous phase decanted and washed with diethyl ether (3×20 mL) and neutralized with 5N HCl to pH 1. The precipitate was filtered and washed with water to give 2.7 g of white solid as a pure product. The filtrate was extracted with ethyl acetate and the combined organic layers were evaporated to dryness. The residue was recrystallized from ethanol to give another batch of pure product. (The filtrate was evaporated to small volume and the precipitate was filtered and washed with water to give another batch of product); m.p. 227-229° C. (from ethanol); 4.2 g (87%).

1H NMR (400 MHz, DMSO-d6): ä 14.43 (brs, 1H, exchangeable with D2O), 8.64 (s, 1 H), 7.87 (s, 1H).

2-(2,5-Dichloro-pyridine-4-carbonyl)-3-((S)-1-hydroxymethyl-2-methyl-propylamino)-acrylic acid ethyl ester was synthesized using the same procedure described in example 1A, (91% yield).

1H NMR (DMSO-d6, 400 MHz): δ 10.94 (dd, J=9.6 and 13.8 Hz, 1H, NH, exchangeable with D2O), 8.48 (s, 1H), 8.28 (d, J=14.3 Hz, 1H, it becomes singlet after D2O exchange), 7.51 (s, 1H), 5.07 (t, J=5.1 Hz, 1H, OH, exchangeable with D2O), 3.92 (q, J=7.0 Hz, 2H), 3.62 (m, 2H), 3.40 (m, 1H), 1.95 (m, 1H), 0.94 (d, J=6.6 Hz, 3H), 0.91 (d, J=6.6 Hz, 3H), 0.89 (t, J=7.0 Hz, 3H). MS: 375, 377 (M+1).

### Step B: 6-Chloro-1-((S)-1-hydroxymethyl-2-methyl-propyl)-4-oxo-1,4-dihydro-[1,7]naphthyridine-3-carboxylic acid ethyl ester

This compound was synthesized using the same procedure described in example 1A, (69%, yield).

¹H NMR (DMSO-d₆, 400 MHz): δ 9.41 (s, 1H), 8.76 (s, 1H), 8.06 (s, 1H), 5.17 (brs, 1H, OH, exchangeable with D₂O), 4.82 (m, 1H), 4.26 (q, J=7.1 Hz, 2H), 3.90 (m, 1H), 3.84 (m, 1H), 2.32 (m, 1H), 1.29 (t, J=7.1 Hz, 3H), 1.12 (d, J=6.2 Hz, 3H), 0.76 (d, J=6.2 Hz, 3H).

MS: 339 (M+1).

### Step C: 1-[(S)-1-(tert-Butyl-dimethyl-silanyloxymethyl)-2-methyl-propyl]-6-chloro-4-oxo-1,4-dihydro-[1,7]naphthyridine-3-carboxylic acid ethyl ester

This compound was synthesized using the same procedure described in example 1A, (94% yield).

¹H NMR (CDCl₃, 400 MHz): δ 8.91 (s, 1H), 8.76 (s, 1H), 8.37 (s, 1H), 4.45 (q, J=7.1 Hz, 2H), 4.41 (m, 1H), 4.09 (m, 1H), 4.03 (m, 2.50 (m, 1H), 1.44 (t, J=7.1 Hz, 3H), 1.24 (d, J=6.2 Hz, 3H), 0.91 (d, J=6.21 Hz, 3H), 0.79 (s, 9H), 0.03 (s, 6H).

MS: 453 (M+1).

### Step D: 1-[(S)-1-(tert-Butyl-dimethyl-silanyloxymethyl)-2-methyl-propyl]-6-(3-chloro-2-fluoro-benzyl)-4-oxo-1,4-dihydro-[1,7]naphthyridine-3-carboxylic acid ethyl ester

This compound was synthesized using the same procedure described in example 1A.

¹H NMR (CD₃OD, 400 MHz): δ 9.41 (s, 1H), 8.93 (s, 1H), 8.16 (s, 1H), 7.41 (t, J=7.8 Hz, 1H), 7.32 (t, J=7.8 Hz, 1H), 7.16 (t, J=7.8 Hz, 1H), 4.41 (s, 2H), 4.15 (q, J=7.1 Hz, 2H), 4.09 (m, 1H), 4.02 (m, 1H), 2.53 (m, 1H), 1.41 (t, J=7.1 Hz, 3H), 1.27 (d, J=6.2 Hz, 3H), 0.92 (d, J=6.2 Hz; 3H), 0.74 (s, 9H), 0.03 (s, 6H).

MS: 561 (M+1).

### Step E: 6-(3-Chloro-2-fluoro-benzyl)-1-((S)-1-hydroxymethyl-2-methyl-propyl)-4-oxo-1,4-dihydro-[1,7]naphthyridine-3-carboxylic acid

This compound was synthesized using the same procedure described in example 1A.

¹H NMR (DMSO-d₆, 400 MHz): δ 14.62 (brs, 1H, OH, exchangeable with D₂O), 9.68 (s, 1H), 8.99 (s, 1H), 8.11 (s, 1H), 7.49 (t, J=7.8 Hz, 1H), 7.40 (t, J=7.8 Hz, 1H), 7.20 (t, J=7.8 Hz, 1H), 5.20 (brs, 1H, OH, exchangeable with D₂O), 5.03 (m, 1H), 4.42 (s, 2H), 3.96 (m, 1H), 3.84 (m, 1H), 2.38 (m, 1H), 1.13 (d, J=6.4 Hz, 3H), 0.73 (d, J=6.4 Hz, 3H).

MS: 419 (M+1).

### Example 5B

### 6-(3,4-Difluoro-benzyl)-1-((S)-1-hydroxymethyl-2-methyl-propyl)-4-oxo-1,4-dihydro-[1,7]naphthyridine-3-carboxylic acid

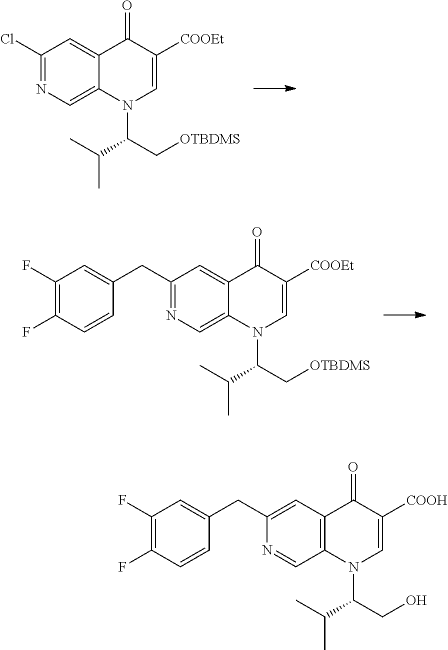

The title compound was prepared according to the above scheme, via similar procedures to those described herein, using commercially available 3,4-difluorobenzylzine bromide (0.5M in THF, Aldrich).

### 1-[(s)-1-(tert-Butyl-dimethyl-silanyloxymethyl)-2-methyl-propyl]-6-(3,4-difluoro-benzyl)-4-oxo-1,4-dihydro-[1,7]naphthyridine-3-carboxylic acid ethyl ester

¹H NMR (CD₃OD, 400 MHz): δ 9.43 (s, 1H), 8.93 (s, 1H), 8.18 (s, 1H), 7.22 (m, 3H), 4.44 (q, J=7.1 Hz, 2H), 4.32 (s, 2H), 4.16 (m, 1H), 4.07 (m, 1H), 2.54 (m, 1H), 1.41 (t, J=7.1 Hz, 3H), 1.28 (d, J=6.2 Hz, 3H), 0.92 (d, J=6.2 Hz, 3H), 0.75 (s, 9H), 0.02 (s, 6H).

MS: 545 (M+1).

### 6-(3,4-Difluoro-benzyl)-1-((S)-1-hydroxymethyl-2-methyl-propyl)-4-oxo-1,4-dihydro-[1,7]naphthyridine-3-carboxylic acid

¹H NMR (DMSO-d₆, 400 MHz): δ 14.68 (brs, 1H, OH, exchangeable with D₂O), 9.69 (s, 1H), 8.99 (s, 1H), 8.15 (s, 1H), 7.45 (m, 1H), 7.37 (m, 1H), 7.21 (m, 1H), 5.21 (brs, 1H, OH, exchangeable with D₂O), 5.05 (m, 1H), 4.32 (s, 2H), 3.96 (m, 1H), 3.84 (m, 1H), 2.38 (m, 1H), 1.13 (d, J=6.4 Hz, 3H), 0.73 (d, J=6.4 Hz, 3H).

MS: 403 (M+1).

### Example 5C

### 6-(3-Chloro-benzyl)-1-((S)-1-hydroxymethyl-2-methyl-propyl)-4-oxo-1,4-dihydro-[1,7]naphthyridine-3-carboxylic acid

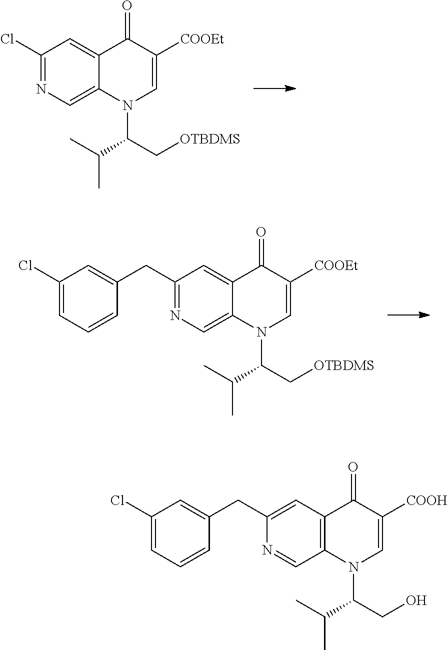

The title compound was prepared according to the above scheme, via similar procedures to those described herein, using commercially available 3-chlorobenzylzinc bromide (0.5M in THF, Aldrich).

### 1-[(S)-1-(tert-Butyl-dimethyl-silanyloxymethyl)-2-methyl-propyl]-6-(3-chloro-benzyl)-4-oxo-1,4-dihydro-[1,7]naphthyridine-3-carboxylic acid ethyl ester

¹H NMR (CDCl₃, 400 MHz): δ 9.11 (s, 1H), 8.77 (s, 1H), 8.26 (s, 1H), 7.32 (m, 2H), 7.25 (m, 2H), 4.44 (q, J=7.1 Hz, 2H), 4.31 (s, 2H), 4.08 (m, 1H), 4.02 (m, 1H), 2.51 (m, 1H), 1.45 (t, J=7.1 Hz, 3H), 1.25 (d, J=6.2 Hz, 3H), 0.93 (d, J=6.2 Hz, 3H), 0.78 (s, 9H), 0.03 (s, 6H).

MS: 543 (M+1).

### 6-(3-Chloro-benzyl)-1-((S)-1-hydroxymethyl-2-methyl-propyl)-4-oxo-1,4-dihydro-[1,7]naphthyridine-3-carboxylic acid

1H NMR (DMSO-d6, 400 MHz): δ 14.65 (brs, 1H, OH, exchangeable with D2O), 9.69 (s, 1H), 8.99 (s, 1H), 8.15 (s, 1H), 7.46 (s, 1H), 7.34 (m, 3H), 5.20 (brs, 1H, OH, exchangeable with D₂O), 5.05 (m, 1H), 4.34 (s, 2H), 3.96 (m, 1H), 3.84 (m, 1H), 2.38 (m, 1H), 1.13 (d, J=6.4 Hz, 3H), 0.73 (d, J=6.4 Hz, 3H).

MS: 401 (M+1).

### Example 5D

### 6-(3-Chloro-2-fluoro-benzyl)-1-((S)-1-hydroxymethyl-2-methyl-propyl)-8-methoxy-4-oxo-1,4-dihydro-1,7-naphthyridine-3-carboxylic acid and

### Example 5E

### (S)-8-(3-chloro-2-fluoro-benzyl)-3-isopropyl-6-oxo-2,3-dihydro-6H-1-oxa-3a,9-diaza-phenalene-5-carboxylic acid

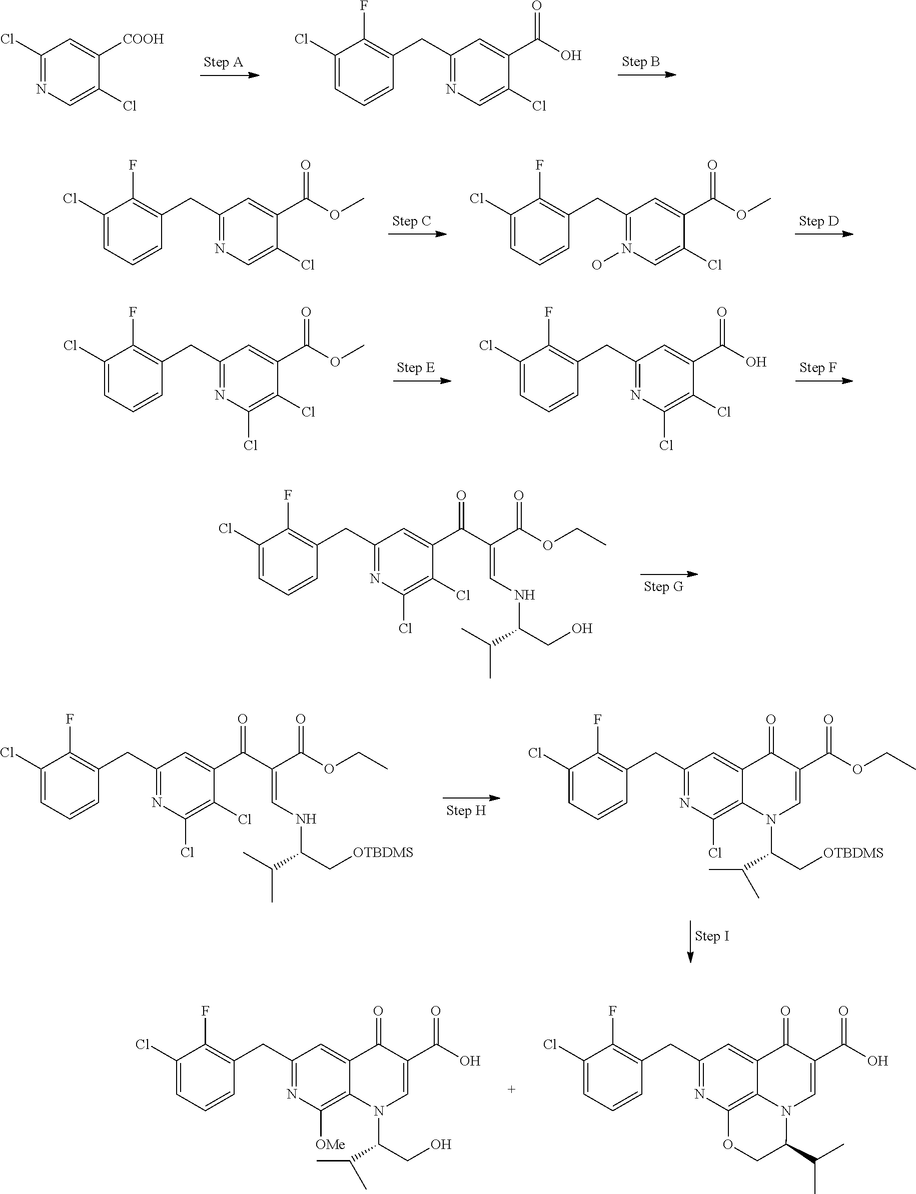

### Step A: 3-Chloro-6-(3-chloro-2-fluoro-benzyl)-isonicotinic acid

This compound was synthesized using the similar procedures to those described herein from 2,5-dichloroisonicotinic acid.

### Step B: 3-Chloro-6-(3-chloro-2-fluoro-benzyl)-isonicotinic acid methyl ester

A mixture of 3-chloro-6-(3-chloro-2-fluoro-benzyl)-isonicotinic acid (30 g, 0.1 mol) and thionyl chloride (15 mL, 0.2 mol) in 200 mL of anhydrous toluene and 1 mL of anhydrous DMF was refluxed for 2 h. Solvents were removed under reduced pressure to give a mobile oil residue which was azeoptoped with toluene (2×50 mL). The residue was dissolved in 200 mL of anhydrous methanol, heated to reflux for 30 min and cooled to room temperature. The solvent was removed under reduced pressure to give a white solid, which is pure enough for next step.

### Step C: 5-Chloro-2-(3-chloro-2-fluoro-benzyl)-1-oxy-isonicotinic acid methyl ester

A mixture of 3-chloro-6-(3-chloro-2-fluoro-benzyl)-isonicotinic acid methyl ester (10 g, 31.8 mmol) and 3-chloroperbenzoic acid (16 g, 92.7 mmol) in 200 mL of dichloromethane and 20 mL of methanol was stirred at room temperature overnight. The reaction mixture was washed with water, saturated sodium bicarbonate, brine and evaporated under reduced pressure. The residue was purified by silica gel chromatography (ISCO, hexane/EtOAc, 0%, 10 min, 0-10%, 30 min) to give a white solid (8.9 g, 85%).

¹H NMR (DMSO-d₆, 400 MHz): δ 8.71 (s, 1H), 7.95 (s, 1H), 7.52 (dt, J=1.5 and 7.8 Hz, 1H), 7.24 (dt, J=1.5 and 7.8 Hz, 1H), 7.18 (dt, J=1.0 and 7.8 Hz, 1H), 4.24 (s, 2H), 3.89 (s, 3H).

### Step D: 2,3-Dichloro-6-(3-chloro-2-fluoro-benzyl)-isonicotinic acid methyl ester

A mixture of 5-chloro-2-(3-chloro-2-fluoro-benzyl)-1-oxy-isonicotinic acid methyl ester (6.6 g, 20 mmol) in 30 mL of POCl₃ was refluxed at 130° C. for 6 hours and then evaporated under reduced pressure. The residue was purified by silica gel chromatography (ISCO, hexane/EtOAc, 0%, 10 min, 0-10%, 30 min) to give a yellow oily compound (4.7 g, 67%). ¹H NMR (CDCl₃, 400 MHz): δ 7.36 (s, 1H), 7.35 (dt, J=1.5 and 7.8 Hz, 1H), 7.21 (dt, j=1.5 and 7.8 Hz, 1H), 7.09 (dt, J=1.0 and 7.8 Hz, 1H), 4.20 (s, 2H), 3.98 (s, 3H).

### Step E: 2,3-Dichloro-6-(3-chloro-2-fluoro-benzyl)-isonicotinic acid

A mixture of 2,3-dichloro-6-(3-chloro-2-fluoro-benzyl)-isonicotinic acid methyl ester (3.5 g, 10 mmol) and 20 mL of 1 N LiOH in 60 mL of THF was stirred overnight at room temperature. The reaction mixture was evaporated in vacuo and the residue was dissolved in water and neutralized by 6 N HCl. The precipitate was filtered and washed with water to give the desired product as a white solid in quantitative yield.

¹H NMR (DMSO-d₆, 400 MHz): δ 14.47 (brs, 1H, OH, exchangeable with D₂O), 7.67 (s, 1H), 7.52 (dt, J=1.5 and 7.8 Hz, 1H), 7.36 (dt, J=1.5 and 7.8 Hz, 1H), 7.23 (dt, J=1.0 and 7.8 Hz, 1H), 4.25 (s, 2H).

### Step F: 2-[2,3-Dichloro-6-(3-chloro-2-fluoro-benzyl)-pyridine-4-carbonyl]-3-((S)-1-hydroxymethyl-2-methyl-propylamino)-acrylic acid ethyl ester

A mixture of 2,3-dichloro-6-(3-chloro-2-fluoro-benzyl)-isonicotinic acid (3.34 g, 10 mmol) and thionyl chloride (1.46 mL, 20 mmol) in 40 mL of anhydrous toluene and 0.1 mL of anhydrous DMF was refluxed for 2 h. The solvent was removed under reduced pressure to give a mobile oil residue which was azeoptoped with toluene (2×20 mL). The residue was dissolved in 10 mL of anhydrous THF and the resulting solution was added dropwise to a solution of ethyl 3-(dimethylamino)acrylate (1.56 g, 11 mmol) and triethylamine (1.22 g, 14.4 mmol) in 40 mL of anhydrous THF under nitrogen and heated under reflux for 7 hours. The reaction mixture was allowed to cool to room temperature and L-valinol (1.14 g, 11 mmol) was added with stirring at room temperature. The reaction mixture was stirred for an additional 30 min at room temperature and evaporated to dryness under reduced pressure. Water (50 mL) and ethyl acetate (50 mL) were added to allow partitioning. The organic layer was separated and washed successively with saturated aqueous sodium bicarbonate (×2), water, brine, dried over sodium sulfate and was concentrated under reduced pressure. The crude material was purified by silica gel chromatography (ISCO, hexane/EtOAc, 330 g, 0-40%, 30 min; 40-100%, 10 min; 100%, 30 min) to give a yellow oily compound.

### Step G: 3-[(S)-1-(tert-Butyl-dimethyl-silanyloxymethyl)-2-methyl-propylamino]-2-[2,3-dichloro-6-(3-chloro-2-fluoro-benzyl)-pyridine-4-carbonyl]-acrylic acid ethyl ester

A mixture of (Z)-2-[2,3-dichloro-6-(3-chloro-2-fluoro-benzyl)-pyridine-4-carbonyl]-3-((S)-1-hydroxymethyl-2-methyl-propylamino)-acrylic acid ethyl ester (1.5 g, 2.9 mmol), imidazole (1.97 g, 29 mmol), and tert-butyldimethylsilyl chloride (2.18 g, 14.5 mmol) in 15 mL of anhydrous DMF was stirred overnight under argon at room temperature. The mixture was evaporated to dryness under reduced pressure and the residue was purified by silica chromatography (ISCO, hexane/EtOAc, 0-30%, 20 min, 30-100%, 10 min, 100%, 10 min) to give the desired compound as a yellow foam in a quantitative yield.

¹H NMR (CDCl₃, 400 MHz): δ 11.05 (dd, J=9.6 and 13.8 Hz, 1H, NH, exchangeable with D₂O), 8.19 (d, J=13.9 Hz, 1H, it becomes singlet after D₂O exchange), 7.31 (dt, J=1.5 and 7.8 Hz, 1H), 7.21 (dt, J=1.5 and 7.8 Hz, 1H), 7.05 (dt, J=1.0 and 7.8 Hz, 1H), 4.17 (s, 2H), 3.98 (m, 2H), 3.81 (dd, J=3.8 and 11.3 Hz, 1H), 3.69 (dd, J=7.6 and 11.3 Hz, 1H), 3.18 (m, 1H), 2.03 (m, 1H), 1.06 (d, J=6.6 Hz, 3H), 1.05 (d, J=6.6 Hz, 3H), 0.93 (t, J=7.0 Hz, 3H), 0.90 (s, 9H), 0.96 (s, 6H).

MS: 633 (M+1).

### Step H: 1-[(S)-1-(tert-Butyl-dimethyl-silanyloxymethyl)-2-methyl-propyl]-8-chloro-6-(3-chloro-2-fluoro-benzyl)-4-oxo-1,4-dihydro-1,7-naphthyridine-3-carboxylic acid ethyl ester

A mixture of 3-[(S)-1-(tert-butyl-dimethyl-silanyloxymethyl)-2-methyl-propylamino]-2-[2,3-dichloro-6-(3-chloro-2-fluoro-benzyl)-pyridine-4-carbonyl]-acrylic acid ethyl ester (0.46 g, 0.73 mmol) and potassium carbonate (0.2 g, 1.46 mmol) in anhydrous DMF (5 mL) was stirred at 120° C. for 90 min. The solvent was evaporated under reduced pressure and the residue was purified by silica chromatography (ISCO, hexane/EtOAc, 0-40%, 20 min, 40-100%, 10 min, 100%, 10 min) to give the pure product as an yellow oil (0.13 g, 30%) and the recovered starting materials.

¹H NMR (CDCl₃, 400 MHz): δ 8.91 (s, 1H), 8.08 (s, 1H), 7.33 (dt, J=1.5 and 7.8 Hz, 1H), 7.19 (dt, J=1.5 and 7.8 Hz, 1H), 7.15 (dt, J=1.0 and 7.8 Hz, 1H), 5.24 (ddd, J=2.5, 3.9 and 10.4 Hz, 1H), 4.40 (q, J=7.0 Hz, 2H), 4.27 (s, 2H), 4.12 (m, 2H), 2.48 (m, 1H), 1.41 (t, J=7.1 Hz, 3H), 1.17 (d, J=6.6 Hz, 3H), 0.86 (s, 9H), 0.79 (d, J=6.6 Hz, 3H), 0.05 (s, 6H).

MS: 597 (M+1).

### Step I: 6-(3-Chloro-2-fluoro-benzyl)-1-((S)-1-hydroxymethyl-2-methyl-propyl)-8-methoxy-4-oxo-1,4-dihydro-1,7-naphthyridine-3-carboxylic acid

A mixture of 1-[(S)-1-(tert-butyl-dimethyl-silanyloxymethyl)-2-methyl-propyl]-8-chloro-6-(3-chloro-2-fluoro-benzyl)-4-oxo-1,4-dihydro-1,7-naphthyridine-3-carboxylic acid ethyl ester (100 mg, 0.17 mmol) and 28% sodium methoxide (2 mL) in anhydrous methanol (15 mL) under argon was stirred at 80° C. for 5 hour. The solvent was evaporated under reduced pressure and the residue was dissolved in 10 mL of water and filtered. The filtrate was neutralized with 6 N HCl and the precipitate was filtered and washed with water to give a white solid as a mixture of (S)-8-(3-chloro-2-fluoro-benzyl)-3-isopropyl-6-oxo-2,3-dihydro-6H-1-oxa-3a,9-diaza-phenalene-5-carboxylic acid and 6-(3-Chloro-2-fluoro-benzyl)-1-((S)-1-hydroxymethyl-2-methyl-propyl)-8-methoxy-4-oxo-1,4-dihydro-1,7-naphthyridine-3-carboxylic acid, which was purified by preparative HPLC.

¹H NMR (DMSO-d₆, 400 MHz): δ 14.72 (brs, 1H, OH, exchangeable with D₂O), 8.97 (s, 1H), 7.69 (s, 1H), 7.52 (dt, J=1.5 and 7.8 Hz, 1H), 7.45 (dt, J=1.5 and 7.8 Hz, 1H), 7.25 (dt, J=1.0 and 7.81 Hz, 1H), 5.62 (m, 1H), 5.22 (brs, 1H, OH, exchangeable with D₂O), 4.28 (s, 2H), 4.02 (s, 3H), 3.94 (dd, J=5.8 and 12.6 Hz, 1H), 3.83 (dd, J=2.7 and 12.6 Hz, 1H), 2.37 (m, 1H), 1.11 (d, J=6.7 Hz, 3H), 0.79 (d, J=6.7 Hz, 3H).

MS: 449 (M+1).

### Examples 5B-5P

Examples 5B-5P were prepared according to the procedure described above for example 5A.

| Compound | Structure | Analytical Data |
|---|---|---|
| 5A | 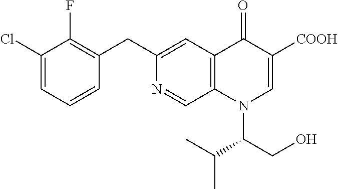 | ¹H NMR (DMSO-d₆, 400 MHz): δ 14.62 (brs, 1H, OH, exchangeable with D₂O), 9.68 (s, 1H), 8.99 (s, 1H), 8.11 (s, 1H), 7.49 (t, J = 7.8 Hz, 1H), 7.40 (t, J = 7.8 Hz, 1H), 7.20 (t, J = 7.8 Hz, 1H), 5.20 (brs, 1H, OH, exchangeable with D₂O), 5.03 (m, 1H), 4.42 (s, 2H), 3.96 (m, 1H), 3.84 (m, 1H), 2.38 (m, 1H), 1.13 (d, J = 6.4 Hz, 3H), 0.73 (d, J = 6.4 Hz, 3H). MS: 419 (M + 1). |
| 5B | 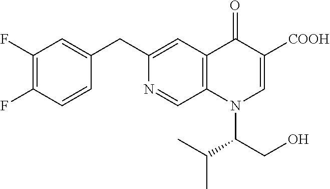 | ¹H NMR (DMSO-d₆, 400 MHz): δ 14.68 (brs, 1H, OH, exchangeable with D₂O), 9.69 (s, 1H), 8.99 (s, 1H), 8.15 (s, 1H), 7.45 (m, 1H), 7.37 (m, 1H), 7.21 (m, 1H), 5.21 (brs, 1H, OH, exchangeable with D₂O), 5.05 (m, 1H), 4.32 (s, 2H), 3.96 (m, 1H), 3.84 (m, 1H), 2.38 (m, 1H), 1.13 (d, J = 6.4 Hz, 3H), 0.73 (d, J = 6.4 Hz, 3H). MS: 403 (M +1). |
| 5C | 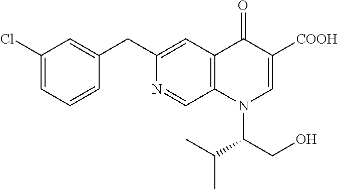 | ¹H NMR (CDCl₃, 400 MHz): δ 9.11 (s, 1H), 8.77 (s, 1H), 8.26 (s, 1H), 7.32 (m, 2H), 7,25 (m, 2H), 4.44 (q, J = 7.1 Hz, 2H), 4.31 (s, 2H), 4.08 (m, 1H), 4.02 (m, 1H), 2.51 (m, 1H), 1.45 (t, J = 7.1 Hz, 3H), 1.25 (d, J = 6.2 Hz, 3H), 0.93 (d, J = 6.2 Hz, 3H), 0.78 (s, 9H), 0.03 (s, 6H). MS: 543 (M + 1). |
| 5D | 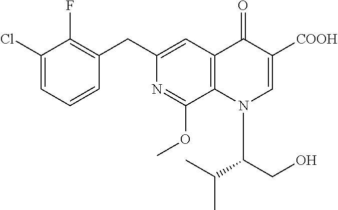 | ¹H NMR (DMSO-d₆, 400 MHz): δ 14.72 (brs, 1H, OH, exchangeable with D₂O), 8.97 (s, 1H), 7.69 (s, 1H), 7.52 (dt, J = 1.5 and 7.8 Hz, 1H), 7.45 (dt, J =1.5 and 7.8 Hz, 1H), 7.25 (dt, J = 1.0 and 7.8 Hz, 1H), 5.62 (m, 1H), 5.22 (brs, 1H, OH, exchangeable with D₂O), 4.28 (s, 2H), 4.02 (s, 3H), 3.94 (dd, J = 5.8 and 12.6 Hz, 1H), 3.83 (dd, J = 2.7 and 12.6 Hz, 1H), 2.37 (m, 1H), 1.11 (d, J = 6.7 Hz, 3H), 0.79 (d, J = 6.7 Hz, 3H). MS: 449 (M + 1). |
| 5E | 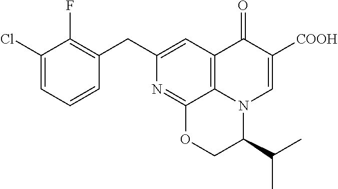 | MS: 417 (M + H)⁺ |
| 5F | 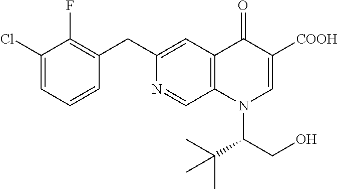 | MS: 437 (M + H)⁺ |
| 5G | 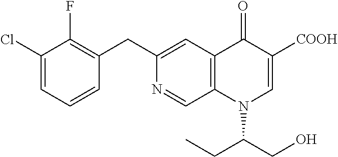 | MS: 405 (M + H)⁺ |
| 5H | 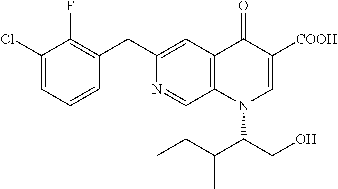 | MS: 433 (M + H)⁺ |
| 5I | 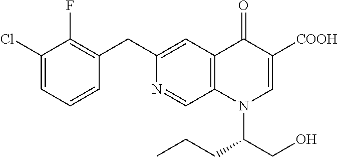 | MS: 418 (M + H)⁺ |
| 5J | 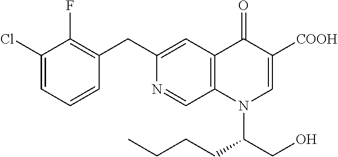 | MS: 433 (M + H)⁺ |
| 5K | 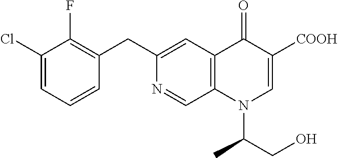 | MS: 391 (M + H)⁺ |
| 5L | 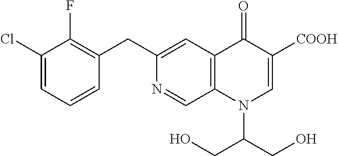 | MS: 407 (M + H)⁺ |
| 5M | 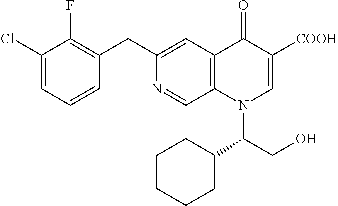 | MS: 459 (M + H)⁺ |
| 5N | 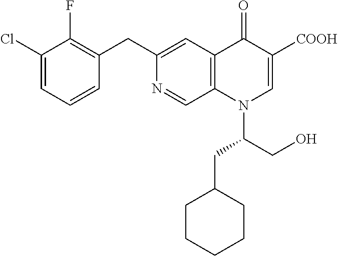 | MS: 473 (M + H)⁺ |
| 5O | 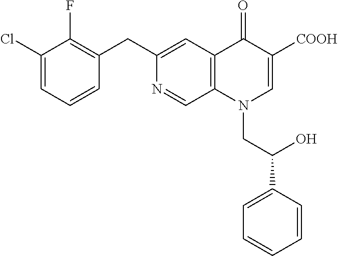 | MS: 453 (M + H)⁺ |
| 5P | 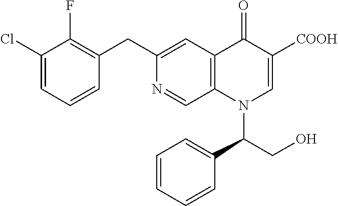 | MS: 453 (M + H)⁺ |
| 5Q | 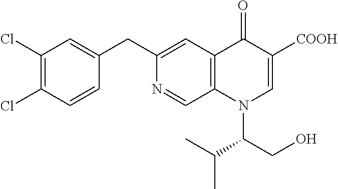 | MS: 436 (M + H)⁺ |

### Example 6

### Compounds of Formula (VI)

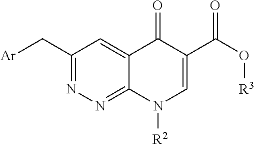

Compounds of formula (VI) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

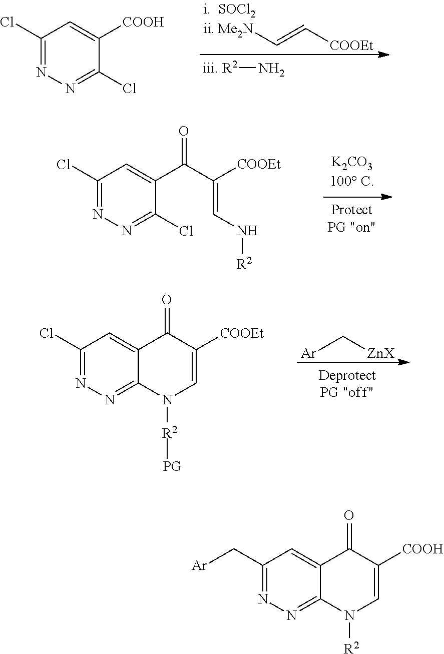

### Example 6A

### 3-(3-chloro-2-fluorobenzyl)-8-(1-hydroxy-3-methylbutan-2-yl)-5-oxo-5,8-dihydropyrido[2,3-c]pyridazine-6-carboxylic acid

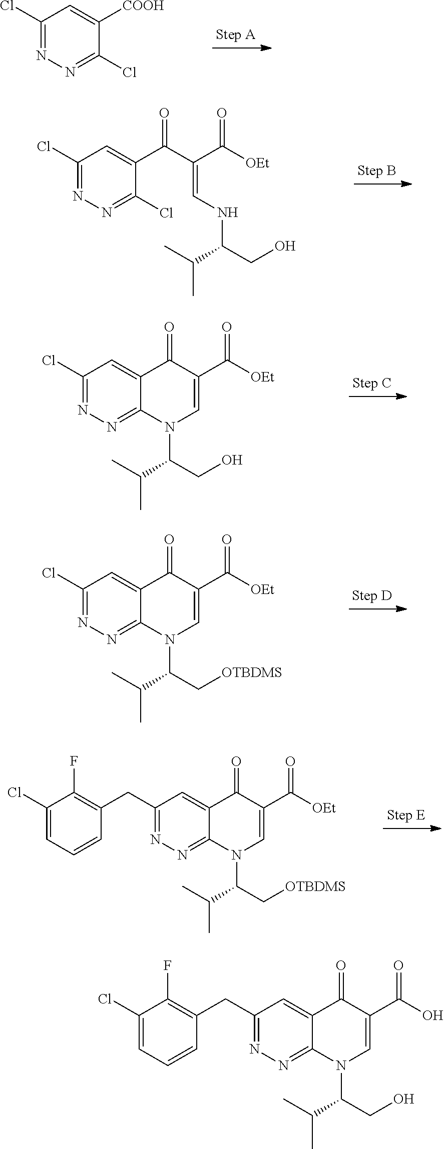

### Step A: (S)-Ethyl 2-(3,6-dichloropyridazine-4-carbonyl)-3-(1-hydroxy-3-methylbutane-2-ylamino)acrylate

This Compound was synthesized using the same procedure described in example 1A.

### Step B: (S)-Ethyl 3-chloro-8-(1-hydroxy-3-methylbutan-2-yl)-5-oxo-5,8-dihydropyrido-[2,3-c]pyridazine-6-carboxylate

This compound was synthesized using the same procedure described in example 1A.

### Step C: (S)-Ethyl 8-(1-(tert-butyldimethylsilyloxy)-3-methylbutan-2-yl)-3-chloro-5-oxo-5,8-dihydropyrido[2,3-c]pyridazine-6-carboxylate

This compound was synthesized using the same procedure described in example 1A.

### Step D: 8-[(S)-1-(tert-Butyl-dimethyl-silanyloxymethyl)-2-methyl-propyl]-3-(3-chloro-2-fluoro-benzyl)-5-oxo-5,8-dihydro-pyrido[2,3-c]pyridazine-6-carboxylic acid ethyl ester

Under an argon stream, zinc powder (346 mg, 5.3 mmol) was suspended in 1 mL of dry tetrahydrofuran, 1,2-dibromoethane (1.4 μl, 0.016 mmol) and trimethylsilyl chloride (4.0 μl, 0.032 mmol) were added at 60° C. and the mixture was stirred with heating for 30 min. A solution of 2-fluoro-3-chloro-benzyl bromide (177 mg, 0.79 mmol) in 2 mL of dry tetrahydrofuran was added dropwise at 60° C. The mixture was stirred with heating for 1 hour and allowed to cool to room temperature to give a solution of 2-fluoro-3-chloro-benzylzinc bromide in tetrahydrofuran. This was used in the next step.

8-[(S)-1-(tert-Butyl-dimethyl-silanyloxymethyl)-2-methyl-propyl]-3-chloro-5-oxo-5,8-dihydro-pyrido[2,3-c]pyridazine-6-carboxylic acid ethyl ester (240 mg, 0.53 mmol) was dissolved in 10 mL of dry tetrahydrofuran under an argon stream. Dichlorobis(triphenylphosphine)palladium(II) (34 mg, 0.048 mmol) was added followed by the addition a solution of the above-mentioned 2-fluoro-3-chloro-benzylzinc bromide in tetrahydrofuran at 60° C. The mixture was stirred with heating at the same temperature for an additional hour. The reaction mixture was allowed to cool to room temperature, 1 N hydrochloric acid was added and the mixture was extracted three times with ethyl acetate. The organic layer was washed with water, brine, dried over anhydrous sodium sulfate and concentrated under reduced pressure. The crude material was purified by silica gel chromatography (ISCO, 12 g of column, chloroform/methanol, 0-30%, 25 min; 30-80%, 10 min; 80%, 5 min) to give a major product as an yellow foam 200 mg (67%).

1H NMR (CDCl3, 400 MHz): δ 9.02 (s, 1H), 8.33 (s, 1H), 7.36 (dt, J=2.0 and 7.8 Hz, 1H), 7.29 (t, J=7.8 Hz, 1H), 7.10 (dt, J=1.2 and 7.8 Hz, 1H), 5.86 (d, J=9.1 Hz, 1H), 4.61 (s, 2H), 4.43 (q, J=7.1 Hz, 2H), 4.17 (dd, J=3.6 and 12.0, 1H), 3.88 (d, J=10.9 Hz, 1H), 2.59 (m, 1H), 1.43 (t, J=7.1 Hz, 3H), 1.24 (d, J=6.2 Hz, 3H), 0.89 (d, J=6.2 Hz, 3H), 0.86 (s, 9H), 0.04 (s, 6H).

MS: 562 (M+1).

### Step E: 3-(3-Chloro-2-fluoro-benzyl)-8-((S)-1-hydroxymethyl-2-methyl-propyl)-5-oxo-5,8-dihydro-pyrido[2,3-c]pyridazine-6-carboxylic acid

1-[(S)-1-(tert-butyl-dimethyl-silanyloxymethyl)-2-methyl-propyl]-7-(3-chloro-2-fluoro-benzyl)-6-methoxy-4-oxo-1,4-dihydro-[1,5]naphthyridine-3-carboxylic acid ethyl ester (100 mg, 0.18 mmol) was dissolved in 20 mL of methanol, 2 mL of 25% sodium methoxide in methanol and 4 mL of water. The resulting mixture was refluxed for 4 hours, allowed to cool to room temperature and evaporated to a small volume under reduced pressure. Water (10 mL) was added, the mixture was filtered and the filtrate was neutralized with 1 N hydrochloric acid. The solid was filtered and washed with water to give a pure product as an yellow solid (45 mg, 60%).

¹H NMR (DMSO-d₆, 400 MHz): δ 13.81 (brs, 1H, OH, exchangeable with D₂O), 9.16 (s, 1H), 8.45 (s, 1H), 7.52 (dt, J=1.4 and 7.8 Hz, 1H), 7.47 (dt, J=1.4 and 7.8 Hz, 1H), 7.24 (t, J=7.8 Hz, 1H), 5.84 (brs, 1H, OH, exchangeable with D₂O), 5.20 (m, 1H), 4.68 (s, 2H), 4.06 (m, 1H), 3.82 (m, 1H), 2.44 (m, 1H), 1.14 (d, J=6.6 Hz, 3H), 0.75 (d, J=6.6 Hz, 3H).

MS: 420 (M+1).

### Example 7

### Compounds of Formula (VII)

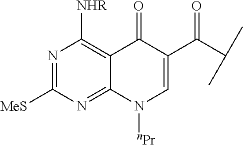

### Examples 7A-7D

These compounds were prepared according to the scheme shown below:

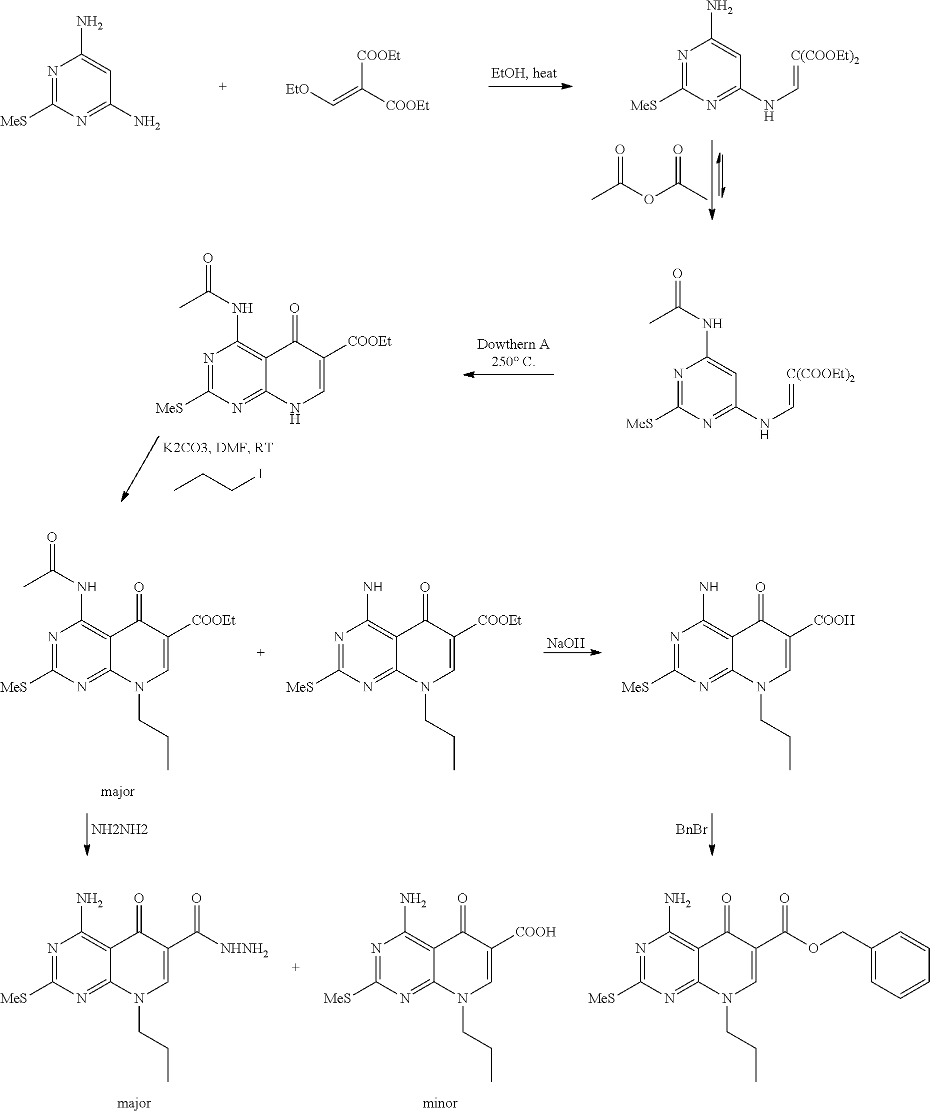

| Compound | Structure | ¹H NMR |
|---|---|---|
| 7A | 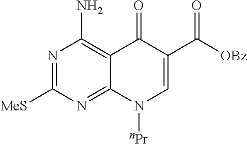 | ¹H NMR (DMSO-d₆, 400 MHz): δ 9.62 (s, 1H), 8.63 (s, 1H), 7.49 (m, 5H), 5.28 (s, 2H), 4.23 (t, J = 4 Hz, 2H), 2.53 (s, 3H), 1.78 (m, 2H), 0.89 (t, J = 4 Hz, 3H). |
| 7B | 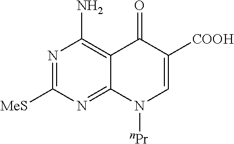 | ¹H NMR (DMSO-d₆, 400 MHz): δ 9.9 (s, 1H), 8.29 (s, 1H), 8.07 (s, 1H), 4.18 (t, J = 8 Hz, 2H), 2.53 (s, 3H), 1.78 (m, 2H), 0.89 (t, J = 4 Hz, 3H). |
| 7C | 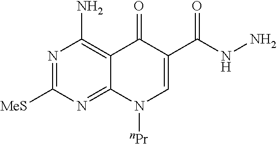 | ¹H NMR (DMSO-d₆, 400 MHz): δ 10.35 (t, J = 4 Hz, 1H), 9.44 (s, 1H), 8.73 (s, 1H), 8.41 (s, 1H), 4.61 (d, J = 4 Hz, 2H), 4.33 (t, J = 8 Hz, 2H), 2.53 (s, 3H), 1.78 (m, 2H), 0.89 (t, J = 4 Hz, 3H). |
| 7D | 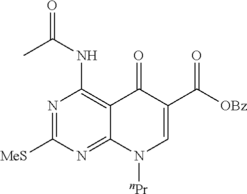 | ¹H NMR (DMSO-d₆, 400 MHz): δ 13.0 (s, 1H), 8.81 (s, 1H), 7.49 (m, 5H), 5.31 (s, 2H), 4.31 (t, J = 8 Hz, 2H), 2.58 (s, 3H), 2.53 (s, 3H), 1.78 (m, 2H), 0.89 (t, J = 4 Hz, 3H). |

### Example 8

### Compounds of Formula (VIII)

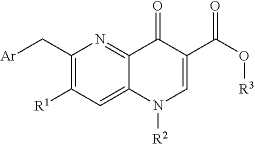

Compounds of formula (VIII) were prepared according to the following general synthetic schemes. When appropriate, protecting groups are used as needed according to established synthetic procedures known to those of skill in the art, and may or may not be removed upon completion of the synthesis. Starting materials are synthesized according to methods known in the art or are commercially available.

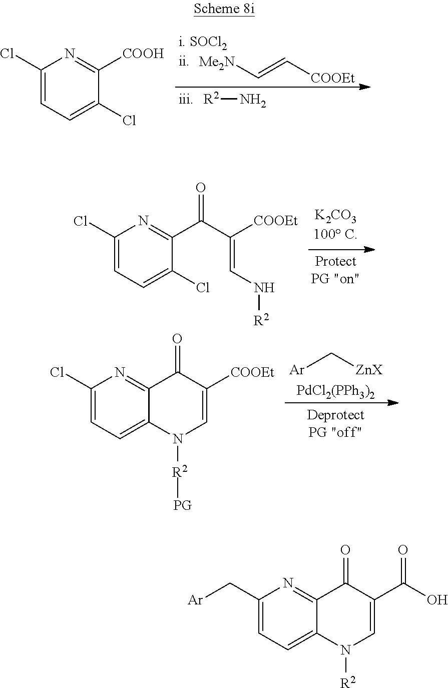

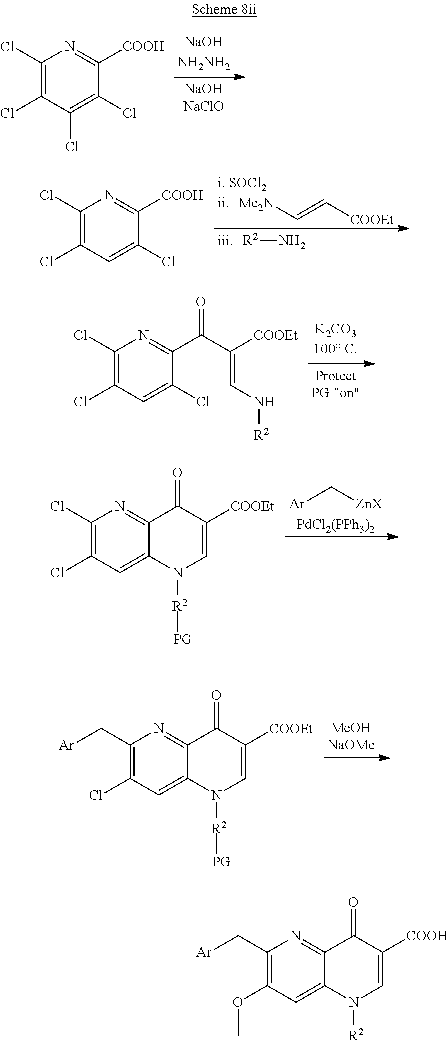

### Example 8A

### 6-(3-Chloro-2-fluoro-benzyl)-1-((S)-1-hydroxymethyl-2-methyl-propyl)-4-oxo-1,4-dihydro-[1,5]naphthyridine-3-carboxylic acid

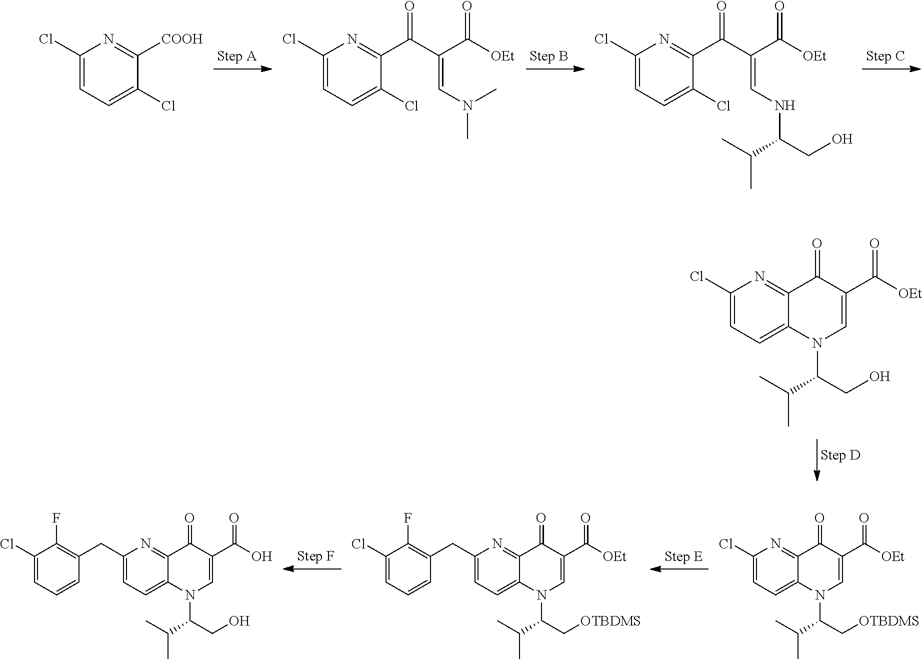

### Step A: 2-(3,6-Dichloro-pyridine-2-carbonyl)-3-dimethylamino-acrylic acid ethyl ester

A mixture of 3,6-dichloro-pyridine-2-carboxylic acid (5.76 g, 30 mmol) and thionyl chloride (4.4 mL, 60 mmol) was dissolved in a mixture of 50 mL of anhydrous toluene and 0.5 mL of anhydrous DMF. The mixture was refluxed for 2 h and the solvent was removed under reduced pressure to give an oil which was azeoptoped with toluene (20 mL). The residue was dissolved in 20 mL of anhydrous THF and this solution was added dropwise to a solution of ethyl 3-(dimethylamino)acrylate (4.7 g, 33 mmol) and triethylamine (3.64 g, 16 mmol) in 20 mL of anhydrous THF under nitrogen. The mixture was heated under reflux for 7 hours and was allowed to cool to room temperature and concentrated under reduced pressure. Water (100 mL) and ethyl acetate (100 mL) were added to allow partitioning. The organic layer was separated and washed successively with saturated aqueous sodium bicarbonate (×2), water, brine, dried over sodium sulfate and concentrated under reduced pressure to give a crude product as yellow oil, which was used for next step without further purification.

### Step B: 2-(3,6-Dichloro-pyridine-2-carbonyl)-3-((S)-1-hydroxymethyl-2-methyl-propylamino)-acrylic acid ethyl ester

A solution of the above product and L-valinol (3.09 g, 30 mmol) in anhydrous THF (100 mL) was stirred for 30 min at room temperature, and the mixture was evaporated to dryness to give a crude product in a quantitative yield, which was used for next step without further purification. An analytically pure sample was prepared by silica gel chromatography (ISCO, Chloroform/methanol, 0-40%, 40 min) to give the pure compound as yellow oil.

1H NMR (DMSO-d6, 400 MHz): δ 10.91 (dd, J=9.6 and 13.8 Hz, 1H, NH, exchangeable with D2O), 8.27 (d, J=14.3 Hz, 1H, it becomes singlet after D2O exchange), 8.01 (d, J=8.4 Hz, 1H), 7.55 (d, J=8.4 Hz, 1H), 5.08 (brs, 1H, OH, exchangeable with D2O), 1.87 (q, J=7.0 Hz, 2H), 3.62 (m, 2H), 3.40 (m, 1H), 1.95 (m, 1H), 0.95 (d, J=6.6 Hz, 3H), 0.91 (d, J=6.6 Hz, 3H), 0.90 (t, J=7.0 Hz, 3H).

MS: 375 (M+1), 373 (M−1).

### Step C: 6-Chloro-1-(1-hydroxymethyl-2-methyl-propyl)-4-oxo-1,4-dihydro-[1,5]naphthyridine-3-carboxylic acid ethyl ester

A mixture of 2-(3,6-dichloro-pyridine-2-carbonyl)-3-((S)-1-hydroxymethyl-2-methyl-propylamino)-acrylic acid ethyl ester (8.9 g, 23.7 mmol) and potassium carbonate (6.5 g, 47.4 mmol) in anhydrous DMF (100 mL) was stirred at 100° C. overnight, the mixture was evaporated to dryness under reduced pressure and the residue was purified by ISCO (Chloroform/methanol, 0-40%, 40 min) to give the pure compound as a yellow solid (3.8 g, 47%).

1H NMR (DMSO-d6, 400 MHz): δ 8.68 (s, 1H), 8.67 (d, J=9.2 Hz, 1H), 7.86 (d, J=9.2 Hz, 1H), 5.13 (t, J=5.1 Hz, 1H, OH, exchangeable with D2O), 4.63 (m, 1H), 4.25 (q, J=7.01 Hz, 2H), 3.88 (m, 1H), 3.80 (m, 1H), 2.29 (m, 1H), 1.29 (t, J=7.0 Hz, 3H), 1.10 (d, J=6.6 Hz, 3H), 0.72 (d, J=6.6 Hz, 3H).

MS: 337 (M−1).

### Step D: 1-[(S)-1-(tert-Butyl-dimethyl-silanyloxymethyl)-2-methyl-propyl]-6-chloro-4-oxo-1,4-dihydro-[1,5]naphthyridine-3-carboxylic acid ethyl ester

To a mixture of 6-chloro-1-(1-hydroxymethyl-2-methyl-propyl)-4-oxo-1,4-dihydro[1,5]naphthyridine-3-carboxylic acid ethyl ester (1.0 g, 2.95 mmol) and imidazole (2.01 g, 29.5 mmol) in 10 mL of anhydrous DMF was added tert-butyldimethylsilyl chloride (2.22 g, 14.8 mmol) under argon at room temperature. The mixture was stirred overnight at room temperature and evaporated to dryness under reduced pressure. The crude material was purified by ISCO (chloroform/methanol, 0-30%, 40 min) to give the pure compound as yellow oil.

1H NMR (DMSO-d6, 400 MHz): δ 8.78 (d, J=9.0 Hz, 1H), 8.72 (s, 1H), 7.96 (d, J=9.0 Hz, 1H), 4.81 (m, 1H), 4.31 (q, J=7.1 Hz, 2H), 4.10 (dd, J=5.8 and 11.6 Hz, 1H), 3.99 (dd, J=2.0 and 11.6 Hz, 1H), 2.41 (m, 1H), 1.35 (t, J=7.1 Hz, 3H), 1.20 (d, J=6.6 Hz, 3H), 0.83 (d, J=6.6 Hz, 3H), 0.77 (s, 9H), 0.03 (s, 6H).

MS: 453 (M+1).

### Step E: 1-[(S)-1-(tert-Butyl-dimethyl-silanyloxymethyl)-2-methyl-propyl]-6-(3-chloro-2-fluoro-benzyl)-4-oxo-1,4-dihydro-[1,5]naphthyridine-3-carboxylic acid ethyl ester

Under an argon stream, zinc powder (480 mg, 7.34 mmol) was suspended in 1 mL of dry tetrahydrofuran. 1,2-Dibromoethane (1.4 μl, 0.016 mmol) and trimethylsilyl chloride (4.0 μl, 0.032 mmol) were added at 60° C., and the mixture was stirred with heating for 30 min. A solution of 2-fluoro-3-chloro-benzyl bromide (352 mg, 1.58 mmol) in 2 mL of dry tetrahydrofuran was added dropwise at 60° C. The mixture was stirred with heating for 1 hour and allowed to cool to room temperature to give a solution of 1M 2-fluoro-3-chloro-benzylzinc bromide in tetrahydrofuran. This was used in the next step.

1-[(S)-1-(tert-Butyl-dimethyl-silanyloxymethyl)-2-methyl-propyl]-6-chloro-4-oxo-1,4-dihydro-[1,5]naphthyridine-3-carboxylic acid ethyl ester (553 mg, 1.22 mmol) was dissolved in 20 mL of dry tetrahydrofuran under an argon stream. Dichlorobis(triphenylphosphine)palladium(II) (34 mg, 0.048 mmol) was added followed by the addition dropwise of the above-mentioned 2-fluoro-3-chloro-benzylzinc bromide solution at 60° C. After the completion of the addition, the mixture was stirred with heating at the same temperature for 1.5 hour. The reaction mixture was allowed to cool to room temperature, 1 N hydrochloric acid was added and the mixture was extracted three times with ethyl acetate. The organic layers were combined and washed successively with water, brine, dried over anhydrous sodium sulfate and concentrated under reduced pressure. The resulting crude material was purified by silica gel chromatography (ISCO, 12 g of column, chloroform/methanol, 0-30%, 25 min; 30-80%, 10 min; 80%, 5 min) to give a major product as an yellow foam 500 mg (73%).

1H NMR (DMSO-d6, 400 MHz): δ 8.70 (s, 1H), 8.62 (d, J=9.2 Hz, 1H), 7.72 (d, J=9.2 Hz, 1H), 7.57 (dt, J=2.0 and 7.8 Hz, 1H), 7.41 (dt, J=2.0 and 7.8 Hz, 1H), 7.28 (t, J=7.8 Hz, 1H), 4.77 (m, 1H), 4.42 (s, 2H), 4.32 (q, J=7.1 Hz, 2H), 4.11 (m, 1H), 3.99 (m, 1H), 2.42 (m, 1H), 1.36 (t, J=7.1 Hz, 3H), 1.21 (d, J=6.2 Hz, 3H), 0.83 (d, J=6.2 Hz, 3H), 0.75 (s, 9H), 0.03 (s, 6H).

MS: 562 (M+1).

### Step F: 6-(3-Chloro-2-fluoro-benzyl)-1-((S)-1-hydroxymethyl-2-methyl-propyl)-4-oxo-1,4-dihydro-[1,5]naphthyridine-3-carboxylic acid

The above intermediate (500 mg) was dissolved in 20 mL of methanol. Sodium methoxide (2 mL of 25% in methanol) and water (4 mL) were added. The mixture was refluxed for 4 hours, allowed to cool to room temperature and evaporated to a small volume under reduced pressure. Water (10 mL) was added and filtered. The filtrate was neutralized with 1 N hydrochloric acid and the solid was filtered and washed with water to give a pure product as an yellowish solid (365 mg, 71%).

¹H NMR (DMSO-d₆, 400 MHz): δ 15.38 (brs, 1H, OH, exchangeable with D₂), 8.97 (s, 1H), 8.76 (d, J=8.6 Hz, 1H), 7.84 (d, J=8.6 Hz, 1H), 7.50 (t, J=7.8 Hz, 1H), 7.38 (t, J=7.8 Hz, 1H), 7.22 (t, J=7.8 Hz, 1H), 5.18 (brs, 1H, OH, exchangeable with D₂O), 4.83 (m, 1H), 4.42 (s, 2H), 3.98 (m, 1H), 3.79 (m, 1H), 2.38 (m, 1H), 1.12 (d, J=6.2 Hz, 3H), 0.71 (d, J=6.2 Hz, 3H).

MS: 419 (M+1).

### Example 8B

### 6-(3,4-Difluoro-benzyl)-1-((S)-1-hydroxymethyl-2-methyl-propyl)-4-oxo-1,4-dihydro-[1,5]naphthyridine-3-carboxylic acid

This compound was synthesized using procedures described herein from commercially available 3,4-difluorobenzylzine bromide (0.5M in THF, Aldrich).

¹H NMR (DMSO-d₆, 400 MHz): δ 15.41 (brs, 1H, OH, exchangeable with D₂O), 8.75 (s, 1H), 8.76 (d, J=8.6 Hz, 1H), 7.88 (d, J=8.6 Hz, 1H), 7.46 (ddd, J=2.2, 8.0 and 11.8 Hz, 1H), 7.38 (dt, J=8.6 and 11.0 Hz, 1H), 7.19 (m, 1H), 5.16 (brs, 1H, OH, exchangeable with D₂O), 4.83 (m, 1H), 4.33 (s, 2H), 3.96 (m, 1H), 3.79 (m, 1H), 2.37 (m, 1H), 1.12 (d, J=6.2 Hz, 3H), 0.70 (d, J=6.2 Hz, 3H).

MS: 403 (M+1).

### Example 8C

### 6-(3-Chloro-benzyl)-1-((S)-1-hydroxymethyl-2-methyl-propyl)-4-oxo-1,4-dihydro-[1,5]naphthyridine-3-carboxylic acid

This compound was synthesized using procedures described herein from commercially available 3-chlorobenzylzinc bromide (0.5M in THF, Aldrich).

¹H NMR (DMSO-d₆, 400 MHz): δ 15.43 (brs, 1H, OH, exchangeable with D₂O), 8.97 (s, 1H), 8.76 (d, J=8.6 Hz, 1H), 7.88 (d, J=8.6 Hz, 1H), 7.46 (d, J=2.0 Hz, 1H), 7.32 (m, 3H), 5.16 (brs, 1H, OH, exchangeable with D₂O), 4.83 (m, 1H), 4.34 (s, 2H), 3.95 (m, 1H), 3.79 (m, 1H), 2.37 (m, 1H), 1.12 (d, J=6.2 Hz, 3H), 0.70 (d, J=6.2 Hz, 3H).

MS: 401 (M+1).

### Example 8D

### 6-(3-Chloro-2-fluoro-benzyl)-1-((S)-1-hydroxymethyl-2-methyl-propyl)-7-methoxy-4-oxo-1,4-dihydro-[1,5]naphthyridine-3-carboxylic acid

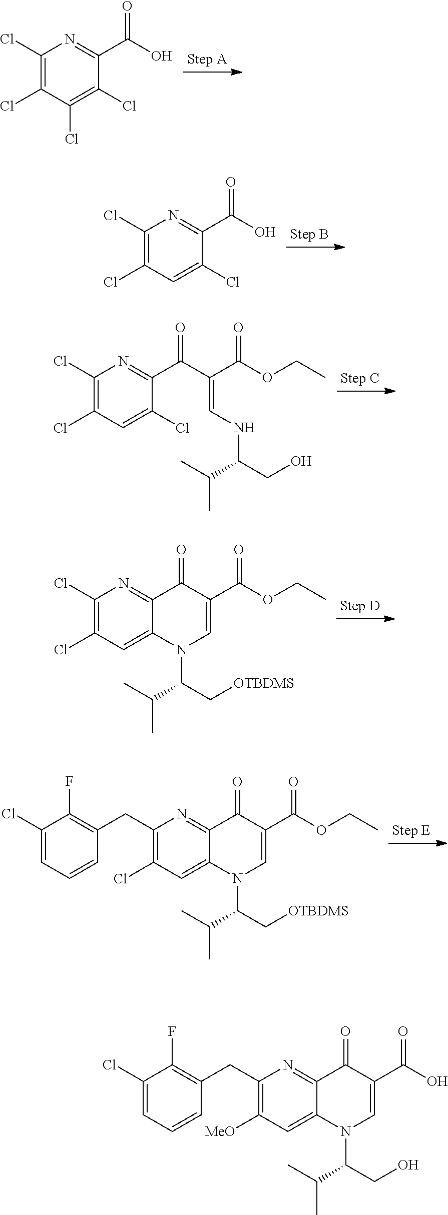

### Step A: 3,5,6-Trichloropicolinic acid

To a reaction flask containing 200 mL of boiling water was added 26.1 g (0.1 mol) of tetrapicolinic acid, 4.1 g (0.103 mol) of sodium hydroxide previously dissolved in 25 mL of water and 3.47 g (0.105 mol) of anhydrous hydrazine. The reaction mixture was stirred under reflux for 30 min. An additional 4.1 g (0.103 mol) of sodium hydroxide in 25 mL of water was slowly added to the reaction mixture over a 25 minute period and the mixture refluxed for 45 min. The reaction mixture was cooled to room temperature and 25 mL of 5N HCl was added. The solid 3,5,6-trichloro-4-hydrazinopicolinic acid (as the monohydrate) which precipitated was recovered by filtration in a yield of 22.9 g (83%). Mp: 166°-168° C.

To a mixture (clear solution) of 3,5,6-trichloro-4-hydrazinopicolinic acid (22.9 g, 78 mmol), 115 mL of 20% sodium hydroxide and 150 mL of water was added 100 mL of 10-13% sodium hypochlorite solution at 30° C. Immediate gas evolution was noted which ceased after about 3 min. Five minutes after the addition of the sodium hypochlorite solution, the reaction mixture was acidified to a pH of about 2 with concentrated hydrochloric acid (150 mL). The mixture was extracted with methylene chloride. The methylene chloride was removed from the extract by evaporation leaving the crude 3,5,6-trichloropicolinic acid. The crude product was dissolved in 1 N NaOH to form a clear solution and cooled to 0° C. and neutralized with 5 N HCl with stirring at 0° C. The solid was filtered and washed with water to give the pure enough product (15.5 g, 88%). Total yield: 68%. Mp. 147-151° C.

¹H NMR (DMSO-d₆, 400 MHz): δ 14.31 (brs, 1H, OH, exchangeable with D₂O), 8.60 (s, 1H).

### Step B: 2-(3,5,6-Trichloro-pyridine-2-carbonyl)-3-((S)-1-hydroxymethyl-2-methyl-propylamino)-acrylic acid ethyl ester

A mixture of 3,5,6-trichloro-pyridine-2-carboxylic acid (6.79 g, 30 mmol) and thionyl chloride (4.4 mL, 60 mmol) in 50 mL of anhydrous toluene and 0.5 mL of anhydrous DMF was refluxed for 2 h. The solvent was removed under reduced pressure to give a mobile oil residue which was azeoptoped with toluene (20 mL). The residue was dissolved in 20 mL of anhydrous THF and this solution was added dropwise to a solution of ethyl 3-(dimethylamino)acrylate (4.7 g, 33 mmol) and triethylamine (3.64 g, 36 mmol) in 30 mL of anhydrous THF under nitrogen. The resulting solution was heated under reflux for 7 hours. The reaction mixture was allowed to cool to room temperature and L-valinol (3.40 g, 33 mmol) in anhydrous THF (40 mL) was added with stirring at room temperature. The reaction mixture was stirred for 30 min at room temperature and evaporated to dryness under reduced pressure. Water (100 mL) and ethyl acetate (100 mL) were added to allow partitioning. The organic layer was separated and washed successively with saturated aqueous sodium bicarbonate (×2), water, brine, dried over sodium sulfate and was concentrated under reduced pressure. The crude product was purified by silica gel chromatography (ISCO, hexane/EtOAc, 330 g, 0-40%, 30 min; 40-100%, 10 min; 100%, 30 min) to give the pure compound as a yellow oil (10.9 g, 88.8%).

¹H NMR (CDCl₃, 400 MHz): δ 11.08 (dd, J=9.6 and 13.8 Hz, 1H, NH, exchangeable with D₂O), 8.35 (d, J=14.6 Hz, 1H, it becomes singlet after D₂O exchange), 7.81 (s, 1H), 5.08 (brs, 1H, OH, exchangeable with D₂O), 4.01 (q, J=7.2 Hz, 2H), 3.85 (dd, J=3.8 and 11.4 Hz, 1H), 3.75 (dd, J=7.6 and 11.4 Hz, 1H), 3.22 (m, 1H), 2.02 (m, 1H), 1.05 (d, J=6.6 Hz, 3H), 1.02 (d, J=6.6 Hz, 3H), 1.01 (t, J=7.0 Hz, 3H).

MS: 409, 411 (M+1).

### Step C: 1-[(S)-1-(tert-Butyl-dimethyl-silanyl oxymethyl)-2-methyl-propyl]-6,7-dichloro-4-oxo-1,4-dihydro-[1,5]naphthyridine-3-carboxylic acid ethyl ester

A mixture of 3-((S)-1-hydroxymethyl-2-methyl-propylamino)-2-(3,5,6-trichloro-pyridine-2-carbonyl)-acrylic acid ethyl ester (2 g, 4.88 mmol) and potassium carbonate (1.35 g, 9.76 mmol) in anhydrous DMF (15 mL) was stirred at 130° C. for 90 min. The mixture was filtered and washed with DMF. The filtrate was evaporated to dryness under reduced pressure and dried at 40° C. in vacuo. The dried residue was dissolved in 15 mL, of dry DMF and imidazole (3.32 g, 48.8 mmol) and tert-butyldimethylsilyl chloride (3.68 g, 24.4 mmol) were added under argon at room temperature. The resulting solution was stirred overnight at room temperature and was evaporated to dryness under reduced pressure. The residue was purified by silca gel chromatography (ISCO, hexane/EtOAc, 0-30%, 20 min, 30-100%, 10 min, 100%, 10 min) to give the pure compound as an yellow foam (0.35 g, 15%).

¹H NMR (CDCl₃, 400 MHz): δ 8.71 (s, 1H), 8.32 (s, 1H), 4.45 (q, J=7.1 Hz, 2H), 4.27 (m, 1H), 4.07 (m, 2H), 2.50 (m, 1H), 1.45 (t, J=7.1 Hz, 3H), 1.27 (d, J=6.6 Hz, 3H), 0.93 (d, J=6.6 Hz, 3H), 0.86 (s, 9H), 0.04 (s, 6H).

MS: 487 (M+1).

### Step D: 1-[(S)-1-(tert-Butyl-dimethyl-silanyloxymethyl)-2-methyl-propyl]-7-chloro-6-(3-chloro-2-fluoro-benzyl)-4-oxo-1,4-dihydro-[1,5]naphthyridine-3-carboxylic acid ethyl ester

Under an argon stream, zinc powder (346 mg, 5.3 mmol) was suspended of dry tetrahydrofuran (1 mL). 1,2-Dibromoethane (1.4 μl, 0.016 mmol) and trimethylsilyl chloride (4.0 μl, 0.032 mmol) were added at 60° C. and the mixture was stirred with heating for 30 min. A solution of 2-fluoro-3-chloro-benzyl bromide (179 mg, 0.79 mmol) in 2 mL of dry tetrahydrofuran was added dropwise at 60° C. The mixture was stirred with heating for 1 hour and allowed to cool to room temperature to give a solution of 2-fluoro-3-chloro-benzylzinc bromide in tetrahydrofuran. This was used in the next step.

1-[(S)-1-(tert-Butyl-dimethyl-silanyloxymethyl)-2-methyl-propyl]-6,7-d chloro-4-oxo-1,4-dihydro-[1,5]naphthyridine-3-carboxylic acid ethyl ester (300 mg, 0.62 mmol) was dissolved in 10 mL of dry tetrahydrofuran under an argon stream. Dichlorobis(triphenylphosphine)palladium(II) (34 mg, 0.048 mmol) was added followed by the addition of the above-mentioned 2-fluoro-3-chloro-benzylzinc bromide in tetrahydrofuran at 60° C. The mixture was stirred for an additional hour at this temperature and the reaction mixture was allowed to cool to room temperature. 1 N hydrochloric acid was added and the mixture was extracted three times with ethyl acetate. The organic layer were combined and washed successively with water, brine, dried over anhydrous sodium sulfate and concentrated under reduced pressure. The crude residue was purified by silica gel chromatography (ISCO, 12 g of column, hexane/EtOAc, 0-30%, 25 min; 30-80%, 10 min; 80%, 5 min) to give a major product as an yellow foam 270 mg (73%).

¹H NMR (CDCl₃, 600 MHz): δ 8.64 (s, 1H), 7.99 (s, 1H), 7.26 (t, J=7.8 Hz, 1H), 7.11 (t, J=7.8 Hz, 1H), 6.94 (t, J=7.8 Hz, 1H), 4.57 (s, 2H), 4.42 (q, J=7.1 Hz, 2H), 4.11 (m, 1H), 3.99 (m, 2H), 2.43 (m, 1H), 1.43 (t, J=7.1 Hz, 3H), 1.22 (d, J=6.2 Hz, 3H), 0.89 (d, J=6.2 Hz, 3H), 0.75 (s, 9H), 0.03 (s, 6H).

MS: 595 (M+1).

### Step E: 6-(3-Chloro-2-fluoro-benzyl)-1-((S)-1-hydroxymethyl-2-methyl-propyl)-7-methoxy-4-oxo-1,4-dihydro-[1,5]naphthyridine-3-carboxylic acid

1-[(S)-1-(tert-Butyl-dimethyl-silanyloxymethyl)-2-methyl-propyl]-7-(3-chloro-2-fluoro-benzyl)-6-methoxy-4-oxo-1,4-dihydro-[1,5]naphthyridine-3-carboxylic acid ethyl ester (100 mg, 0.17 mmol) was dissolved in 20 mL of methanol and 2 mL of 25% sodium methoxide in methanol was added. The mixture was refluxed for 4 hours, allowed to cool to room temperature and evaporated to a small volume under reduced pressure. Water (10 mL) was added and filtered. The filtrate was neutralized with 1 N hydrochloric acid. The solid was filtered and washed with water to give the pure product as an white solid (65 mg, 85%).

¹H NMR (DMSO-d₆, 400 MHz): δ 15.60 (brs, 1H, OH, exchangeable with D₂O), 8.91 (s, 1H), 7.88 (s, 1H), 7.63 (dt, J=1.5 and 7.8 Hz, 1H), 7.21 (dt, J=1.5 and 7.8 Hz, 1H), 7.15 (t, J=7.8 Hz, 1H), 5.20 (brs, 1H, OH, exchangeable with D₂O), 4.88 (m, 1H), 4.32 (s, 2H), 4.06 (s, 3H), 3.97 (m, 1H), 3.80 (m, 1H), 2.39 (m, 1H), 1.16 (d, 3H), 0.73 (d, J=6.6 Hz, 3H).

MS: 449 (M+1).

| Compound | Structure | Analytical data |
|---|---|---|
| 8A | 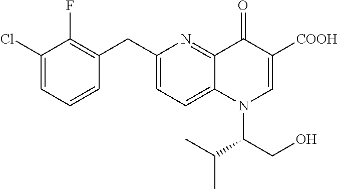 | ¹H NMR (DMSO-d₆, 400 MHz): δ 15.38 (brs, 1H, OH, exchangeable with D₂O), 8.97 (s, 1H), 8.76 (d, J = 8.6 Hz, 1H), 7.84 (d, J = 8.6 Hz, 1H), 7.50 (t, J = 7.8 Hz, 1H), 7.38 (t, J = 7.8 Hz, 1H), 7.22 (t, J = 7.8 Hz, 1H), 5.18 (brs, 1H, OH, exchangeable with D₂O), 4.83 (m, 1H), 4.42 (s, 2H), 3.98 (m, 1H), 3.79 (m, 1H), 2.38 (m, 1H), 1.12 (d, J = 6.2 Hz, 3H), 0.71 (d, J = 6.2 Hz, 3H). MS: 419 (M + 1). |
| 8B | 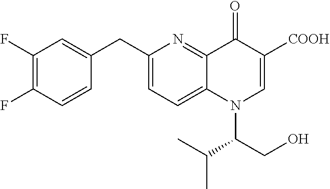 | ¹H NMR (DMSO-d₆, 400 MHz): δ 15.41 (brs, 1H, OH, exchangeable with D₂O), 8.75 (s, 1H), 8.76 (d, J = 8.6 Hz, 1H), 7.88 (d, J = 8.6 Hz, 1H), 7.46 (ddd, J = 2.2, 8.0 and 11.8 Hz, 1H), 7.38 (dt, J = 8.6 and 11.0 Hz, 1H), 7.19 (m, 1H), 5.16 (brs, 1H, OH, exchangeable with D₂O), 4.83 (m, 1H), 4.33 (s, 2H), 3.96 (m, 1H), 3.79 (m, 1H), 2.37 (m, 1H), 1.12 (d, J = 6.2 Hz, 3H), 0.70 (d, J = 6.2 Hz, 3H). MS: 403 (M + 1). |
| 8C | 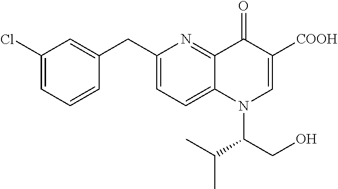 | ¹H NMR (DMSO-d₆, 400 MHz): δ 15.43 (brs, 1H, OH, exchangeable with D₂O), 8.97 (s, 1H), 8.76 (d, J = 8.6 Hz, 1H), 7.88 (d, J = 8.6 Hz, 1H), 7.46 (d, J = 2.0 Hz, 1H), 7.32 (m, 3H), 5.16 (brs, 1H, OH, exchangeable with D₂O), 4.83 (m, 1H), 4.34 (s, 2H), 3.95 (m, 1H), 3.79 (m, 1H), 2.37 (m, 1H), 1.12 (d, J = 6.2 Hz, 3H), 0.70 (d, J = 6.2 Hz, 3H). MS: 401 (M + 1). |
| 8D | 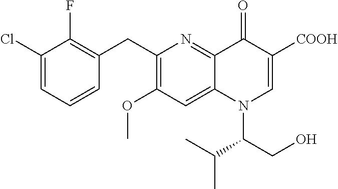 | ¹H NMR (DMSO-d₆, 400 MHz): δ 15.60 (brs, 1H, OH, exchangeable with D₂O), 8.91 (s, 1H), 7.88 (s, 1H), 7.63 (dt, J = 1.5 and 7.8 Hz, 1H), 7.21 (dt, J = 1.5 and 7.8 Hz, 1H), 7.15 (t, J = 7.8 Hz, 1H), 5.20 (brs, 1H, OH, exchangeable with D₂O), 4.88 (m, 1H), 4.32 (s, 2H), 4.06 (s, 3H), 3.97 (m, 1H), 3.80 (m, 1H), 2.39 (m, 1H), 1.16 (d, J = 6.6 Hz, 3H), 0.73 (d, J = 6.6 Hz, 3H). MS: 449 (M + 1). |

### Example 9

### Compounds of Formula (IX)

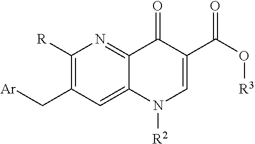

Compounds of formula (IX) were prepared according to the following general synthetic schemes. When appropriate, protecting groups are used as needed according to established synthetic procedures known to those of skill in the art, and may or may not be removed upon completion of the synthesis. Starting materials are synthesized according to methods known in the art or are commercially available.

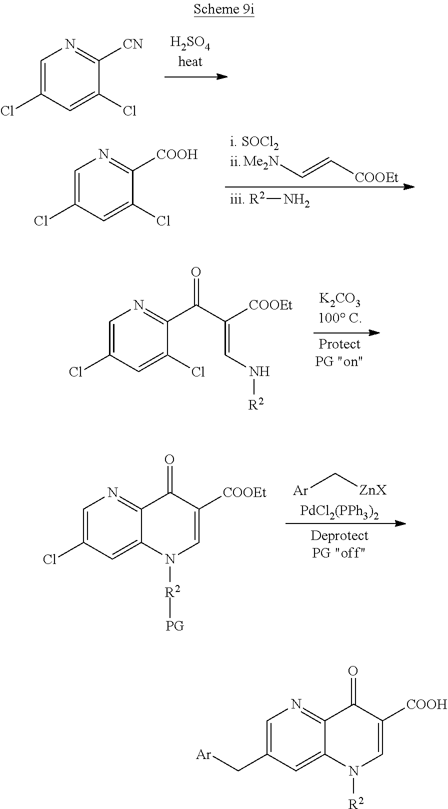

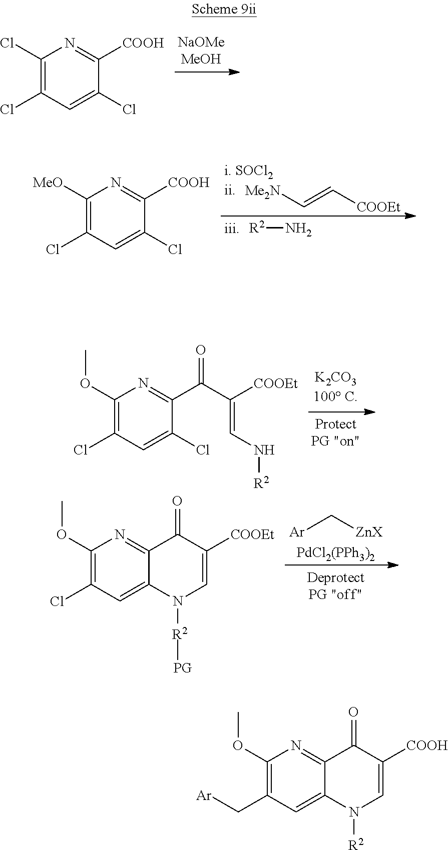

### Example 9A

### 7-(3,4-Difluoro-benzyl)-1-((S)-1-hydroxymethyl-2-methyl-propyl)-4-oxo-1,4-dihydro-[1,5]naphthyridine-3-carboxylic acid

1-[(S)-1-(tert-Butyl-dimethyl-silanyloxymethyl)-2-methyl-propyl]-7-chloro-4-oxo-1,4-dihydro-[1,5]naphthyridine-3-carboxylic acid ethyl ester (0.34 g, 0.75 mmol) was dissolved in 10 mL of dry tetrahydrofuran under an argon stream. Dichlorobis(triphenylphosphine)palladium(II) (77 mg, 0.11 mmol) was added followed by the addition of the solution of 3,4-difluorobenzylzinc bromide in tetrahydrofuran (0.5M in THF, 3.0 mL, 1.5 mmol) at 60° C. After the completion of the dropwise addition, the mixture was stirred with heating at the same temperature for 1.5 hour. The reaction mixture was allowed to cool to room temperature, 1 N hydrochloric acid was added and the mixture was extracted three times with ethyl acetate. The organic layer were combined, washed successively with water, brine, dried over anhydrous sodium sulfate and concentrated under reduced pressure. The crude residue was purified by silica gel chromatography (ISCO, 40 g of column, hexane/ethyl acetate, 0-40%, 25 min; 40-80%, 5 min; 80%, 10 min) to give the intermediate 1-[(S)-1-(tert-butyl-dimethyl-silanyloxymethyl)-2-methyl-propyl]-7-(3,4-difluoro-benzyl)-4-oxo-1,4-dihydro-[1,5]naphthyridine-3-carboxylic acid ethyl ester as a yellow solid.

This intermediate was dissolved in 10 mL of methanol and 25% of sodium methoxide in methanol and water were added. The mixture was refluxed overnight. The reaction mixture was allowed to cool to room temperature and filtered. The yellow filtrate was evaporated to a small volume under reduced pressure and neutralized with 1 N hydrochloric acid. The solid was filtered and washed with water. The crude product was washed with hot ethyl acetate to obtain the desired product as a yellowish solid.

¹H NMR (DMSO-d₆, 400 MHz): δ 15.37 (brs, 1H, OH, exchangeable with D₂O), 8.98 (s, 1H), 8.87 (d, J=2.6 Hz, 1H), 8.76 (d, J=2.6 Hz, 1H), 7.49 (ddd, J=2.1, 7.8, 11.8 Hz, 1H), 7.40 (dt, J=8.6 and 10.8 Hz, 1H), 7.22 (m, 1H), 5.22 (brs, 1H, OH, exchangeable with D₂O), 4.88 (m, 1H), 4.25 (s, 2H), 4.01 (m, 1H), 3.84 (m, 1H), 2.37 (m, 1H), 1.14 (d, J=6.4 Hz, 3H), 0.71 (d, J=6.4 Hz, 3H).

MS: 403 (M+1), 401 (M−1).

### Example 9B

### 7-(3-Chloro-2-fluoro-benzyl)-1-((S)-1-hydroxymethyl-2-methyl-propyl)-4-oxo-1,4-dihydro-[1,5]naphthyridine-3-carboxylic acid

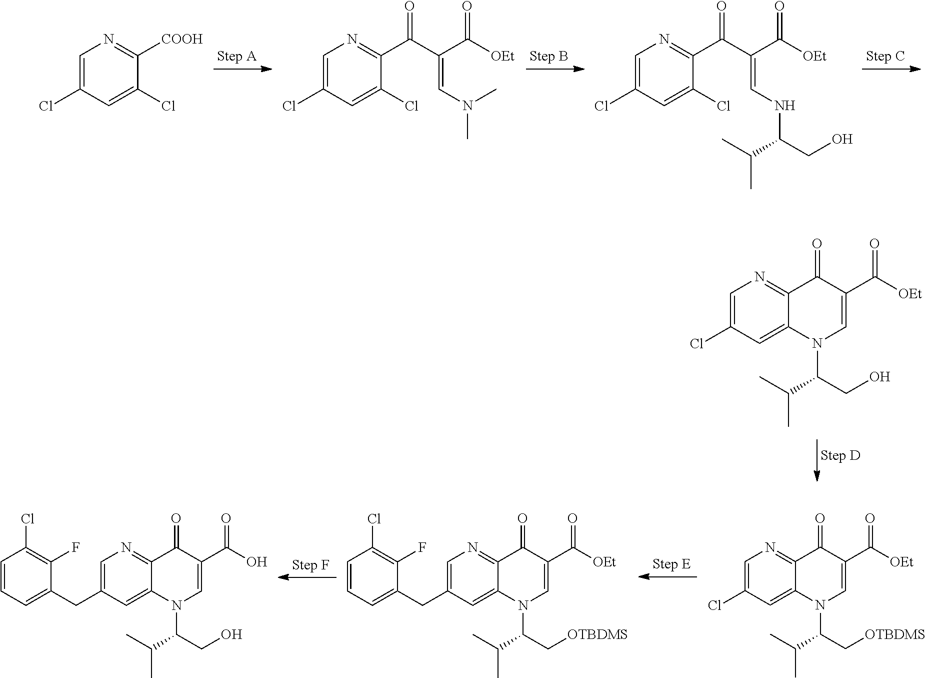

### Step A: 2-(3,5-Dichloro-pyridine-2-carbonyl)-3-dimethylamino-acrylic acid ethyl ester

3,5-Dichloro-pyridine-2-carbonitrile (10 g, 57.8 mmol) was dissolved in 100 mL of 95% concentrated sulfuric acid and this mixture was heated to 115° C. overnight. The reaction mixture was then cooled, poured over ice with strong stirring. The resulting solid was filtered, washed with water and dried at 40° C. under reduced pressure to give 9.4 g (85%) of pure product as a white solid.

A mixture of 3,5-dichloro-pyridine-2-carboxylic acid (5.76 g, 30 mmol) and thionyl chloride (4.4 mL, 60 mmol) in 50 mL of anhydrous toluene and 0.5 mL of anhydrous DMF was refluxed for 2 h. The solvent was removed under reduced pressure to give a mobile oil residue which was azeoptoped with toluene (20 mL). The residue was dissolved in 20 mL of anhydrous THF and this solution was added dropwise to a solution of ethyl 3-(dimethylamino)acrylate (4.7 g, 33 mmol) and triethylamine (3.64 g, 36 mmol) in 20 mL of anhydrous THF under nitrogen, and the mixture was heated under reflux for 7 hours. The reaction mixture was allowed to cool to room temperature and concentrated under reduced pressure. Water (100 mL) and ethyl acetate (100 mL) was added to allow partitioning. The organic layer was washed successively with saturated aqueous sodium bicarbonate (×2), water, brine, dried over sodium sulfate and was concentrated under reduced pressure. The crude product was purified by ISCO (hexane/EtOAc, 0-40%, 30 min; 100%, 20 min) to give 5.9 g (62%) of pure product as yellow oil.

### Step B: 2-(3,5-Dichloro-pyridine-2-carbonyl)-3-((S)-1-hydroxymethyl-2-methyl-propylamino)-acrylic acid ethyl ester

A solution of 2-(3,5-dichloro-pyridine-2-carbonyl)-3-dimethylamino-acrylic acid ethyl ester (3.17 g, 10 mmol) and L-valinol (1.03 g, 10 mmol) in anhydrous THF (40 mL) was stirred for 30 min at room temperature. The mixture was evaporated to dryness to give a crude product in a quantitative yield, which was used for next step without further purification.

¹H NMR (DMSO-d₆, 400 MHz): δ 10.91 (dd, J=9.6 and 13.8 Hz, 1H, NH, exchangeable with D₂O), 8.52 (d, J=2.0 Hz, 1H), 8.25 (d, J=14.2 Hz, 1H, it becomes singlet after D₂O exchange), 8.25 (d, J=2.0 Hz, 1H), 5.08 (t, J=5.1 Hz, 1H, OH, exchangeable with D₂O), 3.85 (q, J=7.0 Hz, 2H), 3.60 (m, 2H), 3.39 (m, 1H), 1.97 (m, 1H), 0.94 (d, J=6.6 Hz, 3H), 0.90 (d, J=6.6 Hz, 3H), 0.89 (t, J=7.0 Hz, 3H).

MS: 375 (M+1), 373 (M−1).

### Step C: 7-Chloro-1-(1-hydroxymethyl-2-methyl-propyl)-4-oxo-1,4-dihydro-[1,5]naphthyridine-3-carboxylic acid ethyl ester

A mixture of 2-(3,5-dichloro-pyridine-2-carbonyl)-3-((S)-1-hydroxymethyl-2-methyl-propylamino)-acrylic acid ethyl ester (1.78 g, 4.7 mmol) and potassium carbonate (1.31 g, 9.5 mmol) in anhydrous DMF (20 mL) was stirred at 100° C. overnight. The mixture was evaporated to dryness under reduced pressure and the residue was purified by ISCO (Chloroform/methanol, 0-40%, 40 min) to give the desired compound as a yellow solid.

¹H NMR (DMSO-d₆, 400 MHz): δ 8.81 (d, J=2.6 Hz, 1H), 8.74 (d, J=2.6 Hz, 1H), 8.66 (s, 1H), 5.12 (t, J=5.1 Hz, 1H, OH, exchangeable with D₂O), 4.65 (m, 1H), 4.25 (q, J=7.0 Hz, 2H), 3.85 (m, 1H), 3.80 (m, 1H), 2.29 (m, 1H), 1.29 (t, J=7.0 Hz, 3H), 1.12 (d, J=6.6 Hz, 3H), 0.73 (d, J=6.6 Hz, 3H).

MS: 337 (M−1).

### Step D: 1-[(S)-1-(tert-Butyl-dimethyl-silanyloxymethyl)-2-methyl-propyl]-7-chloro-4-oxo-1,4-dihydro[1,5]naphthyridine-3-carboxylic acid ethyl ester

To a mixture of 7-chloro-1-(1-hydroxymethyl-2-methyl-propyl)-4-oxo-1,4-dihydro[1,5]naphthyridine-3-carboxylic acid ethyl ester (1.0 g, 2.95 mmol) and imidazole (2.01 g, 29.5 mmol) in 10 mL of anhydrous DMF was added tert-butyldimethylsilyl chloride (2.22 g, 14.8 mmol) under argon at room temperature. The reaction mixture was stirred overnight at room temperature and evaporated to dryness under reduced pressure. The residue was purified by ISCO (Chloroform/methanol, 0-30%, 40 min) to give the pure compound as an yellow oil.

¹H NMR (DMSO-d₆, 400 MHz): δ 8.94 (d, J=1.9 Hz, 1H), 8.85 (d, J=1.9 Hz, 1H), 8.73 (s, 1H), 4.86 (m, 1H), 4.34 (q, J=7.1 Hz, 2H), 4.09 (m, 1H), 4.02 (m, 1H), 2.43 (m, 1H), 1.41 (t, J=7.1 Hz, 3H), 1.24 (d, J=6.6 Hz, 3H), 0.87 (d, J=6.6 Hz, 3H), 0.77 (s, 9H), 0.03 (s, 6H).

MS: 453 (M+1).

### Step E: 1-[(S)-1-(tert-Butyl-dimethyl-silanyloxymethyl)-2-methyl-propyl]-7-(3-chloro-2-fluoro-benzyl)-4-oxo-1,4-dihydro-[1,5]naphthyridine-3-carboxylic acid ethyl ester

Under an argon stream, zinc powder (480 mg, 7.34 mmol) was suspended in 1 mL of dry tetrahydrofuran. 1,2-Dibromoethane (1.4 μl, 0.016 mmol) and trimethylsilyl chloride (4.0 μl, 0.032 mmol) were added at 60° C. and the mixture was stirred with heating for 30 min. A solution of 2-fluoro-3-chloro-benzyl bromide (352 mg, 1.58 mmol) in 2 mL of dry tetrahydrofuran was added dropwise. The resulting mixture was stirred for 1 hour at 60° C. and was allowed to cool to room temperature to give a solution of 1 M 2-fluoro-3-chloro-benzylzinc bromide in tetrahydrofuran. This was used in the next step.

1-[(S)-1-(tert-Butyl-dimethyl-silanyloxymethyl)-2-methyl-propyl]-7-chloro-4-oxo-1,4-dihydro-[1,5]naphthyridine-3-carboxylic acid ethyl ester (553 mg, 1.22 mmol) was dissolved in 20 mL of dry tetrahydrofuran under an argon stream. Dichlorobis(triphenylphosphine)palladium(II) (34 mg, 0.048 mmol) was added and a solution of 2-fluoro-3-chloro-benzylzinc bromide in tetrahydrofuran was added dropwise at 60° C. After completion of the dropwise addition, the mixture was stirred with heating at the same temperature for 1.5 hour. The reaction mixture was allowed to cool to room temperature, 1 N hydrochloric acid was added and the mixture was extracted three times with ethyl acetate. The organic layer were combined and washed successively with water, brine, dried over anhydrous sodium sulfate and concentrated under reduced pressure. The crude residue was purified by silica gel chromatography (ISCO, 12 g of column, chloroform/methanol, 0-30%, 25 min; 30-80%, 10 min; 80%, 5 min) to give a major product as an yellow foam 500 mg (73%).

¹H NMR (DMSO-d₆, 400 MHz): δ 8.72 (s, 1H), 8.72 (s, 1H), 8.61 (s, 1H), 7.59 (t, J=7.8 Hz, 1H), 7.44 (t, J=7.8 Hz, 1H), 7.30 (t, J=7.8 Hz, 1H), 4.80 (m, 1H), 4.36 (s, 2H), 4.33 (q, J=7.1 Hz, 2H), 4.14 (dd, J=6.8 and 12.2 Hz, 1H), 4.05 (dd, J=3.4 and 12.2 Hz, 1H), 2.44 (m, 1H), 1.38 (t, J=7.1 Hz, 3H), 1.23 (d, J=6.2 Hz, 3H), 0.84 (d, J=6.2 Hz, 3H), 0.73 (s, 9H), 0.03 (s, 6H).

MS: 562 (M+1).

### Step F: 7-(3-Chloro-2-fluoro-benzyl)-1-((S)-1-hydroxymethyl-2-methyl-propyl)-4-oxo-1,4-dihydro-[1,5]naphthyridine-3-carboxylic acid

The above intermediate (500 mg) was dissolved in 20 mL of methanol and 2 mL of 25% sodium methoxide in methanol and 4 mL of water were added. The mixture was refluxed for 4 hours. The reaction mixture was allowed to cool to room temperature and evaporated to a small volume under reduced pressure. Water (10 mL) was added and the mixture was filtered. The filtrate was neutralized with 1 N hydrochloric acid. The solid was filtered and washed with water to give a pure product as an yellowish solid (365 mg, 71%).

¹H NMR (DMSO-d₆, 400 MHz): δ 15.36 (brs, 1H, OH, exchangeable with D₂O), 9.03 (s, 1H), 8.85 (s, 1H), 8.78 (s, 1H), 7.55 (dt, J=1.4 and 7.8 Hz, 1H), 7.39 (t, J=7.8 Hz, 1H), 7.25 (t, J=7.8 Hz, 1H), 5.24 (brs, 1H, OH, exchangeable with D2O), 4.89 (m, 1H), 4.39 (s, 2H), 4.03 (dd, J=6.8 and 12.2 Hz, 1H), 3.86 (dd, J=3.4 and 12.2 Hz, 1H), 2.54 (m, 1H), 1.17 (d, J=6.2 Hz, 3H), 0.74 (d, J=6.2 Hz, 3H).

MS: 419 (M+1).

### Example 9C

### 7-(3-Chloro-2-fluoro-benzyl)-1-((S)-1-hydroxymethyl-2-methyl-propyl)-6-methoxy-4-oxo-1,4-dihydro-[1,5]naphthyridine-3-carboxylic acid

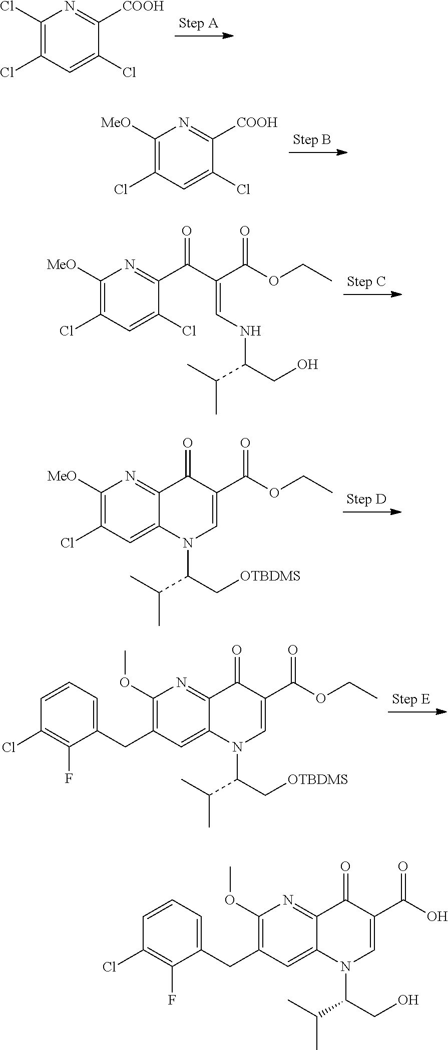

### Step A: 3,5-Dichloro-6-methoxypyridine-2-carboxylic acid

A mixture of 3,5,6-trichloropyridine-2-carboxylic acid (6 g, 26.5 mmol), 25% MeONa in MeOH (18 mL) and MeOH (100 mL) was heated to reflux overnight and cooled to room temperature. After evaporation of the solvent, the residue was diluted with water and acidified with 5N aqueous HCl. The resulting solid was collected by filtration, washed with water, and dried to give 5.1 g (86.7%) of 3,5-dichloro-6-methoxypyridine-2-carboxylic acid as a white solid.

¹H NMR (DMSO-d₆, 400 MHz): δ 13.93 (brs, 1H, OH, exchangeable with D₂O), 8.27 (s, 1H), 3.96 (s, 3H).

### Step B: 2-(3,5-Dichloro-6-methoxy-pyridine-2-carbonyl)-3-((S)-1-hydroxymethyl-2-methyl-propylamino)-acrylic acid ethyl ester

A mixture of 3,6-dichloro-pyridine-2-carboxylic acid (2.22 g, 10 mmol) and thionyl chloride (1.47 mL, 20 mmol) in 30 mL of anhydrous toluene and 0.25 mL of anhydrous DMF was refluxed for 2 h. The solvent was removed under reduced pressure to give a mobile oil residue which was azeoptoped with toluene (20 mL). The residue was dissolved in 20 mL of anhydrous THF and this solution was added dropwise to a solution of ethyl 3-(dimethylamino)acrylate (1.57 g, 11 mmol) and triethylamine (1.2 g, 12 mmol) in 20 mL of anhydrous THF under nitrogen. The resulting mixture was heated under reflux for 7 hours and allowed to cool to room temperature. L-valinol (1.24 g, 12 mmol) was added and after stirred 30 min at room temperature the mixture was evaporated to dryness. Water and ethyl acetate were added to allow partitioning. The organic layer was washed successively with saturated aqueous sodium bicarbonate (×2), water, brine, dried over sodium sulfate and was concentrated under reduced pressure. The crude product, which was purified by silica gel chromatography (ISCO, Hexane/EtOAc, 0-40%, 40 Min) to give the pure compound as a yellow oil (3.18 g, 78.5%)

¹H NMR (DMSO-d₆, 400 MHz): δ 10.91 (dd, J=9.6 and 13.8 Hz, 1H, NH, exchangeable with D₂O), 8.24 (d, J=14.3 Hz, 1H, it becomes singlet after D₂O exchange), 8.15 (s, 1H), 5.08 (brs, 1H, OH, exchangeable with D₂O), 3.88 (q, J=7.0 Hz, 2H), 3.87 (s, 2H), 3.59 (m, 2H), 3.37 (m, 1H), 1.95 (m, 1H), 0.94 (d, J=6.6 Hz, 3H), 0.91 (d, J=6.6 Hz, 3H), 0.90 (t, J=7.0 Hz, 3H).

MS: 405 (M+1).

### Step C: 1-[(S)-1-(tert-Butyl-dimethyl-silanyloxymethyl)-2-methyl-propyl]-7-chloro-6-methoxy-4-oxo-1,4-dihydro-[1,5]naphthyridine-3-carboxylic acid ethyl ester

A mixture of 2-(3,5-dichloro-6-methoxy-pyridine-2-carbonyl)-3-((S)-1-hydroxymethyl-2-methyl-propylamino)-acrylic acid ethyl ester (2 g, 4.88 mmol) and potassium carbonate (1.35 g, 9.76 mmol) in anhydrous DMF (15 mL) was stirred at 130° C. for 90 min. The mixture was filtered and washed with DMF. The filtrate was evaporated to dryness under reduced pressure and dried at 40° C. in vacuo. The dried residue was dissolved in 15 mL of dry DMF and imidazole (3.32 g, 48.8 mmol) and tert-butyldimethylsilyl chloride (3.68 g, 24.4 mmol) were added under argon at room temperature. The resulting mixture was stirred overnight at room temperature and evaporated to dryness under reduced pressure. The crude material was purified by ISCO (hexane/EtOAc, 0-30%, 20 min, 30-100%, 10 min, 100%, 10 min) to give the pure compound as an yellow foam.

¹H NMR (CDCl₃, 400 MHz): δ 8.67 (s, 1H), 8.10 (s, 1H), 4.48 (q, J=7.1 Hz, 2H), 4.30 (s, 3H), 4.18 (m, 1H), 4.07 (m, 2H), 2.51 (m, 1H), 1.49 (t, J=7.1 Hz, 3H), 1.28 (d, J=6.6 Hz, 3H), 0.92 (d, J=6.6 Hz, 3H), 0.85 (s, 9H), 0.06 (s, 6H).

MS: 483 (M+1).

### Step D: 1-[(S)-1-(tert-Butyl-dimethyl-silanyloxymethyl)-2-methyl-propyl]-7-(3-chloro-2-fluoro-benzyl)-6-methoxy-4-oxo-1,4-dihydro-[1,5]naphthyridine-3-carboxylic acid ethyl ester

Under an argon stream, zinc powder (346 mg, 5.3 mmol) was suspended in 1 mL of dry tetrahydrofuran. 1,2-Dibromoethane (1.4 μl, 0.016 mmol) and trimethylsilyl chloride (4.0 μl, 0.032 mmol) were added at 60° C., and the mixture was stirred with heating for 30 min. A solution of 2-fluoro-3-chloro-benzyl bromide (177 mg, 0.79 mmol) in 2 mL of dry tetrahydrofuran was added dropwise at 60° C. The mixture was stirred with heating for 1 hour and allowed to cool to room temperature to give a solution of 2-fluoro-3-chloro-benzylzinc bromide in tetrahydrofuran. This was used in the next step.

1-[(S)-1-(tert-Butyl-dimethyl-silanyloxymethyl)-2-methyl-propyl]-7-chloro-6-methoxy-4-oxo-1,4-dihydro-[1,5]naphthyridine-3-carboxylic acid ethyl ester (256 mg, 0.53 mmol) was dissolved in 10 mL of dry tetrahydrofuran under an argon stream. Dichlorobis(triphenylphosphine)palladium(II) (34 mg, 0.048 mmol) was added followed by the addition at 60° C. of the solution of the above-mentioned 2-fluoro-3-chloro-benzylzinc bromide in tetrahydrofuran. After the completion of the addition, the mixture was stirred at the same temperature for an additional hour. The reaction mixture was allowed to cool to room temperature, 1 N hydrochloric acid was added and the mixture was extracted three times with ethyl acetate. The organic layer was washed successively with water, brine, dried over anhydrous sodium sulfate and was concentrated under reduced pressure. The residue was purified by silica gel chromatography (ISCO, 12 g of column, hexane/EtOAc; 0-30%, 25 min; 30-100%, 10 min; 100%, 10 min) to give a major product as an yellow foam 220 mg (70%).

¹H NMR (CDCl₃, 400 MHz): δ 8.97 (s, 1H), 8.09 (s, 1H), 7.36 (d, J=7.8 Hz, 1H), 7.18 (t, J=7.8 Hz, 1H), 7.10 (t, J=7.8 Hz, 1H), 4.74 (s, 2H), 4.42 (q, J=7.1 Hz, 2H), 4.15 (s, 3H), 4.13 (m, 1H), 3.93 (d, J=11.4 Hz, 1H), 2.44 (m, 1H), 1.40 (t, J=7.1 Hz, 3H), 1.19 (d, J=6.2 Hz, 3H), 0.80 (s, 9H), 0.79 (d, J=6.2 Hz, 3H), 0.01 (s, 6H).

MS: 591 (M+1).

### Step E: 7-(3-Chloro-2-fluoro-benzyl)-1-((S)-1-hydroxymethyl-2-methyl-propyl)-6-methoxy-4-oxo-1,4-dihydro-[1,5]naphthyridine-3-carboxylic acid

1-[(S)-1-(tert-Butyl-dimethyl-silanyloxymethyl)-2-methyl-propyl]-7-(3-chloro-2-fluoro-benzyl)-6-methoxy-4-oxo-1,4-dihydro-[1,5]naphthyridine-3-carboxylic acid ethyl ester (100 mg, 0.17 mmol) was dissolved in 20 mL of methanol and 2 mL of 25% sodium methoxide in methanol and 4 mL of water were added. The mixture was refluxed for 4 hours. The reaction mixture was allowed to cool to room temperature and evaporated to a small volume under reduced pressure. Water (10 mL) was added and the mixture was filtered. The filtrate was neutralized with 1 N hydrochloric acid. The solid was filtered and washed with water to give a pure product as an yellowish solid (52 mg, 68%).

¹H NMR (DMSO-d₆, 400 MHz): δ 15.74 (brs, 1H, OH, exchangeable with D₂O), 8.93 (s, 1H), 8.71 (s, 1H), 7.63 (t, J=7.8 Hz, 1H), 7.56 (t, J=7.8 Hz, 1H), 7.17 (t, J=7.8 Hz, 1H), 5.22 (brs, 1H, OH, exchangeable with D₂O), 4.80 (m, 1H), 4.19 (s, 2H), 4.01 (m, 1H), 3.99 (s, 3H), 3.84 (m, 1H), 2.35 (m, 1H), 1.11 (d, J=6.2 Hz, 3H), 0.71 (d, J=6.2 Hz, 3H).

MS: 449 (M+1).

| Compound | Structure |
|---|---|
| 9A | 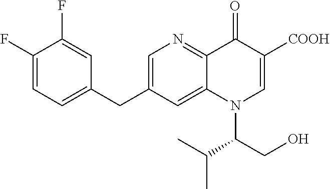 |
| 9B | 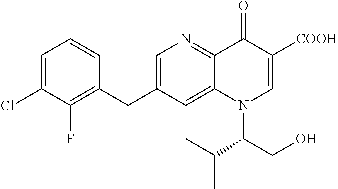 |
| 9C | 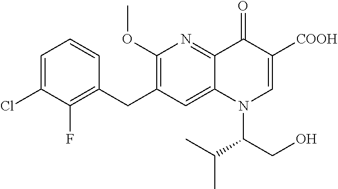 |

### Example 10

### Compounds of Formula (X)

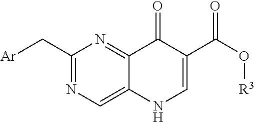

### Example 10A

### 2-(3-chlorobenzyl)-8-oxo-5,8-dihydropyrido[3,2-d]pyrimidine-7-carboxylic acid

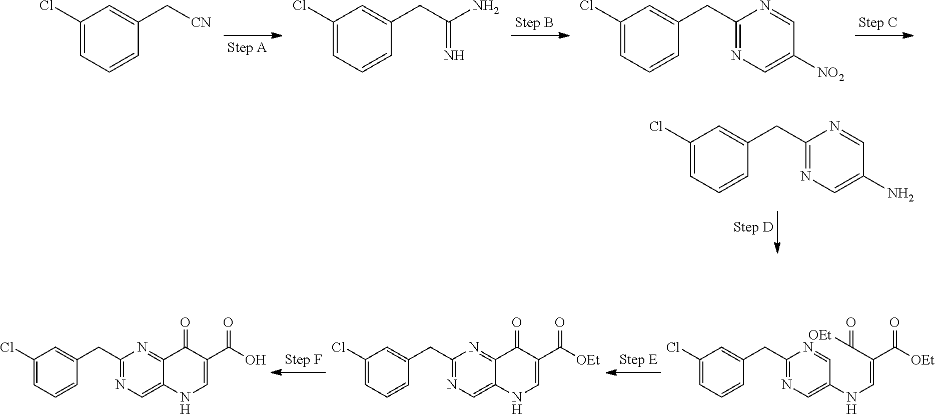

### Step A: 2-(3-Chloro-phenyl)-acetamidine hydrochloride

A 2M solution of Me₃Al in toluene (51 mL, 102 mmol, Aldrich) was slowly added to a magnetically stirred suspension of ammonium chloride (5.78 g, 108 mmol) in 50 mL of anhydrous toluene at 5° C. under argon. After the addition, the mixture was warmed to room temperature and stirred for 2 hours until gas evolution (CH₄) has ceased. Then, 3-chlorophenylacetonitrile (9.06 g, 60 mmol) was added and the solution was heated to 80° C. for 16 hours under argon. The reaction mixture was slowly poured into a slurry of 30 g of silica gel in 100 mL of chloroform and stirred for 5 min. The silica was filtered and washed with methanol. The filtrate and wash were combined and the solvent was stripped to a residue of small volume, which was re-filtered to remove ammonium chloride. Then, 20 mL of methanolic HCl (108 mmol) was added to the filtrate and evaporated to dryness under reduced pressure. The residue was purified by column and eluted with chloroform to remove impurities and then chloroform/methanol (4:1) to obtain the crude product which was dissolved in isopropanol/acetone (4:1) and filtered to remove insoluble ammonium chloride. To the filtrate was added ether with stirring and the solid was filtered and washed with ether to obtain the pure compound as a white solid (9.9 g, 74%).

¹H NMR (DMSO-d₆, 400 MHz): δ 8.85 (brs, 2H, NH₂, exchangeable with D₂O), 7.60 (s, 1H), 7.45 (m, 1H), 7.40 (m, 2H), 7.33 (brs, 2H, NH₂, exchangeable with D₂O), 3.76 (s, 2H).

MS: 169 (M+1).

### Step B: 2-(3-Chloro-benzyl)-6-nitro-pyrimidine

2-(3-Chloro-phenyl)-acetamidine hydrochloride (1.66 g, 8.1 mmol) and sodium nitromalonaldehyde (Na[C(NO₂)(CHO)₂]) (1.53 g, 9.71 mmol) was mixed in 10 mL of water at room temperature to form a salt. This salt was heated overnight at 70° C. in aq. Triton B. The solution became dark. This mixture was cooled in a ice-water bath for 30 min and then filtered and washed with cold water and alcohol to crystals (1.08 g, 54%).

¹H NMR (CDCl₃, 400 MHz): 9.00 (d, J=2.0 Hz, 1H), 8.18 (d, J=2.0 Hz, 1H), 7.47 (m, 3H), 7.35 (m, 1H), 5.47 (s, 2H). MS: 250 (M+1).

### Step C: 2-(3-Chloro-benzyl)-5-amino-pyrimidine

2-(3-Chloro-benzyl)-5-nitro-pyrimidine (3.7 g, 14.9 mmol) was dissolved in concentrated hydrochloric acid (30 mL) at 5° C., tin chloride (10 g) was added and stirred at 5° C. for 15 min and then heated at 80° C. for 1 hour. The reaction mixture was cooled and neutralized with 20% sodium hydroxide until pH 8 and extracted with ethyl acetate, washed with water, dried with sodium sulfate and evaporated to obtain a brown solid which was purified by silica gel column to give pure product.

¹H NMR (CDCl₃, 400 MHz): 7.65 (d, J=2.0 Hz, 1H), 7.38 (m, 4H), 6.93 (d, J=2.0 Hz, 1H), 4.5 (s, 2H), 2.96 (brs, 2H, NH2, exchangeable with D₂O).

MS: 220 (M+1).

### Step D: 2-{[2-(3-Chloro-benzyl)-pyrimidin-5-ylamino]-methylene}-malonic acid diethyl ester

A mixture of 2-(3-chloro-benzyl)-5-amino-pyrimidine (0.43 g, 1.97 mmol) and diethyl ethoxymethylene-malonate (0.43 g, 1.97 mmol) was heated at 140° C. for 2 hours and cooled to room temperature. The reaction mixture was purified by silica gel column (ISCO, hexane/EtOAc, 0-30%, 25 min, 30-80%, 5 min, 80%, 10 min) to give the pure product as a white solid in a quantitative yield.

¹H NMR (CD₃OD, 400 MHz): 8.37 (d, J=2.0 Hz, 1H), 7.96 (d, J=2.0 Hz, 1H), 7.48 (m, 5H), 4.81 (s, 2H), 4.27 (q, J=7.1 Hz, 2H), 4.18 (q, J=7.1 Hz, 2H), 1.32 (t, J=7.1 Hz, 3H), 1.25 (t, J=7.1 Hz, 3H).

MS: 391 (M+1).

### Step E: 2-(3-Chloro-benzyl)-8-oxo-5,8-dihydro-pyrido[3,2-d]pyrimidine-7-carboxylic acid ethyl ester

A solution of 2-{[2-(3-chloro-benzyl)-pyrimidin-5-ylamino]-methylene}-malonic acid diethyl ester (0.47 g, 1.2 mmol) in Dowtherm A (5 g) was heated at 240″C for 20 min and cooled to room temperature and diluted with 30 mL of hexane. The precipitates were filtered and washed with hexane and ethanol to give the crude product which was purified by silica gel column CISCO, Chloroform/methanol, 0-30%, 30 min) to give yellow solid products.

1 NMR (CD3OD, 400 MHz): 8.38 (d, J=2.0 Hz, 1H), 7.96 (d, J=2.0 Hz, 1H), 7.48 (m, 5H), 4.81 (s, 2H), 4.27 (q, J=7.1 Hz, 2H), 4.18 (q, J=7.1 Hz, 2H), 1.32 (t, J=7.1 Hz, 3H), 1.25 (t, J=7.1 Hz, 3H).

MS: 344 (M+1).

### Step F: 2-(3-Chloro-benzyl)-8-oxo-5,8-dihydro-pyrido[3,2-d]pyrimidine-7-carboxylic acid

A mixture of 2-(3-chloro-benzyl)-8-oxo-5,8-dihydro-pyrido[3,2-d]pyrimidine-7-carboxylic acid ethyl ester (0.61 g, 1.77 mmol), glacial acetic acid (7 mL), and 1 N hydrochloric acid (3.5 mL) was refluxed overnight. After cooling, the solvent was removed in vacuo and the residue was re-crystallized from ethanol to give the pure compound.

1H NMR (CD3OD, 400 MHz): 8.37 (d, J=2.0 Hz, 1H), 7.96 (d, J=2.01 Hz, 1H), 7.48 (m, 5H), 4.81 (s, 2H), 4.27 (q, J=7.1 Hz, 2H), 4.18 (q, J=7.1 Hz, 2H), 1.32 (t, J=7.1 Hz, 3H), 1.25 (t, J=7.1 Hz, 3H).

MS: 391 (M+1).

### Example 11

### Compounds of Formula (XI)

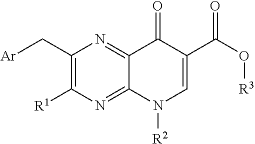

Compounds of formula (XI) were prepared according to the following general synthetic scheme. When appropriate, protecting, groups are used as needed according to established synthetic procedures known to those of skill in the art, and may or may not be removed upon completion of the synthesis. Starting materials are synthesized according to methods known in the art or are commercially available.

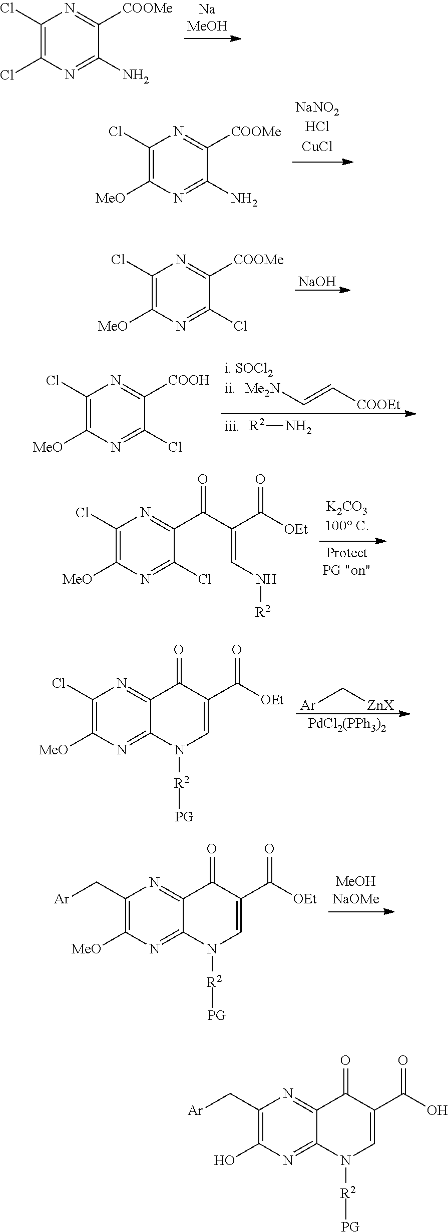

### Example 11A

### (S)-2-(3-chloro-2-fluorobenzyl)-3-hydroxy-5-(1-hydroxy-3-methylbutan-2-yl)-8-oxo-5,8-dihydropyrido[2,3-b]pyrazine-7-carboxylic acid

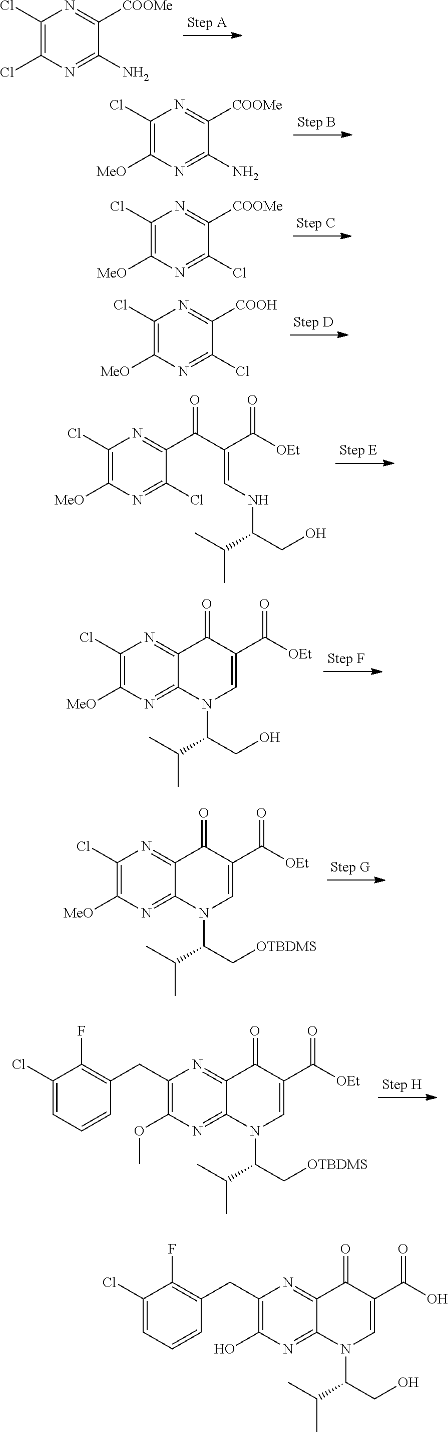

### Step A: 3-Amino-6-chloro-5-methoxy-pyrazine-2-carboxylic acid methyl ester

Methyl 3-amino-5,6-dichloropyrazinoate (1.1 g, 5 mmol) was dissolved in 200 mL of boiling anhydrous methanol containing metallic sodium (115 mg, 5 mmol). The product which separates on cooling, is filtered, washed with water and methanol and dried to give 1.0 g (92%) of methyl 3-amino-5-methoxy-6-chloro-pyrazinoate which was recrystallized from acetonitrile. MP. 255-257° C.

¹H NMR (DMSO-d₆, 400 MHz): δ 7.61 (brs, 2H, NH₂, exchangeable with D₂O), 3.97 (s, 3H), 3.80 (s, 3H).

### Step B: 3,6-Dichloro-5-methoxy-pyrazine-2-carboxylic acid methyl ester

In a mixture of 3 mL of 12 mol/L hydrochloric acid and 10 mL of tetrahydrofuran was suspended 3-amino-6-chloro-5-methoxy-pyrazine-2-carboxylic acid methyl ester (0.81 g, 4.18 mmol). After adding 0.58 g (8.36 mmol) of sodium nitrite at 5-12° C., the mixture was stirred in an ice-cooled bath for 50 minutes and 0.83 g (8.36 mmol) of cuprous (I) chloride suspended in 5 mL of 6 mol/L hydrochloric acid was added. The mixture was stirred at 5-12° C. for 10 minutes and was poured into a mixture of 20 mL of ethyl acetate and 20 mL of water. The organic layer was separated and was washed with water, brine; dried over-sodium sulfate and concentrated under reduced pressure. The residue was purified by ISCO (hexane/EtOAc, 0-30%, 40 min) to give recovered starting material and two products:

3,6-Dichloro-5-methoxy-pyrazine-2-carboxylic acid methyl ester white solids; Rf, 0.66 (hexane/EtOAc, 3:1). ¹H NMR (DMSO-d₆, 400 MHz): δ 4.07 (s, 3H), 3.89 (s, 3H).

6-Chloro-3-hydroxy-5-methoxy-pyrazine-2-carboxylic acid methyl ester: white crystals; Rf, 0.29 (hexane/EtOAc, 3:1). ¹H NMR (DMSO-d₆, 400 MHz): δ 12.24 (brs, 1H, OH, exchangeable with D₂O), 4.02 (s, 3H), 3.83 (s, 3H).

### Step C: 3,6-Dichloro-5-methoxy-pyrazine-2-carboxylic acid

In 20 mL of methanol was dissolved 0.65 g (2.7 mmol) of 3,6-dichloro-5-methoxy-pyrazine-2-carboxylic acid methyl ester and 10 mL of 1 N NaOH was added a 0° C. The mixture was allowed to warm up at room temperature and stirred for an additional 4 hours. The reaction mixture was evaporated to a small volume, diluted with water to give a yellow clear solution which was neutralized with 5N HCl. The solid was filtered and washed with water to give 0.42 g (69.7%) of pure product as a white solid after drying at 40° C. under reduced pressure.

¹H NMR (DMSO-d₆, 400 MHz): δ 13.85 (brs, 1H, OH, exchangeable with D₂O), 4.05 (s, 3H).

MS: 221, 223 (M−1).

### Step D: 2-(3,6-Dichloro-5-methoxy-pyrazine-2-carbonyl)-3-((S)-1-hydroxymethyl-2-methyl-propylamino)-acrylic acid ethyl ester

A mixture of 3,6-dichloro-5-methoxy-pyrazine-2-carboxylic acid (0.65 g, 2.9 mmol) and thionyl chloride (0.42 mL, 5.8 mmol) in 20 mL of anhydrous toluene and 0.1 mL of anhydrous DMF was refluxed for 2 h. The solvent was removed under reduced pressure to give a mobile oil residue which was azeoptoped with toluene (20 mL). The residue was dissolved in 10 mL of anhydrous THF and this solution was added dropwise to a Mixture of ethyl 3-(dimethylamino)acrylate (0.46 g, 3.2 mmol) and triethylamine (0.49 mL, 3.5 mmol) in 10 mL of anhydrous THF under nitrogen. The reaction mixture was heated under reflux for 7 hours, allowed to cool to room temperature and concentrated under reduced pressure. Water (100 mL) and ethyl acetate (100 L) were added to allow partitioning. The organic layer was washed successively with saturated aqueous sodium bicarbonate (×2), water, brine, dried over sodium sulfate and concentrated under reduced pressure. The crude product was used for next step without further purification.

A solution of the above product and L-valinol (0.3 g, 2.9 mmol) in anhydrous THF (20 mL) was stirred for 30 min at room temperature and the mixture was evaporated to dryness. The resulting crude material was purified by silica gel chromatography (ISCO, hexane/EtOAc, 0-40%, 40 min) to give 0.4.5 g (38%) of the pure compound as a yellow oil.

¹H NMR (CDCl₃, 400 MHz): δ 11.10 (dd, J=9.6 and 13.8 Hz, 1H, NH, exchangeable with D₂O), 8.32 (d, J=14.6 Hz, 1H, it becomes singlet after D₂O exchange), 4.12 (q, J=7.2 Hz, 2H), 4.08 (s, 3H), 3.86 (dd, J=3.8 and 11.3 Hz, 1H), 3.76 (dd, J=7.6 and 11.3 Hz, 1H), 3.22 (m, 1H), 2.01 (m, 1H), 1.05 (t, J=7.0 Hz, 3H); 1.04 (d, J=6.6 Hz, 3H), 1.02 (d, J=6.6 Hz, 3H).

MS: 406 (M+1).

### Step E: 2-Chloro-5-((S)-1-hydroxymethyl-2-methyl-propyl)-3-methoxy-8-oxo-5,8-dihydro-pyrido[2,3-b]pyrazine-7-carboxylic acid ethyl ester

A mixture of 2-(3,6-dichloro-pyridine-2-carbonyl)-3-((S)-1-hydroxymethyl-2-methyl-propylamino)-acrylic acid ethyl ester (0.365 g, 0.9 mmol) and potassium carbonate (0.25 g, 1.8 mmol) in anhydrous DMF (10 mL) was stirred at 100° C. for 1 hour, the mixture was filtered and washed with anhydrous DMF and the filtrate was evaporated to dryness under reduced pressure. The crude product was used for next step without further purification. An analytically pure sample was obtained by ISCO (Chloroform/methanol, 0-40%, 40 min) as a yellow solid.

¹H NMR (DMSO-d₆, 400 MHz): δ 8.69 (s, 1H), 5.11 (brs, J=5.1 Hz, 1H, OH, exchangeable with D₂O), 4.25 (q, J=7.0 Hz, 2H), 4.13 (s, 3H). 3.93 (m, 2H), 3.82 (m, 1H), 2.29 (m, 1H), 1.29 (t, j=7.0 Hz, 3H), 1.09 (d, J=6.6 Hz, 3H), 0.75 (d, J=6.6 Hz, 3H).

MS: 370 (M+1).

### 2-Chloro-3-hydroxy-5-((S)-1-hydroxymethyl-2-methyl-propyl)-8-oxo-5,8-dihydro-pyrido[2,3-b]pyrazine-7-carboxylic acid ethyl ester.

¹H NMR (DMSO-d₆, 400 MHz): δ 8.40 (s, 1H), 5.09 (brs, J=5.1 Hz, 1H, OH, exchangeable with D₂O), 4.20 (q, J=7.0 Hz, 2H), 3.89 (m, 2H), 3.61 (m, 1H), 2.26 (m, 1H), 1.26 (t, J=7.0 Hz, 3H), 1.06 (d, J=6.6 Hz, 3H), 0.68 (d, J=6.6 Hz, 3H). MS: 356 (M+1).

### Step F: 5-[(S)-1-(tert-Butyl-dimethyl-silanyloxymethyl)-2-methyl-propyl]-2-chloro-3-methoxy-8-oxo-5,8-dihydro-pyrido[2,3-b]pyrazine-7-carboxylic acid ethyl ester

A mixture of the above crude product and imidazole (0.61 g, 9.0 mmol) in 10 mL of anhydrous DMF was added tart-butyldimethylsilyl chloride (0.68 g, 4.5 mmol) under argon at room temperature and stirred overnight at room temperature. The mixture was evaporated to dryness under reduced pressure and the residue was purified by ISCO (Hexane/EtOAc, 0-30%, 40 min) to give the pure compound as a white solid.

¹H NMR (DMSO-d₆, 400 MHz): δ 8.73 (s, 1H), 5.08 (m, 1H), 4.39 (q, J=7.1 Hz, 2H), 4.15 (s, 3H), 4.09 (dd, J=4.8 and 11.6 Hz, 1H), 3.83 (d, J=11.6 Hz, 1H), 2.43 (m, 1H), 1.39 (t, J=7.1 Hz, 3H), 1.17 (d, J=6.6 Hz, 3H), 0.82 (s, 9H), 0.81 (d, J=6.6 Hz, 3H), 0.01 (s, 6H).

MS: 484 (M+1).

### 5-[(S)-1-(tert-Butyl-dimethyl-silanyloxymethyl)-2-methyl-propyl]-2-chloro-3-hydroxy-8-oxo-5,8-dihydro-pyrido[2,3-b]pyrazine-7-carboxylic acid ethyl ester

### Step G: 5-[(S)-1-(tert-Butyl-dimethyl-silanyloxymethyl)-2-methyl-propyl]-2-(3-chloro-2-fluoro-benzyl)-3-methoxy-8-oxo-5,8-dihydro-pyrido[2,3-b]pyrazine-7-carboxylic acid ethyl ester

Under an argon stream, zinc powder (346 mg, 5.3 mmol) was suspended in 1 mL of dry tetrahydrofuran. 1,2-Dibromoethane (1.4 μl, 0.016 mmol) and trimethylsilyl chloride (4.0 μl, 0.032 mmol) were added at 60° C. and the mixture was stirred with heating for 30 min. A solution of 2-fluoro-3-chloro-benzyl bromide (177 mg, 0.79 mmol) in 2 mL of dry tetrahydrofuran was added dropwise at 60° C. The mixture was stirred with heating for 1 hour and allowed to cool to room temperature to give a solution of 2-fluoro-3-chloro-benzylzinc bromide in tetrahydrofuran. This was used in the next step.

5-[(S)-1-(tert-Butyl-dimethyl-silanyloxymethyl)-2-methyl-propyl]-2-chloro-3-methoxy-8-oxo-5,8-dihydro-pyrido[2,3-b]pyrazine-7-carboxylic acid ethyl ester (256 mg, 0.53 mmol) was dissolved in 10 mL of dry tetrahydrofuran under an argon stream.

Dichlorobis(triphenylphosphine)palladium(II) (34 mg, 0.048 mmol) was added followed by the addition of a solution of 2-fluoro-3-chloro-benzylzinc bromide in tetrahydrofuran at 60° C. After the completion of the dropwise addition, the mixture was stirred an additional hour at 60° C. The reaction mixture was allowed to cool to room temperature, 1 N hydrochloric acid was added and the mixture was extracted three times with ethyl acetate. The organic layer were combined, washed successively with water, brine, dried over anhydrous sodium sulfate and concentrated under reduced pressure. The obtained residue was purified by silica gel chromatography (ISCO, 12 g column, hexane/EtOAc, 0-30%, 25 min; 30-100%, 10 min; 100%, 10 min) to give a major product as an yellow foam 220 mg (70%).

¹H NMR (CDCl₃, 400 MHz): δ 8.73 (s, 1H), 7.55 (dt, J=2.0 and 7.8 Hz, 1H), 7.24 (dt, J=1.2 and 7.8 Hz, 1H), 6.94 (t, J=7.8 Hz, 1H), 5.13 (d, J=10.1 Hz, 1H), 4.41 (s, 2H), 4.40 (q, J=7.1 Hz, 2H), 4.06 (dd, J=6.4 and 13.8 Hz, 1H), 4.02 (s, 3H), 3.81 (d, J=10.7 Hz, 1H), 2.45 (m, 1H), 1.40 (t, J=7.1 Hz, 3H), 1.16 (d, J=6.2 Hz, 3H), 0.81 (d, J=6.2 Hz, 3H), 0.80 (s, 9H), 0.02 (s, 6H).

MS: 592 (M+1).

### Step H: 2-(3-Chloro-2-fluoro-benzyl)-3-hydroxy-5-((S)-1-hydroxymethyl-2-methyl-propyl)-8-oxo-5,8-dihydro-pyrido[2,3-b]pyrazine-7-carboxylic acid

1-[(S)-1-(tert-Butyl-dimethyl-silanyloxymethyl)-2-methyl-propyl]-7-(3-chloro-2-fluoro-benzyl)-6-methoxy-4-oxo-1,4-dihydro-[1,5]naphthyridine-3-carboxylic acid ethyl ester (20 mg, 0.03 mmol) was dissolved in 10 mL of methanol and 25% sodium methoxide in methanol (1 mL) and water (2 mL) were added. The mixture was refluxed for 4 hours, allowed to cool to room temperature and evaporated to a small volume under reduced pressure. Water (10 mL) was added and the mixture was filtere. The filtrate was neutralized with 1 N hydrochloric acid. The solid was filtered and washed with water to give a pure product as an white solid.

¹H NMR (DMSO-d₆, 400 MHz): δ 15.60 (brs, 1H, OH, exchangeable with D₂O), 8.95 (s, 1H), 7.70 (dt, J=1.4 and 7.8 Hz, 1H), 7.56 (dt, J=1.4 and 7.8 Hz, 1H), 7.25 (t, J=7.8 Hz, 1H), 5.39 (brs, 1H, OH, exchangeable with D₂O), 5.30 (m, 1H), 4.36 (s, 2H), 4.06 (m, 1H), 3.83 (m, 1H), 2.42 (m, 1H), 1.15 (d, J=6.6 Hz, 3H), 0.76 (d, J=6.6 Hz, 3H). MS: 436 (M+1).

### Example 12

### Compounds of Formula (XII)

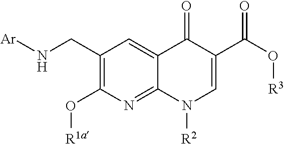

Compounds of formula (XII) were prepared according to the general synthetic scheme shown below. When appropriate, protecting groups are used as needed according to established synthetic procedures known to those of skill in the art, and may or may not be removed upon completion of the synthesis. Starting materials are synthesized according to methods known in the art or are commercially available.

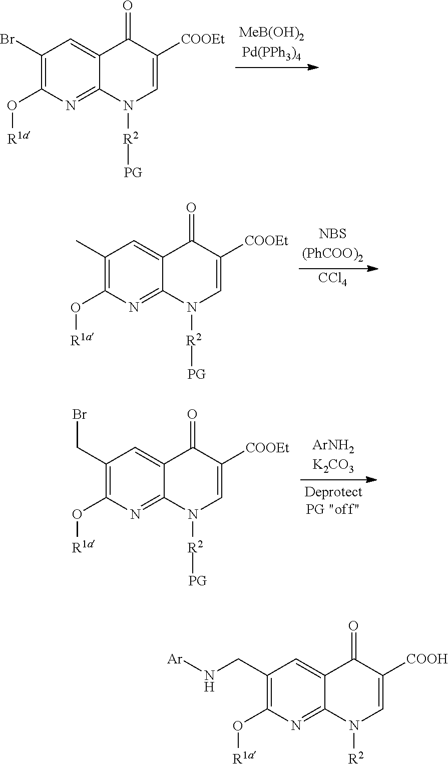

### Example 12A

### (S)-1-(1-hydroxy-3-methylbutan-2-yl)-7-methoxy-4-oxo-6-((2,4,6-trifluorophenylamino)methyl)-1,4-dihydro-1,8-naphthyridine-3-carboxylic acid

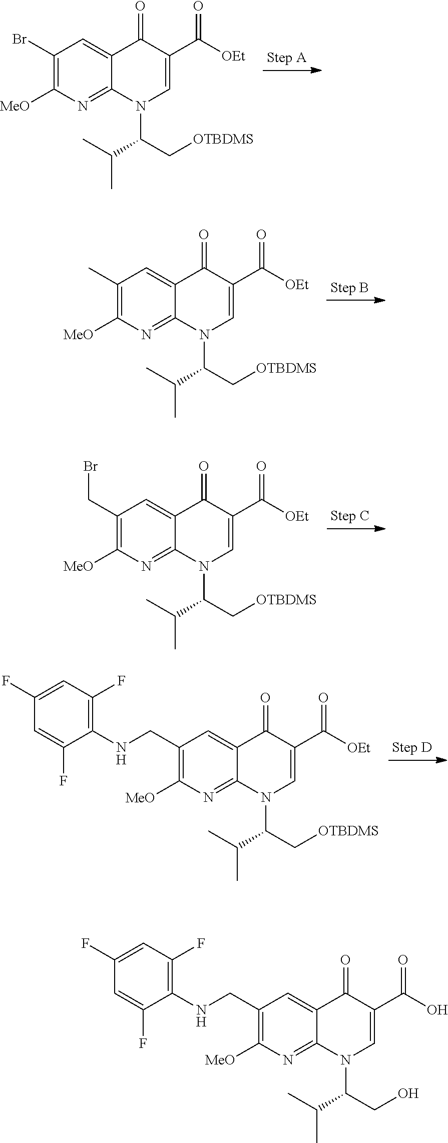

### Step A: (S)-Ethyl 1-(1-(tert-butyldimethylsilyloxy)-3-methylbutan-2-yl)-7-methoxy-6-methyl-4-oxo-1,4-dihydro-1,8-naphthyridine-3-carboxylate

(S)-Ethyl 6-bromo-1-(1-(tert-butyldimethylsilyloxy)-3-methylbutan-2-yl)-7-methoxy-4-oxo-1,4-dihydro-1,8-naphthyridine-3-carboxylate (500 mg, 0.95 mmol), methyl boronic acid (85 mg, 1.4 mmol, prepared via procedures described herein) and palladium tetrakis(triphenylphosphine) palladium(0) (110 mg, 0.095 mmol) were combined in a vial and flushed with nitrogen. Degassed THF (6 mL) and sodium carbonate 2M solution (2.8 mL, 5.6 mmol) were added and the mixture stirred at 70° C. over night. The reaction mixture was cooled to room temperature, diluted with ethyl acetate and washed with sodium carbonate saturated solution. The organic layer was dried over sodium sulfate and concentrated. Purification by preparative thin layer chromatography (50% ethyl acetate/50% hexane) afforded (S)-ethyl 1-(1-(tert-butyldimethylsilyloxy)-3-methylbutan-2-yl)-7-methoxy-6-methyl-4-oxo-1,4-dihydro-1,8-naphthyridine-3-carboxylate as a white solid (44%).

¹H NMR (DMSO-d₆, 400 MHz): δ 8.72 (s, 1H); 8.29 (s, 1H), 5.33 (m, 1H), 4.26 (q, J=7.1 Hz, 2H), 4.05 (s, 3H), 3.97 (m, 1H), 3.8 (m, 1H), 2.36 (m, 1H), 2.25 (s, 3H), 1.30 (t, J=7.1 Hz, 3H), 1.16 (d, J=6.2 Hz, 3H), 0.79 (d, J=6.2 Hz, 3H), 0.77 (s, 9H), 0.02 (s, 6H).

### Step B: (S)-Ethyl 6-(bromomethyl)-1-(1-(tert-butyldimethylsilyloxy)-3-methylbutan-2-yl)-7-methoxy-4-oxo-1,4-dihydro-1,8-naphthyridine-3-carboxylate

A mixture of (S)-ethyl 1-(1-(tert-butyldimethylsilyloxy)-3-methylbutan-2-yl)-7-methoxy-6-methyl-4-oxo-1,4-dihydro-1,8-naphthyridine-3-carboxylate (500 mg, 1.08 mmol), N-bromosuccinimide (213 mg, 1.2 mmol), and benzoyl peroxide (26 mg, 0.11 mmol) in carbon tetrachloride (10 mL) was stirred at 77° C. for 18 hours. The mixture was the cooled to room temperature, concentrated, dissolved in ethyl acetate and washed with sodium bicarbonate (saturated solution). The organic layer was dried over sodium sulfate and concentrated. Purification by preparative TLC (50% ethyl acetate/50% hexanes) afforded (S)-ethyl 6-(bromomethyl)-1-(1-(tert-butyldimethylsilyloxy)-3-methylbutan-2-yl)-7-methoxy-4-oxo-1,4-dihydro-1,8-naphthyridine-3-carboxylate as a solid.

¹H NMR (DMSO-d₆, 400 MHz): δ 8.7 (s, 1H), 8.6 (s, 1H), 5.4 (m, 1H), 4.8 (s, 2H), 4.26 (q, J=7.1 Hz, 2H), 4.05 (s, 3H), 3.97 (m, 1H), 3.8 (m, 1H), 2.36 (m, 1H), 2.25 (t, J=7.1 Hz, 3H), 1.16 (d, J=6.2 Hz, 3H), 0.79 (d, J=6.2 Hz, 3H), 0.77 (s, 9H), 0.02 (s, 6H).

### Step C: (S)-Ethyl 1-(1-(tert-butyldimethylsilyloxy)-3-methylbutan-2-yl)-7-methoxy-4-oxo-6-((2,4,6-trifluorophenylamino)methyl)-1,4-dihydro-1,8-naphthyridine-3-carboxylate

(S)-Ethyl 6-(bromomethyl)-1-(1-(tert-butyldimethylsilyloxy)-3-methylbutan-2-yl)-7-methoxy-4-oxo-1,4-dihydro-1,8-naphthyridine-3-carboxylate (58 mg, 0.11 mmol), 2,4,6-trifluoroaniline (16 mg, 0.11 mmol) and potassium carbonate (30 mg, 0.22 mmol) in DMF (2 mL) were stirred at room temperature for 18 hours. Water was then added to the mixture, neutralized with 1 N HCl and extracted with ethyl acetate. The organic layer was dried over sodium sulfate and concentrated. Purification by preparative TLC (90% dichlolromethane/10% methanol) afforded (S)-ethyl 1-(1-(tert-butyldimethylsilyloxy)-3-methylbutan-2-yl)-7-methoxy-4-oxo-6-((2,4,6-trifluorophenylamino)methyl)-1,4-dihydro-1,8-naphthyridine-3-carboxylate as a solid.

¹H NMR (DMSO-d₆, 400 MHz): δ 8.7 (s, 1H), 8.3 (s, 1H), 7.0 (t, J=8 Hz, 2H), 5.7 (bt, 1H), 5.4 (m, 1H), 4.4 (m, 2H), 4.26 (q, J=7.1 Hz, 2H), 4.1 (m, 1H), 4.05 (s, 3H), 3.97 (m, 1H), 2.36 (m, 1H), 2.25 (t, J=7.1 Hz, 3H), 1.16 (d, J=6.2 Hz, 3H), 0.79 (d, J=6.2 Hz, 3H), 0.77 (s, 9H), 0.02 (s, 6H).

### Step D: (S)-1-(1-Hydroxy-3-methylbutan-2-yl)-7-methoxy-4-oxo-6-((2,4,6-trifluorophenyl amino)methyl)-1,4-dihydro-1,8-naphthyridine-3-carboxylic acid

A mixture of (S)-ethyl 1-(1-(tert-butyldimethylsilyloxy)-3-methylbutan-2-yl)-7-methoxy-4-oxo-6-((2,4,6-trifluorophenylamino)methyl)-1,4-dihydro-1,8-naphthyridine-3-carboxylate (30 mg, 0.05 mmol) and sodium methoxide (25% in methanol) (0.5 mL) in methanol (2 mL) and water (1 mL) was stirred at 65° C. for 2 hours. The reaction mixture was then cooled to room temperature and acidified with HCl (1 N) and extracted with ethyl acetate. The organic layer was dried over sodium sulfate and concentrated. Purification by preparative thin layer chromatography (95% dichloromethane/5% methanol) afforded (S)-1-(1-hydroxy-3-methylbutan-2-yl)-7-methoxy-4-oxo-6-((2,4,6-trifluorophenyl amino)methyl)-1,4-dihydro-1,8-naphthyridine-3-carboxylic acid as a solid.

¹H NMR (DMSO-d₆, 400 MHz): δ 15.2 (s, 1H), 8.9 (s, 1H), 8.5 (s, 1H), 7.10 (t, J=9 Hz, 2H), 5.8 (bt, 1H), 5.5 (m, 1H), 5.2 (bs, 1H), 4.5 (s, 2H), 4.2 (s, 3H), 4.05 (m, 1H), 3.9 (m, 1H), 2.33 (m, 1H), 1.14 (d, J=6.2 Hz, 3H), 0.73 (d, J=6.2 Hz, 3H).

### Examples 12B-12J

Examples 12B-12J were prepared according to the procedure described above for example 12A.

| | | NMR |
|---|---|---|
| Compound | Structure | (DMSO-d₆, 400 MHz) |
| 12A | 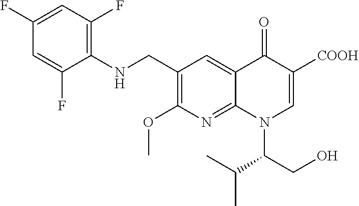 | 15.2 (s, 1H), 8.9 (s, 1H), 8.5 (s, 1H), 7.10 (t, J = 9 Hz, 2H), 5.8 (bt, 1H), 5.5 (m, 1H), 5.2 (bs, 1H), 4.5 (s, 2H), 4.2 (s, 3H), 4.05 (m, 1H), 3.9 (m, 1H), 2.33 (m, 1H), 1.14 (d, J = 6.2 Hz, 3H), 0.73 (d, J = 6.2 Hz, 3H). |
| 12B | 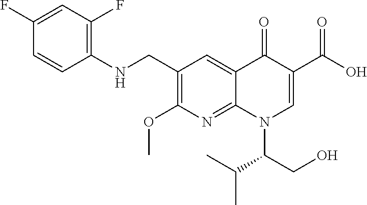 | 15.2 (s, 1H), 8.9 (s, 1H), 8.5 (s, 1H), 6.9 (m, 2H), 6.7 (m, 1H), 5.8 (bt, 1H), 5.5 (m, 1H), 5.2 (bs, 1H), 4.5 (s, 2H), 4.2 (s, 3H), 4.05 (m, 1H), 3.9 (m, 1H), 2.33 (m, 1H), 1.14 (d, J = 6.2 Hz, 3H), 0.73 (d, J = 6.2 Hz, 3H). |
| 12C | 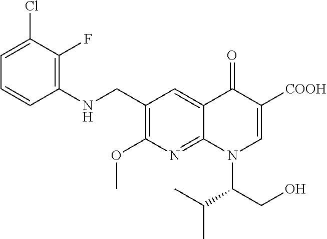 | Not available |
| 12D | 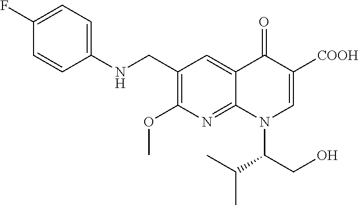 | Not available |
| 12E | 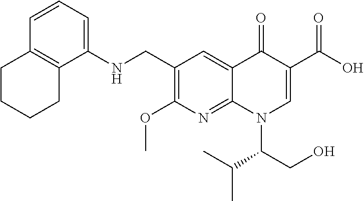 | 15.2 (s, 1H), 9.0 (s, 1H), 8.4 (s, 1H), 6.8 (t, J = 8 Hz, 1H), 6.4 (d, J = 8 Hz, 1H), 6.1 (d, J = 8 Hz, 1H), 5.7 (t, J = 8 Hz, 1H), 5.5 (m, 1H), 5.2 (bs, 1H), 4.4 (d, J = 8 Hz, 2H), 4.1 (s, 3H), 4.0 (m, 1H), 3.9 (m, 1H), 2.7 (t, J = 4 Hz, 2H), 2.4 (m, 1H), 1.9 (m, 2H), 1.7 (m, 2H), 1.14 (d, J = 6.2 Hz, 3H), 0.75 (d, J = 6.2 Hz, 3H) |
| 12F | 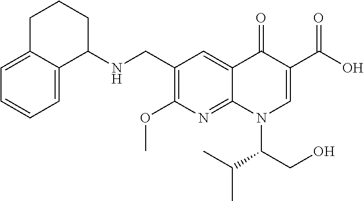 | 15.2 (s, 1H), 9.0 (s, 1H), 8.75 (s, 1H), 7.5 (m, 1H), 7.2 (m, 2H), 7.1 (m, 1H), 5.5 (m, 1H), 5.2 (bs, 1H), 4.1 (s, 3H), 4.0 (m, 1H), 3.8 (m, 4H), 2.8 (m, 1H), 2.7 (m, 1H), 2.4 (m, 1H), 2.0 (m, 1H), 1.9 (q, J = 4 Hz, 2H), 1.7 (m, 1H), 1.14 (d, J = 6.2 Hz, 3H), 0.75 (d, J = 6.2 Hz, 3H) |
| 12G | 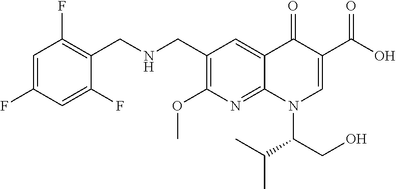 | 15.4 (s, 1H), 9.0 (s, 1H), 8.6 (s, 1H), 7.2 (t, J = 9 Hz, 2H), 5.5 (m, 1H), 5.2 (bs, 1H), 4.1 (s, 3H), 4.0 (m, 1H), 3.9 (m, 1H), 3.7 (d, 4H), 2.5 (m, 1H), 1.14 (d, J = 6.2 Hz, 3H), 0.73 (d, J = 6.2 Hz, 3H) |
| 12H | 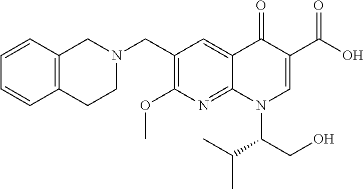 | 15.4 (s, 1H), 9.0 (s, 1H), 8.6 (s, 1H), 7.2 (m, 3H), 7.1 (m, 1H), 5.5 (m, 1H), 5.2 (bs, 1H), 4.1 (s, 3H), 4.0 (m, 1H), 3.9 (m, 1H), 3.8 (s, 2H), 3.7 (s, 2H), 2.9 (t, J = 4 Hz, 2H), 2.7 (t, J = 4 Hz, 2H), 2.5 (m, 1H), 1.14 (d, J = 6.2 Hz, 3H), 0.73 (d, J = 6.2 Hz, 3H) |

### Example 13

### Compounds of Formula (XIII)

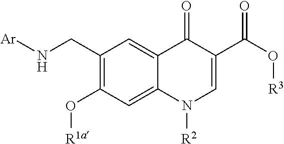

Compounds of formula (XII) were prepared according to the general synthetic scheme shown below. When appropriate, protecting groups are used as needed according to established synthetic procedures known to those of skill in the art, and may or may not be removed upon completion of the synthesis. Starting materials are synthesized according to methods known in the art or are commercially available.

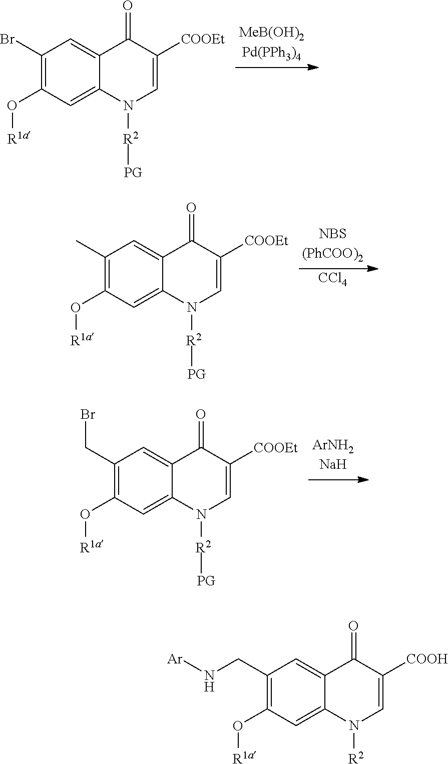

### Example 13A

### (S)-6-((4-fluorophenylamino)methyl)-1-(1-hydroxy-3-methylbutan-2-yl)-7-methoxy-4-oxo-1,4-dihydroquinoline-3-carboxylic acid

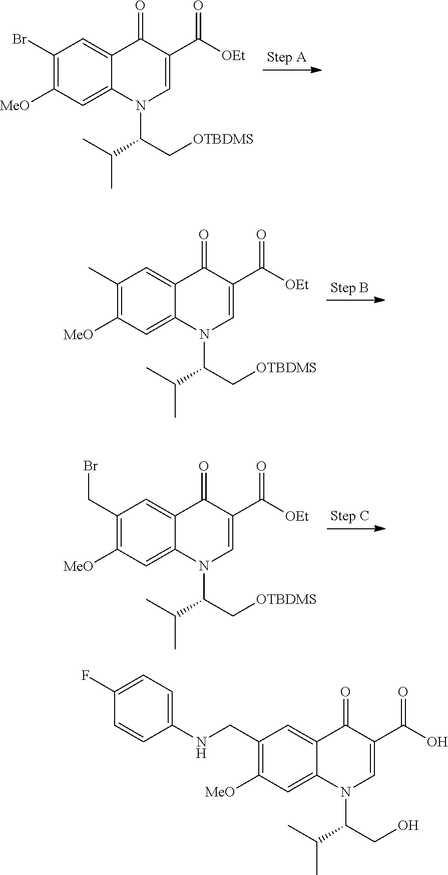

The title compound was prepared according to the scheme above and procedures for related compounds, as described herein.

| | | NMR |
|---|---|---|
| Compound | Structure | (DMSO-d₆, 400 MHz) |
| 13A | 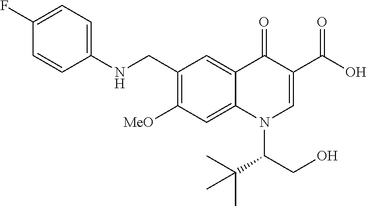 | 15.5 (s, 1H), 8.8 (s, 1H), 8.3 (s, 1H), 7.6 (s, 1H), 6.9 (t, J = 12, 2H), 6.6 (m, 2H), 6.3 (m, 1H), 5.2 (m, 1H), 5.16 (m, 1H), 4.3 (d, J = 4 Hz, 2H), 4.15 (m, 1H), 4.1 (s, 3H), 4.05 (m, 1H), 1.02 (s, 9H) |

### Example 14

### Compounds of Formula (XIV)

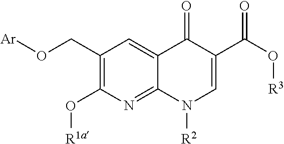

Compounds of formula (XIV) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

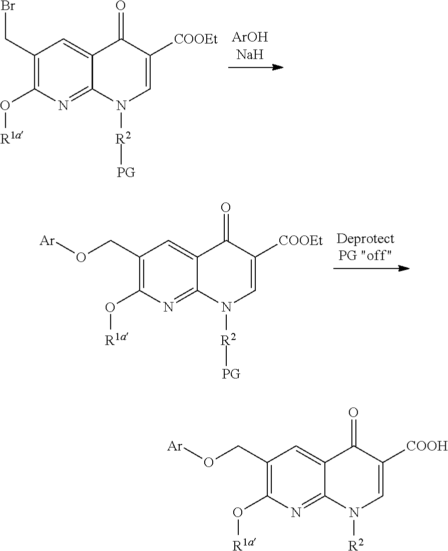

### Example 14A

### (S)-1-(1-hydroxy-3-methylbutan-2-yl)-7-methoxy-4-oxo-6-((2,4,6-trifluorophenoxy)methyl)-1,4-dihydro-1,8-naphthyridine-3-carboxylic acid

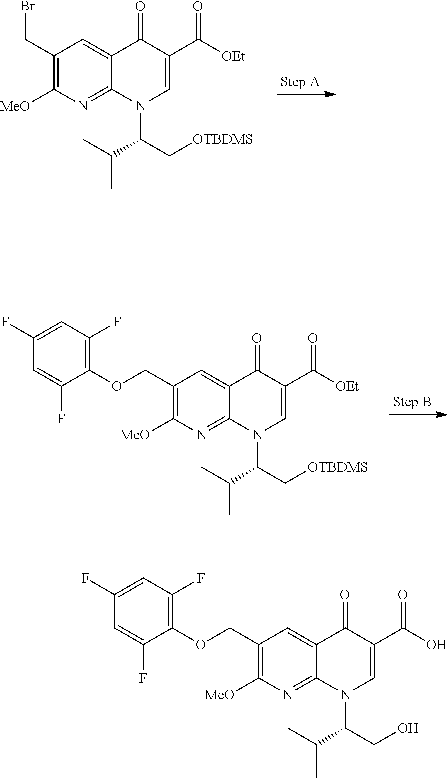

### Step A: (S)-ethyl 1-(1-(tert-butyldimethylsilyloxy)-3-methylbutan-2-yl)-7-methoxy-4-oxo-6-((2,4,6-trifluorophenoxy)methyl)-1,4-dihydro-1,8-naphthyridine-3-carboxylate

(S)-Ethyl 6-(bromomethyl)-1-(1-(tert-butyldimethylsilyloxy)-3-methylbutan-2-yl)-7-methoxy-4-oxo-1,4-dihydro-1,8-naphthyridine-3-carboxylate (87 mg, 0.16 mmol), 2,4,6-trifluorophenol (24 mg, 0.16 mmol) and sodium hydride (8 mg, 0.32 mmol) in DMF (2 mL) were stirred at room temperature for 1 hour. Water was then added to the mixture, neutralized with HCl (1 N) and extracted with ethyl acetate. The organic layer was dried over sodium sulfate and concentrated. Purification by preparative TLC (90% dichlolromethane/10% methanol) afforded (S)-ethyl 1-(1-(tert-butyldimethylsilyloxy)-3-methylbutan-2-yl)-7-methoxy-4-oxo-6-((2,4,6-trifluorophenoxy)methyl)-1,4-dihydro-1,8-naphthyridine-3-carboxylate as a solid.

¹H NMR (DMSO-d₆, 400 MHz): δ 8.7 (s, 1H), 8.5 (s, 1H), 7.3 (t, J=8 Hz, 2H), 5.3 (m, 1H), 5.22 (s, 2H), 5.15 (bs, 1H), 4.29 (q, J=8 Hz, 2H), 4.03 (s, 3H), 3.99 (m, 1H), 3.83 (m, 1H), 2.27 (m, 1H), 1.33 (t, J=8 Hz, 3H), 1.13 (d, J=8 Hz, 3H), 0.75 (d, J=4 Hz, 3H).

### Step B: ((S)-1-(1-hydroxy-3-methylbutan-2-yl)-7-methoxy-4-oxo-6-((2,4,6-trifluorophenoxy)methyl)-1,4-dihydro-1,8-naphthyridine-3-carboxylic acid

A mixture of (S)-ethyl 1-(1-(tert-butyldimethylsilyloxy)-3-methylbutan-2-yl)-7-methoxy-4-oxo-6-((2,4,6-trifluorophenoxy)methyl)-1,4-dihydro-1,8-naphthyridine-3-carboxylate (30 mg, 0.05 mmol) and sodium methoxide (25% in methanol) (0.5 mL) in methanol (2 mL) and water (1 mL) was stirred at 65° C. for 2 hours. The reaction mixture was then cooled to room temperature and acidified with HCl (1 N) and extracted with ethyl acetate. The organic layer was dried over sodium sulfate and concentrated. Purification by preparative thin layer chromatography (95% dichloromethane/5% methanol) afforded (S)-1-(1-hydroxy-3-methylbutan-2-yl)-7-methoxy-4-oxo-6-((2,4,6-trifluoro phenoxy)methyl)-1,4-dihydro-1,8-naphthyridine-3-carboxylic acid as a solid.

¹H NMR (DMSO-d₆, 400 MHz): δ 15.2 (s, 1H), 9.1 (s, 1H), 8.7 (s, 1H), 7.3 (t, J=8 Hz, 2H), 5.3 (m, 1H), 5.22 (s, 2H), 5.15 (bs, 1H), 4.03 (s, 3H), 3.99 (m, 1H), 3.83 (m, 1H), 2.27 (m, 1H), 1.13 (d, J=8 Hz, 3H), 0.75 (d, J=4 Hz, 3H).

### Example 14B

Example 14B was prepared according to the procedure described above for example 14A.

| | | NMR | EC50 | |
|---|---|---|---|---|
| Compound | Structure | (DMSO-d₆, 400 MHz) | (uM) | Rank |
| 14A | 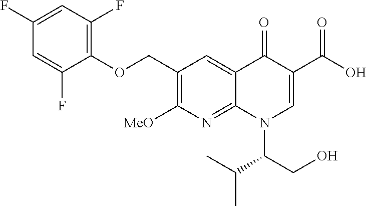 | 15.2 (s, 1H), 9.1 (s, 1H), 8.7 (s, 1H), 7.3 (t, J = 8 Hz, 2H), 5.3 (m, 1H), 5.22 (s, 2H), 5.15 (bs, 1H), 4.03 (s, 3H), 3.99 (m, 1H), 3.83 (m, 1H), 2.27 (m, 1H), 1.13 (d, J = 8 Hz, 3H), 0.75 (d, J = 4 Hz, 3H) | >2500 | C |
| 14B | 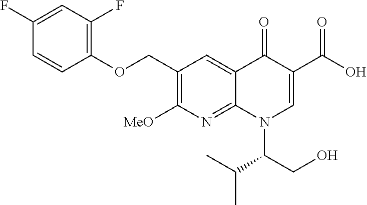 | 15.2 (s, 1H), 9.1 (s, 1H), 8.7 (s, 1H), 7.4 (m, 2H), 7.1 (m, 1H), 5.5 (m, 1H), 5.22 (s, 2H), 4.1 (s, 3H), 3.99 (m, 1H), 3.83 (m, 1H), 2.27 (m, 1H), 1.13 (d, J = 8 Hz, 3H), 0.75 (d, J = 4 Hz, 3H). | >2500 | C |

### Example 15

### Compounds of Formula (XV)

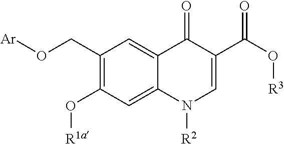

Compounds of formula (XV) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

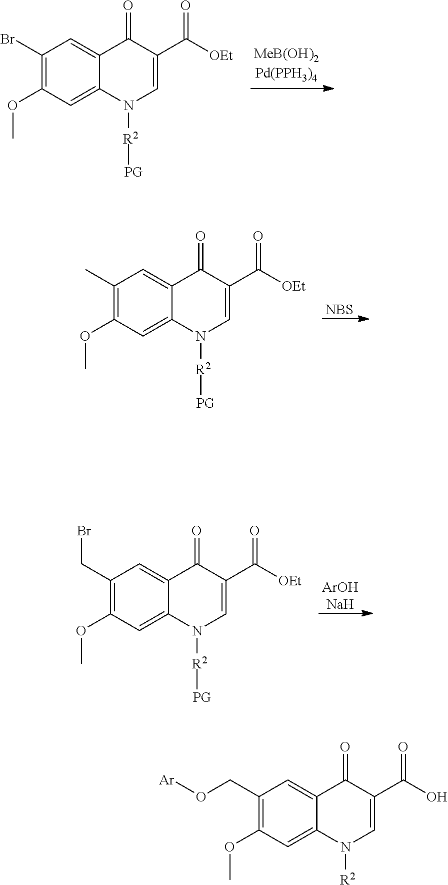

### Example 15A

### (S)-6-((4-fluorophenoxy)methyl)-1-(1-hydroxy-3-methylbutan-2-yl)-7-methoxy-4-oxo-1,4-dihydroquinoline-3-carboxylic acid

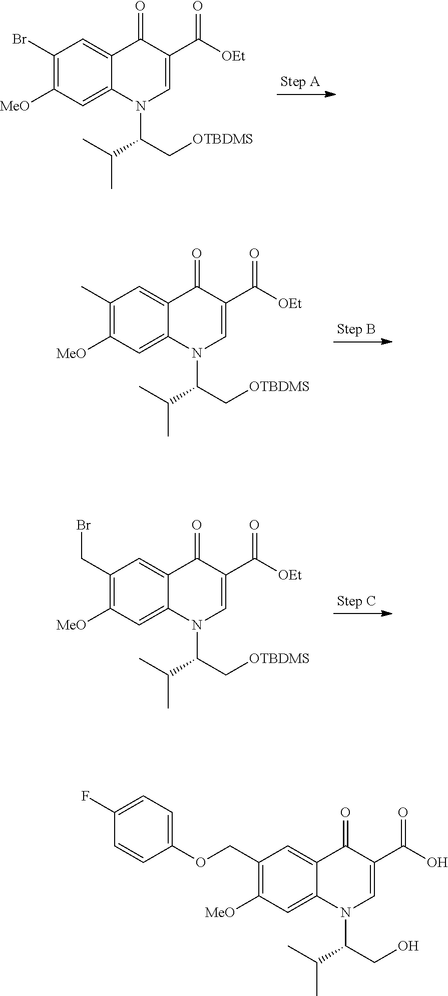

### Step A: (S)-ethyl 1-(1-(tert-butyldimethylsilyloxy)-3-methylbutan-2-yl)-7-methoxy-6-methyl-4-oxo-1,4-dihydroquinoline-3-carboxylate

(S)-Ethyl 6-bromo-1-(1-(tert-butyldimethylsilyloxy)-3-methylbutan-2-yl)-7-methoxy-4-oxo-1,4-dihydroquinoline-3-carboxylate (200 mg, 0.37 mmol), methyl boronic acid (39 mg, 0.6 mmol) and palladium tetrakis(triphenylphosphine)palladium(0) (24 mg, 0.02 mmol) were combined in a vial and flushed with nitrogen. Degassed THF (3 mL) and sodium carbonate 2M solution (0.52 mL, 1.04 mmol) were added and the mixture was stirred at 70° C. over night. The reaction mixture was the cooled to room temperature, diluted with ethyl acetate and washed with sodium carbonate saturated solution. The organic layer was dried over sodium sulfate and concentrated. Purification by preparative thin layer chromatography (50% ethyl acetate/50% hexane) afforded (S)-ethyl 1-(1-(tert-butyldimethylsilyloxy)-3-methylbutan-2-yl)-7-methoxy-6-methyl-4-oxo-1,4-dihydroquinoline-3-carboxylate as a white solid 41% yield.

1H NMR (DMSO-d6, 400 MHz): δ 8.6 (s, 1H), 8.0 (s, 1H), 7.2 (s, 1H), 4.8 (m, 1H), 4.26 (q, J=8 Hz, 2H), 4.1 (m, 1H), 4.0 (s, 3H), 3.8 (m, 1H), 2.4 (m, 1H), 2.25 (s, 3H), 1.30 (t; J=8 Hz, 3H), 1.2 (d, J=8 Hz, 3H), 0.79 (d, J=8 Hz, 3H), 0.77 (s, 9H), 0.02 (s, 6H).

### Step B: (S)-ethyl 6-(bromomethyl)-1-(1-(tert-butyldimethylsilyloxy)-3-methylbutan-2-yl)-7-methoxy-4-oxo-1,4-dihydroquinoline-3-carboxylate

A mixture of (S)-ethyl 1-(1-(tert-butyldimethylsilyloxy)-3-methylbutan-2-yl)-7-methoxy-6-methyl-4-oxo-1,4-dihydroquinoline-3-carboxylate (80 mg, 0.17 mmol), N-bromosuccinimide (31 mg, 0.17 mmol), and 2,2′-azobis isobutyro-nitrile (4 mg, 0.02 mmol) in dichloroethane (2 mL) was stirred at reflux for 18 hours. The mixture was the cooled to room temperature and concentrated. Purification by preparative TLC (90% dichloromethane/5% methanol) afforded compound (S)-ethyl 6-(bromomethyl)-1-(1-(tert-butyldimethylsilyloxy)-3-methylbutan-2-yl)-7-methoxy-4-oxo-1,4-dihydroquinoline-3-carboxylate as a solid.

¹H NMR (DMSO-d₆, 400 MHz): δ 8.6 (s, 1H), 8.0 (s, 1H), 7.2 (s, 1H), 4.8 (s, 2H), 4.75 (m, 1H), 4.26 (q, J=8 Hz, 2H), 4.1 (m, 1H), 4.0 (s, 3H), 3.8 (m, 1H), 2.4 (m, 1H), 1.30 (t, J=8 Hz, 3H), 1.2 (d, J=8 Hz, 3H), 0.8 (d, J=8 Hz, 3H), 0.77 (s, 9H), 0.02 (s, 6H).

### Step C: (S)-6-((4-fluorophenoxy)methyl)-1-(1-hydroxy-3-methylbutan-2-yl)-7-methoxy-4-oxo-1,4-dihydroquinoline-3-carboxylic acid

Compound 3 (30 mg, 0.06 mmol), 4-fluorophenol (7 mg, 0.06 mmol) and sodium hydride (3 mg, 0.11 mmol) in DMF (1 mL) were stirred at room temperature for 4 hours. Water was then added to the mixture, neutralized with 1 N HCl and extracted with ethyl acetate. The organic layer was dried over sodium sulfate and concentrated. Purification by preparative TLC (90% dichlolromethane/5% methanol) afforded (S)-6-((4-fluorophenoxy)methyl)-1-(1-hydroxy-3-methylbutan-2-yl)-7-methoxy-4-oxo-1,4-dihydroquinoline-3-carboxylic acid as a solid.

¹H NMR (DMSO-d₆, 400 MHz): δ 15.5 (s, 1H), 8.9 (s, 1H), 8.4 (s, 1H), 7.5 (s, 1H), 7.2 (m, 2H), 7.1 (m, 2H), 5.3 (m, 1H), 5.2 (s, 2H), 4.9 (m, 1H), 4.1 (m, 1H), 4.1 (s, 3H), 4.0 (m, 1H), 3.8 (m, 1H), 2.4 (m, 1H), 1.2 (d, J=8 Hz, 3H), 0.8 (d, J=8 Hz, 3H).

### Example 16

### Compounds of Formula (XVI)

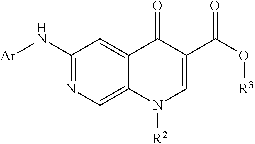

Compounds of formula (XVI) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

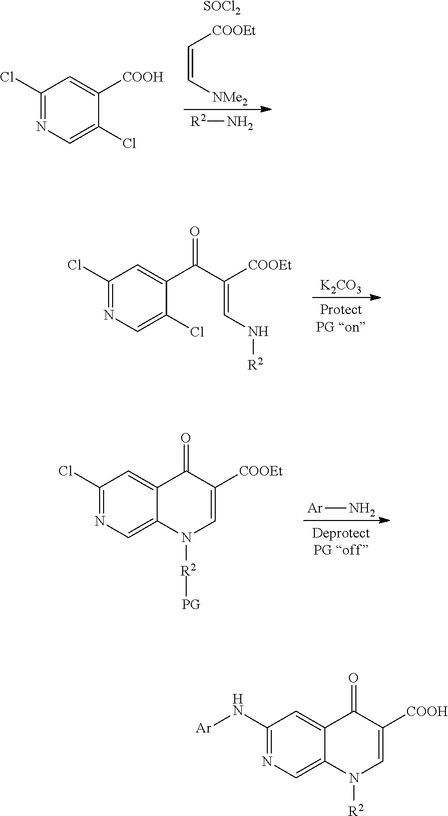

### Example 16A

### (S)-6-(3-chloro-2-fluorophenylamino)-1-(1-hydroxy-3-methylbutan-2-yl)-4-oxo-1,4-dihydro-1,7-naphthyridine-3-carboxylic acid

The title compound was prepared according to the scheme below and procedures similar to those described herein.

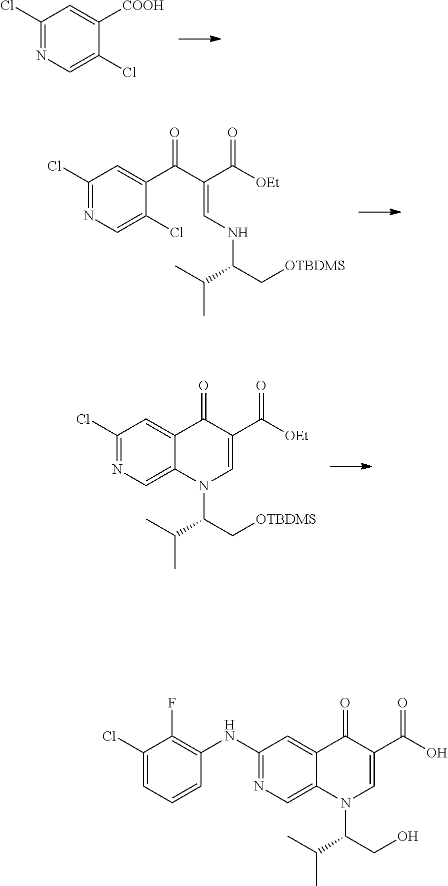

### Example 17

### Compounds of Formula (XVII)

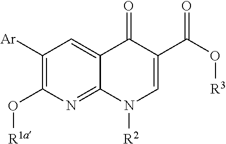

Compounds of formula (XVII) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

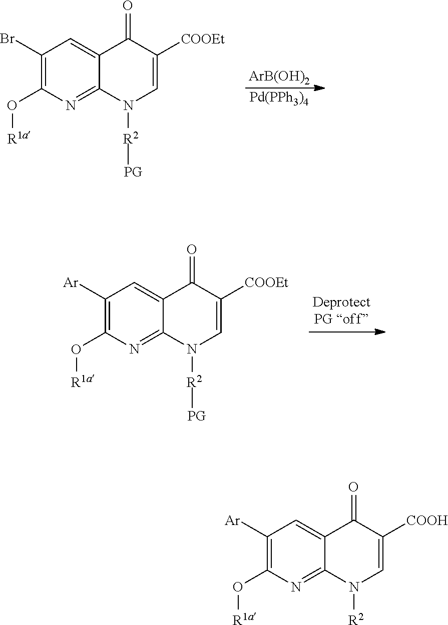

### Examples 17A-17O

Examples 17A-17O were prepared according to the general scheme shown above and the procedures described herein.

| Com- | |
|---|---|
| pounds | Structure |
| 17A | 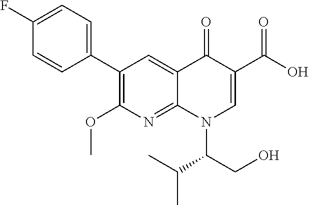 |
| 17B | 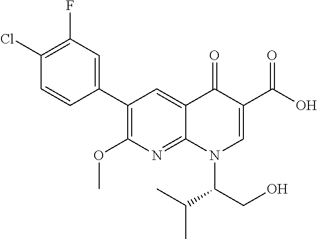 |
| 17C | 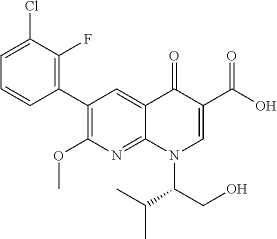 |
| 17D | 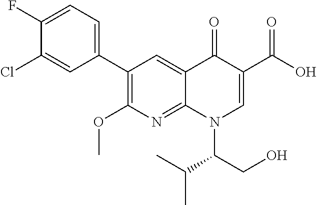 |
| 17E | 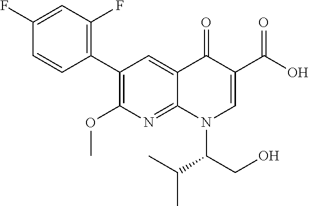 |
| 17F | 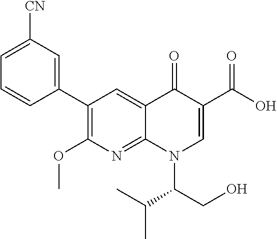 |
| 17G | 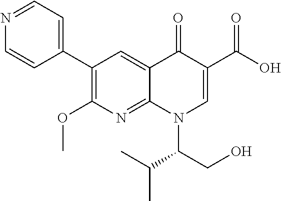 |
| 17H | 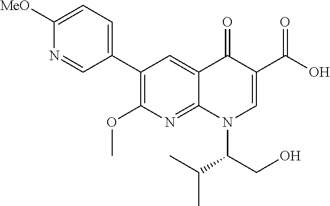 |
| 17I | 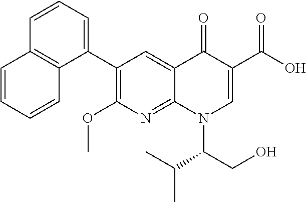 |
| 17J | 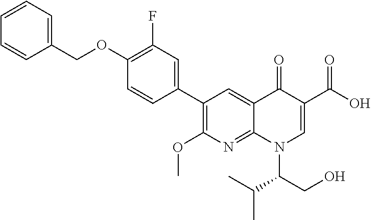 |
| 17K | 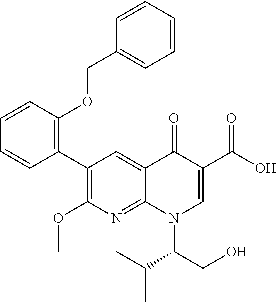 |
| 17L | 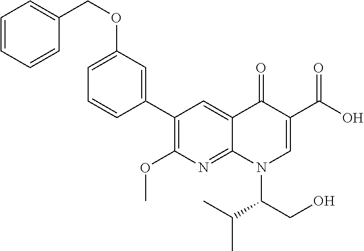 |
| 17M | 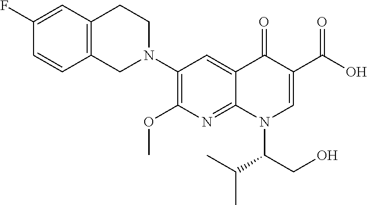 |
| 17N | 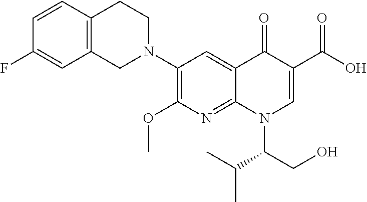 |
| 17O | 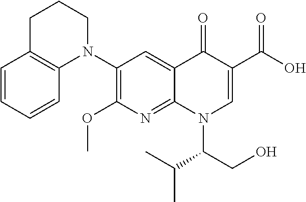 |

### Example 18

### Compounds of Formula (XVIII)

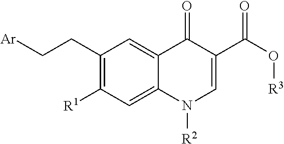

Compounds of formula (XVIII) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

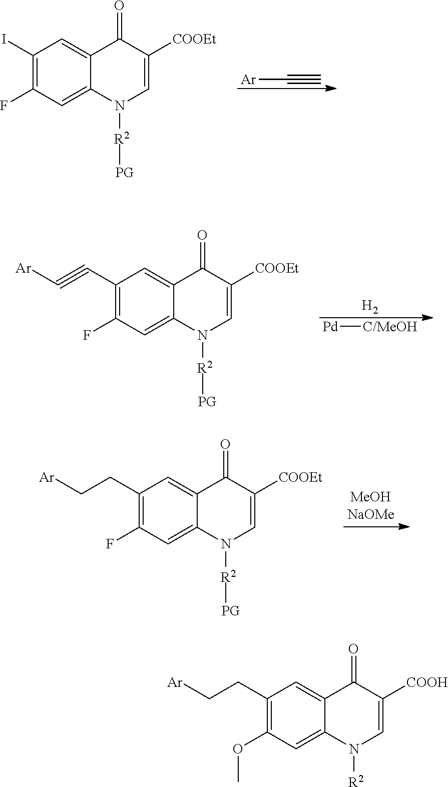

### Example 18A

### 6-[2-(2,4-Difluoro-phenyl)-ethyl]1-((S)-1-hydroxymethyl-2,2-dimethyl-propyl)-7-methoxy-4-oxo-1,4-dihydro-quinoline-3-carboxylic acid

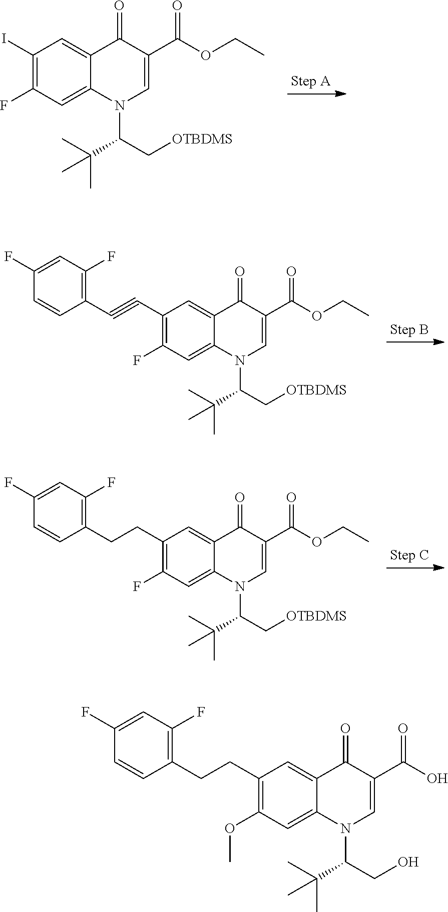

### Step A: 1-[(S)-1-(tert-Butyl-dimethyl-silanyloxymethyl)-2,2-dimethyl-propyl]-6-(2,4-difluoro-phenylethynyl)-7-fluoro-4-oxo-1,4-dihydro-quinoline-3-carboxylic acid ethyl ester

1-[(S)-1-(tert-Butyl-dimethyl-silanyloxymethyl)-2,2-dimethyl-propyl]-6-iodo-7-fluoro-4-oxo-1,4-dihydro-quinoline-3-carboxylic acid ethyl ester (600 mg, 1.04 mmol, prepared according to procedures described in WO20051.13509), 1-ethynyl-2,4-difluorobenzene (172 mg, 1.25 mmol), copper(I) iodide (10 mg, 0.05 mmol) and bis(triphenylphosphine)palladium(II) dichloride (35 mg, 0.05 mmol) in triethylamine (20 mL) was heated at 100° C. under argon atmosphere for 24 hours. After cooling to room temperature and removal of the solvent, the residue was diluted with water and extracted with dichloromethane. The combined organic extracts were dried over anhydrous sodium sulfate, filtered and concentrated in vacuo. The residue was purified by ISCO (Hexane/EtOAc, 0%, 10 min; 0-30%, 20 min; 30-80%, 10 min) to afford the product as an oil (0.6 g, 98%).

¹H NMR (CDCl₃, 400 MHz): δ 8.77 (d, J=8.2 Hz, 1H), 8.69 (s, 1H), 7.58 (m, 1H), 7.39 (d, J=11.7 Hz, 1H), 6.93 (m, 2H), 4.51-4.42 (m, 3H), 4.21-4.11 (m, 2H), 1.44 (t, J=7.1 Hz, 3H), 1.10 (s, 9H), 0.03 (s, 6H).

MS: 586 (M+1).

### Step B: 1-[(S)-1-(tert-Butyl-dimethyl-silanyloxymethyl)-2,2-dimethyl-propyl]-6-[2-(2,4-difluoro-phenyl)-ethyl]-7-fluoro-4-oxo-1,4-dihydro-quinoline-3-carboxylic acid ethyl ester

Pd—C (10%, 100 mg) was added to a solution of 1-[(S)-1-(tert-butyl-dimethyl-silanyloxymethyl)-2,2-dimethyl-propyl]-6-(2,4-difluoro-phenylethynyl)-7-fluoro-4-oxo-1,4-dihydro-quinoline-3-carboxylic acid ethyl ester (400 mg, 0.7 mmol) in methanol (50 mL). The mixture was hydrogenated at room temperature under normal pressure for 10 hours and then filtered through Celite. After washing with methanol, the filtrate was evaporated to dryness to give the desired product in a quantitative yield.

MS: 590 (M+1).

### Step C: 6-[2-(2,4-Difluoro-phenyl)-ethyl]-1-((S)-1-hydroxymethyl-2,2-dimethyl-propyl)-7-methoxy-4-oxo-1,4-dihydro-quinoline-3-carboxylic acid

1-[(S)-1-(tert-Butyl-dimethyl-silanyloxymethyl)-2,2-dimethyl-propyl]-6-[2-(2,4-difluoro-phenyl)-ethyl]-7-fluoro-4-oxo-1,4-dihydro-quinoline-3-carboxylic acid ethyl ester (200 mg, 0.34 mmol) was dissolved in 10 mL of 28% sodium methoxide in methanol and water (0.5 mL) and heated at reflux overnight. After cooling to room temperature, the reaction mixture was concentrated to a small volume under reduced pressure, and water (20 mL) added. The mixture was filtered and the filtrate neutralized with 6 N hydrochloric acid. The resulting solid was collected and washed with water to give the pure product as a white solid (100 mg).

¹H NMR (DMSO-d₆, 400 MHz): δ 15.57 (brs, 1H, OH, exchangeable with D₂O), 8.80 (s, 1H), 8.13 (s, 1H), 7.51 (s, 1H), 7.35 (dt, J=8.6 and 6.8 Hz, 1H), 7.18 (ddd, J=0.9, 2.5 and 9.5 Hz, 1H), 7.01 (ddd, J=0.9, 2.5 and 8.6 Hz, 1H), 5.20 (m, 1H), 5.17 (brs, 1H, OH, exchangeable with D₂O), 4.11 (m, 2H), 4.04 (s, 3H), 2.97 (m, 4H), 1.00 (s, 9H).

MS: 460 (M+1).

### Example 19

### Compounds of Formula (XIX)

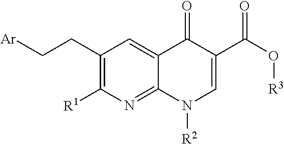

Compounds of formula (XIX) were prepared according to the following general synthetic scheme. When appropriate, protecting groups are used as needed according to established synthetic procedures known to those of skill in the art, and may or may not be removed upon completion of the synthesis. Starting materials are synthesized according to methods known in the art or are commercially available.

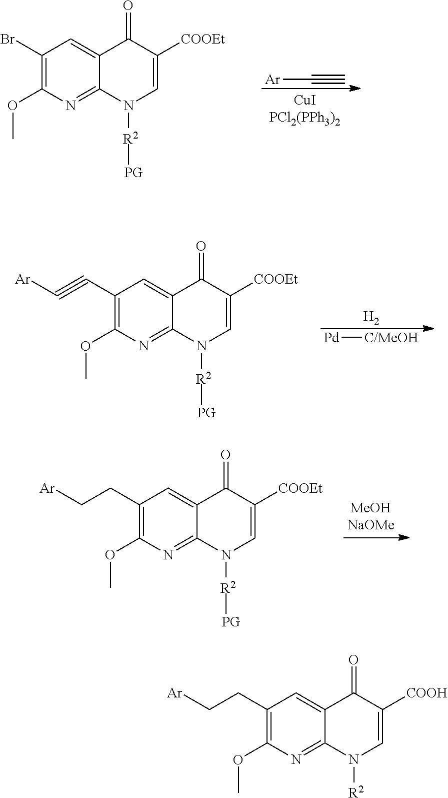

### Example 19A

### (S)-6-(2,4-difluorophenethyl)-1-(1-hydroxy-3-methylbutan-2-yl)-7-methoxy-4-oxo-1,4-dihydro-1,8-naphthyridine-3-carboxylic acid

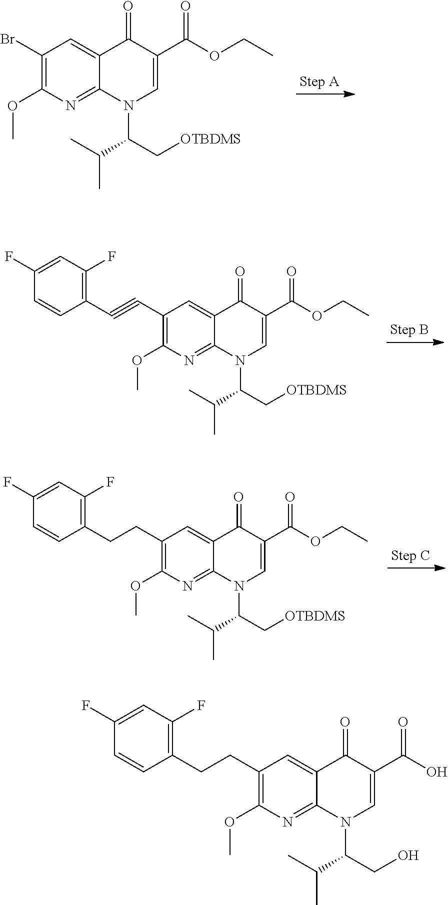

### Step A and B: 1-[(S)-1-(tert-Butyl-dimethyl-silanyloxymethyl)-2-methyl-propyl]-6-[2-(2,4-difluoro-phenyl)-ethyl]-7-methoxy-4-oxo-1,4-dihydro-1,8-naphthyridine-3-carboxylic acid ethyl ester

6-Bromo-1-[(S)-1-(tort-butyl-dimethyl-silanyloxymethyl)-2-methyl-propyl]-7-methoxy-4-oxo-1,4-dihydro-1,8-naphthyridine-3-carboxylic acid ethyl ester (550 mg, 1.04 mmol), 1-ethynyl-2,4-difluorobenzene (172 mg, 1.25 mmol), copper(I) iodide (10 mg, 0.05 mmol), and bis(triphenylphosphine)palladium(II) dichloride (35 mg, 0.05 mmol) in triethylamine (20 mL) was heated at 100° C. under argon atmosphere for 24 hours. After cooling at room temperature and removal of solvent, the residue was diluted with water and extracted with dichloromethane. The combined organic extracts were dried over anhydrous sodium sulfate, filtered and concentrated in vacuo. The residue was purified by ISCO (Hexane/EtOAc, 0%, 10 min; 0-30%, 20 min; 30-80%, 10 min) to afford a mixture of starting material and product (73%:22%) as an oil. The mixture was dissolved in methanol (30 mL) and Pd—C (10%, 50 mg) was added. The mixture was hydrogenated at room temperature under normal pressure for 10 hours and then filtered through Celite After washing with methanol, the filtrate was evaporated to dryness and purified by ISCO (Hexane/EtOAc, 0%, 10 min; 0-30%, 20 min; 30-80%, 10 min) to afford the desired product as an oil.

¹H NMR (CDCl₃, 400 MHz): δ 8.77 (s, 1H), 8.46 (s, 1H), 7.11 (m, 1H), 6.78 (m, 2H), 5.35 (m, 1H), 4.40 (m, 2H), 4.11 (dd, J=4.5 and 11.1 Hz, 1H), 4.03 (s, 3H), 3.85 (dd, J=2.4 and 11.1 Hz, 1H), 2.96 (m, 4H), 2.46 (m, 1H), 1.42 (t, J=7.1 Hz, 3H), 1.20 (d, J=6.5 Hz, 3H), 0.85 (s, 9H), 0.83 (d, J=6.5 Hz, 3H), 0.03 (s, 6H).

MS: 589 (M+1).

### Step C: 6-[2-(2,4-Difluoro-phenyl)-ethyl]-1-((S)-1-hydroxymethyl-2-methyl-propyl)-7-methoxy-4-oxo-1,4-dihydro-1,8-naphthyridine-3-carboxylic acid

Sodium methoxide in methanol (28%, 1 mL) and water (1 mL) were added to a solution of 1-[(S)-1-(tert-butyl-dimethyl-silanyloxymethyl)-2-methyl-propyl]-6-[2-(2,4-difluoro-phenyl)-ethyl]-7-methoxy-4-oxo-1,4-dihydro-1,8-naphthyridine-3-carboxylic acid ethyl ester in methanol (10 mL) and the mixture heated at reflux for 5 hours. After cooling to room temperature, the reaction mixture was concentrated to a small volume under reduced pressure, and water (20 mL) added. The mixture was filtered and the filtrate neutralized with 6 N hydrochloric acid. The resulting solid was collected and washed with water to give the pure product as a white solid.

¹H NMR (DMSO-d₆, 400 MHz): δ 15.29 (brs, 1H, OH, exchangeable with D₂O), 9.00 (s, 1H), 8.38 (s, 1H), 7.38 (dt, J=8.6 and 6.8 Hz, 1H), 7.18 (ddd, J=0.9, 2.5 and 9.5 Hz, 1H), 7.03 (ddd, J=0.9, 2.5 and 8.6 Hz, 1H), 5.50 (m, 1H), 5.23 (brs, 1H, OH, exchangeable with D₂O), 4.08 (s, 3H), 4.04 (m, 1H), 3.82 (m, 1H), 3.00 (m, 4H), 2.36 (m, 1H), 1.14 (d, J=6.5 Hz, 3H), 0.73 (d, J=6.5 Hz, 3H).

MS: 447 (M+1).

### Example 20

### Compounds of Formula (XX)

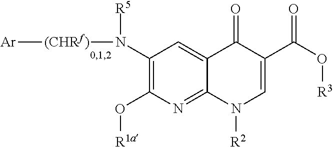

Compounds of formula (XX) were prepared according to the following synthetic scheme.

When appropriate, protecting groups are used as needed according to established synthetic procedures known to those of skill in the art, and may or may not be removed upon completion of the synthesis. The individual starting materials are synthesized according to methods known in the art or are commercially available.

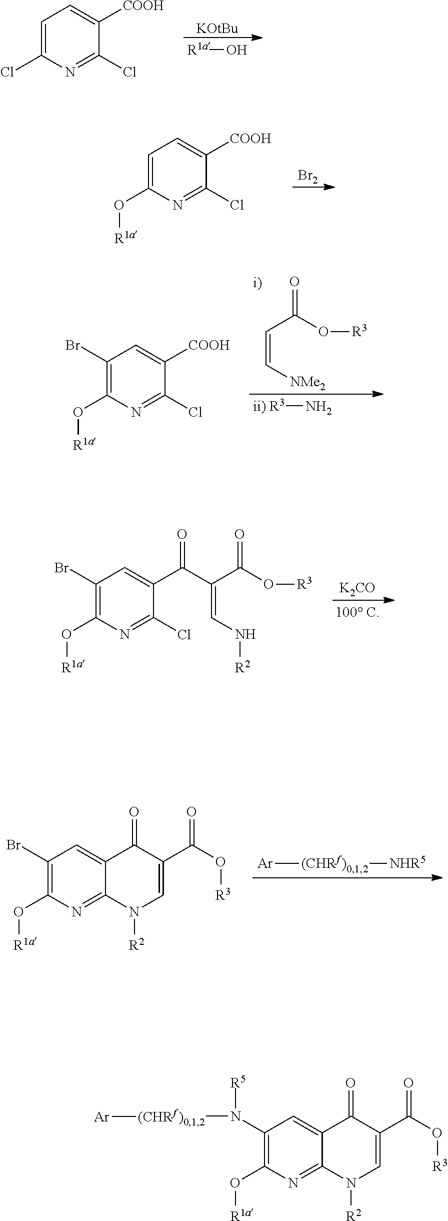

### Example 20A

### (S)-1-(1-hydroxy-3-methylbutan-2-yl)-7-methoxy-6-(4-methylbenzylamino)-4-oxo-1,4-dihydro-1,8-naphthyridine-3-carboxylic acid

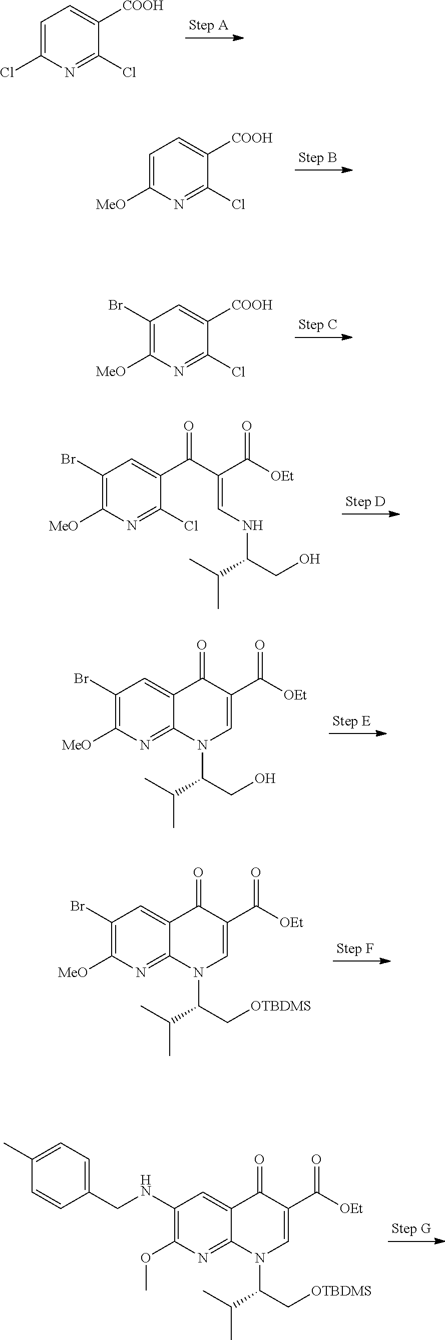

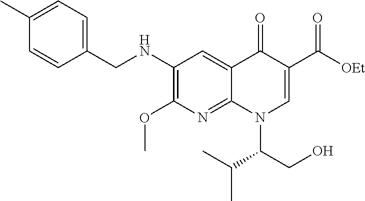

### Step A: 2-Chloro-6-methoxypyridine-3-carboxylic acid

A mixture of 2,6-dichloropyridine-3-carboxylic acid (6.5 g, 33 mmol), potassium tert-butoxide (11.4 g, 0.10 mol), and anhydrous methanol (300 mL) was heated to reflux for 4 days and cooled to room temperature. After evaporation of the solvent, the residue was diluted with water and acidified with 35% aqueous hydrochloric acid. The resulting solid was collected by filtration, washed with water, and dried to give 4.8 g (84%) of 2-chloro-6-methoxypyridine-3-carboxylic acid as a white solid.

1H NMR (DMSO-d6, 400 MHz): δ 13.33 (brs, 1H, OH, exchangeable with D2O), 8.19 (d, J=8.5 Hz, 1H), 6.92 (d, J=8.5 Hz, 1H), 3.92 (s, 3H).

### Step B: 2-Chloro-5-bromo-6-methoxypyridine-3-carboxylic acid

To a suspension of 2-chloro-6-methoxypyridine-3-carboxylic acid (4.69 g, 25 mmol) and sodium acetate (4.10 g, 50 mmol) in 200 ml of glacial acetic acid was added bromine (16.0, 100 mmol) at room temperature. The mixture was warmed to 80° C. overnight, cooled to room temperature and poured into 500 ml of ice-water with strong stirring. The solid was filtered and washed with water to give 5.2 g (78%) of pure product as a white solid.

¹H NMR (DMSO-d₆, 400 MHz): δ 8.51 (s, 1H), 3.93 (s, 3H).

MS: 266 (M−1).

### Step C: 2-(5-Bromo-2-chloro-6-methoxy-pyridine-3-carbonyl)-3-((S)-1-hydroxymethyl-2-methyl-propylamino)-acrylic acid ethyl ester

A mixture of 2-chloro-5-bromo-6-methoxypyridine-3-carboxylic acid (8.0 g, 30 mmol) and thionyl chloride-(4.4 mL, 60 mmol) in 50 ml of anhydrous toluene and 0.5 ml of anhydrous DMF was refluxed for 2 h. The solvent was removed under reduced pressure to give a mobile oil residue which was azeoptoped with toluene (20 mL). The residue was dissolved in 20 ml of anhydrous THF. This solution was added dropwise to a solution of ethyl 3-(dimethylamino)acrylate (4.7 g, 33 mmol) and triethylamine (3.64 g, 36 mmol) in 20 ml of anhydrous THF under nitrogen and heated under reflux for 7 hours The mixture was allowed to cool to room temperature and concentrated under reduced pressure. Water (100 mL) and ethyl acetate (100 mL) was added to allow partitioning. The organic layer was washed with saturated aqueous sodium bicarbonate (×2), water, brine, dried over sodium sulfate and concentrated under reduced pressure. The crude product was purified by flash chromatography (ISCO, chloroform/methanol, 0-40%, 40 min) to give the pure product as yellow oil (7.3 g, 62%).

A solution of the above product (7.3 g, 18.6 mmol) and L-valinol (1.92 g, 18.6 mmol) in anhydrous THF (100 mL) was stirred for 30 min at room temperature and evaporated to dryness to give a crude product in a quantitative yield, which was used for next step without further purification. An analytically pure sample was prepared by silica gel chromatography (ISCO, Chloroform/methanol, 0-40%, 40 min) to give the pure compound as yellow oil.

¹H NMR (DMSO-d₆, 400 MHz): δ 10.95 (dd, J=9.6 and 13.8 Hz, 1H, NH, exchangeable with D₂O), 8.24 (d, J=14.3 Hz, 1H, it becomes singlet after D₂O exchange), 7.98 (s, 1H), 5.05 (t, J=5.1 Hz, 1H, OH, exchangeable with D₂O), 3.95 (s, 3H), 3.91 (q, J=7.0 Hz, 2H), 3.59 (m, 2H), 3.36 (m, 1H), 1.93 (m, 1H), 0.95 (d, J=6.6 Hz, 3H), 0.91 (d, J=6.6 Hz, 3H), 0.90 (t, J=7.0 Hz, 3H).

MS: 449, 451 (M+1).

### Step D: 6-Bromo-1-((S)-1-hydroxymethyl-2-methyl-propyl)-7-methoxy-4-oxo-1,4-dihydro-[1,8]naphthyridine-3-carboxylic acid ethyl ester

A mixture of 2-(5-bromo-2-chloro-6-methoxy-pyridine-3-carbonyl)-3-((S)-1-hydroxymethyl-2-methyl-propylamino)-acrylic acid ethyl ester (1.1 g, 2.5 mmol) and potassium carbonate (0.7 g, 5.0 mmol) in anhydrous DMF (15 mL) was stirred at 100° C. for 2 hours and evaporated to dryness under reduced pressure. The crude material was purified by ISCO (Chloroform/methanol, 0-40%, 40 min) to give the title compound as a yellow solid (0.7 g, 68%). 1H NMR (DMSO-d6, 400 MHz): δ 8.73 (s, 1H), 8.58 (s, 1H), 5.25 (m, 1H), 5.11 (brs, 1H, OH, exchangeable with D2O), 4.24 (q, J=7.1 Hz, 2H), 4.08 (s, 3H), 3.94 (m, 1H), 3.91 (m, 1H), 2.27 (m, 1H), 1.28 (t, J=7.1 Hz, 3H), 1.10 (d, J=6.21 Hz, 3H), 0.74 (d, J=6.2 Hz, 3H).

MS: 413, 415 (M+1).

### Step E: 6-Bromo-1-[(S)-1-(tert-butyl-dimethyl-silanyloxymethyl)-2-methyl-propyl]-7-methoxy-4-oxo-1,4-dihydro-[1,8]naphthyridine-3-carboxylic acid ethyl ester

To a mixture of 6-bromo-1-((S)-1-hydroxymethyl-2-methyl-propyl)-7-methoxy-4-oxo-1,4-dihydro-[1,8]naphthyridine-3-carboxylic acid ethyl ester (0.63 g, 1.5 mmol) and imidazole (1.04 g, 15.0 mmol) in 12 ml of anhydrous DMF was added tert-butyldimethylsilyl chloride (1.28 g, 7.5 mmol) under argon at room temperature. The resulting mixture, was stirred at room temperature overnight and evaporated to dryness under reduced pressure. The resulting crude material was purified by ISCO (hexane/EtOAc, 0-90%, 40 min) to give the title compound as yellow oil (0.7 g, 89%).

1H NMR (DMSO-d6, 400 MHz): δ 8.72 (s, 1H), 8.61 (s, 1H), 5.33 (m, 1H), 4.26 (q, J=7.1 Hz, 2H), 4.07 (s, 3H), 4.05 (m, 1H), 3.94 (m, 1H), 2.36 (m, 1H), 1.30 (t, J=7.1 Hz, 3H), 1.16 (d, J=6.2 Hz, 3H), 0.79 (d, J=6.2 Hz, 3H), 0.77 (s, 9H), 0.02 (s,

MS: 527, 529 (M+1).

### Step F: (S)-ethyl 1-(1-(tert-butyldimethylsilyloxy)-3-methylbutan-2-yl)-7-methoxy-6-(4-methylbenzylamino)-4-oxo-1,4-dihydro-1,8-naphthyridine-3-carboxylate

A solution of (S)-ethyl 6-bromo-1-(1-(tert-butyldimethylsilyloxy)-3-methylbutan-2-yl)-7-methoxy-4-oxo-1,4-dihydro-1,8-naphthyridine-3-carboxylate (300 mg, 0.569 mmol), p-tolylmethanamine (200 mg, 11.646 mmol), Pd(OAc)₂ (15 mg, 0.067 mmol), BINAP (85 mg, 0.137 mmol), and Cs₂CO₃ in dioxane was degassed by bubbling nitrogen for 30 min then heated at 80° C. over night. The reaction mixture was diluted with EtOAc (30 mL), washed with H₂O (2×10 mL), and dried over Na₂SO₄. The solvent was removed under reduced pressure and purified on silica gel column (20-40% EtOAc/hexanes) to yield the desired product as pale yellow foam (260 mg, 80%). NMR (CDCl3): □8.69 (s, 1H), 7.75 (s, 1H), 7.30 (d, 2H), 7.18 (d, 2H), 5.30 (m, 1H), 4.62 (t, 1H), 4.39 (m, 4H), 4.11 (m, 1H), 4.07 (s, 3H), 3.83 (dd, 1H), 2.45 (m, 1H), 2.36 (s, 3H), 1.42 (t, 3H), 1.17 (d, 3H), 0.84 (s, 9H), 0.80 (d, 3H), 0.00 (s, 6H); MS (ESI): m/z 568 (M+1)⁺.

### Step G: (S)-1-(1-hydroxy-3-methylbutan-2-yl)-7-methoxy-6-(4-methylbenzylamino)-4-oxo-1,4-dihydro-1,8-naphthyridine-3-carboxylic acid

A solution of (S)-ethyl 1-(1-(tert-butyldimethylsilyloxy)-3-methylbutan-2-yl)-7-methoxy-6-(4-methylbenzylamino)-4-oxo-1,4-dihydro-1,8-naphthyridine-3-carboxylate (100 mg, 0.176 mmol), NaOCH₃ (1.0 mL, 25% in MeOH), and H₂O (1.0 mL) in MeOH (3.0 mL) was heated at 60° C. for 4 h. The reaction mixture was concentrated under reduced pressure to small volume and diluted with H₂O (10 mL). The pH of the solution was adjusted to 4 with HCl (1 N) and resulting precipitate was collected by filtration to yield the desired compound as an off-white solid (62 mg, 83%). NMR (DMSO-d₆): δ 8.80 (s, 1H), 7.28 (d, 2H), 7.15 (m, 3H), 6.87 (m, 1H), 5.45 (m, 1H), 5.12 (m, 1H), 4.42 (d, 2H), 4.15 (s, 3H), 4.00 (m, 1H), 3.80 (m, 1H), 2.32 (m, 1H), 2.25 (s, 3H), 1.10 (d, 3H), 0.70 (d, 3H); MS (ESI): m/z 426 (M+1)⁺.

### Examples 20B-20SS

Examples 20B-20SS were prepared according to the procedure described above for example 20A, using the appropriate amine indicated.

| | | | | ¹H NMR (400 MHz) |
|---|---|---|---|---|
| | | | | 25° C. δ |
| | | | | ^{a)} CD₃OD ^{b)} CDCl₃ |
| Com- | Compound | | | ^{c)} d6-DMSO |
| pound | Name | Amine starting material | Structure | MS (ESI) |
| 20A | (S)-1-(1- hydroxy-3- methylbutan- 2-yl)-7- methoxy- 6-(4-methyl- benzylamino)- 4-oxo-1,4- dihydro-1,8- naphthyridine- 3-carboxylic acid | | 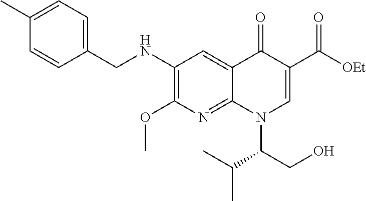 | ^{c)} 8.80 (s, 1H), 7.28 (d, 2H), 7.15 (m, 3H), 6.87 (m, 1H), 5.45 (m, 1H), 5.12 (m, 1H), 4.42 (d, 2H), 4.15 (s, 3H), 4.00 (m, 1H), 3.80 (m, 1H), 2.32 (m, 1H), 2.25 (s, 3H), 1.10 (d, 3H), 0.70 (d, 3H); MS (ESI): m/z 426 (M + 1)⁺ |
| 20B | (S)-6-(3- chloro-2- fluorobenzyl- amino)-1-(1- hydroxy-3- methylbutan- 2-yl)-7- methoxy- 4-oxo-1,4- dihydro-1,8- naphthyridine- 3-carboxylic acid | 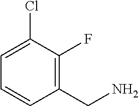 | 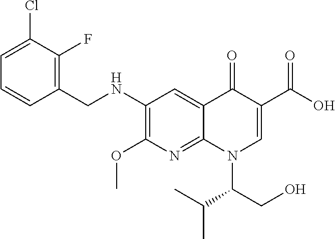 | ^{c)} 15.70 (s, 1H), 8.80 (s, 1H), 7.50 (t, 1H), 7.28 (d, 1H), 7.32 (t, 1H), 7.20 (m, 1H), 6.90 (t, 1H), 5.45 (m, 1H), 5.18 (m, 1H), 4.58 (d, 2H), 4.18 (s, 3H), 4.00 (m, 1H), 3.80 (m, 1H), 2.45 (m, 1H), 1.12 (d, 3H), 0.70 (d, 3H); MS (ESI): m/z 464 (M + 1)⁺. |
| 20C | (S)-6-(4- fluorobenzyl- amino)-1-(1- hydroxy-3- methylbutan- 2-yl)-7- methoxy- 4-oxo-1,4- dihydro-1,8- naphthyridine- 3-carboxylic acid | 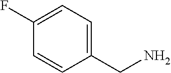 | 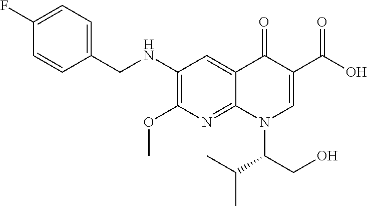 | ^{c)} 15.70 (s, 1H), 8.80 (s, 1H), 7.42 (m, 2H), 7.17 (m, 3H), 6.95 (t, 1H), 5.45 (m, 1H), 5.18 (m, 1H), 4.48 (d, 2H), 4.18 (s, 3H), 4.00 (m, 1H), 3.90 (m, 1H), 2.35 (m, 1H), 1.12 (d, 3H), 0.70 (d, 3H); MS (ESI): m/z 430 (M + 1)⁺. |
| 20D | (S)-6-(2- fluorobenzyl- amino)-1-(1- hydroxy-3- methylbutan- 2-yl)-7- methoxy- 4-oxo-1,4- dihydro-1,8- naphthyridine- 3-carboxylic acid | 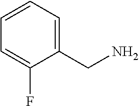 | 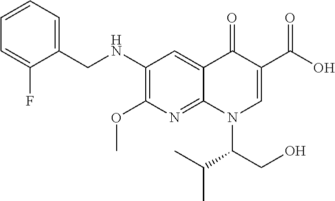 | ^{c)} 15.70 (s, 1H), 8.80 (s, 1H), 7.45 (m, 2H), 7.27 (m, 1H), 7.22 (s, 1H), 7.18 (t, 1H), 6.98 (t, 1H), 5.47 (m, 1H), 5.18 (m, 1H), 4.52 (d, 2H), 4.18 (s, 3H), 4.05 (m, 1H), 3.80 (m, 1H), 2.35 (m, 1H), 1.15 (d, 3H), 0.70 (d, 3H); MS (ESI): m/z 430 (M + 1)⁺. |
| 20E | (S)-6-(3- fluorobenzyl- amino)-1-(1- hydroxy-3- methylbutan- 2-yl)-7- methoxy- 4-oxo-1,4- dihydro-1,8- naphthyridine- 3-carboxylic acid | 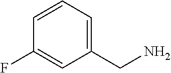 | 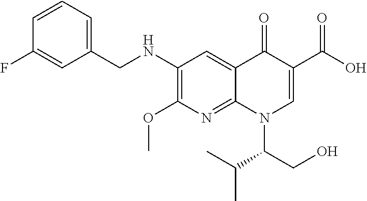 | ^{c)} 15.70 (s, 1H), 8.80 (s, 1H), 7.42 (m, 1H), 7.25 (m, 2H), 7.15 (s, 1H), 7.10 (t, 1H), 6.98 (t, 1H), 5.45 (m, 1H), 5.18 (m, 1H), 4.52 (d, 2H), 4.18 (s, 3H), 4.05 (m, 1H), 3.80 (m, 1H), 2.35 (m, 1H), 1.15 (d, 3H), 0.70 (d, 3H); MS (ESI): m/z 430 (M + 1)⁺. |
| 20F | (S)-6-(3- chloro-4- fluorobenzyl- amino)-1-(1- hydroxy-3- methylbutan- 2-yl)-7- methoxy- 4-oxo-1,4- dihydro-1,8- naphthyridine- 3-carboxylic acid | 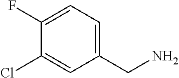 | 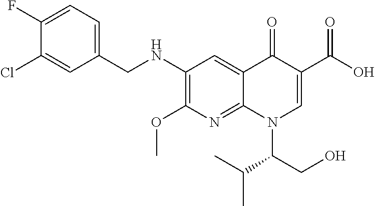 | ^{c)} 15.70 (s, 1H), 8.81 (s, 1H), 7.62 (d, 1H), 7.42 (m, 2H), 7.18 (s, 1H), 6.98 (br s, 1H), 5.45 (m, 1H), 5.18 (m, 1H), 4.48 (d, 2H), 4.18 (s, 3H), 4.02 (m, 1H), 3.80 (m, 1H), 2.35 (m, 1H), 1.12 (d, 3H), 0.72 (d, 3H); MS (ESI): m/z 464 (M + 1)⁺. |
| 20G | (S)-6-(3- chloro- phenethyl- amino)-1-(1- hydroxy-3- methylbutan- 2-yl)-7- methoxy- 4-oxo-1,4- dihydro-1,8- naphthyridine- 3-carboxylic acid | 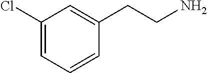 | 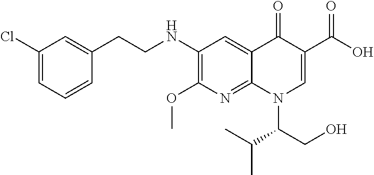 | ^{c)} 15.70 (s, 1H), 8.83 (s, 1H), 7.40 (m, 2H), 7.38 (m, 1H), 7.30 (m, 2H), 6.15 (t, 1H), 5.48 (m, 1H), 5.20 (m, 1H), 4.16 (s, 3H), 4.15 (m, 1H), 3.85 (m, 1H), 3.50 (q, 2H), 2.98 (t, 2H), 2.35 (m, 1H), 1.12 (d, 3H), 0.72 (d, 3H); MS (ESI): m/z 460 (M + 1)⁺. |
| 20H | (S)-6-(4- chloro-3- fluorobenzyl- amino)-1-(1- hydroxy-3- methylbutan- 2-yl)-7- methoxy- 4-oxo-1,4- dihydro-1,8- naphthyridine- 3-carboxylic acid | 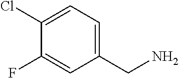 | 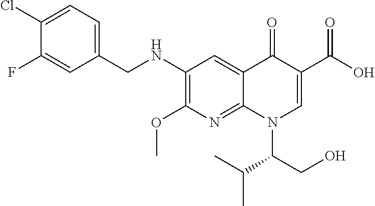 | ^{c)} 15.70 (s, 1H), 8.81 (s, 1H), 7.58 (t, 1H), 7.45 (dd, 1H), 7.18 (dd, 1H), 7.15 (s, 1H), 7.00 (t, 1H), 5.45 (m, 1H), 5.18 (m, 1H), 4.52 (d, 2H), 4.18 (s, 3H), 4.02 (m, 1H), 3.80 (m, 1H), 2.35 m, 1H), 1.12 (d, 3H), 0.72 (d, 3H); MS (ESI): m/z 464 (M + 1)⁺. |
| 20I | (S)-6-(3- chlorobenzyl- amino)-1-(1- hydroxy-3- methylbutan- 2-yl)-7- methoxy- 4-oxo-1,4- dihydro-1,8- naphthyridine- 3-carboxylic acid | 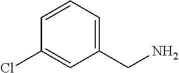 | 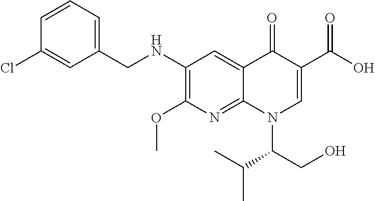 | ^{c)} 15.70 (s, 1H), 8.80 (s, 1H), 7.47 (s, 1H), 7.38 (m, 3H), 7.18 (s, 1H), 6.95 (m, 1H), 5.45 (m, 1H), 5.18 (m, 1H), 4.50 (d, 2H), 4.18 (s, 3H), 4.02 (m, 1H), 3.80 (m, 1H), 2.35 (m, 1H), 1.12 (d, 3H), 0.72 (d, 3H); MS (ESI): m/z 446 (M + 1)⁺. |
| 20J | (S)-6-(4- chlorobenzyl- amino)-1-(1- hydroxy-3- methylbutan- 2-yl)-7- methoxy- 4-oxo-1,4- dihydro-1,8- naphthyridine- 3-carboxylic acid | 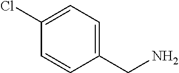 | 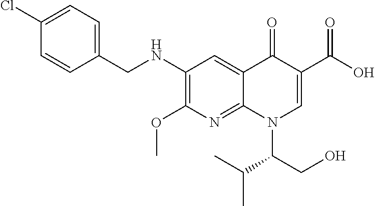 | ^{c)} 15.70 (s, 1H), 8.80 (s, 1H), 7.42 (m, 4H), 7.15 (s, 1H), 6.98 (t, 1H), 5.47 (m, 1H), 5.15 (m, 1H), 4.50 (d, 2H), 4.18 (s, 3H), 4.02 (m, 1H), 3.80 (m, 1H), 2.35 (m, 1H), 1.12 (d, 3H), 0.72 (d, 3H); MS (ESI): m/z 446 (M + 1)⁺. |
| 20K | (S)-6-(3,4- dichloro- benzyl- amino)-1-(1- hydroxy-3- methylbutan- 2-yl)-7- methoxy- 4-oxo-1,4- dihydro-1,8- naphthyridine- 3-carboxylic acid | 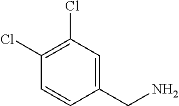 | 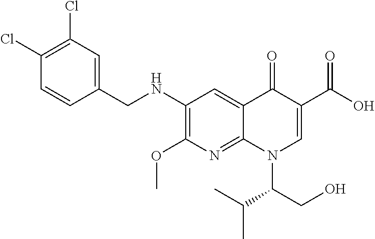 | ^{c)} 15.70 (s, 1H), 8.78 (s, 1H), 7.68 (s, 1H), 7.62 (d, 1H), 7.38 (dd, 1H), 7.18 (s, 1H), 6.90 (br, 1H), 5.42 (m, 1H), 5.13 (m, 1H), 4.50 (d, 2H), 4.18 (s, 3H), 3.98 (m, 1H), 3.80 (m, 1H), 2.30 (m, 1H), 1.12 (d, 3H), 0.72 (d, 3H); MS (ESI): m/z 480 (M + 1)⁺. |
| 20L | (S)-6-(2,3- dichloro- benzyl- amino)-1-(1- hydroxy-3- methylbutan- 2-yl)-7- methoxy- 4-oxo-1,4- dihydro-1,8- naphthyridine- 3-carboxylic acid | 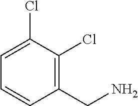 | 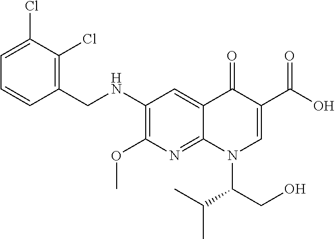 | ^{c)} 15.70 (s, 1H), 8.82 (s, 1H), 7.59 (dd, 1H), 7.32 (t, 1H), 7.30 (dd, 1H), 7.07 (s, 1H), 7.01 (t, 1H), 5.49 (m, 1H), 5.18 (m, 1H), 4.57 (d, 2H), 4.20 (s, 3H), 4.03 (m, 1H), 3.82 (m, 1H), 2.35 (m, 1H), 1.14 (d, 3H), 0.72 (d, 3H); MS (ESI): m/z 480 (M + 1)⁺. |
| 20M | (S)-6-(2- chlorobenzyl- amino)-1-(1- hydroxy-3- methylbutan- 2-yl)-7- methoxy- 4-oxo-1,4- dihydro-1,8- naphthyridine- 3-carboxylic acid | 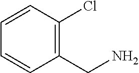 | 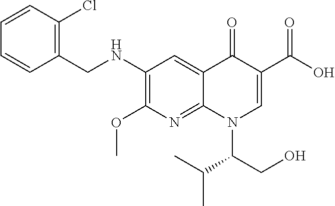 | ^{c)} 15.70 (s, 1H), 8.80 (s, 1H), 7.55 (m, 1H), 7.33 (m, 3H), 7.18 (s, 1H), 6.93 (br s, 1H), 5.47 (m, 1H), 5.18 (m, 1H), 4.55 (d, 2H), 4.18 (s, 3H), 4.02 (m, 1H), 3.82 (m, 1H), 2.35 (m, 1H), 1.15 (d, 3H), 0.72 (d, 3H); MS (ESI): m/z 446 (M + 1)⁺. |
| 20N | (S)-6-(3,4- difluoro- benzyl- amino)-1-(1- hydroxy-3- methylbutan- 2-yl)-7- methoxy- 4-oxo-1,4- dihydro-1,8- naphthyridine- 3-carboxylic acid | 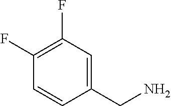 | 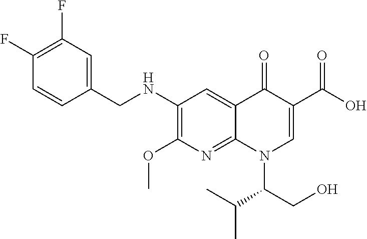 | ^{c)} 15.70 (s, 1H), 8.80 (s, 1H), 7.42 (m, 2H), 7.25 (m, 1H), 7.18 (s, 1H), 6.90 (br, 1H), 5.45 (m, 1H), 5.17 (m, 1H), 4.47 (d, 2H), 4.18 (s, 3H), 4.02 (m, 1H), 3.80 (m, 1H), 2.35 (m, 1H), 1.15 (d, 3H), 0.72 (d, 3H); MS (ESI): m/z 448 (M + 1)⁺. |
| 20O | (S)-6-(benzyl- amino)-1-(1- hydroxy-3- methylbutan- 2-yl)-7- methoxy- 4-oxo-1,4- dihydro-1,8- naphthyridine- 3-carboxylic acid | 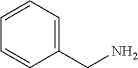 | 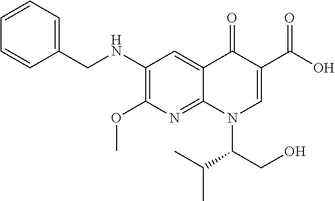 | ^{c)} 15.70 (s, 1H), 8.80 (s, 1H), 7.38 (m, 4H), 7.25 (m, 1H), 7.18 (s, 1H), 6.93 (t, 1H), 5.47 (m, 1H), 5.17 (m, 1H), 4.49 (d, 2H), 4.18 (s, 3H), 4.02 (m, 1H), 3.80 (m, 1H), 2.35 (m, 1H), 1.13 (d, 3H), 0.72 (d, 3H); MS (ESI): m/z 412 (M + 1)⁺. |
| 20P | (S)-6-(6- fluoro- 3,4-dihydro- isoquinolin- 2(1H)-yl)-1- (1-hydroxy-3- methylbutan- 2-yl)-7- methoxy- 4-oxo-1,4- dihydro-1,8- naphthyridine- 3-carboxylic acid | 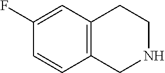 | 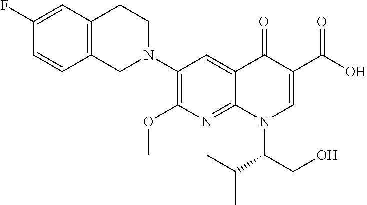 | ^{c)} 15.70 (s, 1H), 8.95 (s, 1H), 7.93 (s, 1H), 7.32 (m, 1H), 7.18 (m, 2H), 5.50 (m, 1H), 5.20 (m, 1H), 4.40 (s, 2H), 4.18 (s, 3H), 4.05 (m, 1H), 3.85 (m, 1H), 3.53 (t, 2H), 2.97 (t, 2H), 2.38 (m, 1H), 1.15 (d, 3H), 0.72 (d, 3H); MS (ESI): m/z 456 (M + 1)⁺. |
| 20Q | (S)-6-(7- fluoro-3,4- dihydro- isoquinolin- 2(1H)-yl)-1- (1-hydroxy-3- methylbutan- 2-yl)-7- methoxy- 4-oxo-1,4- dihydro-1,8- naphthyridine- 3-carboxylic acid | 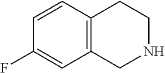 | 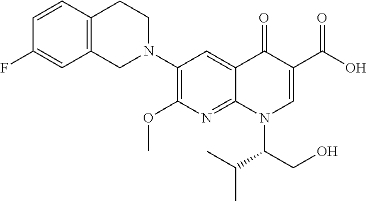 | ^{c)} 15.70 (s, 1H), 8.95 (s, 1H), 7.93 (s, 1H), 7.25 (dd, 1H), 7.18 (dd, 1H), 7.05 (m, 1H), 5.50 (m, 1H), 4.42 (s, 2H), 4.18 (s, 3H), 4.05 (m, 1H), 3.85 (m, 1H), 3.60 (m, 1H), 3.53 (t, 2H), 2.95 (t, 2H), 2.38 (m, 1H), 1.15 (d, 3H), 0.72 (d, 3H); MS (ESI): m/z 456 (M + 1)⁺. |
| 20R | (S)-6-(2,4- difluoro- benzyl- amino)-1-(1- hydroxy-3- methylbutan- 2-yl)-7- methoxy- 4-oxo-1,4- dihydro-1,8- naphthyridine- 3-carboxylic acid | 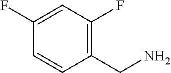 | 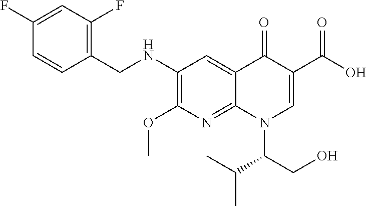 | ^{c)} 15.70 (s, 1H), 8.82 (s, 1H), 7.42 (q, 1H), 7.32 (m, 1H), 7.22 (s, 1H), 7.15 (m, 1H), 6.85 (t, 1H), 5.47 (m, 1H), 5.18 (m, 1H), 4.50 (d, 2H), 4.18 (s, 3H), 4.03 (m, 1H), 3.80 (m, 1H), 2.35 (m, 1H), 1.14 (d, 3H), 0.72 (d, 3H); MS (ESI): m/z 448 (M + 1)⁺. |
| 20S | (S)-6-(2-fluoro- phenethyl- amino)-1-(1- hydroxy-3- methylbutan- 2-yl)-7- methoxy-4- oxo-1,4- dihydro-1,8- naphthyridine- 3-carboxylic acid | 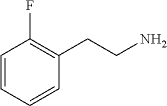 | 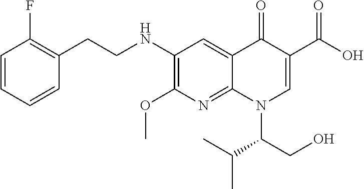 | ^{c)} 15.70 (s, 1H), 8.83 (s, 1H), 7.43 (s, 1H), 7.40 (t, 1H), 7.30 (m, 1H), 7.20 (dd, 2H), 6.28 (t, 1H), 5.48 (m, 1H), 5.18 (m, 1H), 4.16 (s, 3H), 4.05 (m, 1H), 3.83 (m, 1H), 3.45 (q, 2H), 2.98 (t, 2H), 2.35 (m, 1H), 1.15 (d, 3H), 0.72 (d, 3H); MS (ESI): m/z 444 (M + 1)⁺. |
| 20T | (S)-6-(4-fluoro- phenethyl- amino)-1-(1- hydroxy-3- methylbutan- 2-yl)-7- methoxy-4- oxo-1,4- dihydro-1,8- naphthyridine- 3-carboxylic acid | 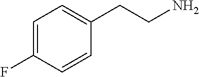 | 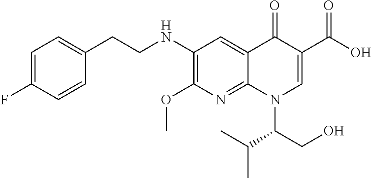 | ^{c)} 15.70 (s, 1H), 8.83 (s, 1H), 7.35 (m, 3H), 7.15 (m, 2H), 6.15 (t, 1H), 5.48 (m, 1H), 5.18 (m, 1H), 4.16 (s, 3H), 4.05 (m, 1H), 3.82 (m, 1H), 3.45 (q, 2H), 2.95 (t, 2H), 2.35 (m, 1H), 1.15 (d, 3H), 0.72 (d, 3H); MS (ESI): m/z 444 (M + 1)⁺. |
| 20U | (S)-6-((2- fluoro- benzyl)(meth- yl)amino)- 1-(1-hydroxy- 3-methyl- butan-2-yl)- 7-methoxy- 4-oxo-1,4- dihydro-1,8- naphthyridine- 3-carboxylic acid | 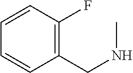 | 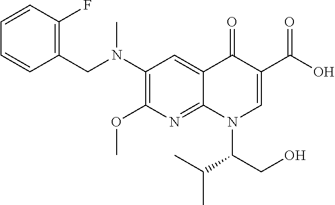 | ^{c)} 15.70 (s, 1H), 8.82 (s, 1H), 7.68 (s, 1H), 7.42 (m, 1H), 7.35 (m, 1H), 7.18 (m, 2H), 5.49 (m, 1H), 5.22 (m, 1H), 4.58 (m, 2H), 4.18 (s, 3H), 4.05 (m, 1H), 3.83 (m, 1H), 2.82 (s, 3H), 2.35 (m, 1H), 1.15 (d, 3H), 0.70 (d, 3H); MS (ESI): m/z 444 (M + 1)⁺. |
| 20V | (S)-6-((4- fluoro- benzyl)(meth- yl)amino)- 1-(1-hydroxy- 3-methyl- butan-2-yl)- 7-methoxy- 4-oxo-1,4- dihydro-1,8- naphthyridine- 3-carboxylic acid | 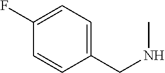 | 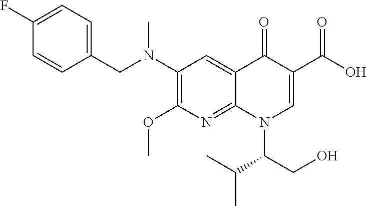 | ^{c)} 15.70 (s, 1H), 8.92 (s, 1H), 7.75 (s, 1H), 7.38 (m, 2H), 7.20 (m, 2H), 5.49 (m, 1H), 5.20 (m, 1H), 4.40 (s, 2H), 4.17 (s, 3H), 4.05 (m, 1H), 3.83 (m, 1H), 2.78 (s, 3H), 2.35 (m, 1H), 1.15 (d, 3H), 0.70 (d, 3H); MS (ESI): m/z 444 (M + 1)⁺. |
| 20W | (S)-1-(1- hydroxy-3- methylbutan- 2-yl)-7- methoxy- 4-oxo-6-(2- (trifluoro- methyl) benzylamino)- 1,4-dihydro- 1,8- naphthyridine- 3-carboxylic acid | 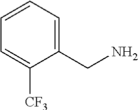 | 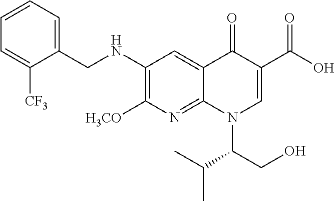 | ^{c)} 15.70 (s, 1H), 8.80 (s, 1H), 7.83 (d, 1H), 7.65 (t, 1H), 7.54 (m, 2H), 7.05 (br s, 2H), 5.48 (m, 1H), 5.18 (m, 1H), 4.65 (d, 2H), 4.20 (s, 3H), 4.05 (m, 1H), 3.92 (m, 1H), 2.35 (m, 1H), 1.12 (d, 3H), 0.73 (d, 3H); MS (ESI): m/z 480 (M + 1)⁺. |
| 20X | (S)-1-(1- hydroxy-3- methylbutan- 2-yl)-7- methoxy- 4-oxo-6-(3- (trifluoro- methyl) benzylamino)- 1,4-dihydro- 1,8- naphthyridine- 3-carboxylic acid | 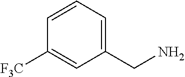 | 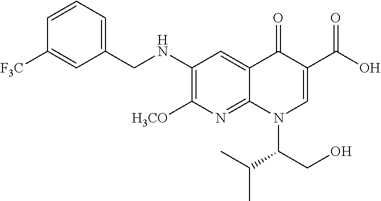 | ^{c)} 15.70 (s, 1H), 8.80 (s, 1H), 7.80 (s, 1H), 7.73 (m, 1H), 7.60 (m, 2H), 7.20 (s, 1H), 7.05 (t, 1H), 5.48 (m, 1H), 5.18 (m, 1H), 4.60 (d, 2H), 4.20 (s, 3H), 4.05 (m, 1H), 3.80 (m, 1H), 2.35 (m, 1H), 1.12 (d, 3H), 0.73 (d, 3H); MS (ESI): m/z 480 (M + 1)⁺. |
| 20Y | (S)-1-(1- hydroxy-3- methylbutan- 2-yl)-7- methoxy- 4-oxo-6-(4- (trifluoro- methyl) benzylamino)- 1,4-dihydro- 1,8- naphthyridine- 3-carboxylic acid | 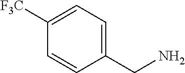 | 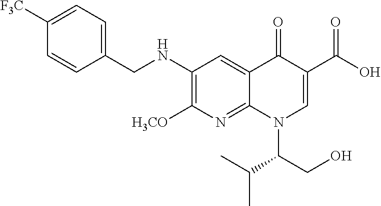 | ^{c)} 15.70 (s, 1H), 8.80 (s, 1H), 7.75 (d, 2H), 7.63 (d, 2H), 7.15 (s, 1H), 7.05 (t, 1H), 5.48 (m, 1H), 5.18 (m, 1H), 4.60 (d, 2H), 4.18 (s, 3H), 4.03 (m, 1H), 3.80 (m, 1H), 2.35 (m, 1H), 1.15 (d, 3H), 0.73 (d, 3H); MS (ESI): m/z 480 (M + 1)⁺. |
| 20Z | (S)-6-(2,6- difluoro- benzyl- amino)-1-(1- hydroxy-3- methylbutan- 2-yl)-7- methoxy- 4-oxo-1,4- dihydro-1,8- naphthyridine- 3-carboxylic acid | 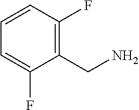 | 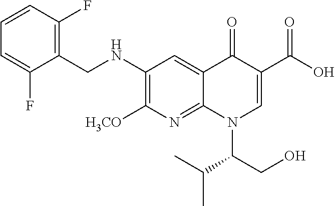 | ^{c)} 15.70 (s, 1H), 8.82 (s, 1H), 7.52 (s, 1H), 7.43 (m, 1H), 7.15 (m, 2H), 6.57 (t, 1H), 5.45 (m, 1H), 5.18 (m, 1H), 4.53 (d, 2H), 4.13 (s, 3H), 4.05 (m, 1H), 3.80 (m, 1H), 2.35 (m, 1H), 1.12 (d, 3H), 0.70 (d, 3H); MS (ESI): m/z 448 (M + 1)⁺. |
| 20AA | (S)-1-(1- hydroxy-3- methylbutan- 2-yl)-7- methoxy- 4-oxo-6-(2,4,6- trifluoro- benzyl- amino)-1,4- dihydro-1,8- naphthyridine- 3-carboxylic acid | 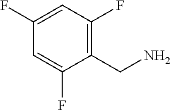 | 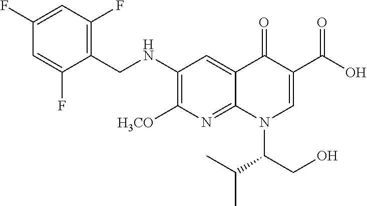 | ^{c)} 15.70 (s, 1H), 8.82 (s, 1H), 7.49 (s, 1H), 7.25 (t, 2H), 6.59 (t, 1H), 5.45 (m, 1H), 5.18 (m, 1H), 4.48 (d, 2H), 4.13 (s, 3H), 4.05 (m, 1H), 3.80 (m, 1H), 2.35 (m, 1H), 1.12 (d, 3H), 0.70 (d, 3H); MS (ESI): m/z 466 (M + 1)⁺. |
| 20BB | (S)-6-(2,3- difluoro- benzyl- amino)-1-(1- hydroxy-3- methylbutan- 2-yl)-7- methoxy- 4-oxo-1,4- dihydro-1,8- naphthyridine- 3-carboxylic acid | 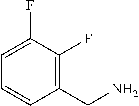 | 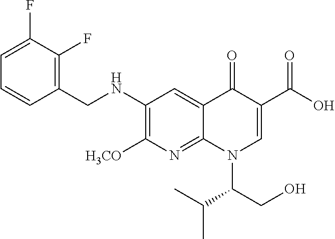 | ^{c)} 15.70 (s, 1H), 8.82 (s, 1H), 7.36 (m, 1H), 7.22 (s, 1H), 7.18 (m, 2H), 6.95 (t, 1H), 5.48 (m, 1H), 5.18 (m, 1H), 4.58 (d, 2H), 4.18 (s, 3H), 4.05 (m, 1H), 3.80 (m, 1H), 2.35 (m, 1H), 1.12 (d, 3H), 0.70 (d, 3H); MS (ESI): m/z 448 (M + 1)⁺. |
| 20CC | (S)-6-(2,5- difluoro- benzyl- amino)-1-(1- hydroxy-3- methylbutan- 2-yl)-7- methoxy- 4-oxo-1,4- dihydro-1,8- naphthyridine- 3-carboxylic acid | 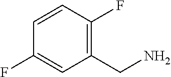 | 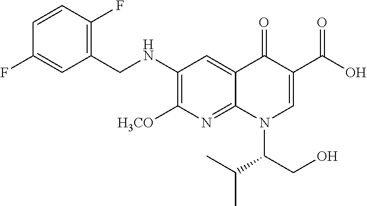 | ^{c)} 8.82 (s, 1H), 7.36 (m, 2H), 7.22 (s, 1H), 7.18 (m, 2H), 6.90 (t, 1H), 5.45 (m, 1H), 5.18 (m, 1H), 4.53 (d, 2H), 4.18 (s, 3H), 4.05 (m, 1H), 3.80 (m, 1H), 2.35 (m, 1H), 1.12 (d, 3H), 0.71 (d, 3H); MS (ESI): m/z 448 (M + 1)⁺. |
| 20DD | (S)-1-(1- hydroxy-3- methylbutan- 2-yl)-7- methoxy-4- oxo-6-(3,4,5- trifluorobenzyl- amino)-1,4- dihydro-1,8- naphthyridine- 3-carboxylic acid | 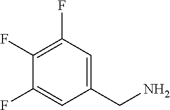 | 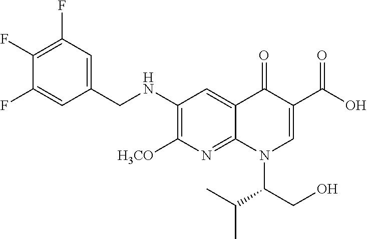 | ^{c)} 15.70 (s, 1H), 8.82 (s, 1H), 7.38 (t, 2H), 7.18 (s, 1H), 6.95 (m, 1H), 5.45 (m, 1H), 5.18 (m, 1H), 4.48 (d, 2H), 4.13 (s, 3H), 4.03 (m, 1H), 3.80 (m, 1H), 2.35 (m, 1H), 1.12 (d, 3H), 0.70 (d, 3H); MS (ESI): m/z 466 (M + 1)⁺. |
| 20EE | (S)-1-(1- hydroxy-3- methylbutan- 2-yl)-7- methoxy-4- oxo-6-(2,3,4- trifluoro- benzyl- amino)-1,4- dihydro-1,8- naphthyridine- 3-carboxylic acid | 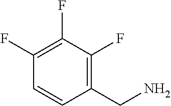 | 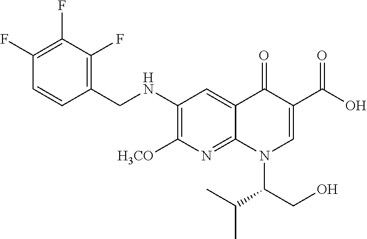 | ^{c)} 15.70 (s, 1H), 8.82 (s, 1H), 7.28 (m, 3H), 6.90 (t, 1H), 5.45 (m, 1H), 5.18 (m, 1H), 4.48 (d, 2H), 4.13 (s, 3H), 4.03 (m, 1H), 3.80 (m, 1H), 2.35 (m, 1H), 1.12 (d, 3H), 0.70 (d, 3H); MS (ESI): m/z 466 (M + 1)⁺. |
| 20FF | (S)-1-(1- hydroxy-3- methylbutan- 2-yl)-7- methoxy-4- oxo-6-(2,3,5- trifluoro- benzyl- amino)-1,4- dihydro-1,8- naphthyridine- 3-carboxylic acid | 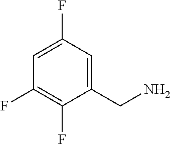 | 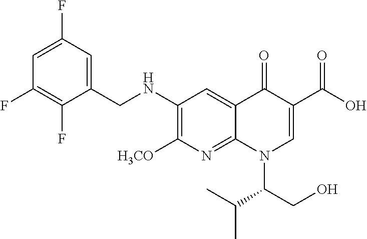 | ^{c)} 15.70 (s, 1H), 8.82 (s, 1H), 7.45 (m, 1H), 7.25 (s, 1H), 7.08 (m, 1H), 6.92 (t, 1H), 5.48 (m, 1H), 5.18 (m, 1H), 4.58 (d, 2H), 4.18 (s, 3H), 4.03 (m, 1H), 3.80 (m, 1H), 2.35 (m, 1H), 1.15 (d, 3H), 0.73 (d, 3H); MS (ESI): m/z 466 (M + 1)⁺. |
| 20GG | (S)-1-(1- hydroxy-3- methylbutan- 2-yl)-7- methoxy-4- oxo-6-(2,3,6- trifluoro- benzyl- amino)-1,4- dihydro-1,8- naphthyridine- 3-carboxylic acid | 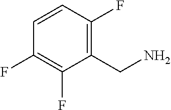 | 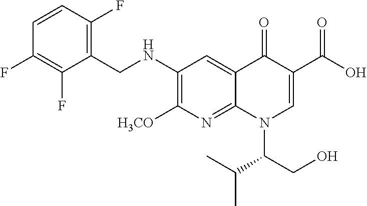 | ^{c)} 15.70 (s, 1H), 8.82 (s, 1H), 7.50 (m, 2H), 7.20 (m, 1H), 6.65 (t, 1H), 5.45 (m, 1H), 5.18 (m, 1H), 4.58 (d, 2H), 4.18 (s, 3H), 4.03 (m, 1H), 3.80 (m, 1H), 2.35 (m, 1H), 1.15 (d, 3H), 0.73 (d, 3H); MS (ESI): m/z 466 (M +)⁺. |
| 20HH | (S)-6-(3,5- difluoro- benzyl- amino)-1-(1- hydroxy-3- methylbutan- 2-yl)-7- methoxy- 4-oxo-1,4- dihydro-1,8- naphthyridine- 3-carboxylic acid | 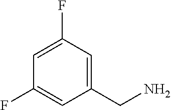 | 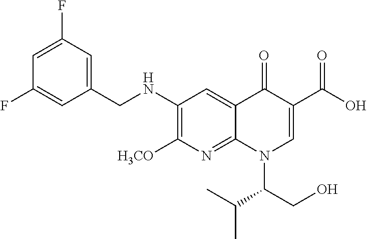 | ^{c)} 15.70 (s, 1H), 8.82 (s, 1H), 7.15 (m, 4H), 7.00 (t, 1H), 5.48 (m, 1H), 5.18 (m, 1H), 4.54 (d, 2H), 4.18 (s, 3H), 4.03 (m, 1H), 3.80 (m, 1H), 2.35 (m, 1H), 1.15 (d, 3H), 0.73 (d, 3H); MS (ESI): m/z 448 (M + 1)⁺. |
| 20II | (S)-1-(1- hydroxy-3- methylbutan- 2-yl)-7- methoxy-4- oxo-6-(2,4,5- trifluoro- benzyl- amino)-1,4- dihydro-1,8- naphthyridine- 3-carboxylic acid | 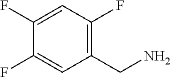 | 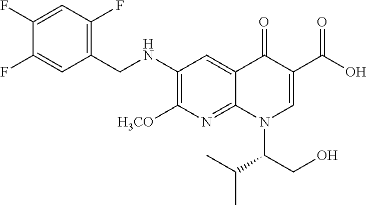 | ^{c)} 15.70 (s, 1H), 8.82 (s, 1H), 7.65 (m, 1H), 7.45 (m, 1H), 7.24 (s, 1H), 6.88 (t, 1H), 5.48 (m, 1H), 5.18 (m, 1H), 4.58 (d, 2H), 4.18 (s, 3H), 4.03 (m, 1H), 3.80 (m, 1H), 2.38 (m, 1H), 1.15 (d, 3H), 0.73 (d, 3H); MS (ESI): m/z 466 (M + 1)⁺. |
| 20JJ | 1-((S)-1- hydroxy-3- methylbutan- 2-yl)-7- methoxy- 4-oxo-6- (1,2,3,4- tetrahydro- naphthalen-1- ylamino)-1,4- dihydro-1,8- naphthyridine- 3-carboxylic acid | 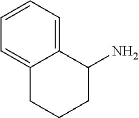 | 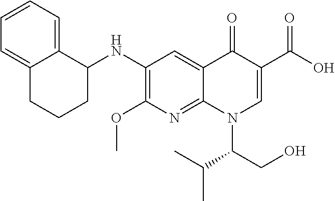 | ^{c)} 15.70 (s, 1H), 8.82 (s, 1H), 7.45 (s, 1H), 7.28 (m, 1H), 7.18 (m, 3H), 6.12 (m, 1H), 5.50 (m, 1H), 5.20 (m, 1H), 4.85 (m, 1H), 4.18 (s, 3H), 4.03 (m, 1H), 3.82 (m, 1H), 2.82 (m, 2H), 2.35 (m, 1H), 1.98 (m, 2H), 1.92 (m, 1H), 1.85 (m, 1H), 1.15 (d, 3H), 0.73 (d, 3H); MS (ESI): m/z 452 (M + 1)⁺. |
| 20KK | 6-((S)-1-(4- fluoro- phenyl)ethyl- amino)-1-((S)- 1-hydroxy-3- methylbutan- 2-yl)-7- methoxy-4- oxo-1,4- dihydro-1,8- naphthyridine- 3-carboxylic acid | 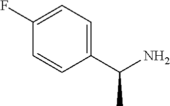 | 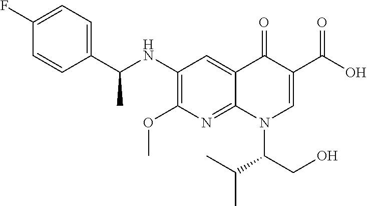 | ^{c)} 15.70 (s, 1H), 8.78 (s, 1H), 7.52 (m, 2H), 7.18 (t, 2H), 7.12 (s, 1H), 6.50 (d, 1H), 5.45 (m, 1H), 5.18 (m, 1H), 4.70 (m, 1H), 4.18 (s, 3H), 4.03 (m, 1H), 3.80 (m, 1H), 2.35 (m, 1H), 1.55 (d, 3H), 1.15 (d, 3H), 0.73 (d, 3H); MS (ESI): m/z 444 (M + 1)⁺. |
| 20LL | 6-((R)-1-(4- fluoro- phenyl)ethyl- amino)-1-((S)- 1-hydroxy- 3-methylbutan- 2-yl)-7- methoxy-4- oxo-1,4- dihydro-1,8- naphthyridine- 3-carboxylic acid | 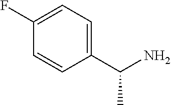 | 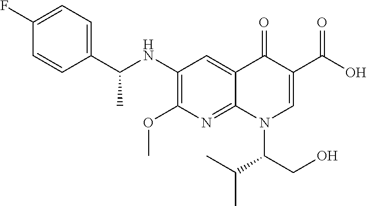 | ^{c)} 15.70 (s, 1H), 8.75 (s, 1H), 7.45 (s, 2H), 7.15 (m, 2H), 7.13 (s, 1H), 6.50 (d, 1H), 5.45 (m, 1H), 5.10 (m, 1H), 4.70 (m, 1H), 4.18 (s, 3H), 3.98 (m, 1H), 3.75 (m, 1H), 2.35 (m, 1H), 1.52 (d, 3H), 1.16 (d, 3H), 0.72 (d, 3H); MS (ESI): m/z 447 (M + 1)⁺. |
| 20MM | (S)-6-(4- fluorobenzyl- amino)-1-(1- hydroxy-3,3- dimethylbutan- 2- yl)-7- methoxy-4- oxo-1,4- dihydro-1,8- naphthyridine- 3-carboxylic acid | 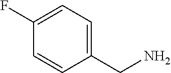 | 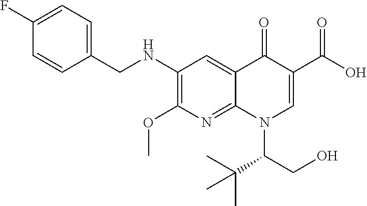 | ^{c)} 15.70 (s, 1H), 8.67 (s, 1H), 7.42 (m, 2H), 7.18 (m, 3H), 6.95 (t, 1H), 5.78 (m, 1H), 5.03 (t, 1H), 4.48 (d, 2H), 4.18 (s, 3H), 4.05 (m, 2H), 0.98 (s, 9H); MS (ESI): m/z 444 (M + 1)⁺. |
| 20NN | (S)-1-(1- hydroxy-3,3- dimethyl- butan-2-yl)- 7-methoxy-4- oxo-6-(2,4,6- trifluoro- benzylamino)- 1,4-dihydro- 1,8- naphthyridine- 3-carboxylic acid | 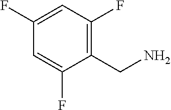 | 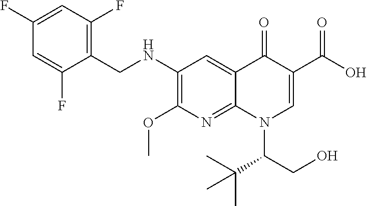 | ^{c)} 15.70 (s, 1H), 8.69 (s, 1H), 7.50 (s, 1H), 7.25 (t, 2H), 6.62 (t, 1H), 5.78 (m, 1H), 5.03 (t, 1H), 4.48 (d, 2H), 4.15 (s, 3H), 4.05 (m, 2H), 0.98 (s, 9H); MS (ESI): m/z 480 (M + 1)⁺. |
| 20OO | (S)-6-(3- chloro-2- fluorophenyl- amino)-1-(1- hydroxy-3- methylbutan- 2-yl)-7- methoxy- 4-oxo-1,4- dihydro-1,8- naphthyridine- 3-carboxylic acid | 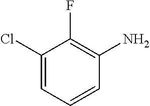 | 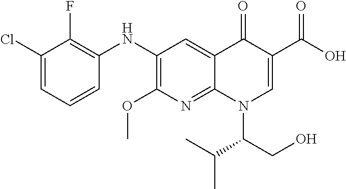 | |
| 20PP | (S)-6-(4- chloro-3- fluorophenyl- amino)-1-(1- hydroxy-3- methylbutan- 2-yl)-7- methoxy- 4-oxo-1,4- dihydro-1,8- naphthyridine- 3-carboxylic acid | 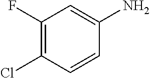 | 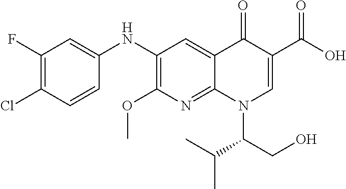 | |
| 20QQ | (S)-6-(4- fluorophenyl- amino)-1-(1- hydroxy-3- methylbutan- 2-yl)-7- methoxy- 4-oxo-1,4- dihydro-1,8- naphthyridine- 3-carboxylic acid | 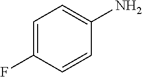 | 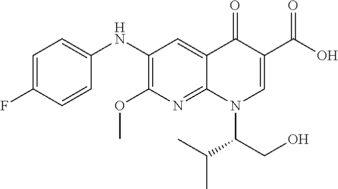 | |
| 20RR | (S)-6-(3- chloro-4- fluorophenyl- amino)-1-(1- hydroxy-3- methylbutan- 2-yl)-7- methoxy- 4-oxo-1,4- dihydro-1,8- naphthyridine- 3-carboxylic acid | 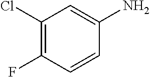 | 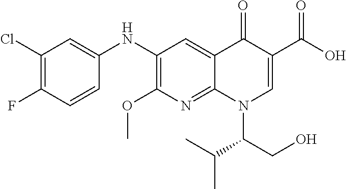 | |
| 20SS | 1-((S)-1- hydroxy-3- methylbutan- 2-yl)-7- methoxy- 4-oxo-6- (1,2,3,4- tetrahydro- naphthalen-1- ylamino)-1,4- dihydro-1,8- naphthyridine- 3-carboxylic acid | 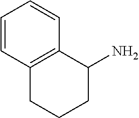 | 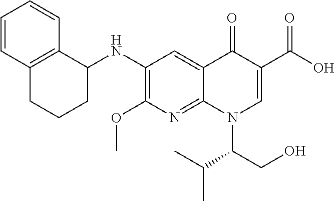 | |

### Example 21

### Compounds of Formula (XXI)

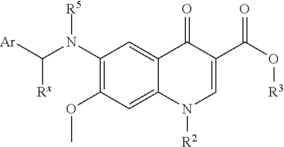

Compounds of formula (XXI) were prepared according to the following synthetic scheme.

When appropriate, protecting groups are used as needed according to established synthetic procedures known to those of skill in the art, and may or may not be removed upon completion of the synthesis. The individual starting materials are synthesized according to methods known in the art or are commercially available.

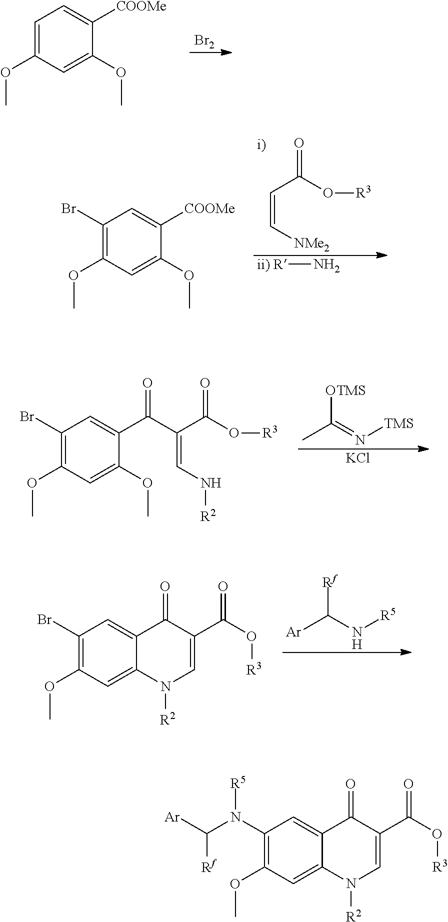

### Example 21A

### (S)-6-(4-Fluorobenzylamino)-1-(1-hydroxy-3-methylbutan-2-yl)-7-methoxy-4-oxo-1,4-dihydroquinoline-3-carboxylic acid

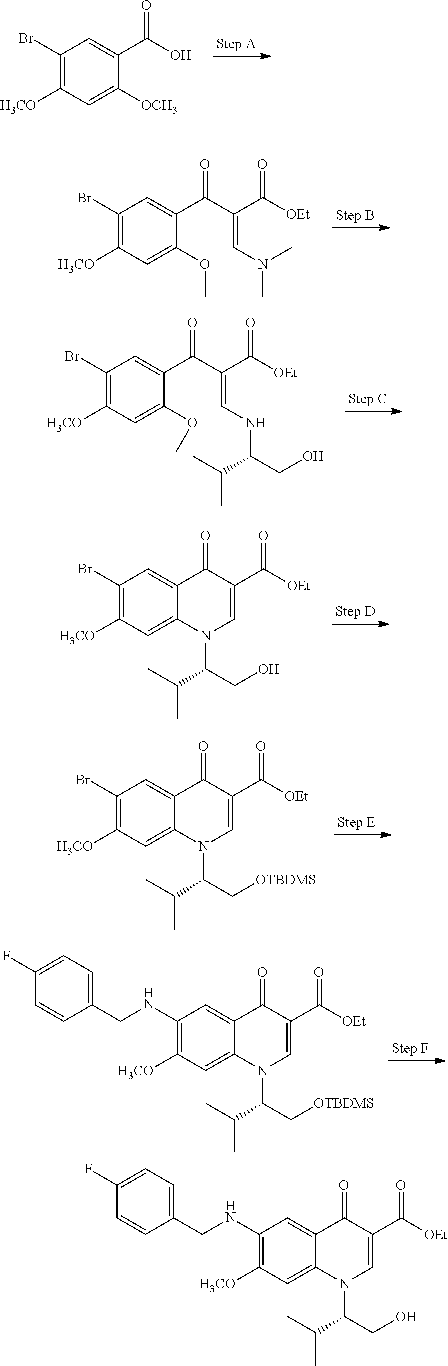

### Step A: Ethyl 2-(5-bromo-2,4-dimethoxybenzoyl)-3-(dimethylamino)acrylate

To a solution of 2,5-dimethoxybenzoic acid (4.60 g, 17.63 mmol) and oxalyl chloride (1.9 mL, 21.78 mmol) in DCM (50 mL) was added DMF (0.1 mL). The reaction mixture was stirred at rt over night and concentrated under reduced pressure. The crude material was dissolved in THF (50 mL) and added ethyl 3-(dimethylamino)acrylate (2.55 g, 17.81 mmol) and TEA (4.9 mL, 35.15 mmol). The mixture was heated at reflux over night, diluted with EtOAc (150 mL) and washed with H10 (2×100 mL). The organic layer was dried over Na₂SO₄ and concentrated. Purification on silica gel column gave the desired product as clear oil which solidified over time (1.85 g). NMR (CDCl3): δ 7.79 (s, 1H), 7.71 (s, 1H), 6.44 (s, 1H), 4.01 (q, 2H), 3.97 (s, 3H), 3.84 (s, 3H), 3.08 (br s, 6H), 1.00 (t, 3H).

### Step B: (S)-Ethyl 2-(5-bromo-2,4-dimethoxybenzoyl)-3-(1-hydroxy-3-methylbutan-2-ylamino)acrylate

A solution of ethyl 2-(5-bromo-2,4-dimethoxybenzoyl)-3-(dimethylamino)acrylate (1.85 g, 4.79 mmol) and (S)-2-amino-3-methylbutan-1-ol (0.60 g, 5.83 mmol) in THF (75 mL) was stirred at rt for 1 h. The reaction mixture was diluted with EtOAc (150 mL), washed with H₂O (2×100 mL) and dried over Na₂SO₄. The solvent was removed under reduced pressure to afford the desired product (2.12 g). NMR (CDCl3): δ 10.90 (t, 1H), 8.04 (d, 1H), 7.51 (br s, 1H), 6.45 (s, 1H), 4.03 (m, 2H), 3.96 (m, 3H), 3.82 (s, 3H), 3.76 (m, 2H), 3.18 (m, 1H), 2.01 (m, 1H), 1.04 (m, 9H).

### Step C: (S)-Ethyl 6-bromo-1-(1-hydroxy-3-methylbutan-2-yl)-7-methoxy-4-oxo-1,4-dihydroquinoline-3-carboxylate

A solution of (S)-ethyl 2-(5-bromo-2,4-dimethoxybenzoyl)-3-(1-hydroxy-3-methylbutan-2-ylamino)acrylate (2.12 g, 4.77 mmol), KCl (180 mg, 2.41 mmol), and trimethylsilyl N-trimethylsilylacetimidate (2.6 mL, 8.47 mmol) in DMF (15 mL) was heated at 100° C. over night. The reaction mixture was acidified with HCl (1 N, 50 mL), and stirred for 1 h. It was extracted with EtOAc (2×100 mL) and the organic layer was washed with brine (50 mL), dried over Na2SO4 and concentrated. Purification on silica gel column gave the desired product as a clear oil (1.72 g). NMR (CDCl3): δ 8.63 (s, 1H), 7.90 (s, 1H), 7.00 (s, 1H), 4.40 (m, 3H), 4.25 (m, 2H), 4.13 (s, 3H), 2.45 (m, 1H), 1.45 (t, 3H), 1.28 (d, 3H), 0.76 (d, 3H).

### Step D: (S)-Ethyl 6-bromo-1-(1-(tert-butyldimethylsilyloxy)-3-methylbutan-2-yl)-7-methoxy-4-oxo-1,4-dihydroquinoline-3-carboxylate

A solution of (S)-ethyl 6-bromo-1-(1-hydroxy-3-methylbutan-2-yl)-7-methoxy-4-oxo-1,4-dihydroquinoline-3-carboxylate (1.72 g, 4.17 mmol), imidazole (2.85 g, 41.86 mmol) and tert-butylchlorodimethylsilane (3.15 g, 20.90 mmol) in DMF (25 mL) was stirred at rt for 1 h. The reaction mixture was diluted with EtOAc (200 mL) and the organic layer washed with H₂O (2×50 mL), dried over Na₂SO₄ and concentrated. Purification on silica gel column gave the desired product as a white foam (1.72 g). NMR (CDCl3): δ 8.78 (s, 1H), 8.76 (s, 1H), 7.18 (s, 1H), 4.47 (m, 1H), 4.45 (m, 2H), 4.14 (m, 1H), 4.13 (s, 3H), 4.00 (m, 1H), 2.45 (m, 1H), 1.45 (t, 3H), 1.27 (d, 3H), 0.90 (d, 3H), 0.82 (s, 9H), −0.00 (d, 6H).

### Step E: (S)-Ethyl 1-(1-(tert-butyldimethylsilyloxy)-3-methylbutan-2-yl)-6-(4-fluorobenzylamino)-7-methoxy-4-oxo-1,4-dihydroquinoline-3-carboxylate

A solution of (S)-ethyl 6-bromo-1-(1-(tert-butyldimethylsilyloxy)-3-methylbutan-2-yl)-7-methoxy-4-oxo-1,4-dihydroquinoline-3-carboxylate (53 mg, 0.101 mmol), (4-fluorophenyl)methanamine (30 mg, 0.239 mmol), Pd(OAc)₂ (5 mg, 0.022 mmol), BINAP (28 mg, 0.045 mmol), and Cs₂CO₃ (70 mg, 0.215 mmol) in toluene (2 mL) was degassed by bubbling nitrogen for 20 min then heated at 110° C. over night. The reaction mixture was diluted with EtOAc (30 mL), washed with H₂O (2×10 mL), and dried over Na₂SO₄. The solvent was removed under reduced pressure and purified on silica gel column to yield the desired product as a foam (25 mg). MS (ESI): m/z 571 (M+1)⁺.

### Step F: (S)-6-(4-Fluorobenzylamino)-1-(1-hydroxy-3-methylbutan-2-yl)-7-methoxy-4-oxo-1,4-dihydroquinoline-3-carboxylic acid

A solution of (S)-Ethyl 1-(1-(tert-butyldimethylsilyloxy)-3-methylbutan-2-yl)-6-(4-fluorobenzylamino)-7-methoxy-4-oxo-1,4-dihydroquinoline-3-carboxylate (25 mg, 0.044 mmol), NaOCH₃ (0.5 mL, 25% in MeOH), and H₂O (0.5 mL) in MeOH (1.0 mL) was heated at 60° C. for 2 h. The reaction mixture was concentrated under reduced pressure to a small volume and diluted with H₂O (10 mL). The pH of the solution was adjusted to 4 with HCl (1 N) and the resulting precipitate was collected by filtration to yield the desired compound as an off-white solid (8 mg). NMR (DMSO-d₆): δ 8.73 (s, 1H), 7.42 (m, 3H), 7.18 (m, 2H), 7.12 (s, 1H), 6.59 (t, 1H), 5.18 (m, 1H), 4.85 (m, 1H), 4.46 (d, 2H), 4.11 (s, 3H), 4.00 (m, 1H), 3.78 (m, 1H), 2.35 (m, 1H), 1.17 (d, 3H), 0.72 (d, 3H); MS (ESI): m/z 429 (M+1)⁺.

### Examples 21B-21D

Examples 21B-21D were prepared according to the procedure described above for example 21A.

| | | | ¹H NMR (400 MHz) 25° C. δ |
|---|---|---|---|
| Compound | Compound Name | Structure | d6-DMSO MS (ESI) |
| 21A | (S)-6-(4- Fluorobenzylamino)- 1-(1-hydroxy-3- methylbutan-2-yl)- 7-methoxy-4-oxo- 1,4-dihydro- quinoline-3- carboxylic acid | 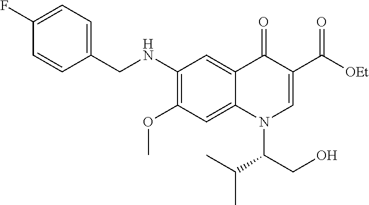 | 8.73 (s, 1H), 7.42 (m, 3H), 7.18 (m, 2H), 7.12 (s, 1H), 6.59 (t, 1H), 5.18 (m, 1H), 4.85 (m, 1H), 4.46 (d, 2H), 4.11 (s, 3H), 4.00 (m, 1H), 3.78 (m, 1H), 2.35 (m, 1H), 1.17 (d, 3H), 0.72 (d, 3H); MS (ESI): m/z 429 (M + 1)⁺ |
| 21B | (S)-1-(1-hydroxy-3- methylbutan-2-yl)- 7-methoxy-4-oxo- 6-(2,4,6-trifluoro- benzylamino)-1,4- dihydroquinoline-3- carboxylic acid | 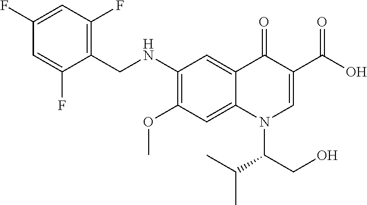 | 15.44 (s, 1H), 8.76 (s, 1H), 7.41 (m, 2H), 7.23 (t, 2H), 6.14 (t, 1H), 5.19 (m, 1H), 4.83 (m, 1H), 4.48 (d, 2H), 4.06 (s, 3H), 4.00 (m, 1H), 3.78 (m, 1H), 2.38 (m, 1H), 1.17 (d, 3H), 0.71 (d, 3H); MS (ESI): m/z 465 (M + 1)⁺ |
| 21C | (S)-6-(2,6- difluorobenzylamino)- 1-(1-hydroxy-3- methylbutan-2-yl)- 7-methoxy-4-oxo- 1,4-dihydroquinoline- 3-carboxylic acid | 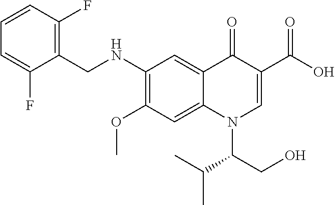 | 15.70 (s, 1H), 8.75 (s, 1H), 8.05 (d, 1H), 7.45 (m, 2H), 7.32 (m, 2H), 7.20 (t, 2H), 7.08 (t, 1H), 6.08 (t, 1H), 5.18 (t, 1H), 4.78 (m, 1H), 4.42 (d, 2H), 3.98 (m, 1H), 3.75 (m, 1H), 2.35 (m, 1H), 1.16 (d, 3H), 0.72 (d, 3H); MS (ESI): m/z 399 (M + l)⁺ |
| 21D | (S)-6-(2,4- difluorobenzylamino)- 1-(1-hydroxy-3- methylbutan-2-yl)- 7-methoxy-4-oxo- 1,4-dihydroquinoline- 3-carboxylic acid | 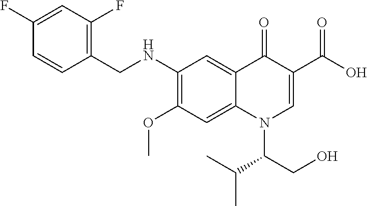 | 15.70 (s, 1H), 8.75 (s, 1H), 7.45 (s, 1H), 7.39 (m, 2H), 7.13 (t, 2H), 6.08 (t, 1H), 5.18 (m, 1H), 4.85 (m, 1H), 4.52 (d, 2H), 4.06 (s, 3H), 3.98 (m, 1H), 3.78 (m, 1H), 2.35 (m, 1H), 1.16 (d, 3H), 0.72 (d, 3H); MS (ESI): m/z 447 (M + 1)⁺ |

### Example 22

### Compounds of Formula (XXII)

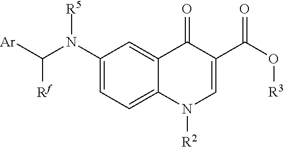

Compounds of formula (XXII) were prepared according to the following synthetic scheme.

When appropriate, protecting groups are used as needed according to established synthetic procedures known to those of skill in the art, and may or may not be removed upon completion of the synthesis. The individual starting materials are synthesized according to methods known in the art or are commercially available.

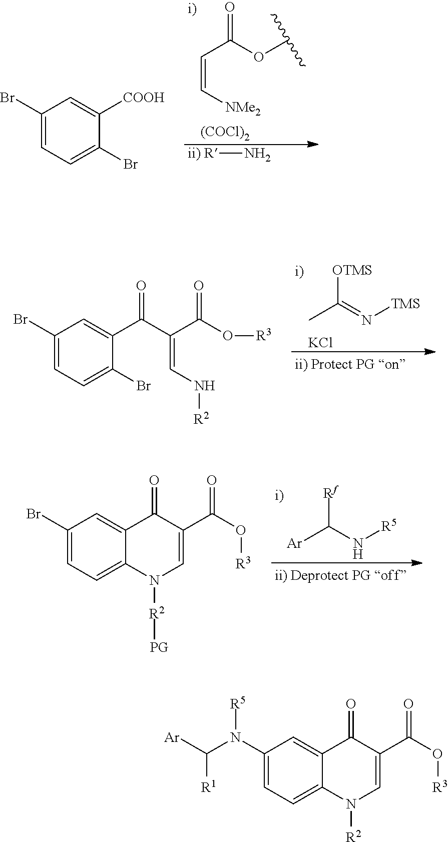

### Example 22A

### S)-6-(4-fluorobenzylamino)-1-(1-hydroxy-3-methylbutan-2-yl)-4-oxo-1,4-dihydroquinoline-3-carboxylic acid

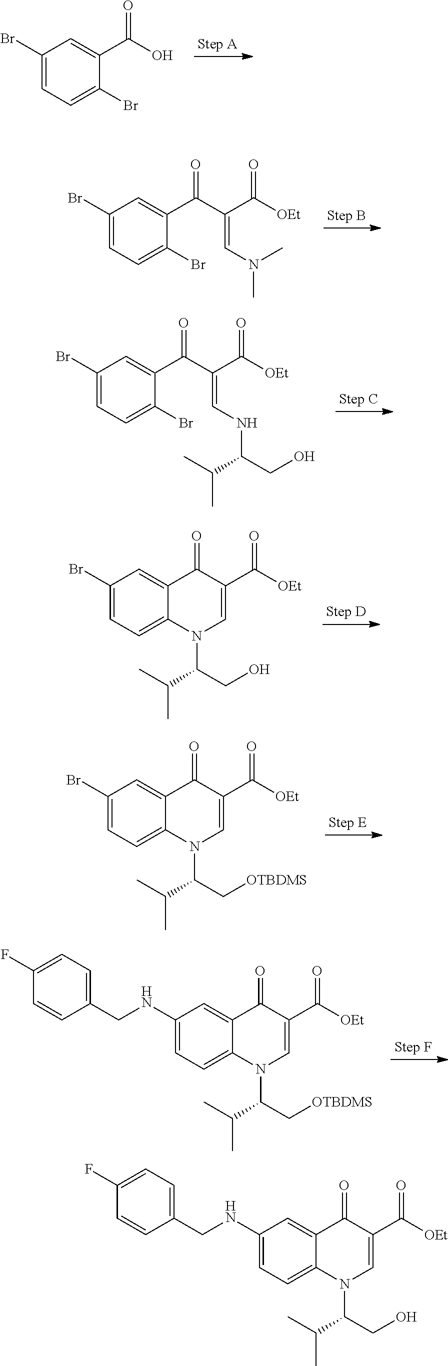

### Step A: (Z)-Ethyl 2-(2,5-dibromobenzoyl)-3-(dimethylamino)acrylate

To a solution of 2,5-dibromobenzoic acid (10.33 g, 36.90 mmol) and oxalyl chloride (4.0 mL, 45.82 mmol) in DCM (100 mL) was added DMF (0.5 mL). The reaction mixture was stirred at rt for 40 min and concentrated under reduced pressure. The crude material was dissolved in THF (100 mL) and added ethyl 3-(dimethylamino)acrylate (5.80 g, 40.51 mmol) and TEA (10.5 mL, 75.33 mmol). The mixture was heated at reflux over night, diluted with EtOAc (250 mL) and washed with H₂O (2×100 mL). The organic layer was dried over Na₂SO₄ and concentrated. Purification on silica gel column gave the desired product as a yellow oil (10.32 g). NMR (CDCl3): δ 7.89 (s, 1H), 7.48 (d, 1H), 7.43 (s, 0.4H), 7.41 (s, 0.6H), 7.35 (d, 0.6H), 7.32 (d, 0.4H), 3.96 (q, 2H), 3.39 (br s, 3H), 3.03 (br s, 3H), 0.91 (t, 3H).

### Step B: (S)-Ethyl 2-(2,5-dibromobenzoyl)-3-(1-hydroxy-3-methylbutan-2-ylamino)acrylate

A solution of (Z)-ethyl 2-(2,5-dibromobenzoyl)-3-(dimethylamino)acrylate (5.26 g, 12.99 mmol) and (S)-2-amino-3-methylbutan-1-ol (1.35 g, 13.09 mmol) in THF (50 mL) was stirred at rt for 2 h. The reaction mixture was diluted with EtOAc (150 mL), washed with H₂O (2×100 mL) and dried over Na₂SO₄. The solvent was removed under reduced pressure to afford the desired product (5.70 g). NMR (CDCl3): δ 11.10 (t, 0.8; H), 9.75 (t, 0.2H), 8.28 (d, 0.2H), 8.23 (d, 0.8; H), 7.35 (m, 3H), 4.00 (m, 2H), 3.89 (m, 1H), 3.80 (m, 1H), 3.25 (m, 1H), 2.04 (m, 1H), 1.06 (m, 6H), 0.97 (t, 3H).

### Step C and D: (S)-Ethyl 6-bromo-1-(1-(tert-butyldimethylsilyloxy)-3-methylbutan-2-yl)-4-oxo-1,4-dihydroquinoline-3-carboxylate

A solution of (S)-ethyl 2-(2,5-dibromobenzoyl)-3-(1-hydroxy-3-methylbutan-2-ylamino)acrylate (2.74 g, 5.92 mmol), KCl (225 mg, 3.02 mmol), and trimethylsilyl N-trimethylsilylacetimidate (3.5 mL, 14.80 mmol) in DMF (20 mL) was heated at 120° C. over night. The reaction mixture was acidified with HCl (1 N, 50 mL), and stirred for 10 min. It was extracted with EtOAc (2×75 mL) and the organic layer was washed with brine (50 mL), dried over Na2SO4 and concentrated. The crude material was dissolved in DMF (50 mL) followed by the addition of imidazole (4.45 g, 65.44 mmol) and tert-butylchlorodimethylsilane (4.00 g, 58.75 mmol). The reaction mixture was stirred at rt for 30 min and diluted with 1-120 (100 mL). It was extracted with EtOAc (3×50 mL) and the organic layer was washed with H2O (2×50 mL), dried over Na2SO4 and concentrated. Purification on silica gel column gave the desired product as a white foam (1.60 g). NMR (CDCl3): δ 8.69 (s, 1H), 8.12 (d, 1H), 7.95 (dd, 1H), 4.77 (m, 1H), 4.24 (m, 2H), 4.07 (dd, 1H), 2.36 (m, 1H), 1.30 (t, 3H), 1.16 (d, 3H), 0.77 (d, 3H), 0.74 (s, 9H), −0.06 (s, 6H).

### Step E: (S)-Ethyl 1-(1-(tert-butyldimethylsilyloxy)-3-methylbutan-2-yl)-6-(4-fluorobenzylamino)-4-oxo-1,4-dihydroquinoline-3-carboxylate

A solution of (S)-ethyl 6-bromo-1-(1-(tert-butyldimethylsilyloxy)-3-methylbutan-2-yl)-4-oxo-1,4-dihydroquinoline-3-carboxylate (125 mg, 0.252 mmol), (4-fluorophenyl)methanamine (70 mg, 0.559 mmol), Pd(OAc)₂ (10 mg, 0.045 mmol), BINAP (50 mg, 0.081 mmol), and Cs₂CO₃ (140 mg, 0.431 mmol) in toluene (2 mL) was degassed by bubbling nitrogen for 15 min then heated at 110° C. over night. The reaction mixture was diluted with EtOAc (30 mL), washed with H₂O (2×10 mL), and dried over Na₂SO₄. The solvent was removed under reduced pressure and purified on silica gel prep TLC (50% EtOAc/hexanes) to yield the desired product as a foam (30 mg). MS (ESI): m/z 541 (M+1)⁺.

### Step F: (S)-6-(4-fluorobenzylamino)-1-(1-hydroxy-3-methylbutan-2-yl)-4-oxo-1,4-dihydroquinoline-3-carboxylic acid

A solution of (S)-ethyl 1-(1-(tert-butyldimethylsilyloxy)-3-methylbutan-2-yl)-6-(4-fluorobenzylamino)-4-oxo-1,4-dihydroquinoline-3-carboxylate (30 mg, 0.055 mmol), NaOCH₃ (1.0 mL, 25% in MeOH), and H₂O (1.0 mL) in MeOH (1.0 mL) was heated at 60° C. for 2 h. The reaction mixture was concentrated under reduced pressure to a small volume and diluted with H₂O (10 mL). The pH of the solution was adjusted to 4 with HCl (1 N) and the resulting precipitate was collected by filtration to yield the desired compound as an off-white solid (11 mg). NMR (DMSO-d₆): δ 15.70 (s, 1H), 8.75 (s, 1H), 8.05 (d, 1H), 7.45 (m, 2H), 7.32 (m, 2H), 7.20 (t, 2H), 7.08 (t, 1H), 5.18 (t, 1H), 4.78 (m, 1H), 4.42 (d, 2H), 3.98 (m, 1H), 3.75 (m, 1H), 2.35 (m, 1H), 1.16 (d, 3H), 0.72 (d, 3H); MS (ESI): m/z 399 (M+1)⁺.

### Examples 22B-22C

Examples 22B-22C were prepared according to the procedure described above for example 22A.

| | | | ¹H NMR (400 MHz) |
|---|---|---|---|
| | | | 25° C. δ d6-DMSO |
| Compound | Compound Name | Structure | MS (ESI) |
| 22A | (S)-6-(4- fluorobenzylamino)-1- (1-hydroxy-3- methylbutan-2-yl)-4- oxo-1,4- dihydroquinoline-3- carboxylic acid | 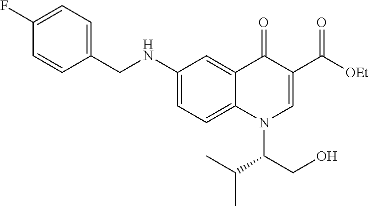 | 15.70 (s, 1H), 8.75 (s, 1H), 8.05 (d, 1H), 7.45 (m, 2H), 7.32 (m, 2H), 7.20 (t, 2H), 7.08 (t, 1H), 5.18 (t, 1H), 4.78 (m, 1H), 4.42 (d, 2H), 3.98 (m, 1H), 3.75 (m, 1H), 2.35 (m, 1H), 1.16 (d, 3H), 0.72 (d, 3H); MS (ESI): m/z 399 (M + 1)⁺ |
| 22B | (S)-6-(4- fluorobenzylamino)-1- (1-hydroxy-3,3- dimethylbutan-2-yl)- 4-oxo-1,4- dihydroquinoline-3- carboxylic acid | 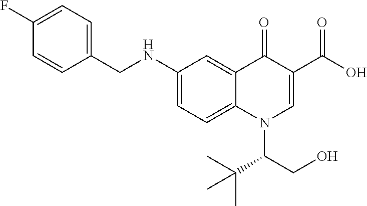 | |
| 22C | (S)-1-(1-hydroxy-3- methylbutan-2-yl)-4- oxo-6-(2,4,6- trifluorobenzylamino)- 1,4-dihydroquinoline- 3-carboxylic acid | 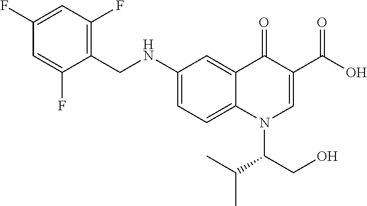 | 15.70 (s, 1H), 8.78 (s, 1H), 8.05 (m, 1H), 7.45 (d, 1H), 7.25 (t, 2H), 6.88 (t, 1H), 5.18 (t, 1H), 4.78 (m, 1H), 4.40 (d, 2H), 3.98 (m, 1H), 3.75 (m, 1H), 2.35 (m, 1H), 1.16 (d, 3H), 0.72 (d, 3H): m/z 435 (M + 1)⁺ |

### Example 23

### Compounds of formula (XXIII)

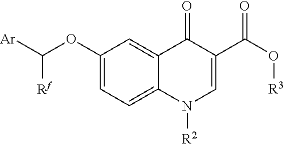

Compounds of formula (XXIII) were prepared according to the following synthetic scheme.

When appropriate, protecting groups are used as needed according to established synthetic procedures known to those of skill in the art, and may or may not be removed upon completion of the synthesis. The individual starting materials are synthesized according to methods known in the art or are commercially available.

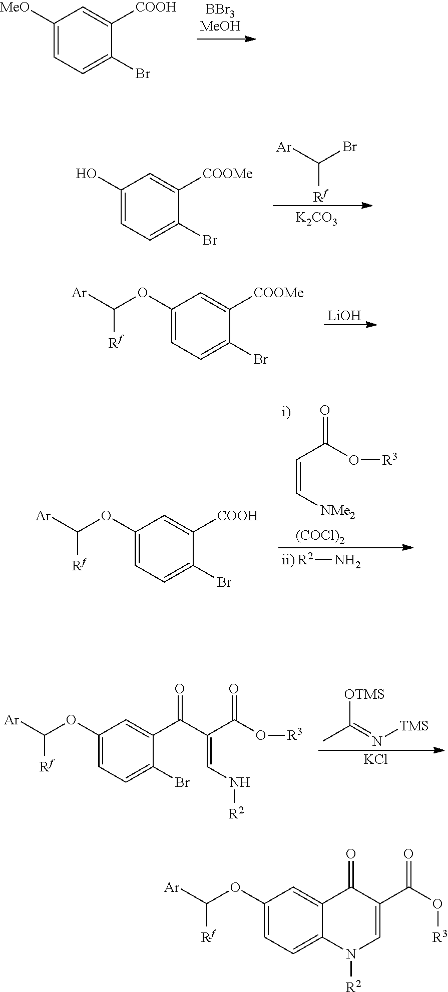

### Example 23A

### (S)-6-(4-fluorobenzyloxy)-1-(1-hydroxy-3-methylbutan-2-yl)-4-oxo-1,4-dihydroquinoline-3-carboxylic acid

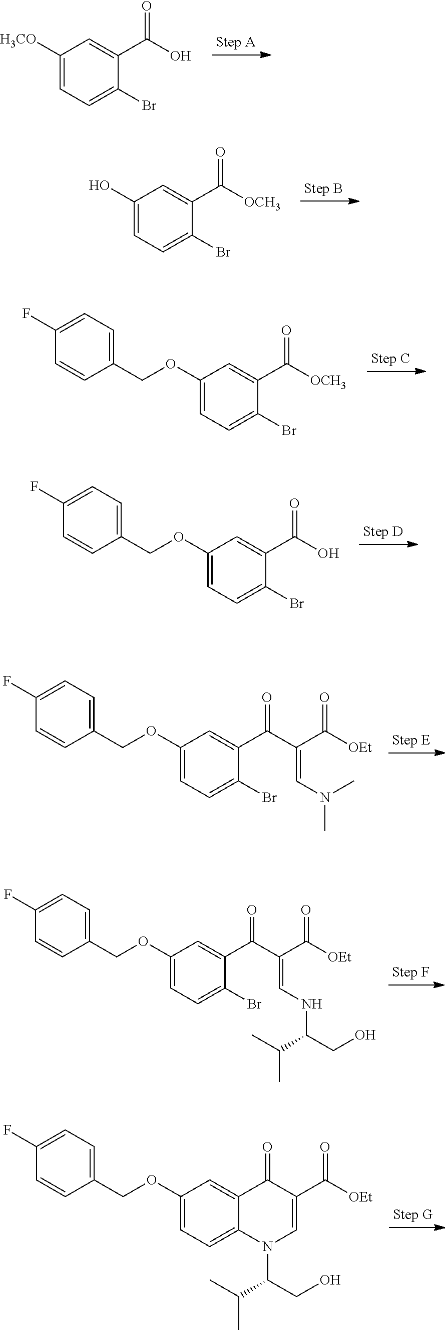

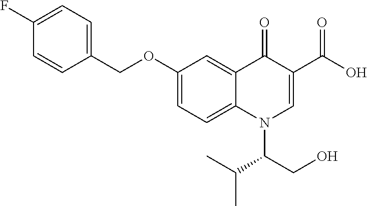

### Step A: Methyl 2-bromo-5-hydroxybenzoate

To a solution of 2-bromo-5-methoxybenzoic acid (1.01 g, 4.39 mmol) in DCM (15 mL) at −78° C. was added boron tribromide in DCM (9.8 mL, 9.8 mmol). The reaction mixture was warmed to rt and stirred for 3 h. Methanol (15 mL) was added to the reaction mixture and stirred for additional 30 min. Concentrated sulfuric acid was then added and heated at 50° C. over night. The reaction was concentrated and then diluted with DCM (100 mL). The organic layer was washed with H₂O (2×50 dried over Na₂SO₄ and concentrated to yield the desired product as an off-white solid (900 mg). NMR (CDCl3): δ 7.53 (d, 1H), 7.33 (d, 1H), 6.88 (dd, 1H), 3.96 (s, 3H).

### Step B: Methyl 2-bromo-5-(4-fluorobenzyloxy)benzoate

A solution of methyl 2-bromo-5-hydroxybenzoate (900 mg, 3.89 mmol), 1-(bromomethyl)-4-fluorobenzene (1.13 g, 5.95 mmol), and potassium carbonate (1.09 g, 7.85 mmol) in DMF (10 mL) was stirred over night at rt. The reaction mixture was diluted with H₂O (20 mL) and extracted with EtOAc (3×20 mL). The combined organic layer was washed with H₂O (2×20 mL), dried over Na₂SO₄ and concentrated. Purification on silica gel column gave the desired product (1.34 g). NMR (CDCl3): δ 7.57 (d, 1H), 7.43 (m, 3H), 7.11 (m, 2H), 6.97 (dd, 1H), 5.06 (s, 2H), 3.96 (s, 3H).

### Step C: 2-Bromo-5-(4-fluorobenzyloxy)benzoic acid

A solution of methyl 2-bromo-5-(4-fluorobenzyloxy)benzoate (1.34 g, 3.94 mmol), and lithium hydroxide (1.65 g, 39.31 mmol) in a mixture of THF (6 mL), MeOH (2 mL), and H₂O (2 mL) was stirred at rt for 90 min. The reaction mixture was diluted with H₂O (50 mL) and acidified (1 N HCl). The resulting precipitate was collected by filtration to yield the desired product as a white solid (1.06 g). NMR (CDCl3): δ 7.60 (m, 2H), 7.43 (m, 2H), 7.12 (m, 2H), 7.02 (dd, 1H), 5.08 (s, 2H).

### Step D: Ethyl 2-(2-bromo-5-(4-fluorobenzyloxy)benzoyl)-3-(dimethylamino)acrylate

To a solution of 2-bromo-5-(4-fluorobenzyloxy)benzoic acid (1.06 g, 3.25 mmol) and oxalyl chloride (0.35 mL, 4.01 mmol) in DCM (40 mL) was added DMF (0.2 mL). The reaction mixture was stirred at rt for 1 h and concentrated under reduced pressure. The crude material was dissolved in THF (50 mL) and added ethyl 3-(dimethylamino)acrylate (0.47 g, 3.29 mmol) and TEA (0.9 mL, 6.46 mmol). The mixture was heated at reflux over night, diluted with H₂O (100 mL) and extracted with EtOAc (2×75 mL). The combined organic layer was washed with brine (100 mL), dried over Na₂SO₄ and concentrated. Purification on silica gel column gave the desired product (0.32 g). NMR (CDCl3): δ 7.84 (s, 1H), 7.40 (m, 3H), 7.08 (m, 2H), 7.00 (d, 1H), 6.83 (dd, 1H), 5.03 (s, 2H), 3.94 (q, 2H), 3.36 (br s, 3H), 3.01 (br s, 3H), 0.89 (t, 3H).

### Step E: (S)-Ethyl 2-(2-bromo-5-(4-fluorobenzyloxy)benzoyl)-3-(1-hydroxy-3-methylbutan-2-ylamino)acrylate

A solution of ethyl 2-(2-bromo-5-(4-fluorobenzyloxy)benzoyl)-3-(dimethylamino)acrylate (0.36 g, 0.80 mmol) and (S)-2-amino-3-methylbutan-1-ol (0.10 g, 0.97 mmol) in THF (15 mL) was stirred at rt over night. The reaction mixture was diluted with EtOAc (100 mL), washed with H₂O (2×50 mL) and dried over Na₂SO₄. The solvent was removed under reduced pressure to afford the desired product (0.37 g). NMR (CDCl3): δ 11.10 (t, 1H), 8.23 (d, 1H), 7.40 (On, 3H), 7.10 (m, 2H), 6.85 (m, 2H), 5.02 (s, 2H), 3.99 (q, 2H), 3.85 (m, 2H), 3.23 (m, 1H), 2.03 (m, 1H), 1.06 (m, 6H), 0.95 (t, 3H).

### Step F: (S)-Ethyl 6-(4-fluorobenzyloxy)-1-(1-hydroxy-3-methylbutan-2-yl)-4-oxo-1,4-dihydroquinoline-3-carboxylate

A solution of (S)-Ethyl 2-(2-bromo-5-(4-fluorobenzyloxy)benzoyl)-3-(1-hydroxy-3-methylbutan-2-ylamino)acrylate (135 mg, 0.27 mmol), KCl (10 mg, 0.13 mmol), and trimethylsilyl N-trimethylsilylacetimidate (0.14 mL, 0.59 mmol) in DMF (3 mL) was heated at 120° C. over night. The reaction mixture was diluted with H₂O (10 mL), acidified with HCl (1 N, 2 mL), and stirred for 10 min. It was extracted with EtOAc (3×10 mL) and the organic layer was washed with brine (5 mL), dried over Na₂SO₄ and concentrated. Purification on silica gel column gave the desired product (70 mg). NMR (CDCl3): δ 8.61 (s, 1H), 7.63 (d, 1H), 7.42 (m, 2H), 7.25 (dd, 1H), 7.18 (d, 1H), 7.08 (m, 2H), 4.83 (s, 2H), 4.35 (m, 4H), 4.15 (q, 2H), 2.50 (m, 1H), 1.29 (m, 6H), 0.79 (d, 3H).

### Step G: (S)-6-(4-fluorobenzyloxy)-1-(1-hydroxy-3-methylbutan-2-yl)-4-oxo-1,4-dihydroquinoline-3-carboxylic acid

A solution of (S)-ethyl 6-(4-fluorobenzyloxy)-1-(1-hydroxy-3-methylbutan-2-yl)-4-oxo-1,4-dihydroquinoline-3-carboxylate (70 mg, 0.16 mmol), and lithium hydroxide (70 mg, 1.67 mmol) in a mixture of THF (3 mL), MeOH (1 mL), and H₂O (1 mL) was stirred at rt over night. The reaction mixture was diluted with H₂O (10 mL) and acidified (1 N HCl). The resulting precipitate was collected by filtration to yield the desired product as a white solid (50 mg). NMR (DMSO-d₆): δ 15.44 (s, 1H), 8.93 (s, 1H), 8.32 (d, 1H), 7.90 (d, 1H), 7.67 (dd, 1H), 7.59 (m, 2H), 7.28 (On, 2H), 5.32 (s, 2H), 5.22 (m, 1H), 4.90 (m, 1H), 4.00 (m, 1H), 3.80 (m, 1H), 2.38 (m, 1H), 1.15 (d, 3H), 0.72 (d, 3H); MS (ESI): m/z 400 (M+1)⁺.

### Examples 23B-23C

Examples 23B-23C were prepared according to the procedure described above for example 23A.

| | | | ¹H NMR (400 MHz) 25° C. δ |
|---|---|---|---|
| Compounds | Compound Name | Structure | CDCl₃ MS (ESI) |
| 23A | (S)-6-(4-fluoro- benzyloxy)-1- (1-hydroxy-3- methylbutan-2- yl)-4-oxo-1,4- dihydroquinoline- 3-carboxylic acid | 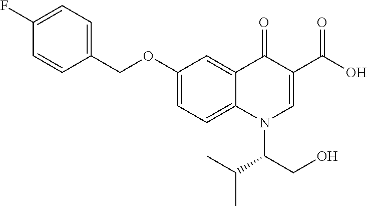 | 15.44 (s, 1H), 8.93 (s, 1H), 8.32 (d, 1H), 7.90 (d, 1H), 7.67 (dd, 1H), 7.59 (m, 2H), 7.28 (m, 2H), 5.32 (s, 2H), 5.22 (m, 1H), 4.90 (m, 1H), 4.00 (m, 1H), 3.80 (m, 1H), 2.38 (m, 1H), 1.15 (d, 3H), 0.72 (d, 3H); MS (ESI): m/z 400 (M + 1)⁺ |
| 23B | (S)-6-(2,4- difluorobenzyloxy)- 1-(1-hydroxy-3,3- dimethylbutan-2- yl)-4-oxo-1,4- dihydroquinoline- 3-carboxylic acid | 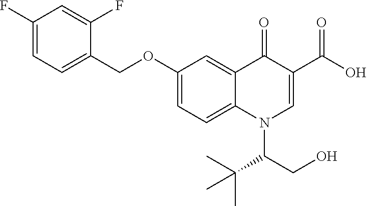 | 15.36 (br. s., 1H), 8.79 (s, 1H), 8.42 (d, J = 9.79 Hz, 1H), 7.91 (d, J = 3.01 Hz, 1H), 7.64-7.73 (m, 1H), 7.61 (dd, J = 9.41, 3.14 Hz, 1H), 7.35 (td, J = 9.91, 2.51 Hz, 1H), 7.16 (td, J = 8.41, 2.01 Hz, 1H), 5.31 (s, 2H), 5.13 (dd, J = 8.78, 4.52 Hz, 1H), 3.98- 4.13 (m, 2H), 3.37 (br. s., 1H), 0.96 (s, 9H) |
| 23C | (S)-1-(1-hydroxy- 3,3-dimethylbutan- 2-yl)-4-oxo-6- (2,4,6-trifluoro- benzyloxy)-1,4- dihydroquinoline- 3-carboxylic acid | 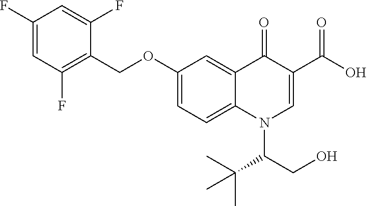 | 8.80-8.86 (m, 1H), 8.04 (d, J = 2.26 Hz, 1H), 7.81-7.88 (m, 1H), 7.44 (d, J = 7.53 Hz, 1H), 6.76 (t, J = 8.03 Hz, 2H), 5.21 (s, 2H), 4.87-4.97 (m, 1H), 4.22-4.43 (m, 2H), 1.04 (s, 9H) |

### Example 24

### Compounds of Formula (XXIV)

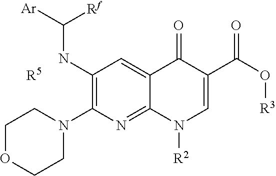

Compounds of formula (XXIV) were prepared according to the following synthetic scheme.

When appropriate, protecting groups are used as needed according to established synthetic procedures known to those of skill in the art, and may or may not be removed upon completion of the synthesis. The individual starting materials are synthesized according to methods known in the art or are commercially available.

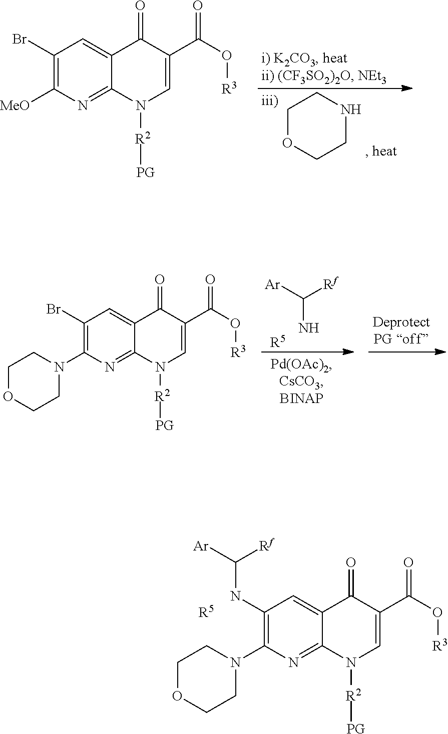

### Example 24A

### (S)-6-(4-fluorobenzylamino)-1-(1-hydroxy-3-methylbutan-2-yl)-7-morpholino-4-oxo-1,4-dihydro-1,8-naphthyridine-3-carboxylic acid

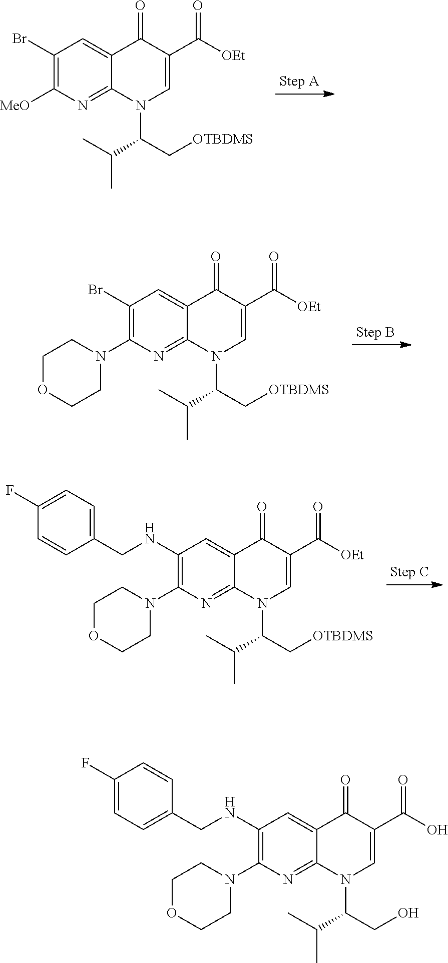

### Step A: (S)-Ethyl 6-bromo-1-(1-(tert-butyldimethylsilyloxy)-3-methylbutane-2-yl)-7-morpholino-4-oxo-1,4-dihydro-1,8-naphthyridine-3-carboxylate

A 48 mL sealed tube was charged with (S)-ethyl 6-bromo-1-(1-(tert-butyldimethylsilyloxy)-3-methylbutan-2-yl)-7-methoxy-4-oxo-1,4-dihydro-1,8-naphthyridine-3-carboxylate (3.10 g, 5.89 mmol), morpholine (1.04 mL, 12.00 mol), and potassium carbonate (1.66 g, 12.00 mmol) in 16 mL dry DMSO. The reaction mixture was stirred at 100° C. for 14 h. After completion, the mixture was cooled to RT. To the mixture was added 1 N HCl aqueous solution yielding, light yellow solids from the solution. The solids were filtered and dried under vacuo providing 2.65 g of (S)-ethyl 6-bromo-1-(1-(tert-butyldimethylsilyloxy)-3-methylbutan-2-yl)-7-hydroxy-4-oxo-1,4-dihydro-1,8-naphthyridine-3-carboxylate as a light yellow solid (88%). The demethylated intermediate was submitted for the next step without further purification. A 100 mL round-bottomed flask was charged with (S)-ethyl 6-bromo-1-(1-(tert-butyldimethylsilyloxy)-3-methylbutan-2-yl)-7-hydroxy-4-oxo-1,4-dihydro-1,8-naphthyridine-3-carboxylate (512 mg, 1.00 mmol) in 15 mL dichloromethane. To the reaction mixture was added triethylamine (415 μL, 3.00 mmol), trifluomethanesulfonic anhydride (252 μL, 1.50 mmol) via syringe for 2 min. The corresponding brown solution, was further stirred at RT for 10 min. The reaction mixture was condensed under reduced pressure yielding an amber residue. The residue was purified by silica-gel chromatography using 0-25% EtOAc in n-hexanes as gradient providing an oily product (420 mg, 67%). The triflate compound (420 mg, 0.65 mmol) was dissolved in dioxane (3.00 mL). To the reaction mixture was added morpholine (113 uL, 1.30 mmol), then heated at 60° C. with stirring for 6 h. The reaction was monitored by LC-MS. After completion, the mixture was cooled to RT. Then the mixture was condensed under reduced pressure yielding amber oily residue. The residue was purified by silica-gel chromatography using gradient of 0˜50% EtOAc in n-hexanes as eluents providing (S)-ethyl 6-bromo-1-(1-(tert-butyldimethylsilyloxy)-3-methylbutan-2-yl)-7-morpholino-4-oxo-1,4-dihydro-1,8-naphthyridine-3-carboxylate as a white foamy solid (246, 63 mg) ¹H NMR (400 MHz, CDCl₃) δ 8.82 (s, 1H), 8.79 (s, 1H), 5.28 (broad d, 1H), 4.40 (q, 2H), 4.09 (d, 1H), 3.92 (m, 4H), 3.83 (d, 1H), 3.58 (m, 4H), 2.45 (m, 1H), 1.43 (t, 3H), 1.19 (d, 3H), 0.85 (s, 9H), 0.82 (d, 3H), 0.02 (d, 6H).

### Step B: (S)-ethyl 1-(1-(tert-butyldimethylsilyloxy)-3-methylbutan-2-yl)-6-(4-fluorobenzylamino)-7-morpholino-4-oxo-1,4-dihydro-1,8-naphthyridine-3-carboxylate

A 15 mL sealed tube was charged with (S)-ethyl 6-bromo-1-(1-(tert-butyldimethylsilyloxy)-3-methylbutan-2-yl)-7-morpholino-4-oxo-1,4-dihydro-1,8-naphthyridine-3-carboxylate (246 mg, 0.41 mmol), 4-fluorobenzylamine (174 μL, 1.53 mmol), Pd(OAc)₂ cesium carbonate (250 mg, 0.77 mmol), and BINAP (49 mg, 0.079 mmol) in 3 mL dry toluene. The reaction mixture was purged with nitrogen for 15 min, the mixture was stirred at 110° C. for 14 h. The reaction mixture was cooled to RT. The mixture was condensed under reduced pressure yielding dark oily residue. The residue was purified by silica-gel chromatography using a gradient of 0˜60% EtOAc in n-hexanes providing (S)-ethyl 1-(1-(tert-butyldimethylsilyloxy)-3-methylbutan-2-yl)-6-(4-fluorobenzylamino)-7-morpholino-4-oxo-1,4-dihydro-1,8-naphthyridine-3-carboxylate as an oil (184 mg, 70%). ¹H NMR (400 MHz, CDCl₃) δ 8.76 (s, 1H), 7.86 (s, 1H), 7.38 (dd, 2H), 7.08 (dd, 2H) 5.39 (broad d, 1H), 4.38 (m, 4H), 4.10 (d, 1H), 3.90 (t, 4H), 3.82 (d, 1H), 3.34 (m, 4H), 2.44 (m, 1H), 1.42 (t, 3H), 1.19 (d, 3H), 0.85 (s, 9H), 0.80 (d, 3H), 0.02 (d, 6H).

### Step C: (S)-6-(4-fluorobenzylamino)-1-(1-hydroxy-3-methylbutan-2-yl)-7-morpholino-4-oxo-1,4-dihydro-1,8-naphthyridine-3-carboxylic acid

A 25 mL round-bottomed flask was charged with (S)-ethyl 1-(1-(tert-butyldimethylsilyloxy)-3-methylbutan-2-yl)-6-(4-fluorobenzylamino)-7-morpholino-4-oxo-1,4-dihydro-1,8-naphthyridine-3-carboxylate (180 mg, 0.29 mmol) on the mixture of 25% NaOMe in MeOH (3.0 mL), MeOH (1.0 mL) and H₂O (1.0 mL). The reaction mixture was stirred at 65° C. for 2 h. After the mixture was cooled to RT, the mixture was condensed under reduced pressure yielding light brown aqueous solution. The pH of the mixture was adjusted below 1 after which a brown solid precipitated. The solid was filtered and dried under vacuo providing 77 mg of (S)-6-(4-fluorobenzylamino)-1-(1-hydroxy-3-methylbutan-2-yl)-7-morpholino-4-oxo-1,4-dihydro-1,8-naphthyridine-3-carboxylic acid as a light brown solid. ¹H NMR (400 MHz, CDCl₃) δ 15.24 (broad s, 1H), 8.86 (s, 1H), 7.64 (s, 1H), 7.44 (dd, 2H) 7.14 (dd, 2H), 5.57 (broad s, 1H), 4.45 (broad in, 3H), 4.25 (broad s, 1H), 3.44 (broad s, 1H), 2.48 (broad s, 1H), 1.27 (d, 2H), 0.81 (d, 2H)

### Example 24B

Example 24B was prepared according to the procedure described above for example 24A.

Example 24C is prepared according to the procedure described above for example 24A.

| | | | ¹H NMR (400 MHz) 25° C.: |
|---|---|---|---|
| Eg | Compound Name | Structure | MS (ESI) |
| 24A | (S)-6-(4-fluoro- benzylamino)-1- (1-hydroxy-3- methylbutan-2-yl)-7- morpholino-4-oxo-1,4- dihydro-1,8- naphthyridine-3- carboxylic acid | 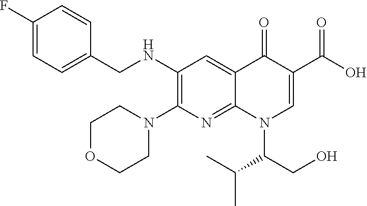 | (CDCl₃) δ 15.24 (broad s, 1H), 8.86 (s, 1H), 7.64 (s, 1H), 7.44 (dd, 2H) 7.14 (dd, 2H), 5.57 (broad s, 1H), 4.45 (broad m, 3H), 4.25 (broad s, 1H), 3.44 (broad s, 1H), 2.48 (broad s, 1H), 1.27 (d, 2H), 0.81 (d, 2H) |
| 24B | (S)-1-(1-hydroxy-3- methylbutan-2-yl)-7- morpholino-4-oxo-6- (2,4,6-trifluoro- benzyloxy)-1,4- dihydro-1,8- naphthyridine-3- carboxylic acid | 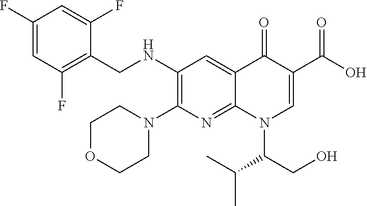 | (DMSO-d6) δ 8.83 (s, 1H), 7.57 (s, 1H), 7.25 (t, 2H), 6.13 (t, 1H) 5.46 (broad, 1H), 4.45 (d, 2H), 3.83 (broad, 5H), 3.40 (broad s, 4H), 2.34 (broad s, 1H), 1.11 (d, 3H), 0.69 (d, 3H) |
| 24C | | 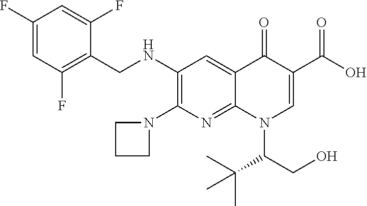 | |

### Example 25

### Compounds of Formula (XXV)

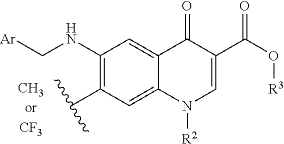

Compounds of formula (XXV) were prepared according to the following synthetic scheme.

When appropriate, protecting groups are used as needed according to established synthetic procedures known to those of skill in the art, and may or may not be removed upon completion of the synthesis. The individual starting materials are synthesized according to methods known in the art or are commercially available.

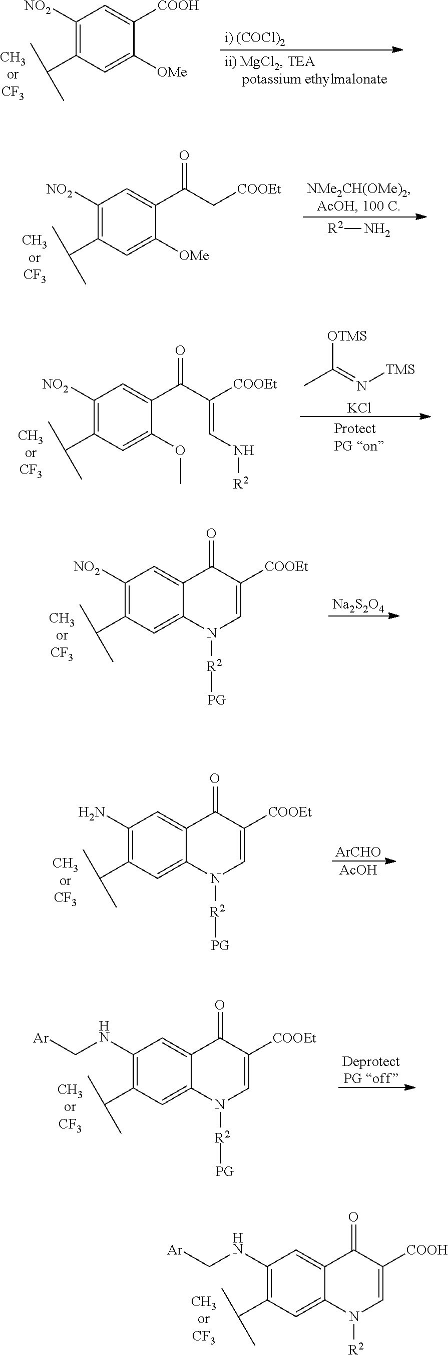

### Example 25A

### (S)-1-(1-hydroxy-3,3-dimethylbutan-2-yl)-4-oxo-6-(2,4,6-trifluorobenzylamino)-7-(trifluoromethyl)-1,4-dihydroquinoline-3-carboxylic acid

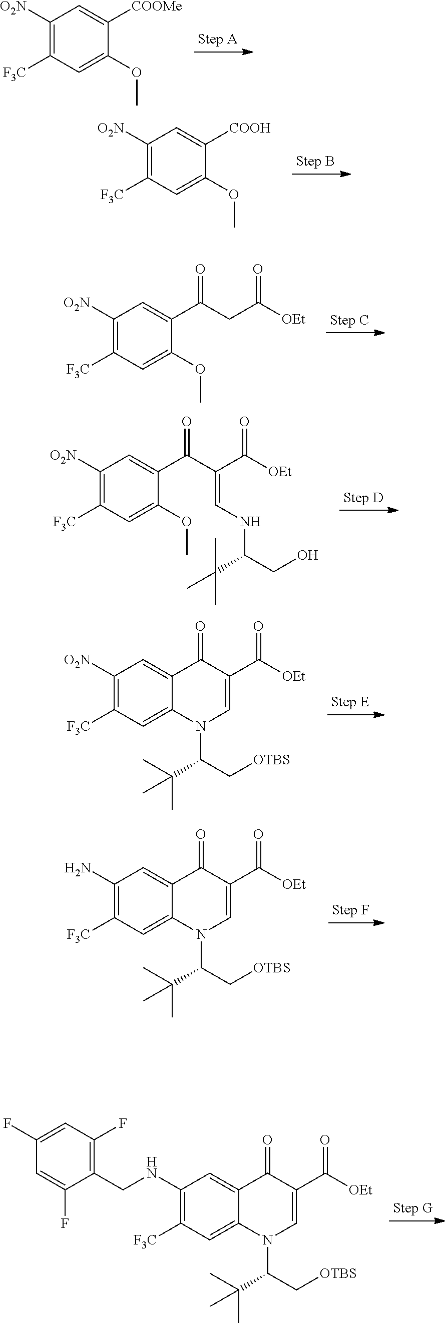

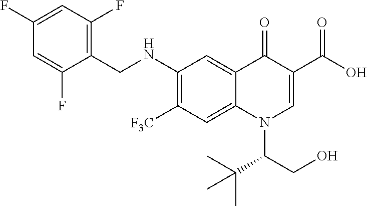

### Step A: 2-Methoxy-5-nitro-4-(trifluoromethyl)benzoic acid

A solution of methyl 2-methoxy-5-nitro-4-(trifluoromethyl)benzoate (1.00 g, 3.58 mmol), and lithium hydroxide (1.52 g, 36.21 mmol) in a mixture of THF (10 mL), MeOH (5 mL), and was stirred at rt for 4 h. The reaction mixture was diluted with H₂O (100 mL) and acidified (1 N HCl). The aqueous layer was extracted with EtOAc (100 mL) and the organic layer was then washed with H₂O (2×50 mL), dried over Na₂SO₄ and concentrated to yield the desired product as a white solid (890 mg). NMR (CDCl3): δ 8.75 (s, 1H), 7.47 (s, 1H), 4.22 (s, 3H).

### Step B: Ethyl 3-(2-methoxy-5-nitro-4-(trifluoromethyl)phenyl)-3-oxopropanoate

To a solution of 2-Methoxy-5-nitro-4-(trifluoromethyl)benzoic acid (1.68 g, 6.32 mmol) and oxalyl chloride (0.7 mL, 8.13 mmol) in DCM (40 mL) was added DMF (0.1 mL). The reaction mixture was stirred at rt for 1 h and concentrated under reduced pressure. A solution of potassium ethylmalonate (2.00 g, 11.75 mmol) and magnesium chloride (2.10 g, 22.06 mmol) in THF (50 mL) was cooled to 0° C. and added above crude in THF (50 mL) followed by TEA (1.8 mL, 12.91 mmol). The mixture was stirred at that temperature for 1 h, diluted with EtOAc (100 mL) and 1 N HCl (50 mL), and stirred at rt for additional 10 min. The layers were separated and the organic layer was washed with satd NaHCO3 (50 mL), dried over Na2SO4 and concentrated. Purification on silica gel column gave the desired product as clear oil (1.71 g). NMR (CDCl3): δ 12.75 (s, 0.4H), 8.61 (s, 0.4H), 8.54 (s, 0.6H), 6.16 (s, 0.4H), 4.33 (q, 0.8; H), 4.22 (q, 1.2H), 4.12 (s, 1.2H), 4.11 (s, 1.8; H), 4.02 (s, 1.2H), 1.39 (t, 1.2H), 1.28 (t, 1.8; H).

### Step C: (S)-Ethyl 3-(1-hydroxy-3,3-dimethylbutan-2-ylamino)-2-(2-methoxy-5-nitro-4-(trifluoromethyl)benzoyl)acrylate

A solution of ethyl 3-(2-methoxy-5-nitro-4-(trifluoromethyl)phenyl)-3-oxopropanoate (1.71 g, 5.10 mmol) and 1,1-dimethoxy-N,N-dimethylmethanamine (0.82 mL, 6.12 mmol) and acetic acid (0.05 mL) in toluene (5 mL) was heated at 100° C. for 30 min. The reaction mixture was cooled to rt and added (S)-2-amino-3,3-dimethylbutan-1-ol (0.73 g, 6.19 mmol) which was then stirred for additional 30 min. Purification on silica gel column gave the desired product as clear oil (2.21 g). NMR (CDCl3): δ 11.25 (t, 0.8; H), 9.65 (t, 0.2H), 8.29 (d, 0.2H), 8.18 (d, 0.8; H), 7.92 (s, 0.8; H), 7.91 (s, 0.2H), 7.24 (s, 0.8H), 7.23 (s, 0.2H), 4.05 (m, 3H), 3.96 (s, 0.6H), 3.94 (s, 2.4H), 3.72 (m, 1H), 3.15 (m, 1H), 1.95 (t, 0.8H), 1.82 (t, 0.2H), 1.06 (m, 11.6H), 0.84 (t, 0.4H).

### Step D: (S)-Ethyl 1-(1-(tert-butyldimethylsilyloxy)-3,3-dimethylbutan-2-yl)-6-nitro-4-oxo-7-(trifluoromethyl)-1,4-dihydroquinoline-3-carboxylate

A solution of (S)-ethyl 3-(1-hydroxy-3,3-dimethylbutan-2-ylamino)-2-(2-methoxy-5-nitro-4-(trifluoromethyl)benzoyl)acrylate (2.21 g, 4.78 mmol), KCl (365 mg, 4.90 mmol), and trimethylsilyl N-trimethylsilylacetimidate (3.0 mL, 12.09 mmol) in DMF (5 mL) was heated at 100° C. for 20 min. The reaction mixture was acidified with HCl (1 N, 50 mL), and stirred for 1 h. It was extracted with EtOAc (100 mL) and the organic layer was washed with satd NaHCO₃ (50 mL), dried over Na₂SO₄ and concentrated. The reaction crude was dissolved in DMF (20 mL) and added imidazole (3.30 g, 48.47 mmol) and tert-butylchlorodimethylsilane (3.62 g, 21.21 mmol). After 30 min, the reaction mixture was diluted with EtOAc (100 mL), washed with H₂O (2×50 mL), dried over Na₂SO₄ and concentrated. Purification on silica gel column gave the desired product as yellow foam (2.32 g). NMR (CDCl3): δ 9.12 (s, 1H), 8.80 (s, 1H), 4.64 (dd, 1H), 4.44 (m, 2H), 4.17 (m, 2H), 1.45 (t, 3H), 1.13 (s, 9H), 0.68 (s, 9H), 0.05 (s, 3H), −0.03 (s; 3H); MS (ESI): m/z 545 (M+1)⁺.

### Step E: (S)-Ethyl 6-amino-1-(1-(tert-butyldimethylsilyloxy)-3,3-dimethylbutan-2-yl)-4-oxo-7-(trifluoromethyl)-1,4-dihydroquinoline-3-carboxylate

A solution of (S)-ethyl 1-(1-(tert-butyldimethylsilyloxy)-3,3-dimethylbutan-2-yl)-6-nitro-4-oxo-7-(trifluoromethyl)-1,4-dihydroquinoline-3-carboxylate (2.32 g, 4.26 mmol) and Na2S2O4 (8.75 g, 42.72 mmol) in THF/H2O (1:1, 100 mL) was stirred at rt for 30 min. The reaction mixture was diluted with EtOAc (100 mL) and the layers were separated. The organic layer was washed with said NaHCO3 (2×50 mL), dried over Na2SO4 and concentrated to yield the desired as a yellow foam (2.07 g). NMR (CDCl3): δ 8.70 (s, 1H), 8.01 (s, 1H), 7.79 (s, 1H), 4.56 (dd, 1H), 4.44 (m, 2H), 4.16 (m, 2H), 1.45 (t, 3H), 1.08 (s, 9H), 0.69 (s, 9H), 0.03 (s, 3H), −0.06 (s, 3H); MS (ESI): m/z 515 (M+1)⁺.

### Step F: (S)-ethyl 1-(1-(tert-butyldimethylsilyloxy)-3,3-dimethylbutan-2-yl)-4-oxo-6-(2,4,6-trifluorobenzylamino)-7-(trifluoromethyl)-1,4-dihydroquinoline-3-carboxylate

A solution of (S)-ethyl 6-amino-1-(1-(tert-butyldimethylsilyloxy)-3,3-dimethylbutan-2-yl)-4-oxo-7-(trifluoromethyl)-1,4-dihydroquinoline-3-carboxylate (100 mg, 0.19 mmol), 2,4,6-trifluorobenzaldehyde (65 mg, 0.41 mmol), and AcOH (1 drop) in MeOH (1 mL) was heated at 65° C. over night. The reaction mixture was cooled to rt, treated with NaCNBH₃ (30 mg, 0.48 mmol), and stirred for additional 30 min. The reaction was diluted with EtOAc (5 mL), washed with H₂O (2 mL), dried over Na₂SO₄ and concentrated. Purification on silica gel column gave the desired product as yellow foam (40 mg). MS (ESI): m/z 659 (M+1)⁺.

### Step G: (S)-1-(1-Hydroxy-3,3-dimethylbutan-2-yl)-4-oxo-6-(2,4,6-trifluorobenzylamino)-7-(trifluoromethyl)-1,4-dihydroquinoline-3-carboxylic acid

A solution (S)-ethyl 1-(1-(tert-butyldimethylsilyloxy)-3,3-dimethylbutan-2-yl)-4-oxo-6-(2,4,6-trifluorobenzylamino)-7-(trifluoromethyl)-1,4-dihydroquinoline-3-carboxylate (70 mg, 0.11 mmol), NaOCH₃ (1 mL, 25% in MeOH), and H₂O (1 mL) in MeOH (1 mL) was heated at 65° C. for 1 h. The reaction mixture was concentrated under reduced pressure to a small volume and diluted with H₂O (10 mL). The pH of the solution was adjusted to 4 with HCl (1 N) and the resulting precipitate was collected by filtration to yield the desired compound (43 mg). NMR (DMSO-d₆): δ 15.00 (s, 1H), 8.75 (s, 1H), 8.36 (s, 1H), 7.67 (s, 1H), 7.23 (t, 2H), 6.57 (t, 1H), 5.14 (m, 1H), 4.62 (d, 2H), 4.05 (m, 2H), 0.97 (s, 9H); MS (ESI): m/z 517 (M+1)⁺.

### Example 25B-25D

Examples 25B-25D were prepared according to the procedure described above for example 25A.

| | | | ¹H NMR (400 MHz) |
|---|---|---|---|
| | | | 25° C. δ DMSO-d6: |
| Compound | Compound Name | Structure | MS (ESI) |
| 25A | (S)-1-(1-Hydroxy-3,3- dimethylbutan-2-yl)-4- oxo-6-(2,4,6- trifluorobenzylamino)- 7-(trifluoromethyl)- 1,4-dihydroquinoline- 3-carboxylic acid | 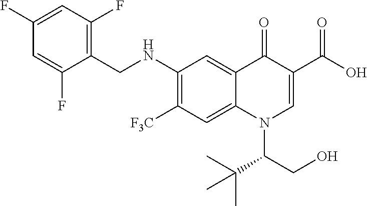 | 15.00 (s, 1H), 8.75 (s, 1H), 8.36 (s, 1H), 7.67 (s, 1H), 7.23 (t, 2H), 6.57 (t, 1H), 5.14 (m, 1H), 4.62 (d, 2H), 4.05 (m, 2H), 0.97 (s, 9H); MS (ESI): m/z 517 (M + 1)⁺ |
| 25B | (S)-6-(4-fluoro- benzylamino)-1-(1- hydroxy-3,3- dimethylbutan-2-yl)-4- oxo-7-(trifluoromethyl)- 1,4-dihydroquinoline- 3-carboxylic acid | 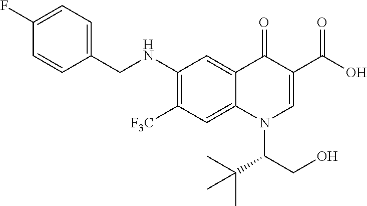 | 15.00 (s, 1H), 8.72 (s, 1H), 8.35 (s, 1H), 7.44 (m, 3H), 7.20 (m, 2H), 6.85 (m, 1H), 5.12 (m, 2H), 4.58 (d, 2H), 4.05 (m, 2H), 0.97 (s, 9H); MS (ESI): m/z 481 (M + 1)⁺ |
| 25C | (S)-6-(4-fluoro- benzylamino)-1-(1- hydroxy-3,3- dimethylbutan-2-yl)-7- methyl-4-oxo-1,4- dihydroquinoline-3- carboxylic acid | 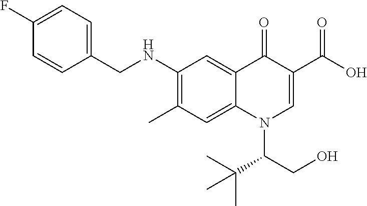 | |
| 25D | (S)-1-(1-hydroxy-3,3- dimethylbutan-2-yl)-7- methyl-4-oxo-6-(2,4,6- trifluorobenzylamino)- 1,4-dihydroquinoline- 3-carboxylic acid | 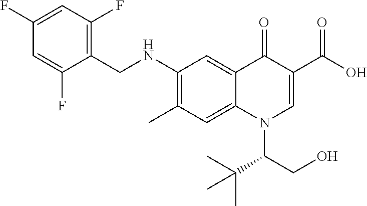 | |

### Example 26

### Compounds of Formula (XXVI)

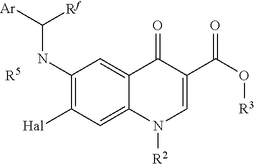

Compounds of formula (XXVI) were prepared according to the following synthetic scheme.

When appropriate, protecting groups are used as needed according to established synthetic procedures known to those of skill in the art, and may or may not be removed upon completion of the synthesis. The individual starting materials are synthesized according to methods known in the art or are commercially available.

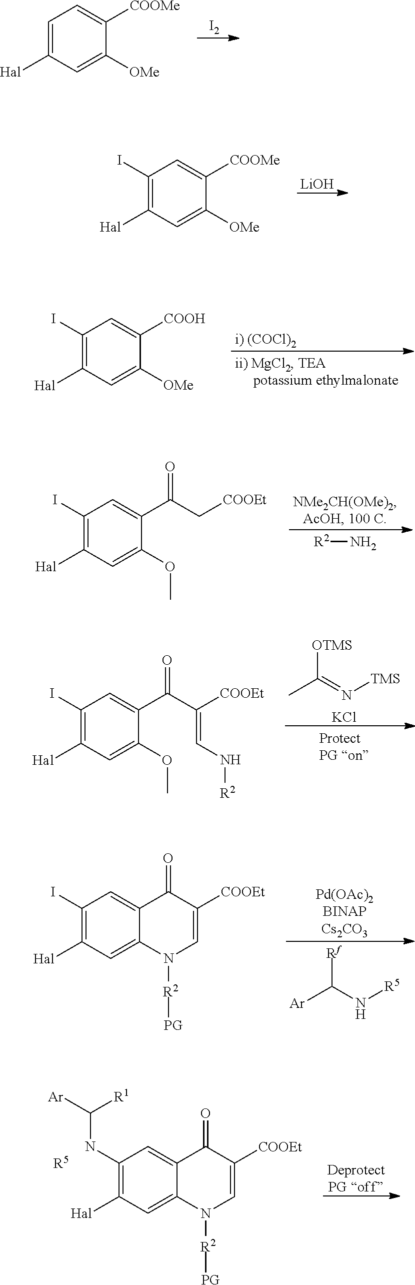

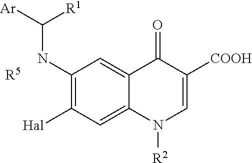

### Example 26A

### (S)-7-fluoro-6-(4-fluorobenzylamino)-1-(1-hydroxy-3,3-dimethylbutan-2-yl)-4-oxo-1,4-dihydroquinoline-3-carboxylic acid

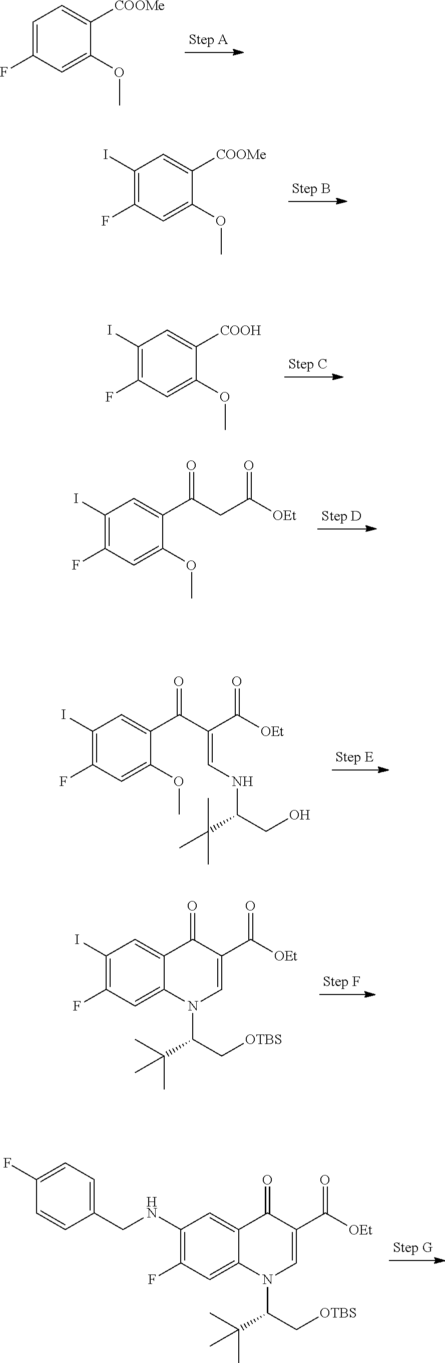

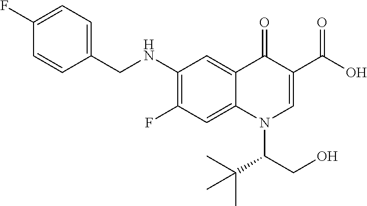

### Step A: Methyl 4-fluoro-5-iodo-2-methoxybenzoate

To a solution of methyl 4-fluoro-2-methoxybenzoate (2.54 g, 13.80 mmol) and silver triflate (5.35 g, 20.82 mmol) in MeOH (4o mL) was added iodine (5.35 g, 21.07 mmol) and stirred at rt for 2 h. The resulting precipitate was filtered off and the mother liquor was diluted with EtOAc (100 mL). The organic layer was washed (Na₂S₂O₄ (100 mL), 1-120 (100 mL), brine (100 mL)), dried over Na₂SO₄, and concentrated to give the desired product as a white solid (4.09 g). NMR (CDCl3): δ 8.23 (d, 1H), 6.75 (d, 1H), 3.92 (s, 3H), 3.91 (s, 3H).

### Step B: 4-Fluoro-5-iodo-2-methoxybenzoic acid

A solution of methyl 4-fluoro-5-iodo-2-methoxybenzoate (4.09 g, 13.19 mmol), and lithium hydroxide (5.55 g, 132.24 mmol) in a mixture of THF (10 mL), MeOH (5 mL), and was stirred at rt over night. The reaction mixture was diluted with H₂O (150 mL) and acidified (conc HCl). The resulting precipitate was collected by filtration to yield the desired compound as a white solid (3.95 g). NMR (CDCl3): δ 10.20 (s, 1H), 8.60 (d, 1H), 6.84 (d, 1H), 4.11 (s, 3H).

### Step C: Ethyl 3-(4-fluoro-5-iodo-2-methoxyphenyl)-3-oxopropanoate

To a solution of 4-Fluoro-5-iodo-2-methoxybenzoic acid (3.95 g, 13.34 mmol) and oxalyl chloride (1.4 mL, 16.27 mmol) in DCM (60 mL) was added DMF (0.2 mL). The reaction mixture was stirred at rt for 1 h and concentrated under reduced pressure. A solution of potassium ethylmalonate (4.12 g, 24.21 mmol) and magnesium chloride (4.32 g, 45.37 mmol) in THF (50 mL) was cooled to 0° C. and added above crude in THF (40 mL) followed by TEA (3.7 mL, 26.54 mmol). The mixture was stirred at that temperature for 30 min and at rt for 2 h. EtOAc (150 mL) and 1 N HCl (50 mL) were added to the reaction and stirred at rt for additional 10 min. The layers were separated and the organic layer was washed with satd NaHCO3 (2×100 mL), dried over Na2SO4 and concentrated. Purification on silica gel column gave the desired product as clear oil (3.44 g). NMR (CDCl3): δ 12.75 (s, 0.1H), 8.31 (s, 0.9H), 8.25 (d, 0.1H), 6.74 (m, 1H), 6.01 (s, 0.1H), 4.30 (q, 0.2H), 4.21 (q, 1.8H), 3.94 (s, 1.8H), 3.92 (s, 3H), 1.37 (t, 0.3H), 1.27 (t, 2.7H).

### Step D: (S)-Ethyl 2-(4-fluoro-5-iodo-2-methoxybenzoyl)-3-(1-hydroxy-3,3-dimethylbutan-2-ylamino)acrylate

A solution of ethyl 3-(4-fluoro-5-iodo-2-methoxyphenyl)-3-oxopropanoate (3.44 g, 9.40 mmol) and 1,1-dimethoxy-N,N-dimethylmethanamine (1.6 mL, 11.95 mmol) and acetic acid (0.05 mL) in toluene (10 mL) was heated at 100° C. for 1 h. The reaction mixture was cooled tort and added (S)-2-amino-3,3-dimethylbutan-1-ol (1.21 g, 10.32 mmol) which was then stirred for additional 30 min. Purification on silica gel column gave the desired product as clear oil (4.48 g). NMR (CDCl3): δ 11.12 (t, 0.8H), 9.50 (t, 0.2H), 8.10 (d, 0.2H), 8.08 (d, 0.8H), 7.62 (d, 0.2H), 7.58 (d, 0.2H), 6.64 (d, 1H), 3.99 (m, 3H), 3.77 (s, 3H), 3.68 (m, 1H), 3.09 (m, 1H), 2.21 (m, 1H), 1.03 (m, 12H), 0.95 (t, 9.6H).

### Step E: (S)-Ethyl 1-(1-(tert-butyldimethylsilyloxy)-3,3-dimethylbutan-2-yl)-7-fluoro-6-iodo-4-oxo-1,4-dihydroquinoline-3-carboxylate

A solution of (S)-ethyl 2-(4-fluoro-5-iodo-2-methoxybenzoyl)-3-(1-hydroxy-3,3-dimethylbutan-2-ylamino)acrylate (4.48 g, 9.09 mmol), KCl (700 mg, 9.39 mmol), and trimethylsilyl N-trimethylsilylacetimidate (5.1 mL, 20.56 mmol) in DMF (10 mL) was heated at 100° C. over night. The reaction mixture was acidified with HCl (1 N, 100 mL), and stirred for 10 min. The resulting precipitate was collected by filtration and re-dissolved in DMF (20 mL). Imidazole (6.19 g, 90.92 mmol) and tert-butylchlorodimethylsilane (6.91 g, 45.85 mmol) were added to the reaction mixture and stirred for additional 1 h. The reaction mixture was diluted with EtOAc (100 mL), washed with H₂O (2×50 mL), dried over Na₂SO₄ and concentrated. Purification on silica gel column gave the desired product as yellow foam (4.30 g). NMR (CDCl3): δ 8.97 (d, 1H), 8.68 (s, 1H), 7.35 (d, 1H), 4.39 (m, 3H), 4.15 (m, 2H), 1.43 (t, 3H), 1.09 (s, 9H), 0.70 (s, 9H), 0.02 (s, 3H), −0.06 (s, 3H); MS (ESI): m/z 576 (M+1)⁺.

### Step F: (S)-Ethyl 1-(1-(tert-butyldimethylsilyloxy)-3,3-dimethylbutan-2-yl)-7-fluoro-6-(4-fluorobenzylamino)-4-oxo-1,4-dihydroquinoline-3-carboxylate

A solution of (S)-ethyl 1-(1-(tert-butyldimethylsilyloxy)-3,3-dimethylbutan-2-yl)-7-fluoro-6-iodo-4-oxo-1,4-dihydroquinoline-3-carboxylate (210 mg, 0.36 mmol), (4-fluorophenyl)methanamine (95 mg, 0.76 mmol), Pd(OAc)₂ (18 mg, 0.08 mmol), BINAP (95 mg, 0.15 mmol), and Cs₂CO₃ (240 mg, 0.74 mmol) in toluene (2 mL) was degassed by bubbling nitrogen for 20 min then heated at 100° C. for 4 h. Purification on silica gel column gave the desired product as yellow oil (150 mg). MS (ESI): m/z 573 (M+1)⁺.

### Step G: (S)-7-fluoro-6-(4-fluorobenzylamino)-1-(1-hydroxy-3,3-dimethylbutan-2-yl)-4-oxo-1,4-dihydroquinoline-3-carboxylic acid

A solution (S)-ethyl 1-(1-(tert-butyldimethylsilyloxy)-3,3-dimethylbutan-2-yl)-7-fluoro-6-(4-fluorobenzylamino)-4-oxo-1,4-dihydroquinoline-3-carboxylate (150 mg, 0.26 mmol), NaOCH₃ (2 mL, 25% in MeOH), and H₂O (2 mL) in MeOH (2 mL) was heated at 65° C. for 4 h. The reaction mixture was concentrated under reduced pressure to a small volume and diluted with H₂O (10 mL). The solution was acidified (1 N HCl) and the resulting precipitate was collected by filtration to yield the desired compound as an off-white solid (110 mg). NMR (DMSO-d₆): δ 15.60 (s, 1H), 8.68 (s, 1H), 8.35 (d, 1H), 7.46 (m, 2H), 7.33 (d, 1H), 7.18 (m, 2H), 7.14 (m, 1H), 5.10 (t, 1H), 4.96 (m, 1H), 4.48 (d, 2H), 4.03 (m, 2H), 0.97 (s, 9H); MS (ESI): m/z 431 (M+1)⁺.

### Examples 26B-26E

Examples 26B-26E were prepared according to the procedure described above for example 26A.

Examples 26F is prepared according to the procedure described above for example 26A.

| | | | ¹H NMR (400 MHz) 25° C. δ |
|---|---|---|---|
| Compound | Compound Name | Structure | DMSO-d6: MS (ESI) |
| 26A | (S)-7-fluoro-6-(4- fluorobenzylamino)-1- (1-hydroxy-3,3- dimethylbutan-2-yl)-4- oxo-1,4-dihydroquinoline- 3-carboxylic acid | 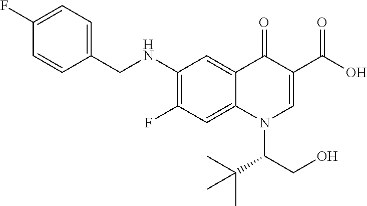 | 15.60 (s, 1H), 8.68 (s, 1H), 8.35 (d, 1H), 7.46 (m, 2H), 7.33 (d, 1H), 7.18 (m, 2H), 7.14 (m, 1H), 5.10 (t, 1H), 4.96 (m, 1H), 4.48 (d, 2H), 4.03 (m, 2H), 0.97 (s, 9H); MS (ESI): m/z 431 (M + 1)⁺ |
| 26B | (S)-6-(2,4-difluoro- benzylamino)-7-fluoro- 1-(1-hydroxy-3,3- dimethylbutan-2-yl)-4- oxo-1,4-dihydroquinoline- 3-carboxylic acid | 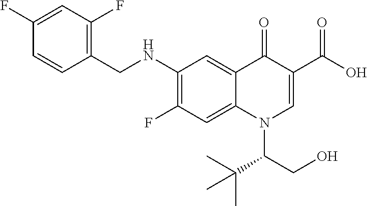 | 15.60 (s, 1H), 8.68 (s, 1H), 8.35 (d, 1H), 7.45 (m, 1H), 7.37 (d, 1H), 7.32 (m, 1H), 6.95 (m, 2H), 5.12 (t, 1H), 4.98 (m, 1H), 4.52 (d, 2H), 4.03 (m, 2H), 0.98 (s, 9H); MS (ESI): m/z 449 (M + 1)⁺ |
| 26C | (S)-6-(2,6-difluoro- benzylamino)-7- fluoro-1-(1-hydroxy- 3,3-dimethylbutan-2-yl)- 4-oxo-1,4- dihydroquinoline-3- carboxylic acid | 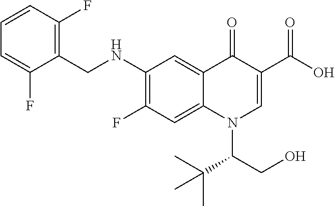 | 15.60 (s, 1H), 8.68 (s, 1H), 8.35 (d, 1H), 7.63 (d, 1H), 7.43 (m, 1H), 7.15 (m, 2H), 6.85 (t, 1H), 5.12 (t, 1H), 4.96 (m, 1H), 4.52 (d, 2H), 4.03 (m, 2H), 0.97 (s, 9H); MS (ESI): m/z 449 (M + 1)⁺ |
| 26D | (S)-7-chloro-1-(1- hydroxy-3,3-dimethyl butan-2-yl)-4-oxo-6- (2,4,6-trifluorobenzyl- amino)-1,4-dihydro- quinoline-3-carboxylic acid | 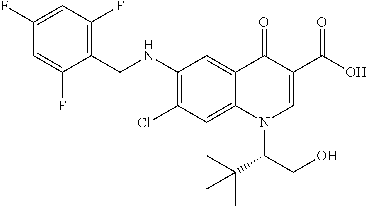 | 8.69 (s, 1H), 8.48 (s, 1H), 7.60 (s, 1H), 7.24 (t, 2H), 6.45 (m, 1H), 5.11 (m, 1H), 5.03 (m, 1H), 4.53 (d, 2H), 4.03 (m, 2H), 0.97 (s, 9H); MS (ESI): m/z 483 (M + 1)⁺ |
| 26E | (S)-7-chloro-6-(4- fluorobenzylamino)-1- (1-hydroxy-3,3- dimethylbutan-2-yl)-4- oxo-1,4-dihydroquinoline- 3-carboxylic acid | 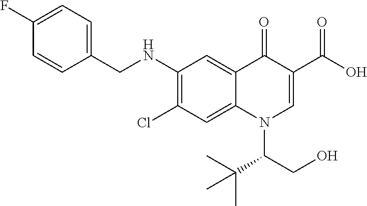 | 15.30 (s, 1H), 8.66 (s, 1H), 8.55 (s, 1H), 7.44 (m, 2H), 7.29 (s, 1H), 7.19 (m, 2H), 6.98 (t, 1H), 5.09 (m, 2H), 4.54 (d, 2H), 4.03 (m, 2H), 0.97 (s, 9H); MS (ESI): m/z 447 (M + 1)⁺ |
| 26F | | 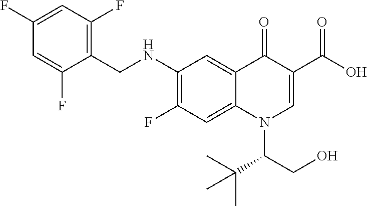 | |

### Example 27

### Compounds of Formula (XXVII)

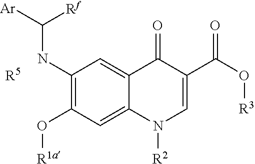

Compounds of formula (XXVII) were prepared according to the following synthetic scheme.

When appropriate, protecting groups are used as needed according to established synthetic procedures known to those of skill in the art, and may or may not be removed upon completion of the synthesis. The individual starting materials are synthesized according to methods known in the art or are commercially available.

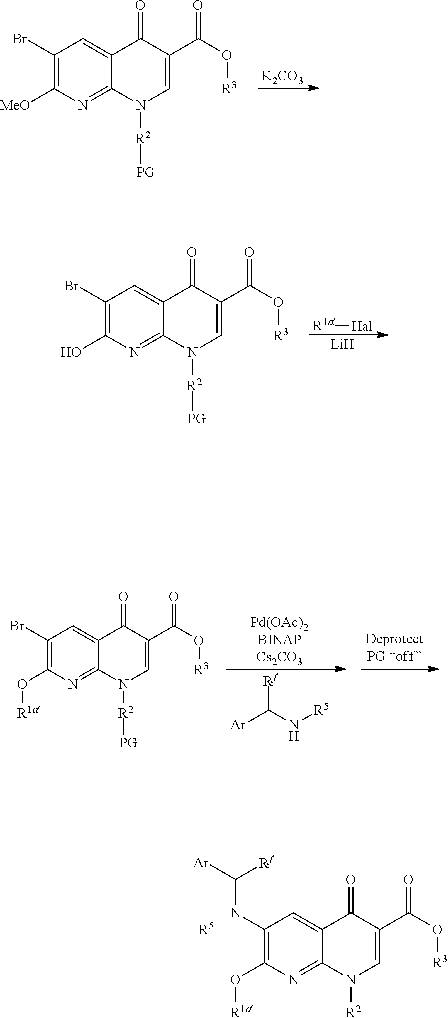

### Example 27A

### (S)-7-Ethoxy-6-(4-fluorobenzylamino)-1-(1-hydroxy-3-methylbutan-2-yl)-4-oxo-1,4-dihydro-1,8-naphthyridine-3-carboxylic acid

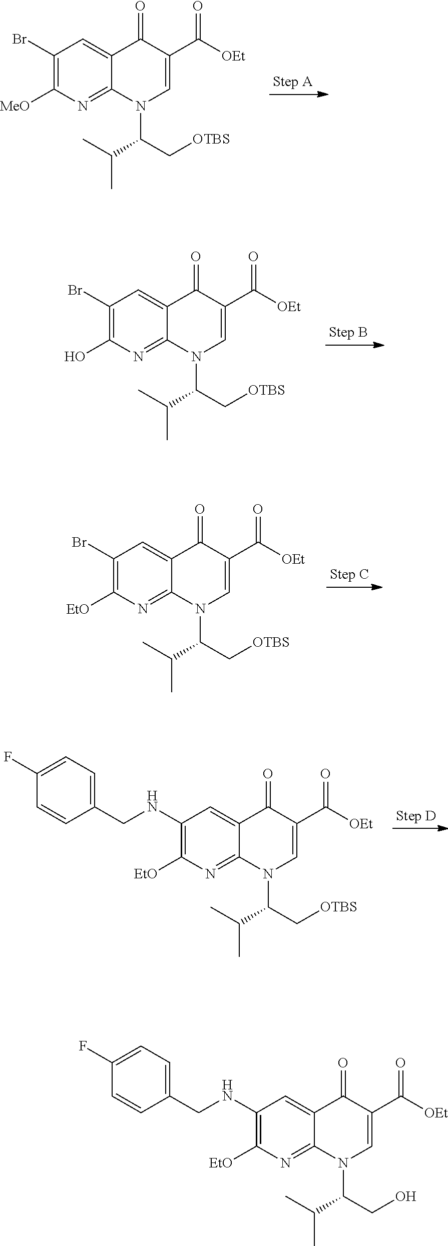

### Step A: (S)-Ethyl 6-bromo-1-(1-(tert-butyldimethylsilyloxy)-3-methylbutan-2-yl)-7-hydroxy-4-oxo-1,4-dihydro-1,8-naphthyridine-3-carboxylate

A solution of (S)-6-bromo-1-(1-(tert-butyldimethylsilyloxy)-3-methylbutan-2-yl)-7-methoxy-4-oxo-1,4-dihydro-1,8-naphthyridine-3-carboxylic acid (1.15 g, 2.18 mmol), morpholine (0.38 mL, 4.34 mmol), and K₂CO₃ (600 mg, 4.34 mmol) in DMSO (5 mL) was heated at 120° C. over night. The reaction mixture was cooled to rt, diluted with H₂O (15 mL), and stirred for additional 10 min. The resulting precipitate was collected by filtration to give, the desired product as a white solid (1.06 g). ¹H NMR (DMSO-d₆): δ 8.40 (s, 1H), 8.06 (s, 1H), 5.31 (m, 1H), 4.16 (m, 2H), 4.06 (dd, 1H), 3.64 (dd, 1H), 2.30 (m, 1H), 1.25 (t, 3H), 1.12 (d, 3H), 0.86 (s, 9H), 0.73 (d, 3H), 0.01 (s, 3H), −0.04 (s, 3H); MS (ESI): m/z 513 (M+1)⁺.

### Step B: (S)-Ethyl 6-bromo-1-(1-(tert-butyldimethylsilyloxy)-3-methylbutan-2-yl)-7-ethoxy-4-oxo-1,4-dihydro-1,8-naphthyridine-3-carboxylate

To a solution of (S)-6-bromo-1-(1-(tert-butyldimethylsilyloxy)-3-methylbutan-2-yl)-7-hydroxy-4-oxo-1,4-dihydro-1,8-naphthyridine-3-carboxylic acid (250 mg, 0.49 mmol) in DMF (2 mL) was added LiH (9 mg, 1.13 mmol) at rt. The mixture was stirred for 20 min and at that point ethyl iodide (0.1 mL, 1.25 mmol) was added and stirred for additional 3 h. The reaction mixture was diluted with EtOAc (20 mL), washed with H₂O (2×5 mL), and dried over Na₂SO₄. The solvent was removed under reduced pressure and purified on silica gel column to yield the desired product. ¹H NMR (DMSO-d₆): δ 8.88 (s, 1H), 8.79 (s, 1H), 5.25 (m, 1H), 4.52 (q, 2H), 4.45 (q, 2H), 4.12 (dd, 1H), 3.84 (dd, 1H), 2.45 (m, 1H), 1.55 (t, 3H), 1.45 (t, 3H), 1.20 (d, 3H), 0.86 (s, 9H), 0.83 (d, 3H), 0.03 (s, 3H), 0.01 (s, 31-1); MS (ESI): m/z 541 (M+1)⁺.

### Step C: (S)-Ethyl 1-(1-(tert-butyldimethylsilyloxy)-3-Methylbutan-2-yl)-7-ethoxy-6-(4-fluorobenzylamino)-4-oxo-1,4-dihydro-1,8-naphthyridine-3-carboxylate

A solution of (S)-Ethyl 6-bromo-1-(1-(tert-butyldimethylsilyloxy)-3-methylbutan-2-yl)-7-ethoxy-4-oxo-1,4-dihydro-1,8-naphthyridine-3-carboxylate (75 mg, 0.14 mmol), (4-fluorophenyl)methanamine (36 mg, 0.28 mmol), Pd(OAc)₂ (6 mg, 0.03 mmol), BINAP (35 mg, 0.06 mmol), and Cs₂CO₃ (90 mg, 0.28 mmol) in dioxane (1.5 mL) was degassed by bubbling nitrogen for 15 min then heated at 100° C. over night. The reaction mixture was diluted with EtOAc (10 mL), washed with H₂O (2×10 mL), and dried over Na₂SO₄. The solvent was removed under reduced pressure and purified on silica gel plate to yield the desired product as foam (30 mg). MS (ESI): m/z 586 (M+1)⁺.

### Step D: (S)-7-Ethoxy-6-(4-fluorobenzylamino)-1-(1-hydroxy-3-methylbutan-2-yl)-4-oxo-1,4-dihydro-1,8-naphthyridine-3-carboxylic acid

A solution (S)-ethyl 1-(1-(tert-butyldimethylsilyloxy)-3-methylbutan-2-yl)-7-ethoxy-6-(4-fluorobenzylamino)-4-oxo-1,4-dihydro-1,8-naphthyridine-3-carboxylate (30 mg, 0.05 mmol), NaOCH₃ (0.5 mL, 25% in MeOH), and H₂O (1 mL) in MeOH (1 mL) was heated at 65° C. for 1 h. The reaction mixture was concentrated under reduced pressure to a small volume and diluted with H₂O (10 mL). The solution was acidified (1 N HCl) and the resulting precipitate was collected by filtration to yield the desired compound as an off-white solid (10 mg). NMR (CDCl3): δ 15.60 (s, 1H), 8.80 (s, 1H), 7.43 (m, 3H), 7.15 (m, 2H), 5.50 (m, 1H), 4.60 (q, 2H), 4.43 (s, 2H), 4.25 (m, 1H), 4.10 (m, 1H), 2.45 (m, 1H), 2.03 (m, 1H), 1.66 (m, 1H), 1.56 (t, 3H), 1.20 (d, 3H), 0.78 (d, 3H); MS (ESI): m/z 444 (M+1)⁺.

### Examples 27B-27E

Examples 27B-27E were prepared according to the procedure described above for example 27A,

Examples 27F is prepared according to the procedure described above for example 27A.

| | | | ¹H NMR (400 MHz) 25° C. δ |
|---|---|---|---|
| Compound | Compound Name | Structure | DMSO-d6: MS (ESI) |
| 27A | (S)-7-Ethoxy-6-(4- fluorobenzylamino)- 1-(1-hydroxy-3- methylbutan-2-yl)-4- oxo-1,4-dihydro-1,8- naphthyridine-3- carboxylic acid | 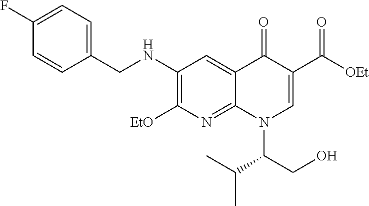 | (CDCl₃): δ 15.60 (s, 1H), 8.80 (s, 1H), 7.43 (m, 3H), 7.15 (m, 2H), 5.50 (m, 1H), 4.60 (q, 1H), 4.43 (s, 2H), 4.25 (m, 1H), 4.10 (m, 1H), 2.45 (m, 1H), 2.03 (m, 1H), 1.66 (m, 1H), 1.56 (t, 3H), 1.20 (d, 3H), 0.78 (d, 3H); MS (ESI): m/z 444 (M + 1)⁺. |
| 27B | (S)-7-Ethoxy-1-(1- hydroxy-3- methylbutan-2-yl)-4- oxo-6-(2,4,6- trifluorobenzylamino)- 1,4-dihydro-1,8- naphthyridine-3- carboxylic acid | 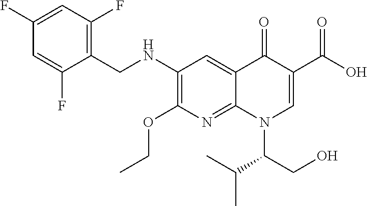 | 15.40 (s, 1H), 8.82 (s, 1H), 7.50 (s, 1H), 7.27 (t, 2H), 6.50 (t, 1H), 5.42 (m, 1H), 5.16 (m, 1H), 4.58 (m, 2H), 4.48 (d, 2H), 4.05 (m, 1H), 3.78 (m, 1H), 2.35 (m, 1H), 1.45 (t, 3H), 1.13 (d, 3H), 0.69 (d, 3H); MS (ESI): m/z 480 (M + 1)⁺ |
| 27C | (S)-6-(4-fluoro- benzylamino)-1- (1-hydroxy-3,3- dimethylbutan-2-yl)- 7-(2-methoxyethoxy)- 4-oxo-1,4- dihydroquinoline-3- carboxylic acid | 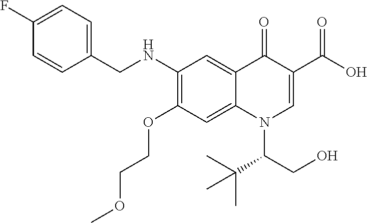 | 8.63 (s, 1H), 7.63 (m, 1H), 7.54 (s, 1H), 7.43 (m, 2H), 7.17 (m, 2H), 7.13 (s, 1H), 6.53 (t, 1H), 5.09 (m, 2H), 4.54 (m, 4H), 4.07 (m, 2H), 3.85 (t, 2H), 3.40 (s, 3H), 0.97 (s, 9H); MS (ESI): m/z 487 (M + 1)⁺ |
| 27D | (S)-1-(1-hydroxy-3,3- dimethylbutan-2- yl)-7-methyl-4- oxo-6-(2,4,6-trifluoro- benzylamino)-1,4- dihydro-1,8- naphthyridine-3- carboxylic acid | 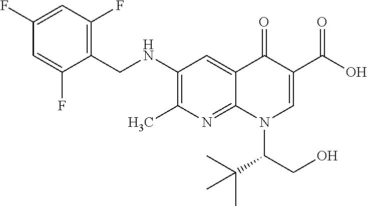 | 15.40 (s, 1H), 8.77 (s, 1H), 7.66 (s, 1H), 7.27 (t, 2H), 6.52 (t, 1H), 5.98 (m, 1H), 5.12 (m, 1H), 4.48 (d, 2H), 4.05 (m, 2H), 2.58 (s, 3H), 0.97 (s, 9H); MS (ESI): m/z 464 (M + 1)⁺ |
| 27E | (S)-1-(1-hydroxy-3,3- dimethylbutan-2- yl)-7-morpholino- 4-oxo-6-(2,4,6- trifluorobenzylamino)- 1,4-dihydro-1,8- naphthyridine-3- carboxylic acid | 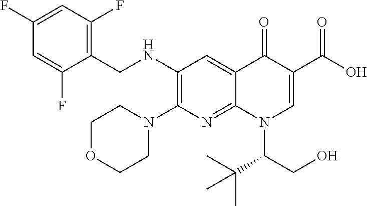 | 15.75 (s, 1H), 8.69 (s, 1H), 7.58 (s, 1H), 7.25 (t, 2H), 6.14 (t, 1H), 5.46 (t, 1H), 5.80 (t, 1H), 5.00 (broad s, 1H), 4.45 (d, 2H), 4.04 (broad, 2H), 3.85 (t, 4H), 3.40 (broad s, 4H), 0.96 (broad s, 9H) |
| 27F | | 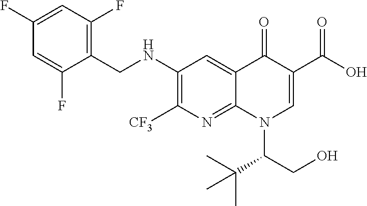 | |

### Example 28

### Compounds of Formula (XXVIII)

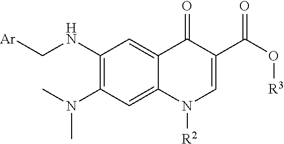

Compounds of formula (XXVII) were prepared according to the following synthetic scheme.

When appropriate, protecting groups are used as needed according to established synthetic procedures known to those of skill in the art, and may or may not be removed upon completion of the synthesis. The individual starting materials are synthesized according to methods known in the art or are commercially available.

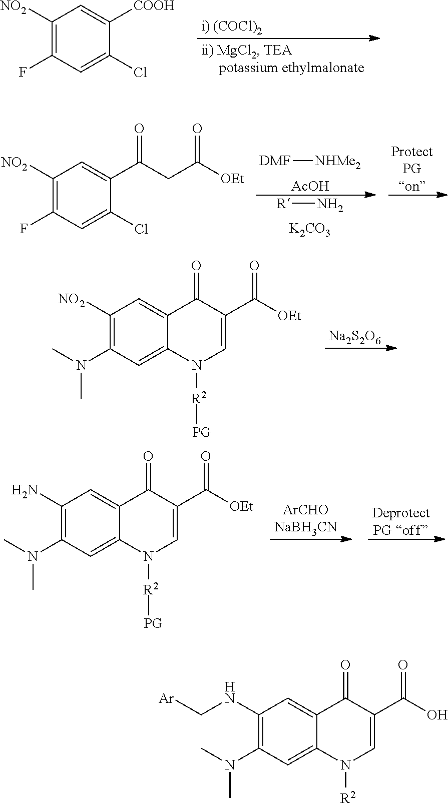

### Example 28A

### (S)-7-(dimethylamino)-1-(1-hydroxy-3-methylbutan-2-yl)-4-oxo-6-(2,4,6-trifluorobenzylamino)-1,4-dihydroquinoline-3-carboxylic acid

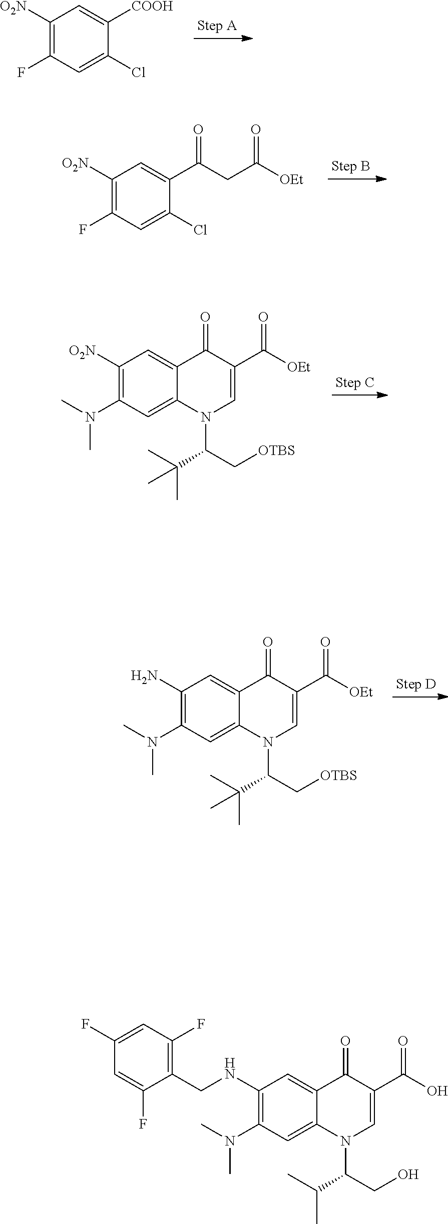

### Step A: Ethyl 3-(2-chloro-4-fluoro-5-nitrophenyl)-3-oxopropanoate

A 100 mL flask was charged with 2-chloro-4-fluoro-5-nitrobenzoic acid (2.20 g, 10.00 mmol) in 10 ml dry methylene chloride. To the suspension was added oxalyl chloride (1.13 mL, 13.00 mmol) and few drops of DMF. The reaction mixture was stirred at RT for 4 h. The mixture was condensed under reduced pressure yielding light yellow solids. An another 250 mL RB flask was charged with magnesium chloride (2.86 g, 30.00 mmol), potassium ethylmalonate (3.06 g, 18.00 mmol), and triethylamine (2.77 mL, 20.00 mmol) in 120 mL dry acetonitrile. To the suspension was added the solids in 40 mL acetonitrile at 0° C. The mixture was stirred at the temperature for 30 min. Then the mixture was warmed to room temperature and further stirred for 14 h. The mixture was condensed under reduced pressure yielding brown residue. The residue was dissolved in 40 mL toluene. To the solution was added 30 mL 1 N HCl solution. The biphasic mixture was stirred at room temperature for 3 h. The organic layer separated and was dried over sodium sulfate. It was condensed under reduced pressure yielding a brown residue. The residue was purified flash chromatography (Biotage 0-20%, Ethyl acetate/hexanes) yielding off-white solids (2.18 g, 75%)

¹H NMR (CDCl₃, 400 MHz): δ 12.58 (s, 1H), 8.47 and 8.41 (2×d, J=7.8 Hz, 1H keto and enol tautomers), 7.47 (dd, J1=9.9 Hz, J2=2.0 Hz1H), 5.66 (s, 1H) 4.33 and 4.31 (q, 2H, tautomers), 1.38 and 1.30 (t, 3H, tautomers)

### Step B: (S)-ethyl 1-(1-(tert-butyldimethylsiloxy)-3,3,-dimethylbutan-2-yl)-7-(dimethylamino)-6-nitro-4-oxo-1,4-dyhydroquinoline-3-carbxylate

A 250 mL RB flask was charged with Ethyl 3-(2-chloro-4-fluoro-5-nitrophenyl)-3-oxopropanoate (2.18 g, 7.54 mmol), DMF-DMA (1.21 mL, 8.80 mmol) in 40 mL dry toluene. To the mixture was added 4 drops of acetic acid by a disposable pipet. The reaction mixture was stirred at 110° C. for 4 h. The mixture was cooled down to room temperature. To the mixture was added tort-Leucinol (0.95 g, 8.00 mmol) as a solid, and further stirred for 30 min. The mixture was condensed under reduced pressure yielding a yellow residue. Without further purification, the residue was dissolved in 24 mL DMF. To the reaction mixture was added potassium carbonate (1.65 g, 12.00 mmol), and stirred at 60° C. for 6 h. The mixture was cooled down to RT. To the mixture was added 1 N HCl solution after which yellow solids precipitated from the solution. The precipitate was filtered and dried under reduced pressure. The solids were dissolved in 15 mL DMF. To the mixture was added TBSCl (2.26 g, 15.00 mmol) and imidazole (1.36 g, 20.00 mmol). The reaction mixture was stirred at room temperature for 14 h. The mixture was condensed under reduced pressure to give an amber residue. The residue was purified by flash Chromatography (0-50% ethyl acetate/Hexanes) providing a yellow foamy solid (1.10 g, 28% for the three steps). 1H NMR (CDCl3, 400 MHz): δ 8.92 (s, 1H), 8.63 (s, 1H), 6.89 (s, 1H), 4.51 (d, 1H), 4.45 (dd, 2H), 4.41 (q, 2H), 3.02 (s, 6H), 1.44 (t, 2H), 1.10 (s, 9H), 0.72 (s, 9H), 0.05, −0.08 (2×s, 6H).

### Step C: (S)-ethyl 6-amino-1-(1-(tert-buthyldimethylsiloxy)-3,3-dimethylbutan-2-yl)-7-(dimethylamino-4-oxo-1,4-dihydroquinoline-3-carboxylate

A 100 mL RB flask was charged with (S)-ethyl 1-(1-(tert-butyldimethylsiloxy)-3,3,-dimethylbutan-2-yl)-7-(dimethylamino)-6-nitro-4-oxo-1,4-dyhydroquinoline-3-carbxylate in 5 mL THF. To the mixture was added sodium dithionate (1.00 g, 4.88 mmol) in 5 mL H₂O. The reaction was stirred at room temperature for 2 h. The mixture was diluted with ethyl acetate. The organic layer was dried under reduced pressure yielding a yellow residue. The residue was purified by flash chromatography (Biotage, 0-85% Ethyl acetate/Hexanes) yielding 60 mg of a yellow foamy solid (65%). ¹H NMR (CDCl₃, 400 MHz): δ 8.60 (s, 1H), 7.82 (s, 1H), 7.13 (s, 1H), 4.58 (dd, 1H), 4.41 (q, 2H), 2.81 (s, 6H), 1.44 (t, 3H), 1.08 (s, 9H), 0.70 (s, 9H), 0.03 and −0.08 (2×s, 6H)

### Step D: (S)-7-(dimethylamino)-1-(1-hydroxy-3-methylbutan-2-yl)-4-oxo-6-(2,4,6-trifluorobenzylamino)-1,4-dihydroquinoline-3-carboxylic acid

A 100 mL RB flask was charged with (S)-ethyl 6-amino-1-(1-(tert-buthyldimethylsiloxy)-3,3-dimethylbutan-2-yl)-7-(dimethylamino-4-oxo-1,4-dihydroquinoline-3-carboxylate (190 mg, 0.39 mmol) and trifluorobenzyl aldehyde (64 mg, 0.40 mmol) in 10 mL methylene chloride. The reaction was stirred at room temperature for 14 h. The solvent was removed by rotavap and the remaining residue was dissolved in 4 mL methanol. To the mixture was added sodium cynanoborohydride (48 mg, 0.78 mmol) and further stirred at the temperature for 30 min. The reaction was quenched with 1 N HCl solution. It was the extracted with ethyl acetate and dried over sodium sulfate. The mixture was condensed under reduced pressure yielding light yellow residue. The residue was purified by flash chromatography (Biotage, 0-60%. Ethyl acetate/Hexanes) to give a foamy solid. (138 mg, 56%). The solid was dissolved in the mixture of 25% MeONa/MeOH (1.0 mL), MeOH (1.0 mL) and H2O (1.0 mL). The reaction mixture was stirred at 65° C. for 2 h. The mixture was condensed under reduced pressure yielding aqueous layer. The layer was acidified by 1 N HCl solution yielding yellow solids from the mixture. The solids were filtered and dried under reduced pressure providing a yellow solid (82 mg, 43% for the two steps). 1H NMR (DMSO-d6, 400 MHz): δ 8.65 (s, 1H), 7.52 (broad s, 1H), 7.45 (s, 1H), 7.24 (t, 2H), 6.00 (broad s, 1H), 5.10 (broad s, 2H), 4.48 (s, 2H), 4.07 (s, 2H), 2.78 (s, 6H), 0.97 (s, 9H) MS: 492, 493 (M+1).

### Examples 28B-28C

Examples 28B-28C are prepared according to the procedure described above for example 28A.

| | | | ¹H NMR (400 MHz) 25° C. δ |
|---|---|---|---|
| Compound | Compound Name | Structure | DMSO-d6: MS (ESI) |
| 28A | (S)-7-(dimethylamino)- 1-(1-hydroxy-3- methylbutan-2-yl)- 4-oxo-6-(2,4,6- trifluorobenzylamino)- 1,4-dihydroquinoline- 3-carboxylic acid | 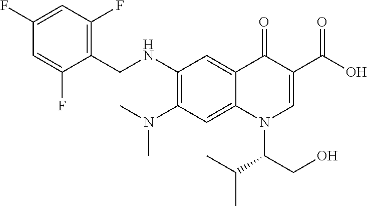 | 8.65 (s, 1H), 7.52 (broad s, 1H), 7.45 (s, 1H), 7.24 (t, 2H), 6.00 (broad s, 1H), 5.10 (broad s, 2H), 4.48 (s, 2H), 4.07 (s, 2H), 2.78 (s, 6H), 0.97 (s, 9H) MS: 492, 493 (M + 1) |
| 28B | | 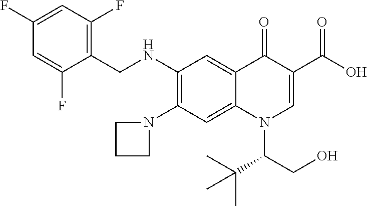 | |
| 28C | | 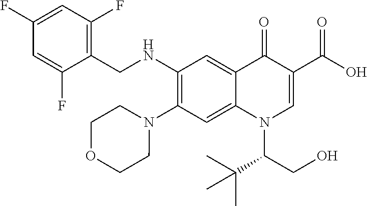 | |

### Example 29

### Compounds of Formula (XXIX)

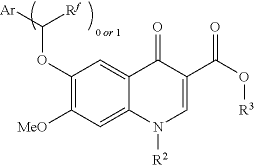

Compounds of formula (XXIX) were prepared according to the following synthetic scheme.

When appropriate, protecting groups are used as needed according to established synthetic procedures known to those of skill in the art, and may or may not be removed upon completion of the synthesis. The individual starting materials are synthesized according to methods known in the art or are commercially available.

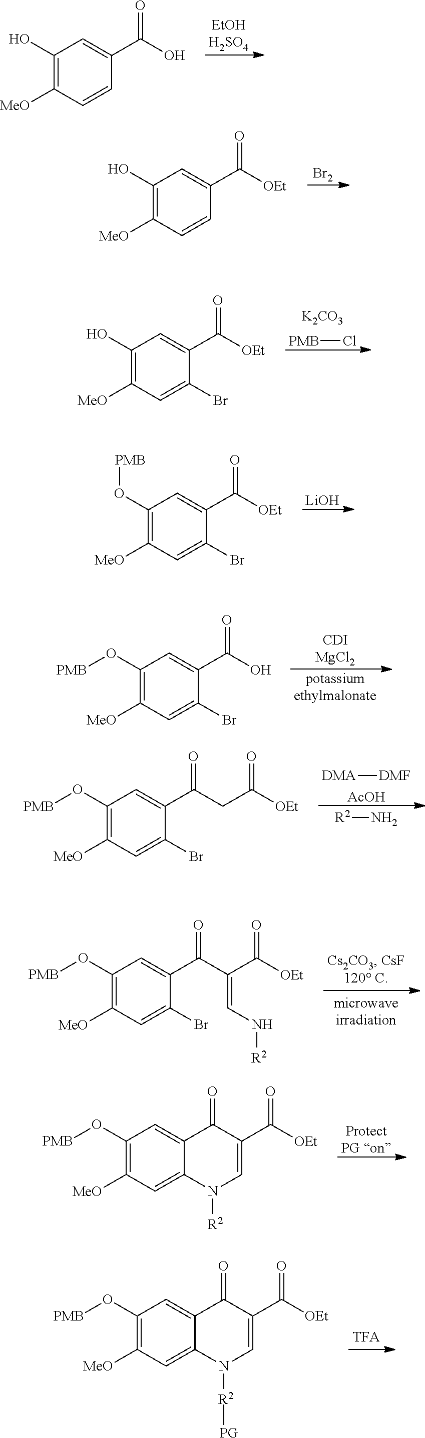

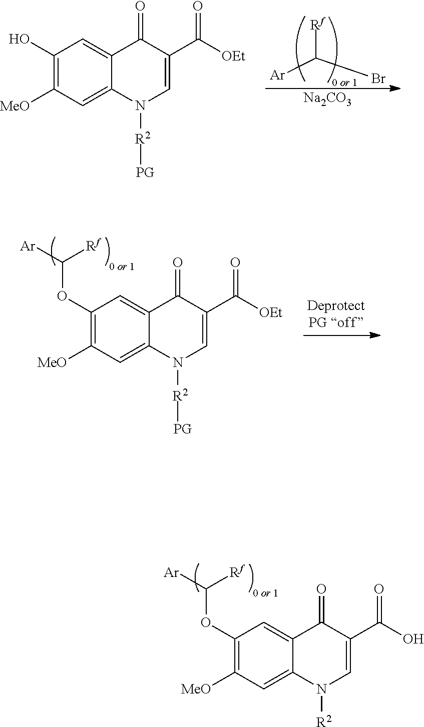

### Example 29A

### (S)-6-(3-chloro-2-fluorobenzyloxy)-1-(1-hydroxy-3,3-dimethylbutan-2-yl)-7-methoxy-4-oxo-1,4-dihydroquinoline-3-carboxylic acid

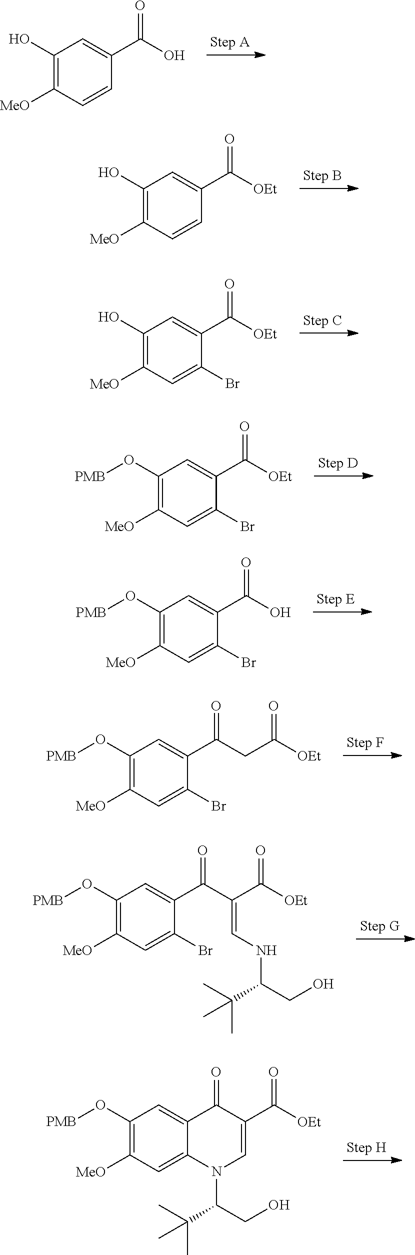

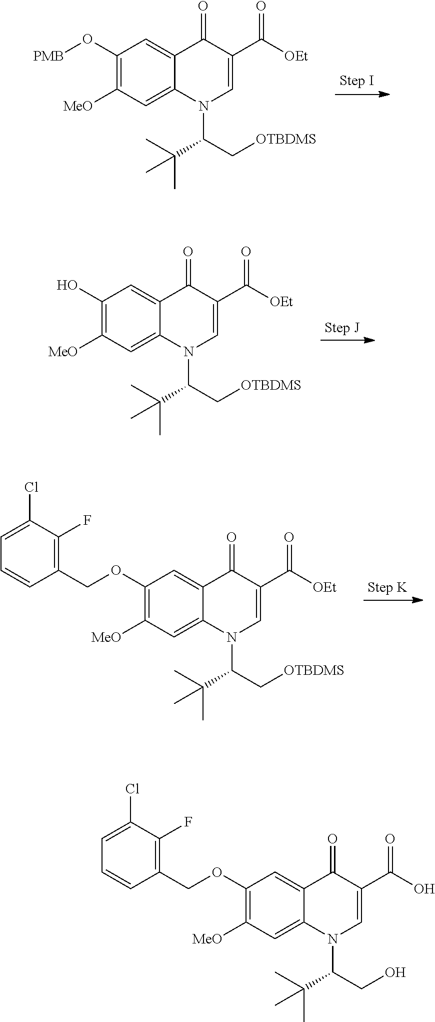

### Step A: Ethyl 3-hydroxy-4-methoxybenzoate

A mixture of 3-hydroxy-4-methoxybenzoic acid (8.40 g, 50 mmol), EtOH (50 ml) and H₂SO₄ (10 ml) was stirred and heated to 80° C. for 15 hours. After cooling to room temperature, the mixture was basified with saturated aqueous NaHCO₃ and extracted with ethyl acetate. The organic layer was dried over MgSO₄, concentrated and chromatographed to obtain ethyl 3-hydroxy-4-methoxybenzoate (8.10 g, 41.3 mmol, 83%).

### Step B: Ethyl 2-bromo-5-hydroxy-4-methoxybenzoate

Bromine (1.24 ml, was added to a cold (5° C.) mixture of ethyl 3-hydroxy-4-methoxybenzoate (6.77 g, 34.5 mmol) in acetic acid (50 ml). The mixture was slowly warmed to room temperature and stirred for 15 hours. The mixture was washed with aqueous Na2S2O4 and extracted with ethyl acetate. The organic layer was dried over MgSO4, concentrated and chromatographed (DMC) to obtain a 2 to 1 mixture of ethyl 2-bromo-5-hydroxy-4-methoxybenzoate and ethyl 6-bromo-5-hydroxy-4-methoxybenzoate (4.87 g, 17.7 mmol, 51%). This mixture can be separated in the subsequent step C. 1H NMR (CDCl3, 400 MHz): δ 7.23 (s, 1H), 7.08 (s, 1H), 4.22 (q, J=7.1 Hz, 2H), 3.92 (s, 3H), 1.27 (t, J=7.1 Hz, 3H).

### Step C: Ethyl 2-bromo-4-methoxy-5-(4-methoxybenzyloxy)benzoate

A mixture of 2-bromo-5-hydroxy-4-methoxybenzoate+ethyl 6-bromo-5-hydroxy-4-methoxybenzoate (2:1, 4.81 g, 17.6 mmol), K₂CO₃ (4.14 g, 30 mmol) and p-methoxybenzoylchloride (3.18 ml, 23.5 mmol) was stirred and heated to 90° C. for 3 hours. The mixture was washed with water, extracted with ethyl acetate. The organic layer was dried over MgSO₄, concentrated and chromatographed (DMC) to obtain ethyl 2-bromo-4-methoxy-5-(4-methoxybenzyloxy)benzoate (4.69 g, 11.8 mmol, 96%) and ethyl 6-bromo-4-methoxy-5-(4-methoxybenzyloxy)benzoate (2.17 g, 5.5 mmol, 96%). ¹H NMR (DMSO-d₆, 400 MHz): δ 7.46 (s, 1H), 7.38 (d, J=8.6 Hz, 2H), 7.26 (s, 1H), 6.95 (d, J=8.6 Hz, 2H), 5.04 (s, 2H), 4.28 (q, J=5.6 Hz, 2H), 3.93 (s, 3H), 3.84 (s, 3H), 1.32 (t, J=5.6 Hz, 3H).

### Step D: 2-Bromo-4-methoxy-5-(4-methoxybenzyloxy)benzoic acid

A mixture of ethyl 2-bromo-4-methoxy-5-(4-methoxybenzyloxy)benzoate(4.69 g, 11.8 mmol), LiOH-1-1.H₂O (2.00 g, 47.5 mmol), water (30 ml), methanol (30 ml) and THF (30 ml) was stirred and heated to 70° C. for 12 hours. After cooling down to room temperature, the mixture became a suspension. The mixture was washed with ethyl acetate. The aqueous layer was collected and acidified with 5 M aqueous HCl to obtain pH=1. Product precipitation occurred. The product was filtered, vacuum-dried to obtain 2-bromo-4-methoxy-5-(4-methoxybenzyloxy)benzoic acid (4.0 g, 10.9 mmol, 92%). ¹H NMR (DMSO-d₆, 400 MHz): δ 7.35 (d, J=8.6 Hz, 2H), 7.08 (s, 1H), 6.94 (d, j=8.6 Hz, 2H), 6.91 (s, 1H), 4.94 (s, 2H), 3.76 (s, 3H), 3.72 (s, 3H).

### Step E: Ethyl 3-(2-bromo-4-methoxy-5-(4-methoxybenzyloxy)phenyl)-3-oxopropanoate

Carbonyl diimidazole (1.89 g, 11.6 mmol) was added to a cold mixture of 2-bromo-4-methoxy-5-(4-methoxybenzyloxy)benzoic acid (2.80 g, 7.6 mmol) in THF (20 ml). The mixture (mixture A) was stirred for 2 hours. On a separate flask, a mixture (mixture B) of potassium ethylmalonate (KEM) (2.55 g, 15 mmol) and MgCl₂ (1.80 g, 19 mmol) in THF was stilled at room temperature for 2 hours. Mixture A was added to mixture B. The new mixture was stirred for 22 hours at 60° C. After cooling down to room temperature, the mixture was washed with an aqueous solution of HCl (1 M) extracted with ethyl acetate. The organic layer was dried over MgSO₄, concentrated and chromatographed to obtain ethyl 3-(2-bromo-4-methoxy-5-(4-methoxybenzyloxy)phenyl)-3-oxopropanoate (1.5 g, 3.4 mmol, 45%). ¹H NMR (CDCl₃, 400 MHz): δ 7.38 (d, J=8.7 Hz, 2H), 7.31 (s, 1H), 7.10 (s, 1H), 6.94 (d, J=8.7 Hz, 2H), 5.09 (s, 2H), 4.22 (q, J=7.1 Hz, 2H), 4.04 (s, 2H) 3.93 (s, 3H), 3.83 (s, 3H), 1.31 (t, J=7.1 Hz, 3H).

### Step F: (S,Z)-Ethyl 2-(2-bromo-4-methoxy-5-(4-methoxybenzyloxy)benzoyl)-3-(1-hydroxy-3,3-dimethylbutan-2-ylamino)acrylate

A mixture of ethyl 3-(2-bromo-4-methoxy-5-(4-methoxybenzyloxy)phenyl)-3-oxopropanoate (0.844 g, 1.93 mmol), DMA-DMF (0.345 g, 2.9 mmol) and acetic acid (0.05 ml) in toluene (5 ml) was stirred and heated to 100° C. for 1 hour. After cooling to room temperate, (s)-tert-leucinol (0.47 g, 4 mmol) was added to the mixture. The mixture was stirred for an additional 5 minutes. Toluene was evaporated and the residue was chromatographed to obtain (S,Z)-ethyl 2-(2-bromo-4-methoxy-5-(4-methoxybenzyloxy)benzoyl)-3-(1-hydroxy-3,3-dimethylbutan-2-ylamino)acrylate (0.956 g, 1.6 mmol, 83%). ¹H NMR (CDCl₃, 400 MHz): δ 8.20 (d, J=13.6 Hz, 1H), 7.38 (d, J=8.7 Hz, 2H), 7.01 (s, 1H), 6.91 (d, J=8.7 Hz, 2H), 6.86 (s, 1H), 5.04 (s, 2H); 3.95-4.09 (m, 4H), 3.92 (s, 3H), 3.82 (s, 3H), 3.69-3.80 (m, 1H), 3.10 (t, J=7.6 Hz, OH), 1.28 (t, J=7.1 Hz, 3H).

### Step G: (S)-Ethyl 1-(1-hydroxy-3,3-dimethylbutan-2-yl)-7-methoxy-6-(4-methoxybenzyloxy)-4-oxo-1,4-dihydroquinoline-3-carboxylate

A mixture of (S,Z)-ethyl 2-(2-bromo-4-methoxy-5-(4-methoxybenzyloxy)benzoyl)-3-(1-hydroxy-3,3-dimethylbutan-2-ylamino)acrylate (0.813 g, 1.44 mmol), Cs₂CO₃ (0.563 g, 1.73 mmol), CsF (0.24 g, 1.58 mmol) in DMF (15 ml) was heated with microwave irradiation to 120° C. for 1 hour. After cooling down to room temperature, the mixture was washed with water and extracted with ethyl acetate. The organic layer was dried over MgSO₄, concentrated and chromatographed to obtain (S)-ethyl 1-(1-hydroxy-3,3-dimethylbutan-2-yl)-7-methoxy-6-(4-methoxybenzyloxy)-4-oxo-1,4-dihydroquinoline-3-carboxylate (0.478 g, 0.99 mmol, 69%). ¹H NMR (DMSO-d₆, 400 MHz): δ 8.56 (s, 1H), 7.77 (s, 1H), 7.43 (d, J=8.4 Hz, 2H), 7.39 (s, 1H), 7.00 (d, J=8.4 Hz; 2H), 5.13 (s, 2H), 5.04 (t, J=5.4 Hz, 1H), 4.95 (t, J=5.4 Hz, 1H), 4.22 (q, J=7.1 Hz, 2H), 3.98 (s, 3H), 3.79 (s, 3H), 3.62 (t, J=5.4 Hz, 1H), 1.31 (t, J=7.1 Hz; 3H), 0.99 (s, 9H)

### Step H: (S)-Ethyl 1-(1-(tert-butyldimethylsilyloxy)-3,3-dimethylbutan-2-yl)-7-methoxy-6-(4-methoxybenzyloxy)-4-oxo-1,4-dihydroquinoline-3-carboxylate

A mixture of (S)-ethyl 1-(1-hydroxy-3,3-dimethylbutan-2-yl)-7-methoxy-6-(4-methoxybenzyloxy)-4-oxo-1,4-dihydroquinoline-3-carboxylate (0.478 g, 0.99 mmol), TBDMSCl (0.597 g, 4 mmol) and imidazole (0.544 g, 8 mmol) in DMF (5 ml) was stirred at room temperature for 3 hours. The mixture was washed with brine and extracted with ethyl acetate. The organic layer was dried over MgSO₄ and concentrated to obtain (S)-ethyl 1-(1-(tert-butyldimethylsilyloxy)-3,3-dimethylbutan-2-yl)-7-methoxy-6-(4-methoxybenzyloxy)-4-oxo-1,4-dihydroquinoline-3-carboxylate (0.567 g, 0.95 mmol, 96%). The product was used in the next step without further purification. 1H NMR (CDCl₃, 400 MHz): δ 8.66 (s, 1H), 8.09 (s, 1H), 7 (d, J=8.6 Hz, 2H), 7.00 (s, 1H), 6.92 (d, J=8.6 Hz, 2H), 5.18 (s, 2H), 4.53-4.58 (m, 1H), 4.42 (q, J=7.1 Hz, 2H), 4.13-4.19 (m, 2H), 3.99 (s, 3H), 3.82 (s, 3H), 1.42 (t, J=7.1 Hz, 3H), 1.07 (s, 9H), 0.70 (s, 9H), 0.02 (s, 3H), 0.06 (s, 3H).

### Step I: (S)-Ethyl 1-(1-(tert-butyldimethylsilyloxy)-3,3-dimethylbutan-2-yl)-6-hydroxy-7-methoxy-4-oxo-1,4-dihydroquinoline-3-carboxylate

A mixture of (S)-ethyl 1-(1-(tert-butyldimethylsilyloxy)-3,3-dimethylbutan-2-yl)-7-methoxy-6-(4-methoxybenzyloxy)-4-oxo-1,4-dihydroquinoline-3-carboxylate (0.567 g, 0.95 mmol), TFA (0.5 ml) and DCM (0.5 ml) was stirred at room temperature for 30 minutes. DCM and TFA were evaporated to obtain a residue which was chromatographed (MeOH:DCM, 5:95) to obtain (S)-ethyl 1-(1-(tert-butyldimethylsilyloxy)-3,3-dimethylbutan-2-yl)-6-hydroxy-7-methoxy-4-oxo-1,4-dihydroquinoline-3-carboxylate (0.416 g, 0.87 mmol, 88%).

### Step J: (S)-ethyl 1-(1-(tert-butyldimethylsilyloxy)-3,3-dimethylbutan-2-yl)-6-(3-chloro-2-fluorobenzyloxy)-7-methoxy-4-oxo-1,4-dihydroquinoline-3-carboxylate

A mixture of (S)-ethyl 141-(tert-butyldimethylsilyloxy)-3,3-dimethylbutan-2-yl)-6-hydroxy-7-methoxy-4-oxo-1,4-dihydroquinoline-3-carboxylate (48 mg, 0.1 mmol), 3-chloro-2-fluorobenzylbromide (73 mg, 0.3 mmol) and Na₂CO₃ (21 mg, 0.2 mmol) in DMF (0.5 ml) was stirred and heated to 60° C. for 2 hours. LCMS indicated that starting material was totally converted to product. The reaction mixture was used for the next step without workup.

### Step K: (S)-6-(3-chloro-2-fluorobenzyloxy)-1-(1-hydroxy-3,3-dimethylbutan-2-yl)-7-methoxy-4-oxo-1,4-dihydroquinoline-3-carboxylic acid

To the reaction mixture from step J was added a solution of NaOMe in methanol (0.5 ml, M). The mixture was stirred and heated to 60° C. for 2 hours. After cooling down to room temperature, the mixture was acidified to pH=1, extracted with ethyl acetate and chromatographed to obtain (S)-6-(3-chloro-2-fluorobenzyloxy)-1-(1-hydroxy-3,3-dimethylbutan-2-yl)-7-methoxy-4-oxo-1,4-dihydroquinoline-3-carboxylic acid (25 mg, 0.052 mmol, 52%). ¹H NMR (DMSO-d₆, 400 MHz): δ 15.69 (s, 1H), 8.79 (s, 1H), 7.89 (s, 1H), 7.67 (dd, J=7.1, 7.1 Hz, 1H), 7.67 (dd, J=7.1, 7.1 Hz, 1H), 7.66 (s, 1H), 7.33 (dd, J=7.1, 7.1 Hz, 1H), 5.38 (d, J=3.6 Hz, 2H), 5.17-5.22 (m, 1H), 5.16 (t, J=5.0 Hz, 1H), 4.05 (s, 3H), 3.60 (t, J=5.0 Hz, 1H), 1.00 (s, 9H). MS (ESI): m/z 478.1 (M+1)⁺.

### Examples 29B-29G

Examples 29B-29G were prepared according to the procedure described above for example 29A.

Examples 29H-1 and 29I are prepared according to the procedure described above for example 29A.

| | | | ¹H NMR (400 MHz) 25° C. δ |
|---|---|---|---|
| Compound | Compound Name | Structure | DMSO-d6: MS (ESI) |
| 29A | (S)-6-(3-chloro-2- fluorobenzyloxy)-1- (1-hydroxy-3,3- dimethylbutan-2-yl)-7- methoxy-4-oxo-1,4- dihydroquinoline-3- carboxylic acid | 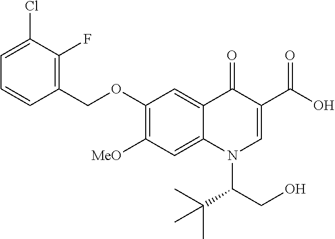 | 15.69 (s, 1H), 8.79 (s, 1H), 7.89 (s, 1H), 7.67 (dd, J = 7.1, 7.1 Hz, 1H), 7.67 (dd, J = 7.1, 7.1 Hz, 1H), 7.66 (s, 1H), 7.33 (dd, J = 7.1, 7.1 Hz, 1H), 5.38 d, J = 3.6 Hz, 2H), 5.17-5.22 (m, 1H), 5.16 (t, J = 5.0 Hz, 1H), 4.05 (s, 3H), 3.60 (t, J = 5.0 Hz, 1H, 1.000 (s, 9H). MS (ESI): m/z 478.1 (M +1)⁺ |
| 29B | (S)-1-(1-hydroxy-3,3- dimethylbutan-2-yl)-7- methoxy-4-oxo-6- (2,4,6- trifluorobenzyloxy)- 1,4-dihydroquinoline- 3-carboxylic acid | 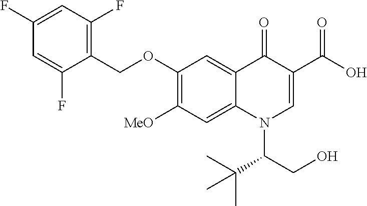 | 15.71 (s, 1H), 8.79 (s, 1H), 7.95 (s, 1H), 7.59 (s, 1H), 7.36 (d, J = 8.4 Hz, 2H), 5.30 (d, J = 3.6 Hz, 2H), 5.18-5.24 (m, 2H), 4.02 (s, 3H), 3.63 (t, J = 4.7 Hz, 1H), 1.00 (s, 9H). MS (ESI): m/z 480.1 (M + 1)⁺ |
| 29C | (S)-1-(1-hydroxy-3,3- dimethylbutan-2-yl)-7- methoxy-4-oxo-6-(2- (trifluoro- methyl)benzyloxy)- 1,4-dihydroquinoline- 3-carboxylic acid | 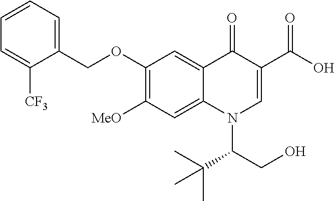 | 15.64 (s, 1H), 8.78 (s, 1H), 7.87 (d, J = 7.8 Hz, 1H), 7.82 (s, 1H), 7.76-7.80 (m, 1H), 7.65 (d, J = 7.7 Hz, 1H), 7.63 (s, 1H), 5.43 (s, 2H), 5.19-5.23 (m, 1H), 5.16 (t, J = 4.7 Hz, 1H), 4.06 (s, 3H), 3.63 (t, J = 4.7 Hz, 1H), 1.01 (s, 9H). MS (ESI): m/z 494.1 (M + 1)⁺ |
| 29D | (S)-6-(5-chloro-2- fluorobenzyloxy)-1- (1-hydroxy-3,3- dimethylbutan-2-yl)-7- methoxy-4-oxo-1,4- dihydroquinoline-3- carboxylic acid | 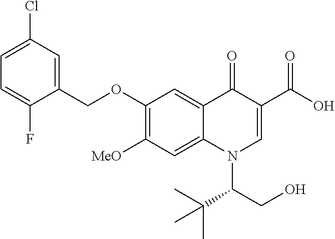 | 15.66 (s, 1H), 8.79 (s, 1H), 7.86 (s, 1H), 7.44 (d, J = 3.1 Hz, 1H), 7.63 (dd, J = 7.7, 3.1 Hz, 1H), 7.62 (s, 1H), 7.54 (d, J = 7.7 Hz, 1H), 5.37 (s, 2H), 5.16-5.23 (m, 2H), 4.09 (s, 3H), 3.63 (t, J = 4.7 Hz, 1H), 1.01 (s, 9H). MS (ESI): m/z 494.16 (M + 1)⁺ |
| 29E | (S)-1-(1-hydroxy-3,3- dimethylbutan-2-yl)-7- methoxy-6-(4- methoxybenzyloxy)-4- oxo-1,4- dihydroquinoline-3- carboxylic acid | 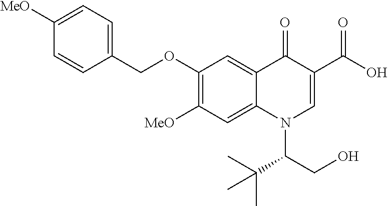 | (CD₃OD): δ 8.90 (s, 1H), 7.92 (s, 1H), 7.51 (s, 1H), 7.45 (d, J = 8.4 Hz, 2H), 6.95 (d, J = 8.4 Hz, 2H), 5.27 (s, 2H), 5.13 (t, J = 4.7 Hz, 2H), 4.08 (s, 3H), 3.82 (s, 3H), 3.63 (t, J = 4.7 Hz, 1H), 1.09 (s, 9H), MS (ESI): m/z 446.1 (M + 1)⁺. |
| 29F | | 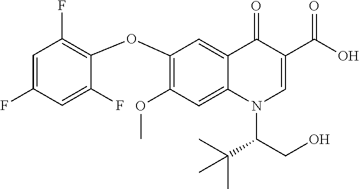 | |
| 29G | | 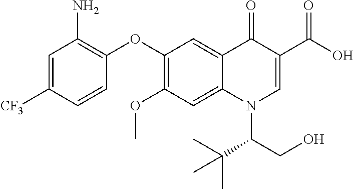 | |
| 29H | | 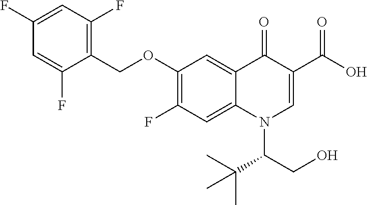 | |
| 29I | | 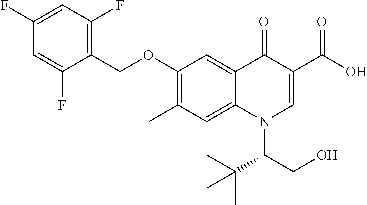 | |

### Example 30

### Compounds of Formula (XXX)

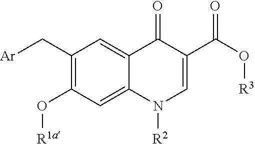

Preparation of compounds of formula (XXX) is described in U.S. Pat. No. 7,176,220, of which, one exemplary synthesis is outlined below.

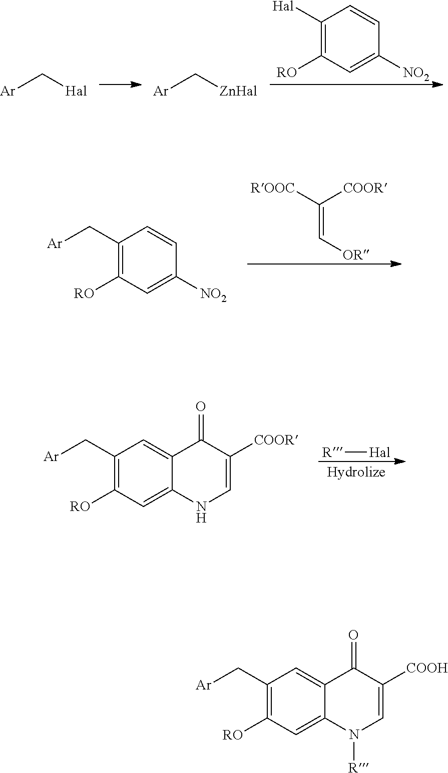

Examples 30A-30C were prepared according to the procedures described in U.S. Pat. No. 7,176,220.

| Compound | Structure |
|---|---|
| 30A | 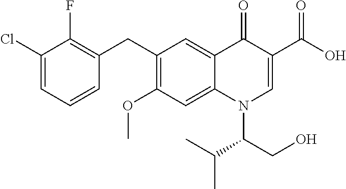 |
| 30B | 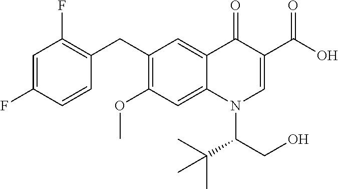 |
| 30C | 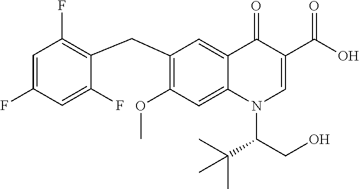 |

### Example 31

Various additional compounds were prepared according to the procedures described herein, and are shown in the table below.

| Compound | Structure | NMR |
|---|---|---|
| 31A | 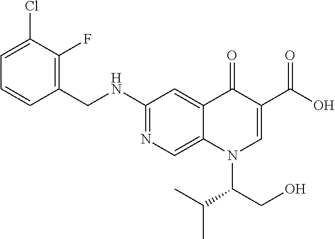 | |
| 31B | 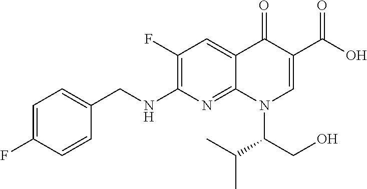 | |
| 31C | 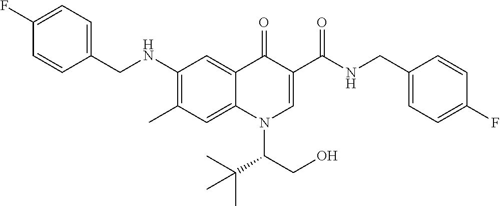 | NMR (DMSO-d₆): δ 14.90 (s, 1H), 9.24 (s, 1H), 8.76 (s, 1H), 7.88 (t, 1H), 7.48 (t, 1H), 7.36 (t, 1H), 7.29 (s, 1H), 7.20 (t, 1H), 5.22 (m, 1H), 4.88 (m, 1H), 4.67 (d, 2H), 3.93 (m, 1H), 3.79 (m, 1H), 2.35 (m, 1H), 1.12 (d, 3H), 0.74 (d, 3H); MS (ESI): m/z 434 (M + 1)⁺ |
| 31D | 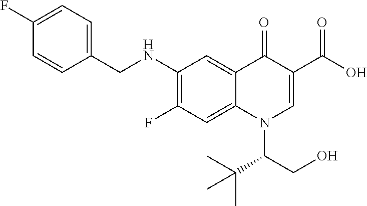 | |
| 31E | 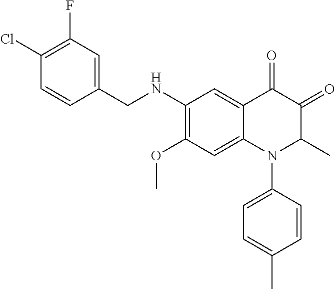 | |
| 31F | 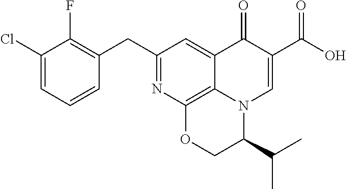 | |
| 31G | 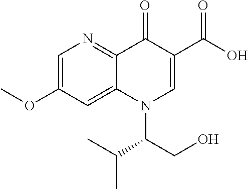 | |
| 31H | 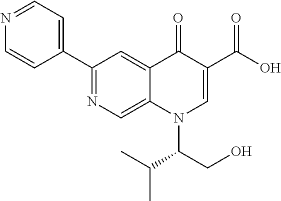 | |

### II Biological Activity

### Example 31

### Generation of EC₅₀ Data

Compounds were screened for inhibitory activity against human immunodeficiency virus type 1 (HIV-1) using a cell-based assay using HIV-1 expressing firefly luciferase as a reporter gene and pseudotyped with vesicular stomatitis virus envelope glycoprotein (VSV-G). Experimental procedures were essentially as previously published (see Connor et al., Journal of Virology, 1996, 70, 5306-5311: Characterization of the functional properties of env genes from long-term survivors of human immunodeficiency virus type 1 infection, and Popik et al., Journal of Virology, 2002, 76, 4709-4722: Human immunodeficiency virus type 1 uses lipid raft-co-localized CD4 and chemokine receptors for productive entry into CD4+ T cells). Virus stocks were generated by co-transfection of plasmid DNA encoding VSV-G with vector pNL4-3Env(−)Luc(+) into 293T cells. Sixty-four hours after transfection, virus-containing medium was collected by centrifugation and stored frozen at −80° C.

HeLa cells were infected with the VSV-G pseudotyped virus in the presence of screening compounds in a 384-well or 96-well microtiter plate format. Forty-eight hours after initial infection, Luciferase Assay Reagent (Promega) was added to the cells and luciferase activity was determined using a LJLAnalyst luminometer. As the luciferase gene is carried in the virus genome, its expression level reflects the virus replication level in the presence of a compound.

To evaluate the activity of the compounds against wild type HIV-1, the HeLa-JC53 cell line that expresses high levels of CD4 and CCR5 (see for example, Platt et al., Journal of Virology 1998, 72, 2855-2864: Effect of CCR5 and CD4 cell surface concentrations on infection by macrophagetropic isolates of human immunodeficiency virus type 1) was modified by isolation of a stable cell line that expresses luciferase under the control of the HIV-1 promoter (long terminal repeat, i.e., LTR). HIV-1 infection of this cell line stimulates the transcription of luciferase from the HIV-1 promoter and the luciferase gene expression level is proportional to the level of virus replication (Harrington et al., Journal of Virology Methods, 2000, 88, 111-115: Direct detection of infection of HIV-1 in blood using a centrifugation-indicator cell assay; and Roos et al., Virology, 2000, 273, 307-315: LuSIV cells: a reporter cell line for the detection and quantitation of a single cycle of HIV and SIV replication). Procedures for virus infection, compound testing and luciferase activity determination were the same as for the VSV-G pseudotyped HIV-1.

Two approaches have been used to evaluate cytotoxicity. The first employed another modified HeLa-JC53 cell line that constitutively expresses high levels of luciferase without virus infection. The level of luciferase expression in these cells serves as an indicator for cell replication in the presence of the compounds. Procedures for compound testing and luciferase activity determination are the same as for the virus infection tests. The other toxicity assay utilizes HeLe-JC53 cells and a commercially available cell viability assay kit (Promega) that measures the ATP levels in the cells.

### Example 32

### Activity Data for Select Compounds

Select compounds prepared as described above were assayed for activity according to the biological procedures described herein and the results are given in the table below.

Activity is given as EC50 (nM):

<10 nm=A;10−100 nM=B,>100 nM=C

| Compound | EC50 (nM) |
|---|---|
| 1A | A |
| 1B | C |
| 1C | B |
| 1D | C |
| 1E | B |
| 1F | C |
| 1G | A |
| 1H | A |
| 1I | B |
| 1J | B |
| 1K | A |
| 1L | C |
| 1M | C |
| 1N | C |
| 1O | A |
| 1P | C |
| 1Q | C |
| 1R | C |
| 2A | A |
| 2B | B |
| 2C | A |
| 2D | B |
| 2E | A |
| 2F | A |
| 2G | B |
| 2H | A |
| 2I | B |
| 2J | B |
| 2K | B |
| 2L | B |
| 2M | B |
| 2N | A |
| 2O | B |
| 2P | B |
| 2Q | C |
| 2R | A |
| 2S | B |
| 2T | A |
| 2U | A |
| 2V | B |
| 2W | B |
| 2X | B |
| 2Y | B |
| 2Z | B |
| 2AA | B |
| 2BB | B |
| 2CC | A |
| 2DD | A |
| 2EE | A |
| 2FF | A |
| 2GG | B |
| 2HH | C |
| 2II | C |
| 2JJ | B |
| 2KK | B |
| 3A | A |
| 3B | B |
| 3C | A |
| 4A | B |
| 4B | C |
| 4C | C |
| 4D | C |
| 4E | C |
| 5A | B |
| 5B | C |
| 5C | C |
| 5D | C |
| 5E | C |
| 5F | B |
| 5G | C |
| 5H | B |
| 5I | C |
| 5J | C |
| 5K | C |
| 5L | C |
| 5M | B |
| 5N | C |
| 5O | C |
| 5P | C |
| 5Q | B |
| 6 | C |
| 7A | C |
| 7B | C |
| 7C | C |
| 7D | C |
| 8A | C |
| 8B | C |
| 8C | C |
| 8A | C |
| 8B | C |
| 8D | C |
| 10A | C |
| 11A | C |
| 12A | C |
| 12B | C |
| 12C | |
| 12D | C |
| 12E | C |
| 12F | C |
| 12G | C |
| 12H | C |
| 13A | C |
| 14A | C |
| 14B | C |
| 15A | C |
| 16A | C |
| 17A | C |
| 17B | C |
| 17C | C |
| 17D | C |
| 17E | C |
| 17F | C |
| 17G | C |
| 17H | C |
| 17I | C |
| 17J | C |
| 17K | C |
| 17L | C |
| 17M | C |
| 17N | C |
| 17O | C |
| 18A | C |
| 19 | C |
| 20A | C |
| 20B | C |
| 20C | B |
| 20D | B |
| 20E | C |
| 20F | C |
| 20G | C |
| 20H | C |
| 20I | C |
| 20J | C |
| 20K | C |
| 20L | C |
| 20M | C |
| 20N | C |
| 20O | C |
| 20P | C |
| 20Q | C |
| 20R | B |
| 20S | C |
| 20T | C |
| 20U | C |
| 20V | C |
| 20W | C |
| 20X | C |
| 20Y | C |
| 20Z | B |
| 20AA | A |
| 20BB | C |
| 20CC | C |
| 20DD | C |
| 20EE | C |
| 20FF | C |
| 20GG | B |
| 20HH | C |
| 20II | C |
| 20JJ | C |
| 20KK | C |
| 20LL | C |
| 20MM | C |
| 20NN | A |
| 20OO | C |
| 20PP | C |
| 20QQ | C |
| 20RR | C |
| 20SS | C |
| 21A | A |
| 21B | A |
| 21C | B |
| 21D | B |
| 22A | C |
| 22B | B |
| 22C | B |
| 23A | C |
| 23B | C |
| 23C | B |
| 24A | C |
| 24B | A |
| 25C | B |
| 25D | A |
| 26A | B |
| 26D | B |
| 26E | C |
| 26F | |
| 27A | C |
| 27B | B |
| 27C | B |
| 27E | B |
| 29A | C |
| 29B | B |
| 29C | C |
| 29D | C |
| 29E | C |
| 29F | C |
| 29G | C |
| 30A | A |
| 30B | A |
| 30C | B |
| 30A | C |
| 30B | C |
| 30C | C |
| 30D | B |
| 30E | C |
| 30F | C |
| 30G | C |
| 30H | C |

### Example 33

### Microsomal Incubation (Method 1)

Compound (10 μM) was incubated with rat, dog, cynomolgus monkey, and human liver microsomes (1 mg protein/mL) in a final volume of 1 mL in 2-mL Eppendorf tubes. The mixture containing enzymes, potassium phosphate buffer (100 mM, pH 7.4), and the compound was pre-incubated at 37° C. for 3 min. The reaction was initiated by the addition of NADPH (final concentration of 1 mM) and incubated for 60 minutes at 37° C. The reaction was terminated by the addition of 1 mL of acetonitrile. After centrifugation at 12000 rpm for 3 minutes, the supernatant was subjected to 15 minutes of concentration (N2 flow, 32° C.). The resulting final extract was transferred to clean vials and analyzed by HPLC.

### Example 34

### Hepatocytes Incubation (Method 1)

Cryopreserved hepatocytes were thawed in a water bath at 37° C. and transferred to a 50-mL tube containing 45 mL of pre-warned incubation medium (In VitroGRO HT medium). The tube was inverted 3 times to ensure resuspension of hepatocytes and centrifuged at 50 g at room temperature for 5 minutes. The supernatant was decanted without disturbing the pellet. The pellet of hepatocytes was resuspended in 1 mL of William's E medium and the viable cell counting was determined by the tryptan blue exclusion method.

The William's E medium was added to the suspension of the hepatocytes pellet to make a final density of 2 million cells/mL. Stock solution of the compound was prepared at the concentration of 1 mM and diluted to 10 μM with William's E medium. The culture plate was incubated at 37° C. under 5% carbon dioxide and 95% air atmosphere for 2 hrs. The metabolic reaction was terminated by transferring the contents of the well into a centrifuge tube containing 1 mL of 0.1% TFAA acetonitrile solution and then vortexing. After centrifugation, the supernatant was subjected to 15 minute of concentration (N2 flow, 32° C.). The resulted final extract was transferred to clean vials for HPLC analysis.

### Example 35

### Stability Data for Select Compounds

Select compounds prepared as described above and Raltegravir (a known HIV integrase inhibitor) as control, were assayed for stability according to the biological procedures described in examples 33 and 34, and the results are given in the table below.

| | | | % Remaining | | | |
|---|---|---|---|---|---|---|
| Eg | Structure | Matrix | Human | Dog | Monkey | Rat |
| 1A | 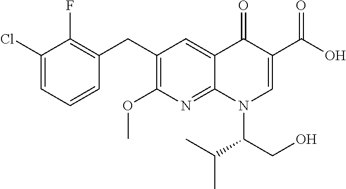 | Microsome Hepatocyte | 101 (3800) * 96.1 (3800) * 83.5 (7800) * 89.6 | 102 NA | 66.3 72.3 | 38.1 66.9 |
| Raltegravir | | Microsome | 99.1 (3800) * | 91.8 | 94.7 | 105 |
| | | Hepatocyte | 32.4 | NA | 20.0 | 43.3 |
| 30A | 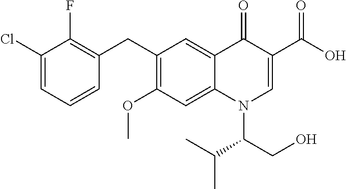 | Microsome Hepatocyte | 40.5 (3800) * NA | 83.8 NA | 4.1 NA | 6.7 NA |

* CYP3A4 activity (pmol/mg protein)

### Example 36

### Hepatocyte Stability (Human and Rat)

### Thawing the Cryovials and Suspending the Cells

Incubation media was pre-warmed to 37° C. in a water bath. 3 vials of hepatocytes were removed from liquid nitrogen storage and placed on ice. The vials were immediately immersed in a 37° C. water bath and gently rocked back and forth until most of the ice was melted and the pellet was completely mixed, (note, care was taken to maintain temperature below 37° C. during this step as the cryo-preservative is cytotoxic at 37° C.) 1 vial of hepatocytes was added to each 25 mL tube of incubation media, and the cells resuspended by gently inverting the tube several times. The cell suspension was centrifuged (468 rpm, 5 mins, ˜25° C.), and the supernatant discarded using a 5 mL pipette, being careful not to disturb the pellet during aspiration. A moderate amount of media was left with the pellet, which was loosened by tipping and gently rolling the tube until the pellet was gone. Incubation media (2 mL, CO₂ bubbled) was added to each tube and the hepatocytes re-suspended by rocking the tube. The total cell count was then determined (see Trypan Blue Exclusion Method described below). The cells were resuspended in the appropriate amount of incubation media to yield 1.25×10⁶ viable cells/mL. The final concentration of cells in the incubation was 1.0×10⁶ after the addition of the compound stock solution.

### Trypan Blue Exclusion Method

800 μL of incubation media, 100 μL of Trypan Blue solution and 100 μL of the cell suspension were mixed in a 2 mL, microcentrifuge tube. 10 μL of the suspension was applied to the hemacytometer using a wide bore pipette tip. Living (yellow) and dead (blue) cells were counted in 4 quadrants of the hemacytometer. The total number of cells/mL and cell viability (living cells/total cells) were calculated, as follows:

Total cells/mL=living cells×0.25×10×10^{4 [}0.25=¼ quadrants counted;10=dilution factor]

### Time Course Incubation

Hepatocytes (40 uL) were added to each tube using wide orifice pipette tips to a final concentration of 1 million cells/mL. Test compound solution (10 μl of 5 μM) was aliquoted into the appropriate tubes, and the incubated (37° C., CO2 incubator uncapped) for 4 hours. Samples were then quenched with internal standard in acetonitrile (200 uL of 100 ng/mL), vortexed (1-2 mins) and centrifuged (10 minutes, 3000 rpm). The supernatant was removed for LC-MS/MS analysis (instrument—MDS-Analyst, API 4000, S/N:J3750206, Agilent 1100, Binary Pump G1312A, S/N DE14910504; HPLC Column—Atlantis dC18 3 um, 4.6×50 mm, P/N: 186001329).

Hepatocytes (human and rat, X00801 and M00005 respectively), buffer (InVitro Gro Buffer, Z99074) and medium (Invitro Gro HT, Z99019) were obtained from Celsis.

Select compounds prepared as described herein, were assayed for stability according to the biological procedures described above, and the results are given in the table below.

| | | % Remaining | |
|---|---|---|---|
| Eg | Structure | (human) | % Remaining (rat) |
| 2V | 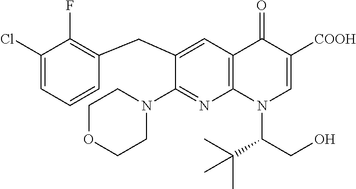 | 97 | 82 |
| 20AA | 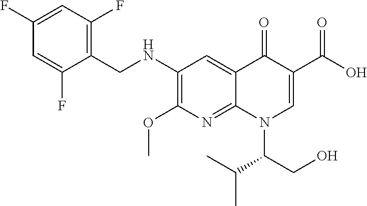 | 75 | 43 |
| 20NN | 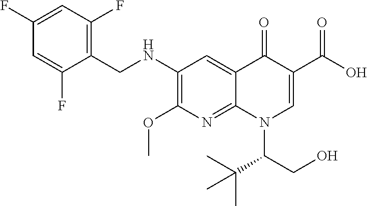 | 61 | 46 |
| 30A | 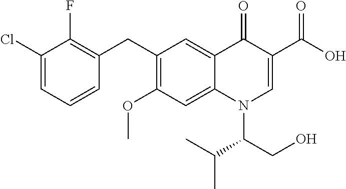 | 54 | 29 |

### Example 37

### Metabolic Stability

Recombinant enzymes: Microsomes from baculovirus-infected insect enzymes cells (Supersomes) expressing CYP1A1, 1A2, 1B1, 2A6, 2B6, 2C8, 2C9, 2C18, 2C19, 2D6, 2E1, 3A4, 3A5, FMO3 and insect cell controls are obtained from BD Biosciences (Gentest Co).

Compound (1 μM) or a positive control (1 μM) are incubated with individual recombinant enzymes (10 pmol) in a final volume of 500 uL. The mixture of enzymes containing potassium phosphate buffer (100 mM, pH 7.4), MgCl₂ (5 mM), EDTA (100 μM), and compound or positive control are pre-incubated at 37° C. for 3 minutes. Tris buffer (50 mM, pH 8) is used for FMO3 incubation without pre-incubation. The reaction is then initiated by the addition of NADPH (final concentration: 1 mM) and incubated at 37° C. for 60 minutes. The reaction is terminated by addition of 200 μL of acidified acetonitrile containing an internal standard or only acetonitrile for the positive controls. After centrifugation at 12000 rpm for 10 minutes, 200 μL of the supernatant are transferred to a clean 96-well plate and analyzed by liquid chromatography coupled with tandem mass spectrometry (LC/MS/MS). The positive controls used are: 7-ethoxyresorufin (CYP1A 1, CYP1A2 and CYP1B1), coumarin (CYP2A6), selegiline (CYP2B6), diclofenac (CYP2C9), omeprazole (CYP2C19), bufuralol (CYP2D6) and testosterone (CYP3A4). P-nitrophenol is incubated with CYP2E1 (50 pmol) at a concentration of 500 μM for 20 Minutes to assess the activity of the enzyme. The formation of p-nitrocatechol is monitored by LC-MS/MS.

### Example 38

### Integrase Inhibition Activity for Select Compounds

A time-of-addition experiment is performed to examine the replication step(s) affected by a compound of formula (I) or (II); formula (III) or (IV); or formula (V)(a), (V)(b) or (V)(c), allowing to classify the mechanism of action of integrase inhibitors and determining how long the addition of a compound can be postponed before it loses antiviral function. The assay is performed according to previously described literature procedures, see Daelemans et al., J. Viral, 2007, 81(8), 4381.

The examples and embodiments described herein are for illustrative purposes only and various modifications or changes suggested to persons skilled in the art are to be included within the spirit and purview of this application and scope of the appended claims. All publications, patents, and patent applications cited herein are hereby incorporated by reference for such subject matter.

1-90. (canceled)

91. A compound of formula (III) or formula (IV): 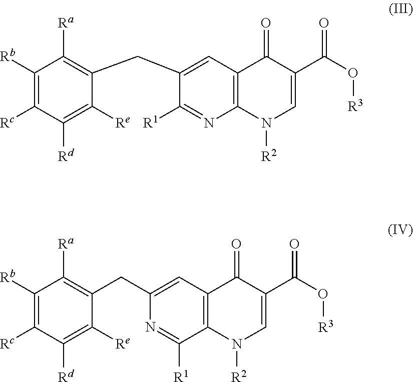
wherein:
R¹ is H, F, Cl, Br, I, CFH₂, CF₂H, CF₃, CN, OH, NO₂, NH₂, NH (optionally substituted alkyl) or N (optionally substituted alkyl)(optionally substituted alkyl), SO₂CH₃, SO₂NH₂, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl, optionally substituted heteroaryl;
R² is optionally substituted alkyl, optionally substituted cycloalkyl, optionally substituted heterocycloalkyl, optionally substituted aryl or optionally substituted heteroaryl;
R³ is H, C₁₋₆ alkyl or a pharmaceutically acceptable cation; and wherein
R^{a}, R^{b}, R^{c}, R^{d} and R^{e} are independently selected from H, F, Cl, Br, I, CF₃, CN, alkyl, cycloalkyl, cyclopropylmethyl, NH₂, NHR′, NR′R″, OH, OR′, SH, SR′, C(O)R′, CO₂H, COOR′, CONH₂, CONHR′, CONR′R″, SO₃H, S(O)₂R′, S(O)₂NH₂, S(O)₂NHR′, S(O)₂NR′R″, aryl, heterocyclyl and heteroaryl; wherein
R′ is methyl, ethyl, n-propyl, i-propyl, n-butyl, i-butyl, s-butyl, t-butyl, cyclopropyl, cyclobutyl, cyclopentyl, cyclohexyl or cyclopropylmethyl;
R″ is methyl, ethyl, n-propyl, i-propyl, n-butyl, i-butyl, s-butyl, t-butyl, cyclopropyl, cyclobutyl, cyclopentyl, cyclohexyl or cyclopropylmethyl; or
R′ and R″ together with the nitrogen atom to which they are attached form an optionally substituted 4-, 5- or 6-membered heterocyclic ring; and
and all alkyl, alkylene, cycloalkyl, heterocyclyl, aryl and heteroaryl moieties may be optionally further substituted; and
provided that the compound is not: 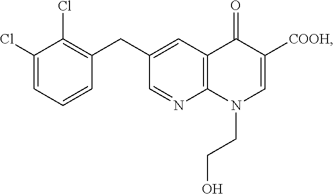
or a metabolite, pharmaceutically acceptable salt, solvate, polymorph, ester, tautomer or prodrug thereof.

92. The compound of claim 91, wherein R¹ is heterocyclyl, substituted alkyl, substituted alkoxy or NH (substituted alkyl), wherein the substituents are selected from hydroxy, hydroxyalkyl, alkoxyalkyl, aryl, aralkyl, heterocyclyl and alkylene-heterocyclyl.

93. The compound of claim 91, wherein R¹ is morpholino.

94. The compound of claim 91, wherein R² is optionally substituted C₅₋₈ alkyl.

95. The compound of claim 94, wherein the C₅₋₈ alkyl is substituted with one OH group.

96. The compound of claim 91, wherein R² is 1-hydroxy-3,3-dimethylbutan-2-yl or 1-hydroxy-3-methylbutan-2-yl:

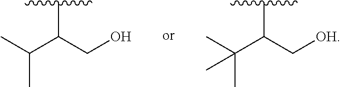

97. The compound of claim 91, wherein R² comprises a chiral center in the (S) configuration.

98. The compound of claim 91, wherein R³ is H.

99. The compound of claim 91, wherein
R¹ is heterocyclyl, substituted alkyl, substituted alkoxy or NH (substituted alkyl);
R² is C₅₋₈ alkyl substituted with one OH group; and
R³ is H.

100. The compound of claim 91, wherein R^{a}, R^{b}, R^{c}, R^{d} and R^{e} are independently selected from H, F and Cl.

101. The compound of claim 91, wherein
one of R^{a}, R^{b}, R^{c}, R^{d} and R^{e} is F;
one of R^{a}, R^{b}, R^{c}, R^{d} and R^{e} is Cl; and
the rest of R^{a}, R^{b}, R^{c}, R^{d} and R^{e} are H.

102. The compound of claim 91, wherein
R^{a} is F;
R^{b} is Cl; and
R^{c}, R^{d} and R^{e} are H.

103. A compound of claim 91, selected from
(S)-6-(3-chloro-2-fluorobenzyl)-1-(1-hydroxy-3-methylbutan-2-yl)-7-methoxy-4-oxo-1,4-dihydro-1,8-naphthyridine-3-carboxylic acid;
6-(3-chloro-2-fluorobenzyl)-1-((2S,3S)-1-hydroxy-3-methylpentan-2-yl)-7-methoxy-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-7-methoxy-4-oxo-1,4-dihydro-1,8-naphthyridine-3-carboxylic acid;
(S)-6-(3-chloro-2-fluorobenzyl)-1-(1-hydroxy-4-methylpentan-2-yl)-7-methoxy-4-oxo-1,4-dihydro-1,8-naphthyridine-3-carboxylic acid;
(S)-6-(3-chloro-2-fluorobenzyl)-1-(1-cyclohexyl-2-hydroxyethyl)-7-methoxy-4-oxo-1,4-dihydro-1,8-naphthyridine-3-carboxylic acid;
(S)-6-(3-chloro-2-fluorobenzyl)-7-ethoxy-1-(1-hydroxy-3,3-dimethylbutan-2-yl)-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-7-(2-hydroxyethoxy)-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-7-(3-hydroxypropoxy)-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-7-(2-methoxyethoxy)-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-4-oxo-7-(pyridin-3-ylmethoxy)-1,4-dihydro-1,8-naphthyridine-3-carboxylic acid;
(S)-6-(3-chloro-2-fluorobenzyl)-1-(1-hydroxy-3,3-dimethylbutan-2-yl)-7-(2-hydroxyethylamino)-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-7-(2-methoxyethylamino)-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-7-(3-methoxypropylamino)-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-7-morpholino-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-7-(2-morpholinoethylamino)-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-7-(3-morpholinopropylamino)-4-oxo-1,4-dihydro-1,8-naphthyridine-3-carboxylic acid;
(S)-6-(3-chloro-2-fluorobenzyl)-1-(1-hydroxy-3,3-dimethylbutan-2-yl)-4-oxo-7-(3-(2-oxopyrrolidin-1-yl)propylamino)-1,4-dihydro-1,8-naphthyridine-3-carboxylic acid;
(S)-6-(3-chloro-2-fluorobenzyl)-1-(1-hydroxy-3,3-dimethylbutan-2-yl)-4-oxo-7-(pyridin-2-ylmethylamino)-1,4-dihydro-1,8-naphthyridine-3-carboxylic acid;
(S)-6-(3-chloro-2-fluorobenzyl)-1-(1-hydroxy-3,3-dimethylbutan-2-yl)-4-oxo-7-(pyridin-2-ylmethylamino)-1,4-dihydro-1,8-naphthyridine-3-carboxylic acid; and
(S)-6-(3-chloro-2-fluorobenzyl)-1-(1-hydroxy-3,3-dimethylbutan-2-yl)-7-(3-hydroxypropyl)-4-oxo-1,4-dihydro-1,8-naphthyridine-3-carboxylic acid:

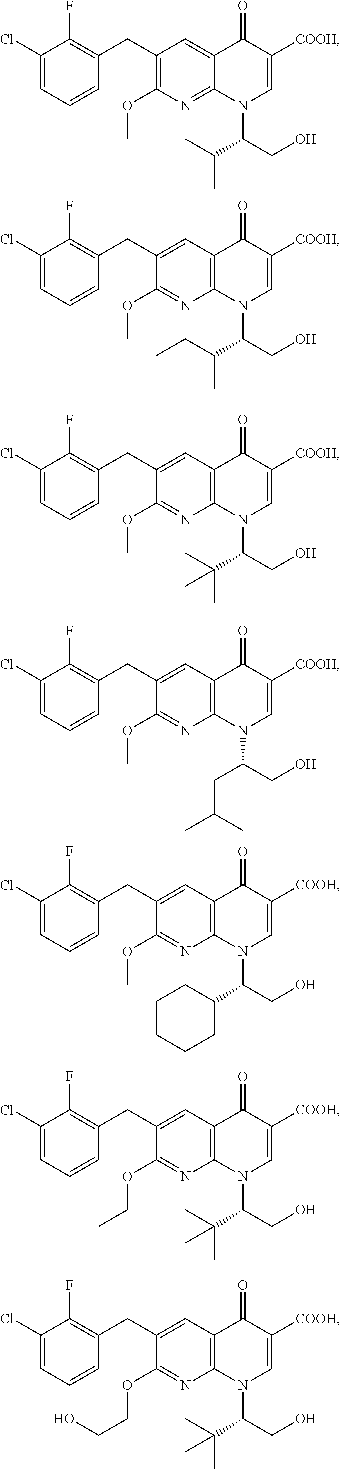

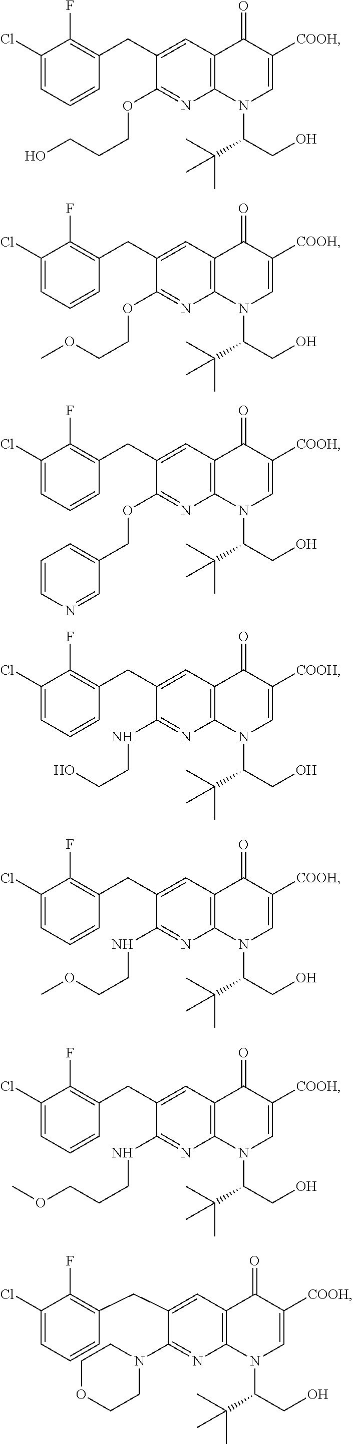

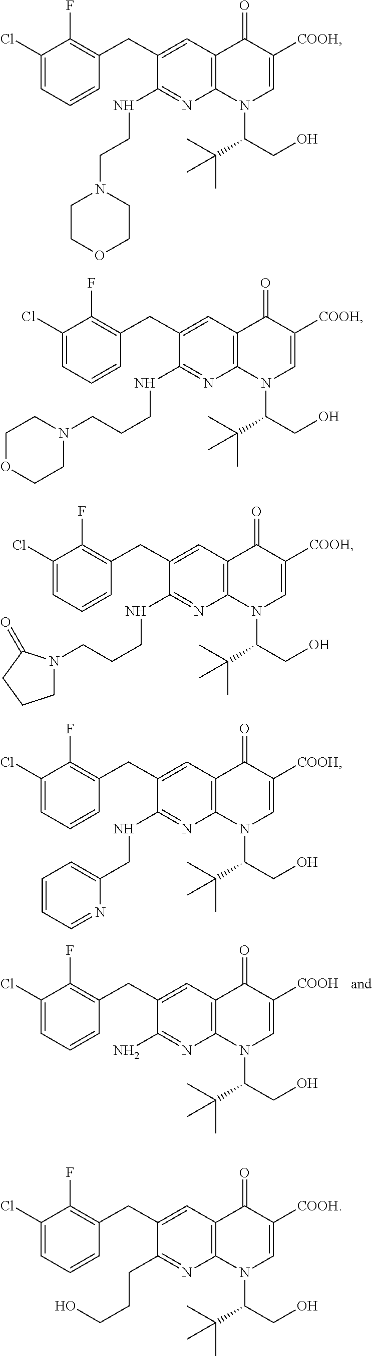

104. A pharmaceutical composition comprising an effective amount a compound of a compound of claim 91, or a metabolite, pharmaceutically acceptable salt, solvate, polymorph, ester, tautomer or prodrug thereof.

105. The pharmaceutical composition of claim 104, further comprising a second therapeutic agent.

106. The pharmaceutical composition of claim 104, further comprising a reverse transcriptase inhibitor, a viral protease inhibitor, a fusion inhibitor, a cytokine, a cytokine inhibitor, a glycosylation inhibitor, a viral mRNA processing inhibitor, an entry inhibitor, an integrase inhibitor or a maturation inhibitor or a combination thereof.

107. A method of treating a viral infection in a patient in need thereof comprising administering to said patient an effective amount of a compound of claim 1, or a metabolite, pharmaceutically acceptable salt, solvate, polymorph, ester, tautomer or prodrug thereof.

108. The method of claim 107, wherein the subject is infected with HIV-1 or HIV-2.

109. The method of claim 107, wherein the subject is infected with a drug resistant strain of HIV.

110. The method of claim 107, wherein the subject is infected with a multidrug resistant strain of HIV.

111. The method of claim 107, further comprising administering an effective amount of an anti HIV or AIDS drug.

112. The method of claim 107, further comprising administering an effective amount of second therapeutic agent selected from the group consisting of reverse transcriptase inhibitors, viral protease inhibitors, cytokines, cytokine inhibitors, glycosylation inhibitors, viral mRNA processing inhibitors, entry inhibitors, integrase inhibitors, maturation inhibitors or a combination of two or more thereof.

113. The method of claim 111, wherein administration of the compound of formula (III) or formula (IV), or metabolite, pharmaceutically acceptable salt, solvate, polymorph, ester, tautomer or prodrug thereof and the second therapeutic agent is sequential.

114. The method of claim 113, wherein the sequential administration is a cycling therapy.

115. A compound of formula (I) or formula (II) or a metabolite, pharmaceutically acceptable salt, solvate, polymorph, ester, tautomer or prodrug thereof 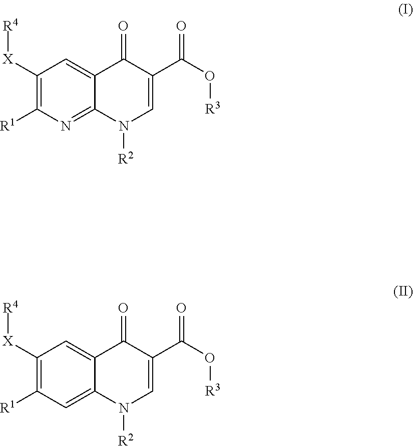
wherein:
R¹ is H, F, Cl, Br, I, CFH₂, CF₂H, CF₃, CN, OH, NO₂, NH₂, NH(alkyl) or N(alkyl)₂, SO₂CH₃, SO₂NH₂, SO₂NHCH₃, CO₂-alkyl, optionally substituted alkyl, optionally substituted alkenyl, optionally substituted alkoxy, optionally substituted S-alkyl, optionally substituted cycloalkyl, optionally substituted heterocycle, optionally substituted aryl or optionally substituted heteroaryl;
R² is optionally substituted alkyl, optionally substituted cycloalkyl, optionally substituted heterocycloalkyl, optionally substituted aryl or optionally substituted heteroaryl;
R³ is H, C₁₋₆ alkyl or a pharmaceutically acceptable cation; and wherein
X is O or N—R⁵;
wherein R⁵ is H or optionally substituted C₁₋₄ alkyl;
R⁴ is 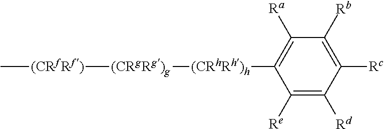
wherein each R^{f}, R^{f′}, R^{g}, R^{g′}, R^{h} and R^{h′} is H or optionally substituted C₁₋₁₀ alkyl;
g is 0 or 1;
h is 0 or 1;
R^{a}, R^{b}, R^{c}, R^{d} and R^{e} are independently selected from H, F, Cl, Br, I, CF₃, CN, alkyl, cycloalkyl, cyclopropylmethyl, NH₂, NHR′, NR′R″, OH, OR′, SH, SR′, C(O)R′, CO₂H, COOR′, CONH₂, CONHR′, CONR′R″, SO₃H, S(O)₂R′, S(O)₂NH₂, S(O)₂NHR′, S(O)₂NR′R″, aryl, heterocyclyl and heteroaryl; wherein
R′ is methyl, ethyl, n-propyl, i-propyl, n-butyl, i-butyl, s-butyl, t-butyl, cyclopropyl, cyclobutyl, cyclopentyl, cyclohexyl or cyclopropylmethyl;
R″ is methyl, ethyl, n-propyl, i-propyl, n-butyl, i-butyl, s-butyl, t-butyl, cyclopropyl, cyclobutyl, cyclopentyl, cyclohexyl or cyclopropylmethyl; or
R′ and R″ together with the nitrogen atom to which they are attached form an optionally substituted 4-, 5- or 6-membered heterocyclic ring; or
X is N and R⁵ and R^{f}, or R⁵ and R^{g}, or R⁵ and R^{h}, together with the N atom form an optionally substituted 4-, 5- or 6-membered heterocyclic ring, optionally containing 1 or 2 additional heteroatoms selected from O, N and S;
and all alkyl, alkylene, cycloalkyl, heterocyclyl, aryl and heteroaryl moieties may be optionally further substituted.

116. A compound of claim 115, selected from
(S)-1-(1-hydroxy-3-methylbutan-2-yl)-7-methoxy-4-oxo-6-(2,4,6-trifluorobenzylamino)-1,4-dihydro-1,8-naphthyridine-3-carboxylic acid;
(S)-1-(1-hydroxy-3-methylbutan-2-yl)-7-methoxy-4-oxo-6-(2,4,6-trifluorobenzylamino)-1,4-dihydroquinoline-3-carboxylic acid;
(S)-1-(1-hydroxy-3,3-dimethylbutan-2-yl)-7-methoxy-4-oxo-6-(2,4,6-trifluorobenzylamino)-1,4-dihydro-1,8-naphthyridine-3-carboxylic acid;
(S)-6-(2,4-difluorobenzylamino)-1-(1-hydroxy-3-methylbutan-2-yl)-7-methoxy-4-oxo-1,4-dihydro-1,8-naphthyridine-3-carboxylic acid;
(S)-1-(1-hydroxy-3-methylbutan-2-yl)-4-oxo-6-(2,4,6-trifluorobenzylamino)-1,4-dihydroquinoline-3-carboxylic acid;
(R)-1-(1-hydroxy-3-methylbutan-2-yl)-7-morpholino-4-oxo-6-(2,4,6-trifluorobenzyloxy)-1,4-dihydro-1,8-naphthyridine-3-carboxylic acid;
(S)-6-(2,6-difluorobenzylamino)-1-(1-hydroxy-3-methylbutan-2-yl)-7-methoxy-4-oxo-1,4-dihydroquinoline-3-carboxylic acid;
(S)-6-(2,6-difluorobenzylamino)-1-(1-hydroxy-3-methylbutan-2-yl)-7-methoxy-4-oxo-1,4-dihydro-1,8-naphthyridine-3-carboxylic acid;
(S)-6-(2,4-difluorobenzylamino)-1-(1-hydroxyl-3-methylbutan-2-yl)-7-methoxy-4-oxo-1,4-dihydroquinoline-3-carboxylic acid; and
(S)-1-(1-hydroxy-3,3-dimethylbutan-2-yl)-7-methyl-4-oxo-6-(2,4,6-trifluorobenzylamino)-1,4-dihydro-1,8-naphthyridine-3-carboxylic acid:

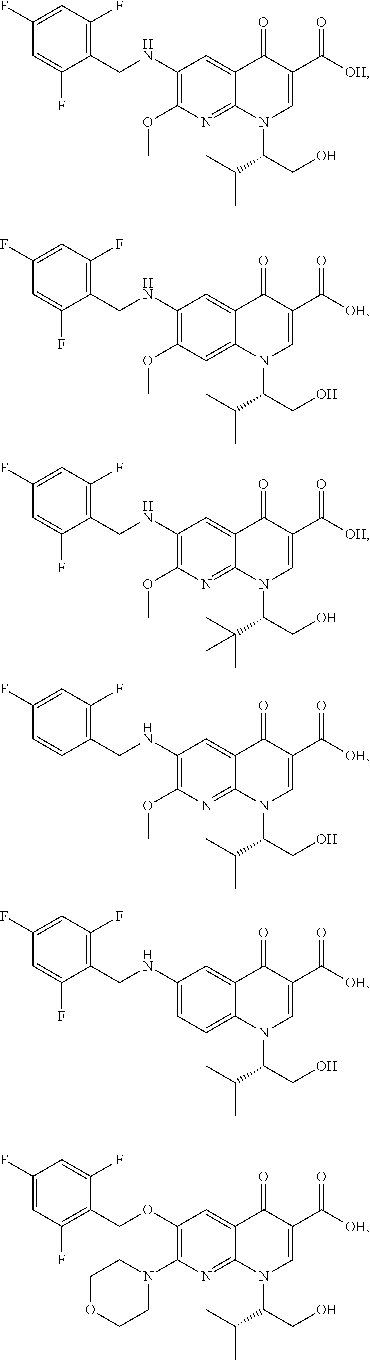

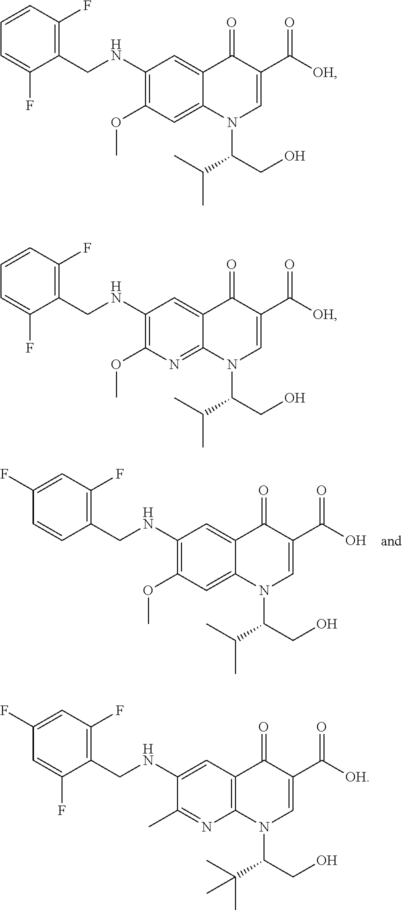

117. A method for treating HIV infection in a subject in need thereof with combination therapy, comprising administering to said patient an effective amount of a combination of at least one compound claim 115 and a second therapeutic agent selected from the group consisting of reverse transcriptase inhibitors, viral protease inhibitors, cytokines, cytokine inhibitors, glycosylation inhibitors, viral mRNA processing inhibitors, entry inhibitors, integrase inhibitors, maturation inhibitors or a combination of two or more thereof.
